(19) United States
(12) Patent Application Publication
(10) Pub. No.: US 2024/0191256 A1
(43) Pub. Date: Jun. 13, 2024

(54) Virus-like Particles with Programmable Tropism and Methods of Use Thereof for Delivery to Cells

(71) Applicant: The General Hospital Corporation, Boston, MA (US)

(72) Inventors: J. Keith Joung, Winchester, MA (US); Peter Cabeceiras, Boston, MA (US)

(21) Appl. No.: 18/511,416

(22) Filed: Nov. 16, 2023

Related U.S. Application Data
(60) Provisional application No. 63/425,894, filed on Nov. 16, 2022.

(51) Int. Cl. C12N 15/86; C07K 14/005; C07K 16/28; C12N 9/22; C12N 15/11

(52) U.S. Cl. CPC C12N 15/86; C07K 14/005; C07K 16/2803; C12N 9/22; C12N 15/11; C07K 2317/622; C07K 2319/02; C07K 2319/03; C07K 2319/33; C12N 2310/20; C12N 2760/20222; C12N 2760/20223; C12N 2760/20252; C12N 2800/80

(57) ABSTRACT

Described herein are programmable tropism virus-like particles (ptVLPs), comprising a membrane comprising a phospholipid bilayer with one or more wild-type or mutant/truncated virus-derived glycoproteins on the external side. The virus-derived envelope glycoprotein(s) can optionally be fused directly to a targeting domain (e.g., peptide, single chain variable fragment (scFv), nanobody, fibronectin type 3 domain (FN3), arginylglycylaspartic acid motif (RGD), single variable domain on a heavy chain/nanobody (VHH), variable domain of new antigen receptor (VNAR), darpin, or other targeting ligand), and/or can be present in combination with a membrane-anchored targeting domain. A biomolecule cargo (preferably fused to a membrane recruitment domain, such as a Pleckstrin homology domain) can be disposed in the core of the ptVLP. Preferably, the ptVLP does not comprise a protein from any human endogenous or exogenous viral gag, pro, pol, or other viral proteins that reside inside of enveloped particles.

### CLAIM OF PRIORITY

This application claims the benefit of U.S. Provisional Patent Application Ser. No. 63/425,894, filed on Nov. 16, 2022. The entire contents of the foregoing are hereby incorporated by reference.

### FEDERALLY SPONSORED RESEARCH OR DEVELOPMENT

This invention was made with Government support under Grant Nos. GM118158 awarded by the National Institutes of Health. The Government has certain rights in the invention.

### SEQUENCE LISTING

The instant application contains a Sequence Listing which has been submitted electronically in XML file format and is hereby incorporated by reference in its entirety. Said XML copy, created on Feb. 2, 2024, is named 29539-0683001 SL.xml and is 300,388 bytes in size.

### TECHNICAL FIELD

Described herein are programmable tropism virus-like particles (ptVLPs), comprising a membrane comprising a phospholipid bilayer with one or more wild-type or mutant/truncated virus-derived envelope glycoproteins on the external side, and a targeting domain (e.g., a peptide, single chain variable fragment (scFv), nanobody, fibronectin type 3 domain (FN3), arginylglycylaspartic acid motif (RGD), single variable domain on a heavy chain/nanobody (VHH), variable domain of new antigen receptor (VNAR), darpin, or other targeting ligand). The targeting domain can optionally be fused directly to the virus-derived envelope glycoproteins(e.g., at the end or internally), and/or can be present in combination with the envelope glycoproteins as a separate membrane-anchored targeting domain fusion protein. Optionally, a biomolecule cargo is disposed in the core of the ptVLP on the inside of the membrane.

### BACKGROUND

The ability to direct delivery of cargo to specific cell types is useful in a number of contexts, particularly in delivery of cargo comprising therapeutic gene editing agents.

### SUMMARY

Described herein are programmable tropism virus-like particles (ptVLPs), comprising a membrane comprising a phospholipid bilayer with one or more wild-type or mutant/truncated virus-derived glycoproteins on the external side. The virus-derived envelope glycoprotein(s) can optionally be fused directly to a targeting domain (e.g., a peptide, single chain variable fragment (scFv), nanobody, fibronectin type 3 domain (FN3), arginylglycylaspartic acid motif (RGD), single variable domain on a heavy chain/nanobody (VHH), variable domain of new antigen receptor (VNAR), darpin, or other targeting ligand), and/or can be present in combination with a membrane-anchored targeting domain. A biomolecule cargo (preferably fused to a membrane recruitment domain, such as a Pleckstrin homology domain) can be disposed in the core of the ptVLP. Preferably, the ptVLP do or do not comprise any human endogenous retroviral (HERV) proteins other than the env, e.g., do or do not comprise gag, pol, or pro (unless the cargo comprises the viral protein(s)). Exogenous virally-derived gag, pol, or pro refers to any gag, pro, pol, gag-pol, gag-pro-pol, and/or pol protein, or any other protein expressed from gag, pro, or pol, from any virus introduced into the cell.

Provided herein are fusion proteins comprising a programmable tropism glycoprotein or envelope protein (ptENV) comprising a virus-derived glycoprotein or envelope protein fused to a targeting domain, optionally wherein the targeting domain is at the C terminus of the glycoprotein or envelope protein, at the N terminus, or is inserted immediately after a signal sequence. Also provided are fusion proteins comprising a membrane-anchored targeting domain comprising a targeting domain fused to a transmembrane domain. In some embodiments, the targeting domain comprises a targeting peptide, e.g., as shown in Table A. In some embodiments, the Targeting Domain comprises a single chain variable fragment (scFv), nanobody, fibronectin type 3 domain (FN3), arginylglycylaspartic acid motif (RGD), single variable domain on a heavy chain/nanobody (VHH), variable domain of new antigen receptor (VNAR), darpin or other targeting ligand. Linkers can be present between any or all of the parts of the fusion proteins.

In some embodiments, the Targeting Domain binds to human CD19, CD4, CD34, ASGR1, TfR1, HER2, CD25, CTLA-4, HB-EGF, ACE2, Aryl hydrocarbon receptor (AhR), keratin 5 (KRT5), KRT13, Fibronectin (FN1), Amyloid precursor protein (APP), neurotrophin receptor (p75NTR), Thy-1/CD90, EpCAM, and/or CFTR.

In some embodiments, the signal sequence comprises MKCLLYLAFLFIGVNCK (SEQ ID NO: 1) or a secretion signal sequence that is derived from VSVG (e.g., MKCLLYLAFLFIGVNC, SEQ ID NO:2), or another signal sequence as known in the art or described herein.

In some embodiments, the ptENV fusion protein comprising a sequence that is at least 95% identical to a sequence set forth herein, e.g., a ptENV comprising a glycoprotein or envelope protein in Table 1, plus a targeting domain.

Further, provided herein are nucleic acids sequence encoding the fusion proteins described herein, as well as vectors comprising the nucleic acid sequence, optionally operably linked to a promoter for expression of the fusion proteins, and host cells comprising the nucleic acid sequences, and optionally expressing the fusion proteins (e.g., producer cells).

Also provided herein are virus-like particles (VLPs) comprising the fusion proteins described herein, and optionally, a cargo disposed in the core of the VLP, wherein the cargo is optionally fused to a phospholipid bilayer recruitment domain.

Additionally provided are programmable tropism virus-like particle (ptVLP), comprising (a) a membrane comprising a phospholipid bilayer and (b) the fusion protein comprising a ptENV as described herein, or a glycoprotein or envelope protein (optionally as listed in Table 1) and the fusion protein comprising a membrane-anchored targeting domain as described herein: and (c) optionally, a cargo disposed in the core of the ptVLP, wherein the cargo is optionally fused to a phospholipid bilayer recruitment domain: and, optionally, wherein the ptVLP does not comprise an exogenous gag, pro and/or pol protein.

In some embodiments, the cargo is a therapeutic or diagnostic protein and/or nucleic acid encoding a therapeutic or diagnostic protein, and/or a chemical, optionally a small molecule therapeutic or diagnostic. In some embodiments, the cargo is a gene editing or epigenetic modulating reagent. In some embodiments, the gene editing or epigenetic modulating reagent comprises a zinc finger (ZF), transcription activator-like effector (TALE), and/or CRISPR-Cas protein, variant, or fusion thereof: a nucleic acid encoding a zinc finger (ZF), transcription activator-like effector (TALE), and/or CRISPR-Cas protein, variant, or fusion thereof: a guide RNA and/or crRNA: or a ribonucleoprotein complex (RNP) comprising a CRISPR-Cas protein, variant, or fusion thereof and/or optionally a guide RNA and/or crRNA.

In some embodiments, the cargo is selected from the proteins listed in Tables 2, 3, 4 & 5, or is at least 95% identical to a sequence set forth herein, e.g., in Table 2, 3, 4, or 5.

In some embodiments, the cargo comprises a CRISPR-Cas protein, and the ptVLP further comprises one or more guide RNAs and/or crRNAs that bind to and direct the CRISPR-Cas protein to a target nucleic acid sequence.

In some embodiments, the cargo comprises a fusion to a phospholipid bilayer recruitment domain, preferably as shown in Table 6, or that is at least 95% identical to a sequence set forth herein in Table 6.

Additionally, provided herein are methods for delivering a cargo to a target cell, optionally a cell in vivo or in vitro, by contacting the cell with a VLP or ptVLP as described herein comprising the cargo.

Further, provided herein are methods of producing a VLP or a ptVLP comprising a cargo by providing a cell expressing (i) a fusion protein as described herein, e.g., ptENV or a glycoprotein or envelope protein (optionally as listed in Table 1) and a separate membrane-anchored targeting domain as described herein: and optionally also expressing a cargo, optionally wherein the cell does not express an exogenous gag, pro, or pol protein: and maintaining the cell under conditions such that the cells produce the VLPs or ptVLPs.

In some embodiments, the methods include harvesting and optionally purifying and/or concentrating the produced VLPs or ptVLPs.

Also provided herein are cells expressing (i) a ptENV fusion protein as described herein, or (ii) a glycoprotein or envelope protein (optionally as listed in Table 1) and a fusion protein comprising the membrane-anchored targeting domain as described herein, part (ii): and optionally a cargo, wherein the cargo is optionally fused to a phospholipid bilayer recruitment domain: and, optionally the cell does not express an exogenous gag, pro and/or pol protein. In some embodiments, the cells are primary or stable human cell lines, e.g., Human Embryonic Kidney (HEK) 293 cells or HEK293 T cells.

In some embodiments, the cargo is a therapeutic or diagnostic protein and/or nucleic acid encoding a therapeutic or diagnostic protein, and/or a small molecule, optionally a therapeutic or diagnostic small molecule. In some embodiments, the cargo is a gene editing or epigenetic modulating reagent. In some embodiments, the gene editing or epigenetic modulating reagent comprises a zinc finger (ZF), transcription activator-like effector (TALE), and/or CRISPR-Cas protein, variant, or fusion thereof: a nucleic acid encoding a zinc finger (ZF), transcription activator-like effector (TALE), and/or CRISPR-Cas protein, variant, or fusion thereof: a guide RNA and/or crRNA: or a ribonucleoprotein complex (RNP) comprising a CRISPR-Cas protein, variant, or fusion thereof and optionally a guide RNA and/or crRNA.

In some embodiments, the cargo reagent is selected from the proteins listed in Tables 2, 3, 4 & 5, or that is at least 95% identical to a sequence set forth herein, e.g., in Table 2, 3, 4, or 5.

In some embodiments, the cargo reagent comprises a CRISPR-Cas protein, variant, or fusion thereof and the ptVLP further comprises one or more guide RNAs and/or crRNAs that bind to and direct the CRISPR-based genome editing or modulating protein to a target sequence.

In some embodiments, the cargo comprises a fusion to a phospholipid bilayer recruitment domain, preferably as shown in Table 6, or that is at least 95% identical to a sequence set forth herein in Table 6.

Unless otherwise defined, all technical and scientific terms used herein have the same meaning as commonly understood by one of ordinary skill in the art to which this invention belongs. Methods and materials are described herein for use in the present invention: other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-D. Exemplary diagrams of ptVLP DNA expression constructs (A & C) that would be transfected into a producer cell, and particle architecture (B & D). The scFv shown in this figure as a membrane-associated targeting moiety is only exemplary and not intended to be limiting.

FIG. 2. HEK293T cells transfected with either a mock control plasmid or a CD19-encoding plasmid were treated with ptVLPs containing human AKT Pleckstrin homology domain fused to SpCas9 and a guide RNA (gRNA) targeted to VEGF site 3. ptVLPs were pseudotyped with an ectodomain-truncated VSVG (VSVG-421, wherein VSVG amino acids 421-511 preceded by a signal sequence are present, as described in Table 1) or mutated (VSVG mut) version of the VSVG envelope protein, and also included a membrane-anchored anti-CD19 scFV. Gene modification (y-axis) was measured by targeted amplicon sequencing of the intended VEGF site 3 on-target site.

FIG. 3. Exemplary gene modification efficiencies induced in cells treated with eVLPs that contained various PH-Cas9/sgRNA (VEGFs3.1-targeted) RNP cargos. HEK293T cells were treated with these eVLPs pseudotyped with VSVG and gene modification efficiencies (y-axis) were determined by targeted amplicon sequencing of the VEGFs3.1 on-target site in those cells.
PKD protein kinase DI (PRKD1)
DAPP dual-adaptor for phosphotyrosine and 3-phosphoinositides-1 (DAPP-1)
FAPP four-phosphate-adaptor protein (FAPP)
OSBP oxysterol-binding protein (OSBP)
SWAP70 switch-associated protein 70 (SWAP70)
GRP cytohesin 3 (CYTH3, formerly GRP1)
BTK Bruton's tyrosine kinase (Btk)
PHLPP Pleckstrin Homology Domain Leucine-rich Repeat Protein Phosphatase (PHLPP)
AKT AKT serine/threonine kinase 1 (AKT1)
PLC phospholipase C delta 1 (PLCδ1)

FIG. 4. Exemplary gene modification efficiencies induced by eVLPs that contained various mutant PH-Cas9/sgRNA (RNF2-targeted) RNP cargos. Primary T cells were treated with eVLPs pseudotyped with either VSVG or VSVG+BaeVTRless and gene modification efficiencies (y-axis) were determined by targeted amplicon sequencing of the RNF2-on-target site in those cells.

### DETAILED DESCRIPTION

Therapeutic proteins and nucleic acids hold great promise, and delivery of proteins and nucleic acids to specific cell types would be of great interest, not least because it provides the possibility of reduced side effects. For example, genome editing reagents such as zinc finger nucleases (ZFNs) or RNA-guided, enzymatically active/inactive DNA binding proteins such as Cas9 have undergone rapid advancements in terms of specificity and the types of edits that can be executed, but the hurdle of safe in vivo delivery still remains an important challenge for gene editing and epigenetic editing therapies.

Virus-like particles (VLPs) have been utilized to deliver mRNA and protein cargo into the cytosol of cells.^{2,3,25-30} VLPs have emerged as an alternative delivery modality to retroviral or lentiviral particles. VLPs can be designed to lack the ability to integrate retroviral DNA, and to package and deliver combinations of protein/RNP/DNA. However, most VLPs, including recently conceived VLPs that deliver genome editing reagents known to date, utilize HIV or other virally-derived gag or gag-pol protein fusions and viral proteases to generate retroviral-like particles.^{25-27,29,30} Some VLPs containing RNA-guided nucleases (RGNs) also must package and express guide RNAs from a lentiviral DNA transcript,²⁷ and some VLPs require a viral protease in order to form functional particles and release genome editing cargo.^{25-27,29} Because this viral protease recognizes and cleaves at multiple amino acid motifs, it can cause damage to the protein cargo or potentially to other endogenous proteins in target recipient cells, which could be hazardous or create challenges for therapeutic applications. Most published VLP modalities that deliver genome editing proteins or RNPs to date exhibit low in vitro and in vivo gene modification efficiencies due to low packaging and transduction efficiency.²⁵⁻²⁷ The complex viral genomes utilized for these VLP components possess multiple reading frames and employ RNA splicing that could result in spurious fusion protein products being delivered.^{25-27,29,30} The presence of reverse transcriptase, integrase, capsid and a virally-derived envelope protein in these VLPs is not ideal for many therapeutic applications because of immunogenicity and off target concerns. In addition, most retroviral particles, such as lentiviral particles, are pseudotyped with VSVG and nearly all described VLPs that deliver genome editing reagents hitherto possess and rely upon VSVG.^{2,3,25-30}

Described herein are various embodiments of virus-like particles with programmable tropism (ptVLPs) that can be used for cell type- or tissue-specific delivery of cargo including genome editing reagents. The ptVLPs include a targeting moiety that is either integrated into the glycoprotein (e.g., as in the sequences shown below) or separate (e.g., on the outer surface of the particle, but membrane-anchored (e.g., by connection to a transmembrane or integral membrane protein(s), GPI anchor(s) or other membrane anchor(s)).

Here, we describe methods and compositions for producing, purifying, and administering ptVLPs for in vitro and in vivo applications, e.g., of genome editing, epigenome modulation, transcriptome editing and proteome modulation. The desired editing or other modulation outcome in the target recipient cell depends on the therapeutic context and will require different gene editing or other cargos to be delivered. Streptococcus pyogenes Cas9 (SpCas9) and Acidaminococcus sp. Cas12a (AsCas12a) are two commonly used RNA-guided enzymes for editing that leverage NHEJ-mediated repair of DNA double-strand breaks (DSBs) induced by these nucleases to introduce stop codons or insertion/deletions (indels) or homology-directed repair (HDR) of the DSBs together with an exogenous DNA donor template that encodes a desired genetic alteration (e.g., precise point mutation(s) or insertions). Cas9-deaminase fusions, also known as base editors, are the current standard for precise editing of a single nucleotide without double stranded DNA cleavage.

### Phospholipid Bilayer Recruitment Domains

Conventional VLPs that have been engineered to encapsulate and deliver protein-based cargo commonly fuse cargo to the INT or GAG polyprotein.^{25-27,29,30,39,40} After transient transfection of production plasmid DNA constructs encoding these proteins and a viral envelope (ENV) protein, the protein fusions are translated in the cytosol of conventional VLP production cell lines, the gag matrix is acetylated and recruited to the cell membrane, and the gag fusions are encapsulated within VLPs as they bud off of the membrane into extracellular space.

In contrast, in some embodiments, proteins can be packaged into ptVLPs by fusing select human protein-derived phospholipid bilayer recruitment domains to protein-based cargo (e.g., as described in WO 2022/020800 or as shown in Table 6).

One such human protein-derived phospholipid bilayer recruitment domain used for this purpose is a human pleckstrin homology (PH) domain. PH domains interact with phosphatidylinositol lipids and proteins within biological membranes, such as PIP2, PIP3, βγ-subunits of GPCRs, and PKC.^{41,42} Alternatively, the human Arc protein can be fused to protein-based cargo to recruit cargo to the cytosolic side of the phospholipid bilayer.⁴³ These human protein-derived phospholipid bilayer recruitment domains, or variants thereof (e.g., as shown in Table 6) can be fused to the N-terminus or C-terminus of protein-based cargo via polypeptide linkers of variable length regardless of the location or locations of one or more nuclear localization sequence(s) (NLS) within the cargo. Preferably, the linker between protein-based cargo and the phospholipid bilayer recruitment domain is a polypeptide linker 5-20, e.g., 8-12, e.g., 10, amino acids in length primarily composed of glycines and serines. The human protein-derived phospholipid bilayer recruitment domain localizes the cargo to the cytosolic face of the phospholipid bilayer and this protein cargo is packaged within ptVLPs that also contain and use an envelope glycoprotein to trigger budding-off of particles from the producer cell into extracellular space. These human protein-derived domains and human proteins can facilitate for localization of cargo to the cytosolic face of the plasma membrane within the ptVLP production cells, and they also allow for the cargo to localize to the nucleus of ptVLP-transduced cells without the utilization of exogenous retroviral gag/pol or chemical and/or light-based dimerization systems. The delivery of Cas9, for example, may be significantly more efficiently loaded as cargo into particles with fusion to a phospholipid bilayer recruitment domain compared to without fusion to a phospholipid bilayer recruitment domain.

### Targeting Domains

Provided herein are VLPs that include targeting domains that bind to antigens on target cells (e.g., ptVLPs) to alter tropism of the VLPs. A number of such antigens are known in the art. Exemplary antigens include CD19,⁷⁰ asialoglycoprotein receptor 1 (ASGR1),⁷¹ Transferrin receptor (TfR),⁷² HER2,⁷³ CD34,⁷⁴ CD4,⁷⁵ CD25,⁷⁶ CTLA-4,⁷⁷ HB-EGF,⁷⁸ ACE2,⁷⁹ Aryl hydrocarbon receptor (AhR),⁸⁰ keratin 5 (KRT5),⁸¹ keratin 17 (KRT17),⁸² keratin 14 (KRT14),⁸³ keratin 13 (KRT13),⁸⁴ Neural cell adhesion molecule L1,⁸⁵ Fibronectin (FN1),^{86,87,88} Amyloid precursor protein (APP),⁸⁹ Programmed cell death protein 1 (PD-1),^{90,91} neurotrophin receptor (p75NTR),⁹² Thy-1/CD90,⁹³ EpCAM,⁹⁴ and/or CFTR.⁹⁵

ptVLP targeting domains can include single chain variable fragment (scFv), nanobody, fibronectin type 3 domain (FN3), arginylglycylaspartic acid motif (RGD), single variable domain on a heavy chain/nanobody (VHH), variable domain of new antigen receptor (VNAR), darpin, or other targeting ligand that binds to an antigen on a target cell.⁴⁷⁻⁵³

Targeting domains can also include peptides, e.g., as shown in Table A.

The targeting domains can be inserted into the sequence of an envelope protein such that it will be displayed on the surface of the ptVLP, as described herein, or can be present as a separate molecule anchored on the outside of the ptVLP membrane. Thus fusion proteins comprising (i) a targeting domain and an envelope glycoprotein, or (ii) a targeting domain and a membrane anchor are provided herein, as well as nucleic acids encoding the fusion proteins. In some embodiments, the targeting domain is inserted into an ENV protein between the signal sequence and the transmembrane domain, optionally replacing some or most of the N terminus of the ENV, including the RBD.

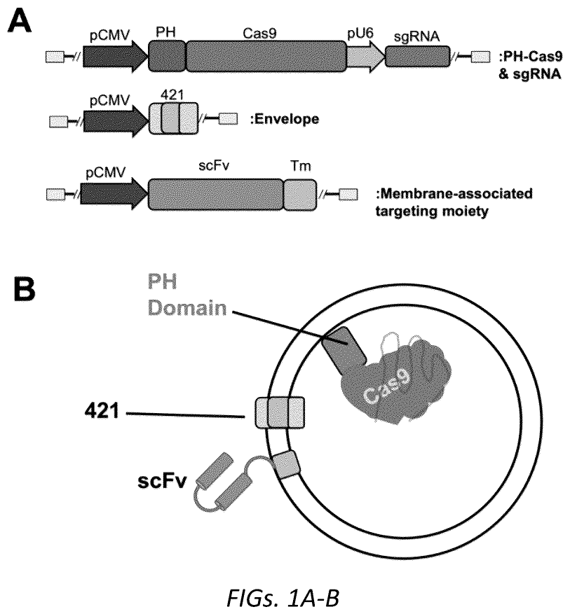

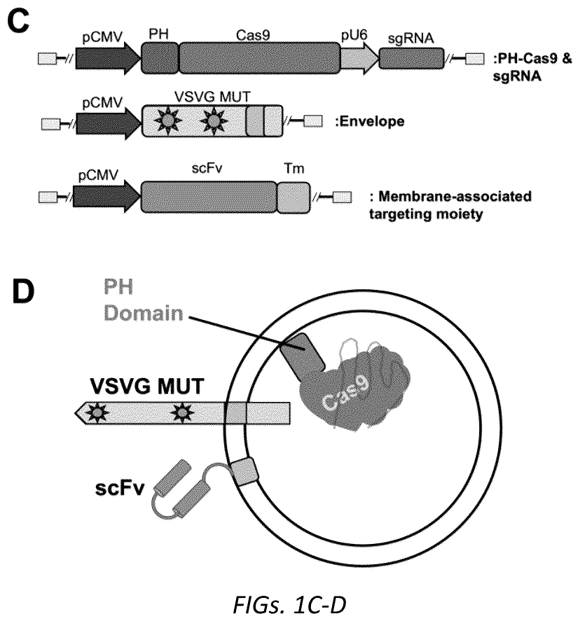

Membrane anchors can be any transmembrane (TM) domain, such as a TM from Platelet-derived growth factor receptor (PDGFR),⁹⁶ CD9,⁹⁷ CD63,⁹⁷ CD81,⁹⁷ CD86, Notch,⁷⁰ CD28.⁹⁸ CD8,⁹⁹ or CD4.¹⁰⁰ In general, the membrane anchored targeting domain fusion proteins will comprise, from N terminus to C terminus, the following a secretion signal sequence—optional linker—targeting domain—optional linker—transmembrane domain (see, e.g., FIG. 1). Preferably, the optional linker between the three domains is a polypeptide linker that is 5-20, e.g., 8-12, e.g., 10, amino acids in length primarily composed of glycines and serines.

TABLE A
Targeting Peptide Sequences

| | | SEQ |
|---|---|---|
| Targeting | | ID |
| Peptide | Sequence | NO: |
| CSP peptide of | CKNEKKNKIERNNKLKQPP | 224 |
| plasmodium | | |
| falciparum | | |
| CSP peptide of | DNEKLRKPKHKKLKQPADG | 225 |
| plasmodium | | |
| falciparum | | |
| peptide in ApoB- | RLTRKRGLK | 226 |
| 100 | RGD | |
| RGD Peptide | | |
| repeating peptide | CGRGDSPC | 227 |
| cyclic peptide 1 | RGDYK | 228 |
| cyclic peptide 2 | RGDFK | 229 |
| cyclic peptide 3 | PHSCNK | 230 |
| cyclic peptide 4 | CSRNLIDC | 231 |
| peptide 431 | VHWDFRQWWQPS | 232 |
| Pep1 | CHPREVDVELYSTVFGH | 233 |
| Pep2 | CEPEAEADAEAGPAGIGAVLKVLTTGLPALISWI | 234 |
| | KRKRQQ | |
| CendR | RPARPAR | 235 |
| IRGD | CRGDKGPDC | 236 |
| LinTT1 | AKRGARSTA | 237 |
| TT1 | CKRGARSTC | 238 |
| Lyp-1 | CGNKRTRGC | 239 |
| GLP-1 | HAEGTFTSDVSSYLEGQAAKEFIAWLVRGRG | 240 |
| HTPP | KNSRSLGENDDGNNEDNEKLR | 241 |
| M27-39 | AQQAANVAATLK | 242 |
| M27-39-HTPP | AQQAANVAATLKKNSRSLGENDDGNNEDNEKL | 243 |
| | R | |
| HSTP1 | CDGRPDRAC | 244 |
| GNSTM-HSTP1 | GNSTMCDGRPDRAC | 245 |

### Signal Sequences

Preferably, the membrane anchored targeting domains and the ptENV comprise an N-terminal signal sequence; the original signal sequence can be used or can be replaced with a heterologous signal sequence. Exemplary signal sequences include the one from the VSV-G protein, e.g., MKCLLYLAFLFIGVNCK (SEQ ID NO: 1) and/or any other secretion signal sequence that is derived from VSVG (e.g., MKCLLYLAFLFIGVNC, SEQ ID NO:2) or a homolog thereof, or from a transmembrane protein and/or a synthetic/engineered signal sequence. A number of secretory signal peptide sequences are known in the art, including human signal sequences, examples of which are shown in Table B (Table adapted from novoprolabs. com/support/articles/commonly-used-leader-peptide-sequences-forefficient-secretion-of-a-recombinant-protein-expressed-in-mammalian-cells-201804211337.html).

TABLE B
Exemplary Human Secretory Signal Peptide Sequences

| Human Signal | | |
|---|---|---|
| sequence | Sequence | SEQ ID NO |
| Oncostatin M | MGVLLTQRTLLSLVLALLFPSMASM | 3. |
| IgG2 H | MGWSCIILFLVATATGVHS | 4. |
| Secrecon* | MWWRLWWLLLLLLLLWPMVWA | 5. |
| IgKVIII | MDMRVPAQLLGLLLLWLRGARC | 6. |
| CD33 | MPLLLLLPLLWAGALA | 7. |
| tPA | MDAMKRGLCCVLLLCGAVFVSPS | 8. |
| Chymotrypsinogen | MAFLWLLSCWALLGTTFG | 9. |
| trypsinogen-2 | MNLLLILTFVAAAVA | 10. |
| Interleukin 2 (IL-2) | MYRMQLLSCIALSLALVTNS | 11. |
| Albumin (HSA) | MKWVTFISLLFSSAYS | 12. |
| insulin | MALWMRLLPLLALLALWGPDPAAA | 13. |
| alpha 1-antitrypsin | MPSSVSWGILLLAGLCCLVPVSLA | 14. |

*, Barash et al., Biochem Biophys Res Commun. 2002 Jun. 21;294(4)835-42.

In some embodiments, another signal sequence that promotes secretion is used, e.g., as described in Table 5 of U.S. Ser. No. 10/993,967; von Heijne, J Mol Biol. 1985 Jul. 5; 184(1)99-105; Kober et al., Biotechnol. Bioeng. 2013; 110 1164-1173; Tsuchiya et al., Nucleic Acids Research Supplement No. 3 261-262 (2003).

In general, the signal peptide is cleaved by a signal peptidase after the nascent protein is inserted into the membrane, as part of the secretory pathway processing inherent to cells.

ptVLP-Mediated Delivery of DNAs, Proteins and RNAs

The ptVLPs described herein can package and deliver biomolecule cargo. ptVLP. “Cargo” refers to a any payload that can be delivered, including chemicals, e.g., small molecule compounds, and biomolecules, including DNA, RNA, peptide nucleic acid (PNA), RNP, proteins, and combinations thereof, including combinations of DNA and RNP, RNP, combinations of DNA and proteins, or proteins, as well as viruses and portions thereof, e.g., for therapeutic or diagnostic use, or for the applications of genome editing, epigenome modulating, and/or transcriptome modulation. RNA in this context includes, for example, single guide RNA (sgRNA), Clustered Regularly Interspaced Palindromic Repeat (CRISPR) RNA (crRNA), and/or mRNA coding for cargo. Other exemplary nucleic acids can include specialty single and/or double-stranded DNA molecules (e.g., plasmid, mini circle, closed-ended linear DNA, AAV DNA, episomes, bacteriophage DNA, homology directed repair templates, etc.), single and/or double-stranded RNA molecules (e.g., single guide RNA, prime editing guide RNA, crRNA, tracrRNA, messenger RNA, transfer RNA, long non-coding RNA, circular RNA, RNA replicon, circular or linear splicing RNA, micro RNA, small interfering RNA, short hairpin RNA, piwi-interacting RNA, toehold switch RNA, RNAs that can be bound by RNA binding proteins, bacteriophage RNA, or internal ribosomal entry site containing RNA). Combinations of the above cargos (e.g., AAV particles and/or ribonucleoprotein (RNP) complexes comprising RNA and protein, e.g., guide RNA/CRISPR Cas protein complexes) can also be included.

As used herein, “small molecules” refers to small organic or inorganic molecules of molecular weight below about 3,000 Daltons. In general, small molecules useful for the invention have a molecular weight of less than 3,000 Daltons (Da). The small molecules can be, e.g., from at least about 100 Da to about 3,000 Da (e.g., between about 100 to about 3,000 Da, about 100 to about 2500 Da, about 100 to about 2,000 Da, about 100 to about 1,750 Da, about 100 to about 1,500 Da, about 100 to about 1,250 Da, about 100 to about 1,000 Da, about 100 to about 750 Da, about 100 to about 500 Da, about 200 to about 1500, about 500 to about 1000, about 300 to about 1000 Da, or about 100 to about 250 Da).

In some embodiments, the cargo is limited by the diameter of the particles, e.g., which in some embodiments can range from 30 nm to 500 nm.

In some embodiments, the cargo can include a combination of DNA and RNA, e.g., when ptVLPs are produced via transient transfection of a production cell line. DNA that is transfected into cells will possess size-dependent mobility such that a fraction of the transfected DNA will remain in the cytosol while another fraction of the transfected DNA will localize to the nucleus.⁴⁴⁻⁴⁶ A fraction of the transfected DNA in the nucleus will express components encoded on these plasmids needed to create ptVLPs and another fraction in the cytosol/near the plasma membrane will be encapsulated and delivered in ptVLPs. See, e.g., FIGS. 1-4 of WO 2022/020800.

Cargo developed for applications of genome or gene editing also includes CRISPR-Cas nucleases and fusions and variants thereof, e.g., prime editors, and base editors. Nucleases include ZFNs and Transcription activator-like effector nucleases (TALENs) that comprise a FokI or AcuI nuclease domain; and CRISPR Cas proteins or a functional derivative thereof (e.g., as shown in Table 2) (ZFNs are described, for example, in United States Patent Publications 20030232410; 20050208489; 20050026157; 20050064474; 20060188987; 20060063231; and International Publication WO 07/014275) (TALENs are described, for example, in United States Patent Publication U.S. Pat. No. 9,393,257B2; and International Publication WO2014134412A1) (CRISPR Cas proteins are described, for example, in United States Patent Publications U.S. Pat. No. 8,697,359B1; US20180208976A1; and International Publications WO2014093661A2; WO2017184786A8).³⁴⁻³⁶ Base editors can include any CRISPR based nuclease orthologs (wt, nickase, or catalytically inactive (CI)), e.g., as shown in Table 2, fused at the N-terminus to a nucleotide deaminase or nucleoside deaminase or a functional derivative thereof (e.g., as shown in Table 3), or comprising a deaminase domain inlaid internally, with or without a fusion at the C-terminus to one or multiple uracil glycosylase inhibitors (UGIs) using polypeptide linkers of variable length (Base editors are described, for example, in United States Patent Publications US20150166982A1; US20180312825A1; U.S. Ser. No. 10/113,163B2; and International Publications WO2015089406A1; WO2018218188A2; WO2017070632A2; WO2018027078A8; WO2018165629A1; WO 2018/218166).³⁷,38 In addition, prime editors are also compatible with mVLP delivery modalities (Prime editors are described, for example, in Anzalone et al., Nature. 2019 December; 576(7785)149-157). Prime editors can be delivered, e.g., as fusions of Cas nickase to a reverse transcriptase or as separate components (see, e.g., Granewald et al., Nat Biotechnol. 2022 Sep. 26. doi 10.1038/s41587-022-01473-1; and Liu et al., Nat Biotechnol. 2022 Sep; 40(9)1388-1393).

Cargo designed for the purposes of epigenome modulating includes CRISPR Cas proteins, zinc fingers (ZFs) and TALEs fused to an epigenome/epigenetic modulating agent or combination of epigenome/epigenetic modulating agent or a functional derivative thereof connected together by one or more variable length polypeptide linkers. Exemplary epigenetic modulating agents include CRISPR-Cas proteins (e.g., nickases or catalytically inactive Cas) fused to DNA methylases, histone acetyltransferases, and deacetylases, as well as transcriptional activators or repressors (see, e.g., Tables 2 & 4). Examples include, e.g., transcriptional repressors (e.g., KRAB, ERD, SID, and others, e.g., amino acids 473-530 of the ets2 repressor factor (ERF) repressor domain (ERD), amino acids 1-97 of the KRAB domain of KOXI, or amino acids 1-36 of the Mad mSIN3 interaction domain (SID); see Beerli et al., PNAS USA 9514628-14633 (1998)) or silencers such as Heterochromatin Protein 1 (HP1, also known as swi6), e.g., HP1α or HP1β; proteins or peptides that could recruit long non-coding RNAs (lncRNAs) fused to a fixed RNA binding sequence such as those bound by the MS2 coat protein, endoribonuclease Csy4, or the lambda N protein; enzymes that modify the methylation state of DNA (e.g., DNA methyltransferase (DNMT) or TET proteins); or enzymes that modify histone subunits (e.g., histone acetyltransferases (HAT), histone deacetylases (HDAC), histone methyltransferases (e.g., for methylation of lysine or arginine residues) or histone demethylases (e.g., for demethylation of lysine or arginine residues)) In some embodiments, the sequence of the cargo is at least 95% identical to a sequence set forth herein.

sgRNAs can complex with genome editing reagents during the packaging process to be co-delivered within ptVLPs. Also, linear or circular RNAs encoding cargo or edits that are to be installed by a prime editor could be co-packaged with genome editing reagents that are fused to RNA binding proteins, such as MS2, PP7, COM, or TAR hairpin binding protein (TBP) or human SLBP. Cargo designed for the purposes of transcriptome editing includes CRISPR Cas proteins or any functional derivatives thereof (e.g., as shown in Table 5) or CRISPR Cas proteins or any functional derivatives thereof (e.g., as shown in Table 5) fused to nucleotide deaminases or nucleoside deaminases (e.g., as shown in Table 3) by one or more variable length polypeptide linkers.

The cargo can also include any therapeutically or diagnostically useful protein, DNA, RNP, or combination of DNA, protein and/or RNP. See, e.g., WO2014005219; U.S. Ser. No. 10/137,206; US20180339166; U.S. Pat. No. 5,892,020A; EP2134841B1; WO2007020965A1. For example, cargo encoding or composed of nuclease or base editor proteins or RNPs or derivatives thereof can be delivered to retinal cells for the purposes of correcting a splice site defect responsible for Leber Congenital Amaurosis type 10. In the mammalian inner ear, ptVLP delivery of base editing reagents or HDR promoting cargo to sensory cells such as cochlear supporting cells and hair cells for the purposes of editing β-catenin (β-catenin Ser 33 edited to Tyr, Pro, or Cys) in order to better stabilize β-catenin could help reverse hearing loss.

In another application, ptVLP delivery of RNA editing reagents or proteome perturbing reagents could cause a transitory reduction in cellular levels of one or more specific proteins of interest (potentially at a systemic level, in a specific organ or a specific subset of cells, such as a tumor), and this could create a therapeutically actionable window when secondary drug(s) could be administered (this secondary drug is more effective in the absence of the protein of interest or in the presence of lower levels of the protein of interest). For example, ptVLP delivery of RNA editing reagents or proteome perturbing reagents could trigger targeted degradation of MAPK and PI3K/AKT proteins and related mRNAs in vemurafenib/dabrafenib-resistant BRAF-driven tumor cells, and this could open a window for the administration of vemurafenib/dabrafenib because BRAF inhibitor resistance is temporarily abolished (resistance mechanisms based in the MAPK/PI3K/AKT pathways are temporarily downregulated by ptVLP cargo). This example is especially pertinent when combined with ptVLPs that are antigen inducible and therefore specific for tumor cells. Alternatively, the transitory reduction in cellular levels of a specific protein of interest may itself have therapeutic benefit.

In some embodiments, ptVLPs could be used deliver factors, e.g., including the Yamanaka factors Oct3/4, Sox2, Klf4, and c-Myc, to cells such as human or mouse fibroblasts, in order to generate induced pluripotent stem cells or to deliver factors that induce forward differentiation or trans-differentiation into a specific cell-type.

In some embodiments, ptVLPs could deliver dominant-negative forms of proteins in order to elicit a therapeutic effect.

ptVLPs that are antigen-specific (e.g., tumor-antigen specific) could be targeted to cancer cells in order to deliver proapoptotic proteins BIM, BID, PUMA, NOXA, BAD, BIK, BAX, BAK and/or HRK in order to trigger apoptosis of cancer cells. Tumor antigens are known in the art.

90% of pancreatic cancer patients present with unresectable disease. Around 30% of patients with unresectable pancreatic tumors will die from local disease progression, so it is desirable to treat locally advanced pancreatic tumors with ablative radiation, but the intestinal tract cannot tolerate high doses of radiation needed to cause tumor ablation. Selective radioprotection of the intestinal tract enables ablative radiation therapy of pancreatic tumors while minimizing damage done to the surrounding gastrointestinal tract. To this end, ptVLPs could be loaded with dCas9 fused to the transcriptional repressor KRAB and guide RNA targeting EGLN. EGLN inhibition has been shown to significantly reduce gastrointestinal toxicity from ablative radiation treatments because it causes selective radioprotection of the gastrointestinal tract but not the pancreatic tumor.⁵⁴ Such fusion proteins, ptVLPs, and methods of making and using the same are provided herein.

Unbound steroid receptors reside in the cytosol. After binding to ligands, these receptors will translocate to the nucleus and initiate transcription of response genes. ptVLPs could deliver single chain variable fragment (scFv) antibodies to the cytosol of cells that bind to and disrupt cytosolic steroid receptors. For example, the scFv could bind to the glucocorticoid receptor and prevent it from binding dexamethasone, and this would prevent transcription of response genes, such as metallothionein IE that has been linked to tumorigenesis.⁵⁵

ptVLPs can be indicated for treatments that involve targeted disruption of proteins. For example, ptVLPs can be utilized for targeting and disrupting proteins in the cytosol of cells by delivering antibodies/scFvs to the cytosol of cells. Classically, delivery of antibodies through the plasma membrane to the cytosol of cells has been notoriously difficult and inefficient. This mode of protein inhibition is similar to how a targeted small molecule binds to and disrupts proteins in the cytosol and could be useful for the treatment of a diverse array of diseases.⁵⁶⁻⁵⁸ Such fusion proteins, ptVLPs, and methods of making and using the same are included herein.

In addition, the targeting of targeted small molecules is limited to proteins of a certain size that contain binding pockets that are relevant to catalytic function or protein-protein interactions. scFvs are not hampered by these limitations because scFvs can be generated that bind to many different moieties of a protein in order to disrupt catalysis and interactions with other proteins. For example, RAS oncoproteins are implicated across a multitude of cancer subtypes, and RAS is one of the most frequently observed oncogenes in cancer. For instance, the International Cancer Genome Consortium found KRAS to be mutated in 95% of their Pancreatic Adenocarcinoma samples. RAS isoforms are known to activate a variety of pathways that are dysregulated in human cancers, like the PI3K and MAPK pathways. Despite the aberrant roles RAS plays in cancer, no efficacious pharmacologic direct or indirect small molecule inhibitors of RAS have been developed and approved for clinical use. One strategy for targeting RAS could be ptVLPs that can deliver specifically to cancer cells scFvs that bind to and disrupt the function of multiple RAS isoforms.⁵⁶⁻⁵⁸

ptVLP Composition, Production, Purification and Applications

ptVLPs can be produced from producer cell lines that are either transiently transfected with at least one plasmid or stably expressing constructs that have been integrated into the producer cell line genomic DNA. This, in some embodiments, the ptVLPs described herein can be produced and package protein-based cargo by integrating all production DNA constructs into the genomic DNA of production cell lines. Once cell lines (e.g., production lines) are created, protein delivery ptVLPs can be produced in a constitutive or inducible fashion.

Alternatively, some or all of the components for producing ptVLPs can be transiently expressed. In some embodiments, for ptVLPs, a single plasmid is used in the transfection that comprises sequences encoding one or more transmembrane envelope glycoproteins (with or without specified mutation(s)/truncation(s) and/or targeting domain fusions, e.g., as described herein) (e.g., unmodified envelopes are shown in Table 1) or a transmembrane envelope glycoprotein with or without specified mutation(s)/truncation(s) with a membrane-anchored targeting domain in trans, cargo (e.g., a therapeutic protein or a gene editing reagent such as a zinc finger, transcription activator-like effector (TALE), and/or CRISPR-based genome editing/modulating protein and/or RNP such as those found in Tables 2, 3, 4 & 5), with or without fusion to a plasma membrane recruitment domain (e.g., as shown in Table 6), and at least one guide RNA, if necessary.

In some embodiments, two to three plasmids are used in the transient transfection. These two to three plasmids can include the following (any two or more components listed here can be combined in a single plasmid)
1. A plasmid comprising sequences encoding cargo, e.g., a therapeutic protein or a genome editing reagent, with or without a fusion to a plasma membrane recruitment domain.
2. A plasmid comprising one or more targeted envelope glycoproteins with specified mutation(s)/truncation(s) and/or targeting domain fusions (e.g., unmodified envelopes are listed in Table 1).
3. If the genome editing reagent from plasmid 1 requires one or more guide RNAs, a plasmid comprising one or more guide RNAs apposite for the genome editing reagent in plasmid 1.
   In addition, three, four or more plasmids could be used in the transient transfection. These four or more plasmids can include the following (any two or more components can be combined in a single plasmid)
1. A plasmid comprising sequences encoding cargo, e.g., a therapeutic protein or a genome editing reagent, with or without a fusion to a plasma membrane recruitment domain.
2. A plasmid comprising one or more envelope glycoproteins with specified mutation(s)/truncation(s) (e.g., as listed in Table 1).
3. A plasmid comprising one or more membrane-anchored targeting domains(s) (e.g., targeting peptide, scFv, nanobody, FN3, RGD, VHH, VNAR, darpin, or other targeting ligand), e.g., when the envelope glycoprotein does not include a targeting domain (though in some embodiments, two or more different targeting domains are used, in the ENV and/or as separate membrane-anchored targeting domains).
4. If the genome editing reagent from plasmid 1 requires one or more guide RNAs, a plasmid comprising one or more guide RNAs apposite for the genome editing reagent in plasmid 1.
   If it is desired to deliver a type of DNA molecule other than plasmid(s), the above-mentioned transfection can be performed with double-stranded closed-end linear DNA, episome, mini circle, double-stranded oligonucleotide and/or other specialty/modified DNA, RNA, AAV, adenovirus, anellovirus, or peptide nucleic acid (PNA) molecules. Alternatively, for ptVLPs, the producer cell line can be made to stably express one or more of the constructs (1 through 3) described in the transfection above.

In some embodiments, the methods for producing ptVLPs can include using cells that have or have not been manipulated to express any exogenous proteins except for a targeted viral envelope protein comprising a targeting domain fusion or viral envelope with associated targeting domain in trans with or without specified mutation(s)/truncation(s) (e.g., as shown in Table 1), and, if desired, a plasma membrane recruitment domain (e.g., as shown in Table 6); in other words, no cargo is expressed. In this embodiment, the “empty” particles that are produced can be loaded with cargo and/or small molecules by utilizing incubation, nucleofection, lipid, polymer, or CaCl₂) transfection, sonication, freeze thaw, and/or heat shock of purified particles mixed with cargo. In all embodiments, producer cells do not express any exogenous gag protein. This type of loading allows for cargo to be unmodified by fusions to plasma membrane recruitment domains and represents a significant advancement from previous VLP technologies.

The plasmids, or other types of specialty DNA molecules known in the art or described herein, can also preferably include other elements to drive expression or translation of the encoded sequences, e.g., a promoter sequence; an enhancer sequence, e.g., 5′ untranslated region (UTR) or a 3′ UTR; a polyadenylation site; IRES; 2A peptide; an insulator sequence; or another sequence that increases or controls expression (e.g., an inducible promoter element).

Appropriate producer cell lines can include primary or stable human cell lines refractory to the effects of transfection reagents and fusogenic effects due to virally-derived glycoproteins. Examples of appropriate cell lines include Human Embryonic Kidney (HEK) 293 cells, HEK293 T/17 SF cells kidney-derived Phoenix-AMPHO cells, and placenta-derived BeWo cells. For example, such cells could be selected for their ability to grow as adherent cells, or suspension cells. In some embodiments, the producer cells can be cultured in classical DMEM under serum conditions, serum-free conditions, or exosome-free serum conditions. ptVLPs can be produced from cells that have been derived from patients (autologous ptVLPs) and other FDA-approved cell lines (allogenic ptVLPs) as long as these cells can be transfected with DNA constructs that encode the aforementioned ptVLP production components by various techniques known in the art.

In addition, if it is desirable, more than one genome editing reagent encoded in polynucleic acid construct(s) can be included in the transfection. The DNA constructs can be designed to overexpress proteins in the producer cell lines. The plasmid backbones, for example, used in the transfection can be familiar to those skilled in the art, such as the pCDNA3 backbone that employs the CMV promoter for RNA polymerase II transcripts or the U6 promoter for RNA polymerase III transcripts. Various techniques known in the art can be employed for introducing polynucleic acid molecules into producer cells. Such techniques include chemical-facilitated transfection using compounds such as calcium phosphate, cationic lipids, cationic polymers, liposome-mediated transfection, such as cationic liposome like LIPOFECTAMINE (LIPOFECTAMINE 2000 or 3000 and TransIT-X2), polyethyleneimine, non-chemical methods such as electroporation, particle bombardment, or microinjection.

A human producer cell line that stably expresses the necessary ptVLP components in a constitutive and/or inducible fashion can be used for production of ptVLPs. ptVLPs can be produced from cells that have been derived from patients (autologous ptVLPs) and other FDA-approved cell lines (allogenic ptVLPs) if these cells have been converted into stable cell lines that express the aforementioned ptVLP components.

Also provided herein are the producer cells themselves.

Production of Cargo-Loaded ptVLPs and Compositions

Preferably ptVLPs are harvested from cell culture medium supernatant 36-48 hours post-transfection, or when ptVLPs are at the maximum concentration in the medium of the producer cells (the producer cells are expelling particles into the media and at some point in time, the particle concentration in the media will be optimal for harvesting the particles). Supernatant can be purified by any known methods in the art, such as centrifugation, ultracentrifugation, precipitation, ultrafiltration, tangential flow filtration, and/or chromatography. In some embodiments, the supernatant is first filtered, e.g., to remove particles larger than 1 μm, e.g., through 0.45 pore size polyvinylidene fluoride hydrophilic membrane (Millipore Millex-HV) or 0.8 μm pore size mixed cellulose esters hydrophilic membrane (Millipore Millex-AA). After filtration, the supernatant can be further purified and concentrated, e.g., using ultracentrifugation, e.g., at a speed of 80,000 to 100,000 xg at a temperature between 1° C. and 5° C. for 1 to 2 hours, or at a speed of 8,000 to 15,000 g at a temperature between 1° C. and 5° C. for 10 to 16 hours. After this centrifugation step, the ptVLPs are concentrated in the form of a centrifugate (pellet), which can be resuspended to a desired concentration, mixed with transduction-enhancing reagents, subjected to a buffer exchange, or used as is. In some embodiments, ptVLP-containing supernatant can be filtered, precipitated, centrifuged and resuspended to a concentrated solution. For example, polyethylene glycol (PEG), e.g., PEG 8000, or antibody-bead conjugates that bind to ptVLP surface proteins or membrane components can be used to precipitate particles. Purified particles are stable and can be stored at 4° C. for up to a week or −80° C. for years without losing appreciable activity.

Preferably, ptVLPs are resuspended or undergo buffer exchange so that particles are suspended in an appropriate carrier. In some embodiments, buffer exchange can be performed by ultrafiltration (e.g., Sartorius Vivaspin 500 MWCO 100,000). An exemplary appropriate carrier for ptVLPs to be used for in vitro applications would preferably be a cell culture medium that is suitable for the cells that are to be transduced by ptVLPs. Transduction-enhancing reagents that can be mixed into the purified and concentrated ptVLP solution for in vitro applications include reagents known by those familiar with the art (e.g., Miltenyi Biotec Vectofusin-1, Millipore Polybrene, Takara Retronectin, Sigma Protamine Sulfate, and the like). After ptVLPs in an appropriate carrier are applied to the cells to be transduced, transduction efficiency can be further increased by centrifugation. Preferably, the plate containing ptVLPs applied to cells can be centrifuged at a speed of 1,150 g at room temperature for 30 minutes. After centrifugation, cells are returned into the appropriate cell culture incubator (e.g., humidified incubator at 37° C. with 5% CO₂).

An appropriate carrier for ptVLPs to be administered to a mammal, especially a human, would preferably be a pharmaceutically acceptable composition. A “pharmaceutically acceptable composition” refers to a non-toxic semisolid, liquid, or aerosolized filler, diluent, encapsulating material, colloidal suspension or formulation auxiliary of any type. Preferably, this composition is suitable for injection. These may be in particular isotonic, sterile, saline solutions (monosodium or disodium phosphate, sodium, potassium, calcium or magnesium chloride and similar solutions or mixtures of such salts), or dry, especially freeze-dried compositions which upon addition, depending on the case, of sterilized water or physiological saline, permit the constitution of injectable solutions. Another appropriate pharmaceutical form would be aerosolized particles for administration by intranasal inhalation or intratracheal intubation.

The pharmaceutical forms suitable for injectable use include sterile aqueous solutions or suspensions. The solution or suspension may comprise additives which are compatible with ptVLPs and do not prevent ptVLP entry into target cells. In all cases, the form must be sterile and must be fluid to the extent that the form can be administered with a syringe. It must be stable under the conditions of manufacture and storage and must be preserved against the contaminating action of microorganisms, such as bacteria and fungi. An example of an appropriate solution is a buffer, such as phosphate buffered saline.

Methods of formulating suitable pharmaceutical compositions are known in the art, see, e.g., Remington The Science and Practice of Pharmacy, 21st ed., 2005; and the books in the series Drugs and the Pharmaceutical Sciences a Series of Textbooks and Monographs (Dekker, NY). For example, solutions or suspensions used for parenteral, intradermal, or subcutaneous application can include the following components a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, NJ) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

Compositions comprising cargo-loaded ptVLPs as described herein can be included in a container, pack, or dispenser together with instructions for administration.

TABLE 1
Exemplary unmodified virus-derived glycoproteins and envelopes.
Virally-derived glycoproteins and envelopes

Vesicular stomatitis virus glycoprotein (VSVG)
Vesicular stomatitis Alagoas glycoprotein (VSAG)
Vesicular stomatitis New Jersey glycoprotein (strain
Ogden subtype Concan) (VSNJG)
Piry glycoprotein
Maraba glycoprotein
Vesicular stomatitis Indiana virus (strain Orsay)
(VSOG)
Chandipura glycoprotein
vesicular stomatitis Glasgow glycoprotein (VSGG)
Isfahan glycoprotein
Radi virus glycoprotein
Jurona glycoprotein
Malpais Spring glycoprotein
Perinet Spring glycoprotein
Morreton glycoprotein
Amphotropic MLV glycoprotein (AMLVG)
10A1 MLV glycoprotein (10A1MLVG)
Influenza A Hemagglutinin
Influenza A Neuraminidase
Sindbis virus glycoprotein (SINVG)
Measles virus Hemagglutinin (MeV H)
Measles virus fusion (MeV F)
Tupaia Paramyxovirus Hemagglutinin (TPMV H)
Tupaia Paramyxovirus fusion (TPMV F)
Canine distemper virus Hemagglutinin (CDV H)
Canine distemper virus fusion (CDV F)
Nipah virus glycoprotein (NiVG)
Nipah virus fusion
Cocal virus glycoprotein (CVG)

TABLE 1b
Exemplary modified virus-derived glycoproteins and envelopes.
Virally-derived glycoproteins and envelopes

Vesicular stomatitis virus Glycoprotein (VSVG) (K47A)
Vesicular stomatitis virus Glycoprotein (VSVG) (K47E)
Vesicular stomatitis virus Glycoprotein (VSVG) (K47G)
Vesicular stomatitis virus Glycoprotein (VSVG) (K47Q)
Vesicular stomatitis virus Glycoprotein (VSVG) (K47W)
Vesicular stomatitis virus Glycoprotein (VSVG) (K47A)(R354A)
Vesicular stomatitis virus Glycoprotein (VSVG) (K47E)(R354A)
Vesicular stomatitis virus Glycoprotein (VSVG) (K47G)(R354A)
Vesicular stomatitis virus Glycoprotein (VSVG) (K47Q)(R354A)
Vesicular stomatitis virus Glycoprotein (VSVG) (K47W)(R354A)
Targeting Domain-VSVG
Vesicular stomatitis virus Glycoprotein (VSVG truncation) (F421 Truncation)
Vesicular stomatitis virus Glycoprotein (VSVG truncation) (F440 Truncation)
Vesicular stomatitis virus Glycoprotein (VSVG truncation) (F448 Truncation)
Targeting Domain-VSVG Truncation fusion 421
Targeting Domain-VSVG Truncation fusion 440
Targeting Domain-VSVG Truncation fusion 448
Amphotropic Murine leukemia virus Glycoprotein (AMLVG) (D86K) Receptor
binding domain mutant
Amphotropic Murine leukemia virus Glycoprotein (AMLVG) (R-domain deletion)
Amphotropic Murine leukemia virus Glycoprotein (AMLVG) (Furin-cleavage
mutant)
Amphotropic Murine leukemia virus Glycoprotein (AMLVG) (L640A)
Amphotropic Murine leukemia virus Glycoprotein (AMLVG) (Y644A) Endocytosis
signal mutant
Targeting Domain-AMLVG
10A1 Murine leukemia virus Glycoprotein (10A1MLVG) (D86K) Receptor binding
domain mutant
10A1 Murine leukemia virus Glycoprotein (10A1MLVG) (R-domain deletion)
10A1 Murine leukemia virus Glycoprotein (10A1MLVG) (Furin-cleavage mutant)
10A1 Murine leukemia virus Glycoprotein (10A1MLVG) (L631A)
10A1 Murine leukemia virus Glycoprotein (10A1MLVG) (Y635A) Endocytosis
signal mutant
Targeting Domain-10A1MLVG
Influenza A (FPV)/Rostock/1934, subtype H7 virus Hemagglutinin
(Y106F)(E199Q)(G237K)
Influenza A (FPV)/Rostock/1934, subtype H7 virus Hemagglutinin (Furin cleavage
mutation)
Influenza A/Puerto Rico/8/34, subtype N1 Neuraminidase (T55A)
Sindbis Virus Glycoprotein (SINVG) triple MUT, E3(61-64del) E2(68SLEQ71 to
68AAAA71) E2(159KE160 to 159AA160)
Sindbis Virus Glycoprotein (SINVG) triple MUT, E3(61-64del) E2(68SLEQ71 to
68AAAA71) E2(159KE160 to 159AA160) with HA TAG
Sindbis Virus Glycoprotein (SINVG) triple MUT, E3(61-64del) E2(68SLEQ71 to
68AAAA71) E2(159KE160 to 159AA160) with targeting domain fusion site
Sindbis Virus Glycoprotein (SINVG) triple MUT version 2, E3(61-64del)
E2(68SLEQ71 to 68AAAA71) E2(159KE160 to 159AA160)
Sindbis Virus Glycoprotein (SINVG) triple MUT version 2, E3(61-64del)
E2(68SLEQ71 to 68AAAA71) E2(159KE160 to 159AA160) with HA TAG
Sindbis Virus Glycoprotein (SINVG) triple MUT version 2, E3(61-64del)
E2(68SLEQ71 to 68AAAA71) E2(159KE160 to 159AA160) with targeting domain
fusion site
Sindbis Virus Glycoprotein (SINVG) quad MUT, E3(61-64del) E2(68SLEQ71 to
68AAAA71) E2(159KE160 to 159AA160) E1(250AK251 to 250SG251)
Sindbis Virus Glycoprotein (SINVG) quad MUT, E3(61-64del) E2(68SLEQ71 to
68AAAA71) E2(159KE160 to 159AA160) E1(250AK251 to 250SG251) with HA
TAG
Sindbis Virus Glycoprotein (SINVG) quad MUT, E3(61-64del) E2(68SLEQ71 to
68AAAA71) E2(159KE160 to 159AA160) E1(250AK251 to 250SG251) with
targeting domain fusion site
Sindbis Virus Glycoprotein (SINVG) quad MUT version 2, E3(61-64del)
E2(68SLEQ71 to 68AAAA71) E2(159KE160 to 159AA160) E1(250AK251 to
250SG251)
Sindbis Virus Glycoprotein (SINVG) quad MUT version 2, E3(61-64del)
E2(68SLEQ71 to 68AAAA71) E2(159KE160 to 159AA160) E1(250AK251 to
250SG251) with HA TAG
Sindbis Virus Glycoprotein (SINVG) quad MUT version 2, E3(61-64del)
E2(68SLEQ71 to 68AAAA71) E2(159KE160 to 159AA160) E1(250AK251 to
250SG251) with targeting domain fusion site
Measles Virus Hemagglutinin (MeV H) delta 18
Measles Virus Hemagglutinin (MeV H) delta 18 double mut (Y463A)(R515A)
Measles Virus Hemagglutinin (MeV H) delta 18 double mut (Y463A)(R515A) with
targeting domain fusion site
Measles Virus Hemagglutinin (MeV H) delta 18 quad mut (Y463A)(R515A)
(530SF531 to 530LS531)
Measles Virus Hemagglutinin (MeV H) delta 18 quad mut (Y463A)(R515A)
(530SF531 to 530LS531) with targeting domain fusion site
Measles Virus Hemagglutinin (MeV H) delta 19
Measles Virus Hemagglutinin (MeV H) delta 19 double mut (Y463A)(R515A)
Measles Virus Hemagglutinin (MeV H) delta 19 double mut (Y463A)(R515A) with
targeting domain fusion site
Measles Virus Hemagglutinin (MeV H) delta 19 quad mut (Y463A)(R515A)
(530SF531 to 530LS531)
Measles Virus Hemagglutinin (MeV H) delta 19 quad mut (Y463A)(R515A)
(530SF531 to 530LS531) with targeting domain fusion site
Measles Virus Hemagglutinin (MeV H) delta 24AAAA
Measles Virus Hemagglutinin (MeV H) delta 24AAAA double mut
(Y463A)(R515A)
Measles Virus Hemagglutinin (MeV H) delta 24AAAA double mut
(Y463A)(R515A) with targeting domain fusion site
Measles Virus Hemagglutinin (MeV H) delta 24AAAA quad mut (Y463A)(R515A)
(530SF531 to 530LS531)
Measles Virus Hemagglutinin (MeV H) delta 24AAAA quad mut (Y463A)(R515A)
(530SF531 to 530LS531) with targeting domain fusion site
Measles Virus Fusion (MeV F) delta 24
Measles Virus Fusion (MeV F) delta 24 (T461I) hyperfusogenic mut
Measles Virus Fusion (MeV F) delta 30
Measles Virus Fusion (MeV F) delta 30 (T461I) hyperfusogenic mut
Tupaia Paramyxovirus Hemagglutinin (TPMV H) delta 32
Tupaia Paramyxovirus Hemagglutinin (TPMV H) delta 32 with targeting domain
fusion site
Tupaia Paramyxovirus Hemagglutinin (TPMV H) delta 80
Tupaia Paramyxovirus Hemagglutinin (TPMV H) delta 80 with targeting domain
fusion site
Tupaia Paramyxovirus Fusion (TPMV F) delta 32
Canine distemper virus Hemagglutinin (CDV H) WT with targeting domain fusion
site
Canine distemper virus Hemagglutinin (CDV H) delta 18
Canine distemper virus Hemagglutinin (CDV H) delta 18 with targeting domain
fusion site
Canine distemper virus Hemagglutinin (CDV H) delta 19
Canine distemper virus Hemagglutinin (CDV H) delta 19 with targeting domain
fusion site
Canine distemper virus Fusion (CDV F) T to I hyperfusogenic mutation
Canine distemper virus Fusion (CDV F) delta 24
Canine distemper virus Fusion (CDV F) delta 24 T to I hyperfusogenic mutation
Canine distemper virus Fusion (CDV F) delta 30
Canine distemper virus Fusion (CDV F) delta 30 T to I hyperfusogenic mutation
Canine distemper virus Fusion (CDV F) WT mini signal sequence d107
Canine distemper virus Fusion (CDV F) WT mini signal sequence d107 T to I
hyperfusogenic mutation
Canine distemper virus Fusion (CDV F) mini signal sequence d107 delta 24
Canine distemper virus Fusion (CDV F) mini signal sequence d107 delta 24 T to I
hyperfusogenic mutation
Canine distemper virus Fusion (CDV F) mini signal sequence d107 delta 30
Canine distemper virus Fusion (CDV F) mini signal sequence d107 delta 30 T to I
hyperfusogenic mutation
Nipah virus Glycoprotein (NiVG) WT with targeting domain fusion site
Nipah virus Glycoprotein (NiVG) delta 33
Nipah virus Glycoprotein (NiVG) delta 33 with targeting domain fusion site
Nipah virus Glycoprotein (NiVG) delta 34
Nipah virus Glycoprotein (NiVG) delta 34 with targeting domain fusion site
Nipah virus Fusion delta 22
Nipah virus Fusion delta 25
Nipah Virus Glycoprotein (NiVG) (E501A)
Nipah Virus Glycoprotein (NiVG) (W504A)
Nipah Virus Glycoprotein (NiVG) (Q530A)
Nipah Virus Glycoprotein (NiVG) (E533A)
Cocal virus glycoprotein (CVG) (K64Q)
Cocal virus glycoprotein (CVG) (R371A)
Cocal virus glycoprotein (CVG) (K64Q) (R371A)

TABLE 2
Exemplary Potential Cas9 and Cas12a orthologs

| DNA-binding Cas | | | |
|---|---|---|---|
| ortholog | Enzyme class | Nickase mutation | CI mutations |
| SpCas9 | Type II-A | D10A | D10A, H840A |
| SaCas9 | Type II-A | D10A | D10A, |
| CjCas9 | Type II-C | D8A | D8A, |
| NmeCas9 | Type II-C | D16A | D16A, H588A |
| asCas12a | Type II-C | | D908A, E993A |
| lbCas12a | Type II-C | | D832A, E925A |

Nickase mutation residues represents a position of the enzyme either known to be required for catalytic activity of the conserved RuvC nuclease domain or predicted to be required for this catalytic activity based on sequence alignment to CjCas9 where structural information is lacking (* indicates which proteins lack sufficient structural information). All positional information refers to the wild-type protein sequences acquired from uniprot.org.

TABLE 3
Exemplary Deaminase domains and their
substrate sequence preferences.

| Deaminase | Nucleotide sequence preference |
|---|---|
| hAID | 5′-WRC |
| rAPOBEC1* | 5′-TC ≥ CC ≥ AC > GC |
| mAPOBEC3 | 5′-TYC |
| hAPOBEC3A | 5′-TCG |
| hAPOBEC3B | 5′-TCR > TCT |
| hAPOBEC3C | 5′-WYC |
| hAPOBEC3F | 5′-TTC |
| hAPOBEC3G | 5′-CCC |
| hAPOBEC3H | 5′-TTCA ~ TTCT ~ TTCG > ACCCA > TGCA |
| E. coli TadA | A |
| hAdar1 | A |
| hAdar2 | A |

Nucleotide positions that are poorly specified or are permissive of two or more nucleotides are annotated according to IUPAC codes, where W = A or T, R = A or G, and Y = C or T. “h” before the deaminase name indicates Homo sapiens origin. “m” before the deaminase name indicates Mus musculus origin. “r” before the deaminase name indicates Rattus origin.

TABLE 4
Exemplary Epigenetic modulator domains.

| Epigenetic modulator | Epigenetic modulation |
|---|---|
| VP16 | transcriptional activation |
| VP64 | transcriptional activation |
| P65 | transcriptional activation |
| RTA | transcriptional activation |
| KRAB | transcriptional repression |
| MeCP2 | transcriptional repression |
| TET1 | Methylation |
| DNMT3A | Methylation |

TABLE 5
Exemplary CRISPR based RNA-guided RNA binding enzymes

| RNA-binding Cas ortholog | Enzyme class |
|---|---|
| LshCas13a | Type-VI |
| LwaCas13a | Type-VI |

TABLE 6
Exemplary Plasma membrane recruitment
domains (sequences provided below)

| Plasma membrane recruitment domain | Substitution(s) |
|---|---|
| Pleckstrin homology domain of human phospholipase | |
| Cδ1 (hPLCδ1) | |
| Pleckstrin homology domain of human phospholipase | R40L⁵⁹ |
| Cδ1 (hPLCδ1) | |
| Pleckstrin homology domain of human Akt1 (hAkt1) | |
| Mutant Pleckstrin homology domain of human Akt1 | E17K⁶⁰ |
| Pleckstrin homology domain of human 3- | |
| phosphoinositide-dependent protein kinase 1 | |
| (hPDPK1) | |
| Mutant Pleckstrin homology domain of human Akt1 | K14R⁶³ |
| Mutant Pleckstrin homology domain of human Akt1 | K8R⁶⁴ |
| Mutant Pleckstrin homology domain of human Akt1 | T72A⁶⁵ |
| Mutant Pleckstrin homology domain of human Akt1 | T92A⁶⁶ |
| Mutant Pleckstrin homology domain of human Akt1 | R25C⁵⁹ |
| Mutant Pleckstrin homology domain of human Akt1 | T34D⁶¹ |
| Mutant Pleckstrin homology domain of human Akt1 | T34F⁶¹ |
| Mutant Pleckstrin homology domain of human Akt1 | T34L⁶¹ |
| Mutant Pleckstrin homology domain of human Akt1 | T81Y⁶² |
| Mutant Pleckstrin homology domain of human Akt1 | K142A, H143A, |
| | R144A⁶⁷ |
| Mutant Pleckstrin homology domain of human Akt1 | T101C⁶⁸ |
| Pleckstrin homology domain of Human Dapp1 | |
| Pleckstrin homology domain of Human GRP1 | |
| Pleckstrin homology domain of Human GRP1 | R284C⁵⁹ |
| Pleckstrin homology domain of Human OSBP1 | |
| Pleckstrin homology domain of Human OSBP1 | R108E⁵⁹ |
| Pleckstrin homology domain of Human ARNO | |
| (CYTH2) | |
| Pleckstrin homology domain of Human ARNO | R279C⁵⁹ |
| (CYTH2) | |
| Pleckstrin homology domain of Human Btk1 | |
| Pleckstrin homology domain of Human Btk1 | R28C⁵⁹ |
| FYVE domain of Human EEA1 | |
| FYVE domain of Human EEA1 | R1375L⁵⁹ |
| PX domain of p40^{phox} (NCF4) | |
| PX domain of p40^{phox} (NCF4) | R58L⁵⁹ |
| Pleckstrin homology domain of Human FAPP1 | |
| Pleckstrin homology domain of Human CERT | |
| Pleckstrin homology domain of Human PHLPP1 | |
| Pleckstrin homology domain of Human SWAP70 | |
| Pleckstrin homology domain of Human SWAP70 | R223E and |
| | R224E⁶⁹ |
| Pleckstrin Homology Domain of Human PKD | |
| Pleckstrin homology domain of Human MAPKAP1 | |
| Pleckstrin homology domain of Human Son Of | |
| Sevenless Homolog 2 | |
| Pleckstrin homology domain of Human Dynamin | |
| Pleckstrin homology domain of Human BCR | |
| Pleckstrin homology domain of Human DBS | |

### Exemplary Sequences

In some embodiments, the sequence of a protein or nucleic acid used in a composition or method described herein is at least 80%, 85%, 90%, 95%, 97%, 98%, or 99% identical to a sequence set forth herein. To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 80% of the length of the reference sequence, and in some embodiments is at least 90% or 100%. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. For example, the percent identity between two amino acid sequences can be determined using the Needleman and Wunsch ((1970) J. Mol. Biol. 48444-453) algorithm which has been incorporated into the GAP program in the GCG software package (available on the world wide web at gcg.com), using the default parameters, e.g., a Blossum 62 scoring matrix with a gap penalty of 12, a gap extend penalty of 4, and a frameshift gap penalty of 5.

Prime Editor spCas9 H840A-MMLV Reverse Transcriptase (delta RNase H
domain)
(SEQ ID NO: 15)
MKRTADGSEFESPKKKRKVDKKYSIGLDIGTNSVGWAVITDEYKVPSKKFKVLGNT
DRHSIKKNLIGALLFDSGETAEATRLKRTARRRYTRRKNRICYLQEIFSNEMAKVDDS
FFHRLEESFLVEEDKKHERHPIFGNIVDEVAYHEKYPTIYHLRKKLVDSTDKADLRLIY
LALAHMIKFRGHFLIEGDLNPDNSDVDKLFIQLVQTYNQLFEENPINASGVDAKAILS
ARLSKSRRLENLIAQLPGEKKNGLFGNLIALSLGLTPNFKSNFDLAEDAKLQLSKDTY
DDDLDNLLAQIGDQYADLFLAAKNLSDAILLSDILRVNTEITKAPLSASMIKRYDEHHQ
DLTLLKALVRQQLPEKYKEIFFDQSKNGYAGYIDGGASQEEFYKFIKPILEKMDGTEE
LLVKLNREDLLRKQRTFDNGSIPHQIHLGELHAILRRQEDFYPFLKDNREKIEKILTFRI
PYYVGPLARGNSRFAWMTRKSEETITPWNFEEVVDKGASAQSFIERMTNFDKNLP
NEKVLPKHSLLYEYFTVYNELTKVKYVTEGMRKPAFLSGEQKKAIVDLLFKTNRKVT
VKQLKEDYFKKIECFDSVEISGVEDRFNASLGTYHDLLKIIKDKDFLDNEENEDILEDI
VLTLTLFEDREMIEERLKTYAHLFDDKVMKQLKRRRYTGWGRLSRKLINGIRDKQSG
KTILDFLKSDGFANRNFMQLIHDDSLTFKEDIQKAQVSGQGDSLHEHIANLAGSPAIK
KGILQTVKVVDELVKVMGRHKPENIVIEMARENQTTQKGQKNSRERMKRIEEGIKEL
GSQILKEHPVENTQLQNEKLYLYYLQNGRDMYVDQELDINRLSDYDVDAIVPQSFLK
DDSIDNKVLTRSDKNRGKSDNVPSEEVVKKMKNYWRQLLNAKLITQRKFDNLTKAE
RGGLSELDKAGFIKRQLVETRQITKHVAQILDSRMNTKYDENDKLIREVKVITLKSKL
VSDFRKDFQFYKVREINNYHHAHDAYLNAVVGTALIKKYPKLESEFVYGDYKVYDVR
KMIAKSEQEIGKATAKYFFYSNIMNFFKTEITLANGEIRKRPLIETNGETGEIVWDKGR
DFATVRKVLSMPQVNIVKKTEVQTGGFSKESILPKRNSDKLIARKKDWDPKKYGGF
DSPTVAYSVLVVAKVEKGKSKKLKSVKELLGITIMERSSFEKNPIDFLEAKGYKEVKK
DLIIKLPKYSLFELENGRKRMLASAGELQKGNELALPSKYVNFLYLASHYEKLKGSPE
DNEQKQLFVEQHKHYLDEIIEQISEFSKRVILADANLDKVLSAYNKHRDKPIREQAENI
IHLFTLTNLGAPAAFKYFDTTIDRKRYTSTKEVLDATLIHQSITGLYETRIDLSQLGGD
SGGSSGGSSGSETPGTSESATPESSGGSSGGSSTLNIEDEYRLHETSKEPDVSLG
STWLSDFPQAWAETGGMGLAVRQAPLIIPLKATSTPVSIKQYPMSQEARLGIKPHIQ
RLLDQGILVPCQSPWNTPLLPVKKPGTNDYRPVQDLREVNKRVEDIHPTVPNPYNL
LSGLPPSHQWYTVLDLKDAFFCLRLHPTSQPLFAFEWRDPEMGISGQLTWTRLPQ
GFKNSPTLFNEALHRDLADFRIQHPDLILLQYVDDLLLAATSELDCQQGTRALLQTLG
NLGYRASAKKAQICQKQVKYLGYLLKEGQRWLTEARKETVMGQPTPKTPRQLREF
LGKAGFCRLFIPGFAEMAAPLYPLTKPGTLFNWGPDQQKAYQEIKQALLTAPALGLP
DLTKPFELFVDEKQGYAKGVLTQKLGPWRRPVAYLSKKLDPVAAGWPPCLRMVAAI
AVLTKDAGKLTMGQPLVILAPHAVEALVKQPPDRWLSNARMTHYQALLLDTDRVQF
GPVVALNPATLLPLPEEGLQHNCLSGGSKRTADGSEPKKKRKVGS
Rattus norvegicus & synthetic APOBEC1-XTEN L8-nspCas9-UGI-SV40 NLS
(SEQ ID NO: 16)
MSSETGPVAVDPTLRRRIEPHEFEVFFDPRELRKETCLLYEINWGGRHSIWRHTSQ
NTNKHVEVNFIEKFTTERYFCPNTRCSITWFLSWSPCGECSRAITEFLSRYPHVTLFI
YIARLYHHADPRNRQGLRDLISSGVTIQIMTEQESGYCWRNFVNYSPSNEAHWPRY
PHLWVRLYVLELYCIILGLPPCLNILRRKQPQLTFFTIALQSCHYQRLPPHILWATGLK
SGSETPGTSESATPESDKKYSIGLAIGTNSVGWAVITDEYKVPSKKFKVLGNTDRHS
IKKNLIGALLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKADLRLIYLALA
HMIKFRGHFLIEGDLNPDNSDVDKLFIQLVQTYNQLFEENPINASGVDAKAILSARLS
KSRRLENLIAQLPGEKKNGLFGNLIALSLGLTPNFKSNFDLAEDAKLQLSKDTYDDDL
DNLLAQIGDQYADLFLAAKNLSDAILLSDILRVNTEITKAPLSASMIKRYDEHHQDLTL
LKALVRQQLPEKYKEIFFDQSKNGYAGYIDGGASQEEFYKFIKPILEKMDGTEELLVK
LNREDLLRKQRTFDNGSIPHQIHLGELHAILRRQEDFYPFLKDNREKIEKILTFRIPYY
VGPLARGNSRFAWMTRKSEETITPWNFEEVVDKGASAQSFIERMTNFDKNLPNEK
VLPKHSLLYEYFTVYNELTKVKYVTEGMRKPAFLSGEQKKAIVDLLFKTNRKVTVKQ
LKEDYFKKIECFDSVEISGVEDRFNASLGTYHDLLKIIKDKDFLDNEENEDILEDIVLTL
TLFEDREMIEERLKTYAHLFDDKVMKQLKRRRYTGWGRLSRKLINGIRDKQSGKTIL
DFLKSDGFANRNFMQLIHDDSLTFKEDIQKAQVSGQGDSLHEHIANLAGSPAIKKGIL
QTVKVVDELVKVMGRHKPENIVIEMARENQTTQKGQKNSRERMKRIEEGIKELGSQI
LKEHPVENTQLQNEKLYLYYLQNGRDMYVDQELDINRLSDYDVDHIVPQSFLKDDSI
DNKVLTRSDKNRGKSDNVPSEEVVKKMKNYWRQLLNAKLITQRKFDNLTKAERGG
LSELDKAGFIKRQLVETRQITKHVAQILDSRMNTKYDENDKLIREVKVITLKSKLVSDF
RKDFQFYKVREINNYHHAHDAYLNAVVGTALIKKYPKLESEFVYGDYKVYDVRKMIA
KSEQEIGKATAKYFFYSNIMNFFKTEITLANGEIRKRPLIETNGETGEIVWDKGRDFA
TVRKVLSMPQVNIVKKTEVQTGGFSKESILPKRNSDKLIARKKDWDPKKYGGFDSP
TVAYSVLVVAKVEKGKSKKLKSVKELLGITIMERSSFEKNPIDFLEAKGYKEVKKDLII
KLPKYSLFELENGRKRMLASAGELQKGNELALPSKYVNFLYLASHYEKLKGSPEDN
EQKQLFVEQHKHYLDEIIEQISEFSKRVILADANLDKVLSAYNKHRDKPIREQAENIIH
LFTLTNLGAPAAFKYFDTTIDRKRYTSTKEVLDATLIHQSITGLYETRIDLSQLGGDSG
GSTNLSDIIEKETGKQLVIQESILMLPEEVEEVIGNKPESDILVHTAYDESTDENVMLL
TSDAPEYKPWALVIQDSNGENKIKMLSGGSPKKKRKV
Homo sapiens AID
(SEQ ID NO: 17))
MDSLLMNRRKFLYQFKNVRWAKGRRETYLCYVVKRRDSATSFSLDFGYLRNKNGC
HVELLFLRYISDWDLDPGRCYRVTWFTSWSPCYDCARHVADFLRGNPNLSLRIFTA
RLYFCEDRKAEPEGLRRLHRAGVQIAIMTFKDYFYCWNTFVENHERTFKAWEGLHE
NSVRLSRQLRRILLPLYEVDDLRDAFRTLGL
Homo sapiensAIDv solubility variant lacking N-terminal RNA-binding region
(SEQ ID NO: 18)
LMDPHIFTSNFNNGIGRHKTYLCYEVERLDSATSFSLDFGYLRNKNGCHVELLFLRYI
SDWDLDPGRCYRVTWFTSWSPCYDCARHVADFLRGNPNLSLRIFTARLYFCEDRK
AEPEGLRRLHRAGVQIAIMTFKDYFYCWNTFVENHERTFKAWEGLHENSVRLSRQL
RRILLPLYEVDDLRDAFRTLGL
Homo sapiens AIDv solubility variant lacking N-terminal RNA-binding region
and the C-terminal poorly structured region
(SEQ ID NO: 19)
MDPHIFTSNFNNGIGRHKTYLCYEVERLDSATSFSLDFGYLRNKNGCHVELLFLRYI
SDWDLDPGRCYRVTWFTSWSPCYDCARHVADFLRGNPNLSLRIFTARLYFCEDRK
AEPEGLRRLHRAGVQIAIMTFKDYFYCWNTFVENHERTFKAWEGLHENSVRLSRQL
RRILLPL
Rattus norvegicus APOBEC1
(SEQ ID NO: 20)
MSSETGPVAVDPTLRRRIEPHEFEVFFDPRELRKETCLLYEINWGGRHSIWRHTSQ
NTNKHVEVNFIEKFTTERYFCPNTRCSITWFLSWSPCGECSRAITEFLSRYPHVTLFI
YIARLYHHADPRNRQGLRDLISSGVTIQIMTEQESGYCWRNFVNYSPSNEAHWPRY
PHLWVRLYVLELYCIILGLPPCLNILRRKQPQLTFFTIALQSCHYQRLPPHILWATGLK
Mus musculus APOBEC3
(SEQ ID NO: 21)
MGPFCLGCSHRKCYSPIRNLISQETFKFHFKNLGYAKGRKDTFLCYEVTRKDCDSP
VSLHHGVFKNKDNIHAEICFLYWFHDKVLKVLSPREEFKITWYMSWSPCFECAEQIV
RFLATHHNLSLDIFSSRLYNVQDPETQQNLCRLVQEGAQVAAMDLYEFKKCWKKFV
DNGGRRFRPWKRLLTNFRYQDSKLQEILRRMDPLSEEEFYSQFYNQRVKHLCYYH
RMKPYLCYQLEQFNGQAPLKGCLLSEKGKQHAEILFLDKIRSMELSQVTITCYLTWS
PCPNCAWQLAAFKRDRPDLILHIYTSRLYFHWKRPFQKGLCSLWQSGILVDVMDLP
QFTDCWTNFVNPKRPFRPWKGLEIISRRTQRRLRRIKESWGLQDLVNDFGNLQLGP
PMSN
Mus musculus APOBEC3 catalytic domain
(SEQ ID NO: 22)
MGPFCLGCSHRKCYSPIRNLISQETFKFHFKNLGYAKGRKDTFLCYEVTRKDCDSP
VSLHHGVFKNKDNIHAEICFLYWFHDKVLKVLSPREEFKITWYMSWSPCFECAEQIV
RFLATHHNLSLDIFSSRLYNVQDPETQQNLCRLVQEGAQVAAMDLYEFKKCWKKFV
DNGGRRFRPWKRLLTNFRYQDSKLQEILRR
Homo sapiens APOBEC3A
(SEQ ID NO: 23)
MEASPASGPRHLMDPHIFTSNFNNGIGRHKTYLCYEVERLDNGTSVKMDQHRGFL
HNQAKNLLCGFYGRHAELRFLDLVPSLQLDPAQIYRVTWFISWSPCFSWGCAGEV
RAFLQENTHVRLRIFAARIYDYDPLYKEALQMLRDAGAQVSIMTYDEFKHCWDTFV
DHQGCPFQPWDGLDEHSQALSGRLRAILQNQGN
Homo sapiens APOBEC3G
(SEQ ID NO: 24)
MKPHFRNTVERMYRDTFSYNFYNRPILSRRNTVWLCYEVKTKGPSRPPLDAKIFRG
QVYSELKYHPEMRFFHWFSKWRKLHRDQEYEVTWYISWSPCTKCTRDMATFLAE
DPKVTLTIFVARLYYFWDPDYQEALRSLCQKRDGPRATMKIMNYDEFQHCWSKFV
YSQRELFEPWNNLPKYYILLHIMLGEILRHSMDPPTFTFNFNNEPWVRGRHETYLCY
EVERMHNDTWVLLNQRRGFLCNQAPHKHGFLEGRHAELCFLDVIPFWKLDLDQDY
RVTCFTSWSPCFSCAQEMAKFISKNKHVSLCIFTARIYDDQGRCQEGLRTLAEAGA
KISIMTYSEFKHCWDTFVDHQGCPFQPWDGLDEHSQDLSGRLRAILQNQEN
Homo sapiens APOBEC3G catalytic domain
(SEQ ID NO: 25)
PPTFTFNFNNEPWVRGRHETYLCYEVERMHNDTWVLLNQRRGFLCNQAPHKHGF
LEGRHAELCFLDVIPFWKLDLDQDYRVTCFTSWSPCFSCAQEMAKFISKNKHVSLCI
FTARIYDDQGRCQEGLRTLAEAGAKISIMTYSEFKHCWDTFVDHQGCPFQPWDGL
DEHSQDLSGRLRAILQNQEN
Homo sapiens APOBEC3H
(SEQ ID NO: 26)
MALLTAETFRLQFNNKRRLRRPYYPRKALLCYQLTPQNGSTPTRGYFENKKKCHAE
ICFINEIKSMGLDETQCYQVTCYLTWSPCSSCAWELVDFIKAHDHLNLGIFASRLYYH
WCKPQQKGLRLLCGSQVPVEVMGFPKFADCWENFVDHEKPLSFNPYKMLEELDK
NSRAIKRRLERIKIPGVRAQGRYMDILCDAEV
Homo sapiens APOBEC3F
(SEQ ID NO: 27)
MKPHFRNTVERMYRDTFSYNFYNRPILSRRNTVWLCYEVKTKGPSRPRLDAKIFRG
QVYSQPEHHAEMCFLSWFCGNQLPAYKCFQITWFVSWTPCPDCVAKLAEFLAEHP
NVTLTISAARLYYYWERDYRRALCRLSQAGARVKIMDDEEFAYCWENFVYSEGQPF
MPWYKFDDNYAFLHRTLKEILRNPMEAMYPHIFYFHFKNLRKAYGRNESWLCFTME
WVKHHSPVSWKRGVFRNQVDPETHCHAERCFLSWFCDDILSPNTNYEVTWYTSW
SPCPECAGEVAEFLARHSNVNLTIFTARLYYFWDTDYQEGLRSLSQEGASVEIMGY
KDFKYCWENFVYNDDEPFKPWKGLKYNFLFLDSKLQEILE
Homo sapiens APOBEC3F catalytic domain
(SEQ ID NO: 28)
KEILRNPMEAMYPHIFYFHFKNLRKAYGRNESWLCFTMEVVKHHSPVSWKRGVFR
NQVDPETHCHAERCFLSWFCDDILSPNTNYEVTWYTSWSPCPECAGEVAEFLARH
SNVNLTIFTARLYYFWDTDYQEGLRSLSQEGASVEIMGYKDFKYCWENFVYNDDEP
FKPWKGLKYNFLFLDSKLQEILE
Escherichia coli TadA
(SEQ ID NO: 30)
MKRTADGSEFESPKKKRKVSEVEFSHEYWMRHALTLAKRAWDEREVPVGAVLVH
NNRVIGEGWNRPIGRHDPTAHAEIMALRQGGLVMQNYRLIDATLYVTLEPCVMCAG
AMIHSRIGRVVFGARDAKTGAAGSLMDVLHHPGMNHRVEITEGILADECAALLSDFF
RMRRQEIKAQKKAQSSTDSGGSSGGSSGSETPGTSESATPESSGGSSGGSSEVE
FSHEYWMRHALTLAKRARDEREVPVGAVLVLNNRVIGEGWNRAIGLHDPTAHAEIM
ALRQGGLVMQNYRLIDATLYVTFEPCVMCAGAMIHSRIGRVVFGVRNAKTGAAGSL
MDVLHYPGMNHRVEITEGILADECAALLCYFFRMPRQVFNAQKKAQSSTD
Homo sapiens Adar1
(SEQ ID NO: 31)
MNPRQGYSLSGYYTHPFQGYEHRQLRYQQPGPGSSPSSFLLKQIEFLKGQLPEAP
VIGKQTPSLPPSLPGLRPRFPVLLASSTRGRQVDIRGVPRGVHLGSQGLQRGFQHP
SPRGRSLPQRGVDCLSSHFQELSIYQDQEQRILKFLEELGEGKATTAHDLSGKLGT
PKKEINRVLYSLAKKGKLQKEAGTPPLWKIAVSTQAWNQHSGVVRPDGHSQGAPN
SDPSLEPEDRNSTSVSEDLLEPFIAVSAQAWNQHSGVVRPDSHSQGSPNSDPGLE
PEDSNSTSALEDPLEFLDMAEIKEKICDYLFNVSDSSALNLAKNIGLTKARDINAVLID
MERQGDVYRQGTTPPIWHLTDKKRERMQIKRNTNSVPETAPAAIPETKRNAEFLTC
NIPTSNASNNMVTTEKVENGQEPVIKLENRQEARPEPARLKPPVHYNGPSKAGYVD
FENGQWATDDIPDDLNSIRAAPGEFRAIMEMPSFYSHGLPRCSPYKKLTECQLKNPI
SGLLEYAQFASQTCEFNMIEQSGPPHEPRFKFQVVINGREFPPAEAGSKKVAKQDA
AMKAMTILLEEAKAKDSGKSEESSHYSTEKESEKTAESQTPTPSATSFFSGKSPVTT
LLECMHKLGNSCEFRLLSKEGPAHEPKFQYCVAVGAQTFPSVSAPSKKVAKQMAA
EEAMKALHGEATNSMASDNQPEGMISESLDNLESMMPNKVRKIGELVRYLNTNPV
GGLLEYARSHGFAAEFKLVDQSGPPHEPKFVYQAKVGGRWFPAVCAHSKKQGKQ
EAADAALRVLIGENEKAERMGFTEVTPVTGASLRRTMLLLSRSPEAQPKTLPLTGST
FHDQIAMLSHRCFNTLTNSFQPSLLGRKILAAIIMKKDSEDMGVVVSLGTGNRCVKG
DSLSLKGETVNDCHAEIISRRGFIRFLYSELMKYNSQTAKDSIFEPAKGGEKLQIKKT
VSFHLYISTAPCGDGALFDKSCSDRAMESTESRHYPVFENPKQGKLRTKVENGEGT
IPVESSDIVPTWDGIRLGERLRTMSCSDKILRWNVLGLQGALLTHFLQPIYLKSVTLG
YLFSQGHLTRAICCRVTRDGSAFEDGLRHPFIVNHPKVGRVSIYDSKRQSGKTKETS
VNWCLADGYDLEILDGTRGTVDGPRNELSRVSKKNIFLLFKKLCSFRYRRDLLRLSY
GEAKKAARDYETAKNYFKKGLKDMGYGNWISKPQEEKNFYLCPV
Homo sapiens Adar2
(SEQ ID NO: 32)
MDIEDEENMSSSSTDVKENRNLDNVSPKDGSTPGPGEGSQLSNGGGGGPGRKRP
LEEGSNGHSKYRLKKRRKTPGPVLPKNALMQLNEIKPGLQYTLLSQTGPVHAPLFV
MSVEVNGQVFEGSGPTKKKAKLHAAEKALRSFVQFPNASEAHLAMGRTLSVNTDF
TSDQADFPDTLFNGFETPDKAEPPFYVGSNGDDSFSSSGDLSLSASPVPASLAQPP
LPVLPPFPPPSGKNPVMILNELRPGLKYDFLSESGESHAKSFVMSVVVDGQFFEGS
GRNKKLAKARAAQSALAAIFNLHLDQTPSRQPIPSEGLQLHLPQVLADAVSRLVLGK
FGDLTDNFSSPHARRKVLAGVVMTTGTDVKDAKVISVSTGTKCINGEYMSDRGLAL
NDCHAEIISRRSLLRFLYTQLELYLNNKDDQKRSIFQKSERGGFRLKENVQFHLYIST
SPCGDARIFSPHEPILEEPADRHPNRKARGQLRTKIESGQGTIPVRSNASIQTWDGV
LQGERLLTMSCSDKIARWNVVGIQGSLLSIFVEPIYFSSIILGSLYHGDHLSRAMYQRI
SNIEDLPPLYTLNKPLLSGISNAEARQPGKAPNFSVNWTVGDSAIEVINATTGKDELG
RASRLCKHALYCRWMRVHGKVPSHLLRSKITKPNVYHESKLAAKEYQAAKARLFTA
FIKAGLGAWVEKPTEQDQFSLTP
Streptococcus pyogenes Cas9 Bipartite NLS
(SEQ ID NO: 33)
MDKKYSIGLDIGTNSVGWAVITDEYKVPSKKFKVLGNTDRHSIKKNLIGALLF
DSGETAEATRLKRTARRRYTRRKNRICYLQEIFSNEMAKVDDSFFHRLEESF
LVEEDKKHERHPIFGNIVDEVAYHEKYPTIYHLRKKLVDSTDKADLRLIYLALA
HMIKFRGHFLIEGDLNPDNSDVDKLFIQLVQTYNQLFEENPINASGVDAKAIL
SARLSKSRRLENLIAQLPGEKKNGLFGNLIALSLGLTPNFKSNFDLAEDAKLQ
LSKDTYDDDLDNLLAQIGDQYADLFLAAKNLSDAILLSDILRVNTEITKAPLSA
SMIKRYDEHHQDLTLLKALVRQQLPEKYKEIFFDQSKNGYAGYIDGGASQEE
FYKFIKPILEKMDGTEELLVKLNREDLLRKQRTFDNGSIPHQIHLGELHAILRR
QEDFYPFLKDNREKIEKILTFRIPYYVGPLARGNSRFAWMTRKSEETITPWNF
EEVVDKGASAQSFIERMTNFDKNLPNEKVLPKHSLLYEYFTVYNELTKVKYV
TEGMRKPAFLSGEQKKAIVDLLFKTNRKVTVKQLKEDYFKKIECFDSVEISGV
EDRFNASLGTYHDLLKIIKDKDFLDNEENEDILEDIVLTLTLFEDREMIEERLKT
YAHLFDDKVMKQLKRRRYTGWGRLSRKLINGIRDKQSGKTILDFLKSDGFAN
RNFMQLIHDDSLTFKEDIQKAQVSGQGDSLHEHIANLAGSPAIKKGILQTVKV
VDELVKVMGRHKPENIVIEMARENQTTQKGQKNSRERMKRIEEGIKELGSQI
LKEHPVENTQLQNEKLYLYYLQNGRDMYVDQELDINRLSDYDVDHIVPQSFL
KDDSIDNKVLTRSDKNRGKSDNVPSEEVVKKMKNYWRQLLNAKLITQRKFD
NLTKAERGGLSELDKAGFIKRQLVETRQITKHVAQILDSRMNTKYDENDKLIR
EVKVITLKSKLVSDFRKDFQFYKVREINNYHHAHDAYLNAVVGTALIKKYPKL
ESEFVYGDYKVYDVRKMIAKSEQEIGKATAKYFFYSNIMNFFKTEITLANGEI
RKRPLIETNGETGEIVWDKGRDFATVRKVLSMPQVNIVKKTEVQTGGFSKES
ILPKRNSDKLIARKKDWDPKKYGGFDSPTVAYSVLVVAKVEKGKSKKLKSVK
ELLGITIMERSSFEKNPIDFLEAKGYKEVKKDLIIKLPKYSLFELENGRKRMLA
SAGELQKGNELALPSKYVNFLYLASHYEKLKGSPEDNEQKQLFVEQHKHYL
DEIIEQISEFSKRVILADANLDKVLSAYNKHRDKPIREQAENIIHLFTLTNLGAP
AAFKYFDTTIDRKRYTSTKEVLDATLIHQSITGLYETRIDLSQLGGDGSGGGG
SGKRTADGSEFEPKKKRKVSSGGDYKDHDGDYKDHDIDYKDDDDK
Staphylococcus aureus Cas9
(SEQ ID NO: 34)
MKRNYILGLDIGITSVGYGIIDYETRDVIDAGVRLFKEANVENNEGRRSKRGA
RRLKRRRRHRIQRVKKLLFDYNLLTDHSELSGINPYEARVKGLSQKLSEEEF
SAALLHLAKRRGVHNVNEVEEDTGNELSTKEQISRNSKALEEKYVAELQLER
LKKDGEVRGSINRFKTSDYVKEAKQLLKVQKAYHQLDQSFIDTYIDLLETRRT
YYEGPGEGSPFGWKDIKEWYEMLMGHCTYFPEELRSVKYAYNADLYNALN
DLNNLVITRDENEKLEYYEKFQIIENVFKQKKKPTLKQIAKEILVNEEDIKGYRV
TSTGKPEFTNLKVYHDIKDITARKEIIENAELLDQIAKILTIYQSSEDIQEELTNL
NSELTQEEIEQISNLKGYTGTHNLSLKAINLILDELWHTNDNQIAIFNRLKLVPK
KVDLSQQKEIPTTLVDDFILSPVVKRSFIQSIKVINAIIKKYGLPNDIIIELAREKN
SKDAQKMINEMQKRNRQTNERIEEIIRTTGKENAKYLIEKIKLHDMQEGKCLY
SLEAIPLEDLLNNPFNYEVDHIIPRSVSFDNSFNNKVLVKQEENSKKGNRTPF
QYLSSSDSKISYETFKKHILNLAKGKGRISKTKKEYLLEERDINRFSVQKDFIN
RNLVDTRYATRGLMNLLRSYFRVNNLDVKVKSINGGFTSFLRRKWKFKKER
NKGYKHHAEDALIIANADFIFKEWKKLDKAKKVMENQMFEEKQAESMPEIET
EQEYKEIFITPHQIKHIKDFKDYKYSHRVDKKPNRELINDTLYSTRKDDKGNTL
IVNNLNGLYDKDNDKLKKLINKSPEKLLMYHHDPQTYQKLKLIMEQYGDEKN
PLYKYYEETGNYLTKYSKKDNGPVIKKIKYYGNKLNAHLDITDDYPNSRNKVV
KLSLKPYRFDVYLDNGVYKFVTVKNLDVIKKENYYEVNSKCYEEAKKLKKISN
QAEFIASFYNNDLIKINGELYRVIGVNNDLLNRIEVNMIDITYREYLENMNDKR
PPRIIKTIASKTQSIKKYSTDILGNLYEVKSKKHPQIIKKG
Campylobacter jejuni Cas9
(SEQ ID NO: 35)
MARILAFDIGISSIGWAFSENDELKDCGVRIFTKVENPKTGESLALPRRLARS
ARKRLARRKARLNHLKHLIANEFKLNYEDYQSFDESLAKAYKGSLISPYELRF
RALNELLSKQDFARVILHIAKRRGYDDIKNSDDKEKGAILKAIKQNEEKLANY
QSVGEYLYKEYFQKFKENSKEFTNVRNKKESYERCIAQSFLKDELKLIFKKQ
REFGFSFSKKFEEEVLSVAFYKRALKDFSHLVGNCSFFTDEKRAPKNSPLAF
MFVALTRIINLLNNLKNTEGILYTKDDLNALLNEVLKNGTLTYKQTKKLLGLSD
DYEFKGEKGTYFIEFKKYKEFIKALGEHNLSQDDLNEIAKDITLIKDEIKLKKAL
AKYDLNQNQIDSLSKLEFKDHLNISFKALKLVTPLMLEGKKYDEACNELNLKV
AINEDKKDFLPAFNETYYKDEVTNPVVLRAIKEYRKVLNALLKKYGKVHKINIE
LAREVGKNHSQRAKIEKEQNENYKAKKDAELECEKLGLKINSKNILKLRLFKE
QKEFCAYSGEKIKISDLQDEKMLEIDHIYPYSRSFDDSYMNKVLVFTKQNQE
KLNQTPFEAFGNDSAKWQKIEVLAKNLPTKKQKRILDKNYKDKEQKNFKDR
NLNDTRYIARLVLNYTKDYLDFLPLSDDENTKLNDTQKGSKVHVEAKSGMLT
SALRHTWGFSAKDRNNHLHHAIDAVIIAYANNSIVKAFSDFKKEQESNSAELY
AKKISELDYKNKRKFFEPFSGFRQKVLDKIDEIFVSKPERKKPSGALHEETFR
KEEEFYQSYGGKEGVLKALELGKIRKVNGKIVKNGDMFRVDIFKHKKTNKFY
AVPIYTMDFALKVLPNKAVARSKKGEIKDWILMDENYEFCFSLYKDSLILIQTK
DMQEPEFVYYNAFTSSTVSLIVSKHDNKFETLSKNQKILFKNANEKEVIAKSI
GIQNLKVFEKYIVSALGEVTKAEFRQREDFKK
Neisseria meningitidis Cas9
(SEQ ID NO: 36)
MAAFKPNSINYILGLDIGIASVGWAMVEIDEEENPIRLIDLGVRVFERAEVPKT
GDSLAMARRLARSVRRLTRRRAHRLLRTRRLLKREGVLQAANFDENGLIKS
LPNTPWQLRAAALDRKLTPLEWSAVLLHLIKHRGYLSQRKNEGETADKELG
ALLKGVAGNAHALQTGDFRTPAELALNKFEKESGHIRNQRSDYSHTFSRKD
LQAELILLFEKQKEFGNPHVSGGLKEGIETLLMTQRPALSGDAVQKMLGHCT
FEPAEPKAAKNTYTAERFIWLTKLNNLRILEQGSERPLTDTERATLMDEPYR
KSKLTYAQARKLLGLEDTAFFKGLRYGKDNAEASTLMEMKAYHAISRALEKE
GLKDKKSPLNLSPELQDEIGTAFSLFKTDEDITGRLKDRIQPEILEALLKHISFD
KFVQISLKALRRIVPLMEQGKRYDEACAEIYGDHYGKKNTEEKIYLPPIPADEI
RNPVVLRALSQARKVINGWVRRYGSPARIHIETAREVGKSFKDRKEIEKRQE
ENRKDREKAAAKFREYFPNFVGEPKSKDILKLRLYEQQHGKCLYSGKEINLG
RLNEKGYVEIDHALPFSRTWDDSFNNKVLVLGSENQNKGNQTPYEYFNGK
DNSREWQEFKARVETSRFPRSKKQRILLQKFDEDGFKERNLNDTRYVNRFL
CQFVADRMRLTGKGKKRVFASNGQITNLLRGFWGLRKVRAENDRHHALDA
WVVACSTVAMQQKITRFVRYKEMNAFDGKTIDKETGEVLHQKTHFPQPWEF
FAQEVMIRVFGKPDGKPEFEEADTLEKLRTLLAEKLSSRPEAVHEYVTPLFV
SRAPNRKMSGQGHMETVKSAKRLDEGVSVLRVPLTQLKLKDLEKMVNRER
EPKLYEALKARLEAHKDDPAKAFAEPFYKYDKAGNRTQQVKAVRVEQVQKT
GVWVRNHNGIADNATMVRVDVFEKGDKYYLVPIYSWQVAKGILPDRAVVQ
GKDEEDWQLIDDSFNFKFSLHPNDLVEVITKKARMFGYFASCHRGTGNINIRI
HDLDHKIGKNGILEGIGVKTALSFQKYQIDELGKEIRPCRLKKRPPVR
Acidaminococcus sp. Cas12a
(SEQ ID NO: 37)
MTQFEGFTNLYQVSKTLRFELIPQGKTLKHIQEQGFIEEDKARNDHYKELKPII
DRIYKTYADQCLQLVQLDWENLSAAIDSYRKEKTEETRNALIEEQATYRNAIH
DYFIGRTDNLTDAINKRHAEIYKGLFKAELFNGKVLKQLGTVTTTEHENALLR
SFDKFTTYFSGFYENRKNVFSAEDISTAIPHRIVQDNFPKFKENCHIFTRLITA
VPSLREHFENVKKAIGIFVSTSIEEVFSFPFYNQLLTQTQIDLYNQLLGGISRE
AGTEKIKGLNEVLNLAIQKNDETAHIIASLPHRFIPLFKQILSDRNTLSFILEEFK
SDEEVIQSFCKYKTLLRNENVLETAEALFNELNSIDLTHIFISHKKLETISSALC
DHWDTLRNALYERRISELTGKITKSAKEKVQRSLKHEDINLQEIISAAGKELSE
AFKQKTSEILSHAHAALDQPLPTTLKKQEEKEILKSQLDSLLGLYHLLDWFAV
DESNEVDPEFSARLTGIKLEMEPSLSFYNKARNYATKKPYSVEKFKLNFQMP
TLASGWDVNKEKNNGAILFVKNGLYYLGIMPKQKGRYKALSFEPTEKTSEGF
DKMYYDYFPDAAKMIPKCSTQLKAVTAHFQTHTTPILLSNNFIEPLEITKEIYD
LNNPEKEPKKFQTAYAKKTGDQKGYREALCKWIDFTRDFLSKYTKTTSIDLS
SLRPSSQYKDLGEYYAELNPLLYHISFQRIAEKEIMDAVETGKLYLFQIYNKD
FAKGHHGKPNLHTLYWTGLFSPENLAKTSIKLNGQAELFYRPKSRMKRMAH
RLGEKMLNKKLKDQKTPIPDTLYQELYDYVNHRLSHDLSDEARALLPNVITK
EVSHEIIKDRRFTSDKFFFHVPITLNYQAANSPSKFNQRVNAYLKEHPETPIIG
IDRGERNLIYITVIDSTGKILEQRSLNTIQQFDYQKKLDNREKERVAARQAWS
WVGTIKDLKQGYLSQVIHEIVDLMIHYQAVVVLENLNFGFKSKRTGIAEKAVY
QQFEKMLIDKLNCLVLKDYPAEKVGGVLNPYQLTDQFTSFAKMGTQSGFLF
YVPAPYTSKIDPLTGFVDPFVWKTIKNHESRKHFLEGFDFLHYDVKTGDFILH
FKMNRNLSFQRGLPGFMPAWDIVFEKNETQFDAKGTPFIAGKRIVPVIENHR
FTGRYRDLYPANELIALLEEKGIVFRDGSNILPKLLENDDSHAIDTMVALIRSV
LQMRNSNAATGEDYINSPVRDLNGVCFDSRFQNPEWPMDADANGAYHIAL
KGQLLLNHLKESKDLKLQNGISNQDWLAYIQELRN
Lachnospiraceae bacterium Cas12a
(SEQ ID NO: 38)
MSKLEKFTNCYSLSKTLRFKAIPVGKTQENIDNKRLLVEDEKRAEDYKGVKK
LLDRYYLSFINDVLHSIKLKNLNNYISLFRKKTRTEKENKELENLEINLRKEIAK
AFKGNEGYKSLFKKDIIETILPEFLDDKDEIALVNSFNGFTTAFTGFFDNRENM
FSEEAKSTSIAFRCINENLTRYISNMDIFEKVDAIFDKHEVQEIKEKILNSDYDV
EDFFEGEFFNFVLTQEGIDVYNAIIGGFVTESGEKIKGLNEYINLYNQKTKQKL
PKFKPLYKQVLSDRESLSFYGEGYTSDEEVLEVFRNTLNKNSEIFSSIKKLEK
LFKNFDEYSSAGIFVKNGPAISTISKDIFGEWNVIRDKWNAEYDDIHLKKKAV
VTEKYEDDRRKSFKKIGSFSLEQLQEYADADLSVVEKLKEIIIQKVDEIYKVYG
SSEKLFDADFVLEKSLKKNDAVVAIMKDLLDSVKSFENYIKAFFGEGKETNR
DESFYGDFVLAYDILLKVDHIYDAIRNYVTQKPYSKDKFKLYFQNPQFMGGW
DKDKETDYRATILRYGSKYYLAIMDKKYAKCLQKIDKDDVNGNYEKINYKLLP
GPNKMLPKVFFSKKWMAYYNPSEDIQKIYKNGTFKKGDMFNLNDCHKLIDF
FKDSISRYPKWSNAYDFNFSETEKYKDIAGFYREVEEQGYKVSFESASKKEV
DKLVEEGKLYMFQIYNKDFSDKSHGTPNLHTMYFKLLFDENNHGQIRLSGG
AELFMRRASLKKEELVVHPANSPIANKNPDNPKKTTTLSYDVYKDKRFSEDQ
YELHIPIAINKCPKNIFKINTEVRVLLKHDDNPYVIGIDRGERNLLYIVVVDGKG
NIVEQYSLNEIINNFNGIRIKTDYHSLLDKKEKERFEARQNWTSIENIKELKAG
YISQWVHKICELVEKYDAVIALEDLNSGFKNSRVKVEKQVYQKFEKMLIDKLN
YMVDKKSNPCATGGALKGYQITNKFESFKSMSTQNGFIFYIPAWLTSKIDPS
TGFVNLLKTKYTSIADSKKFISSFDRIMYVPEEDLFEFALDYKNFSRTDADYIK
KWKLYSYGNRIRIFRNPKKNNVFDWEEVCLTSAYKELFNKYGINYQQGDIRA
LLCEQSDKAFYSSFMALMSLMLQMRNSITGRTDVDFLISPVKNSDGIFYDSR
NYEAQENAILPKNADANGAYNIARKVLWAIGQFKKAEDEKLDKVKIAISNKEW
LEYAQTSVKH
Leptotrichia shahii Cas13a
(SEQ ID NO: 39)
MGNLFGHKRWYEVRDKKDFKIKRKVKVKRNYDGNKYILNINENNNKEKIDN
NKFIRKYINYKKNDNILKEFTRKFHAGNILFKLKGKEGIIRIENNDDFLETEEVV
LYIEAYGKSEKLKALGITKKKIIDEAIRQGITKDDKKIEIKRQENEEEIEIDIRDEY
TNKTLNDCSIILRIIENDELETKKSIYEIFKNINMSLYKIIEKIIENETEKVFENRYY
EEHLREKLLKDDKIDVILTNFMEIREKIKSNLEILGFVKFYLNVGGDKKKSKNK
KMLVEKILNINVDLTVEDIADFVIKELEFWNITKRIEKVKKVNNEFLEKRRNRT
YIKSYVLLDKHEKFKIERENKKDKIVKFFVENIKNNSIKEKIEKILAEFKIDELIKK
LEKELKKGNCDTEIFGIFKKHYKVNFDSKKFSKKSDEEKELYKIIYRYLKGRIE
KILVNEQKVRLKKMEKIEIEKILNESILSEKILKRVKQYTLEHIMYLGKLRHNDID
MTTVNTDDFSRLHAKEELDLELITFFASTNMELNKIFSRENINNDENIDFFGG
DREKNYVLDKKILNSKIKIIRDLDFIDNKNNITNNFIRKFTKIGTNERNRILHAISK
ERDLQGTQDDYNKVINIIQNLKISDEEVSKALNLDVVFKDKKNIITKINDIKISEE
NNNDIKYLPSFSKVLPEILNLYRNNPKNEPFDTIETEKIVLNALIYVNKELYKKLI
LEDDLEENESKNIFLQELKKTLGNIDEIDENIIENYYKNAQISASKGNNKAIKKY
QKKVIECYIGYLRKNYEELFDFSDFKMNIQEIKKQIKDINDNKTYERITVKTSD
KTIVINDDFEYIISIFALLNSNAVINKIRNRFFATSVWLNTSEYQNIIDILDEIMQL
NTLRNECITENWNLNLEEFIQKMKEIEKDFDDFKIQTKKEIFNNYYEDIKNNILT
EFKDDINGCDVLEKKLEKIVIFDDETKFEIDKKSNILQDEQRKLSNINKKDLKK
KVDQYIKDKDQEIKSKILCRIIFNSDFLKKYKKEIDNLIEDMESENENKFQEIYY
PKERKNELYIYKKNLFLNIGNPNFDKIYGLISNDIKMADAKFLFNIDGKNIRKNK
ISEIDAILKNLNDKLNGYSKEYKEKYIKKLKENDDFFAKNIQNKNYKSFEKDYN
RVSEYKKIRDLVEFNYLNKIESYLIDINWKLAIQMARFERDMHYIVNGLRELGII
KLSGYNTGISRAYPKRNGSDGFYTTTAYYKFFDEESYKKFEKICYGFGIDLSE
NSEINKPENESIRNYISHFYIVRNPFADYSIAEQIDRVSNLLSYSTRYNNSTYA
SVFEVFKKDVNLDYDELKKKFKLIGNNDILERLMKPKKVSVLELESYNSDYIK
NLIIELLTKIENTNDTL
Leptotrichia wadei Cas13a
(SEQ ID NO: 40)
MKVTKVDGISHKKYIEEGKLVKSTSEENRTSERLSELLSIRLDIYIKNPDNASE
EENRIRRENLKKFFSNKVLHLKDSVLYLKNRKEKNAVQDKNYSEEDISEYDL
KNKNSFSVLKKILLNEDVNSEELEIFRKDVEAKLNKINSLKYSFEENKANYQKI
NENNVEKVGGKSKRNIIYDYYRESAKRNDYINNVQEAFDKLYKKEDIEKLFFL
IENSKKHEKYKIREYYHKIIGRKNDKENFAKIIYEEIQNVNNIKELIEKIPDMSEL
KKSQVFYKYYLDKEELNDKNIKYAFCHFVEIEMSQLLKNYVYKRLSNISNDKI
KRIFEYQNLKKLIENKLLNKLDTYVRNCGKYNYYLQVGEIATSDFIARNRQNE
AFLRNIIGVSSVAYFSLRNILETENENGITGRMRGKTVKNNKGEEKYVSGEV
DKIYNENKQNEVKENLKMFYSYDFNMDNKNEIEDFFANIDEAISSIRHGIVHF
NLELEGKDIFAFKNIAPSEISKKMFQNEINEKKLKLKIFKQLNSANVFNYYEKD
VIIKYLKNTKFNFVNKNIPFVPSFTKLYNKIEDLRNTLKFFWSVPKDKEEKDAQ
IYLLKNIYYGEFLNKFVKNSKVFFKITNEVIKINKQRNQKTGHYKYQKFENIEK
TVPVEYLAIIQSREMINNQDKEEKNTYIDFIQQIFLKGFIDYLNKNNLKYIESNN
NNDNNDIFSKIKIKKDNKEKYDKILKNYEKHNRNKEIPHEINEFVREIKLGKILK
YTENLNMFYLILKLLNHKELTNLKGSLEKYQSANKEETFSDELELINLLNLDNN
RVTEDFELEANEIGKFLDFNENKIKDRKELKKFDTNKIYFDGENIIKHRAFYNI
KKYGMLNLLEKIADKAKYKISLKELKEYSNKKNEIEKNYTMQQNLHRKYARP
KKDEKFNDEDYKEYEKAIGNIQKYTHLKNKVEFNELNLLQGLLLKILHRLVGY
TSIWERDLRFRLKGEFPENHYIEEIFNFDNSKNVKYKSGQIVEKYINFYKELY
KDNVEKRSIYSDKKVKKLKQEKKDLYIRNYIAHFNYIPHAEISLLEVLENLRKLL
SYDRKLKNAIMKSIVDILKEYGFVATFKIGADKKIEIQTLESEKIVHLKNLKKKK
LMTDRNSEELCELVKVMFEYKALE
Pleckstrin homology domain of Human ARNO
(SEQ ID NO: 41)
NPDREGWLLKLGGGRVKTWKRRWFILTDNCLYYFEYTTDKEPRGIIPLENLS
IREVDDPRKPNCFELYIPNNKGQLIKACKTEADGRVVEGNHMVYRISAPTQE
EKDEWIKSIQAAVS
Pleckstrin homology domain of Human ARNO R279C
(SEQ ID NO: 42)
NPDREGWLLKLGGGRVKTWKCRWFILTDNCLYYFEYTTDKEPRGIIPLENLS
IREVDDPRKPNCFELYIPNNKGQLIKACKTEADGRVVEGNHMVYRISAPTQE
EKDEWIKSIQAAVS
FYVE domain of Human EEA1
(SEQ ID NO: 43)
DNEVQNCMACGKGFSVTVRRHHCRQCGNIFCAECSAKNALTPSSKKPVRV
CDACFNDLQ
FYVE domain of Human EEA1 R1375L
(SEQ ID NO: 44)
DNEVQNCMACGKGFSVTVRRHHCLQCGNIFCAECSAKNALTPSSKKPVRV
CDACFNDLQ
PX domain of p40phox (NCF4)
(SEQ ID NO: 45)
DVAISANIADIEEKRGFTSHFVFVIEVKTKGGSKYLIYRRYRQFHALQSKLEER
FGPDSKSSALACTLPTLPAKVYVGVKQEIAEMRIPALNAYMKSLLSLPVWVL
MDEDVRIFFYQSPYDS
PX domain of p40phox (NCF4) R58L
(SEQ ID NO: 46)
DVAISANIADIEEKRGFTSHFVFVIEVKTKGGSKYLIYLRYRQFHALQSKLEERFGPD
SKSSALACTLPTLPAKVYVGVKQEIAEMRIPALNAYMKSLLSLPVWVLMDEDVRIFFY
QSPYDS
Pleckstrin homology domain of Homo sapiens DAPP1
(SEQ ID NO: 47)
MQTGRTEDDLVPTAPSLGTKEGYLTKQGGLVKTWKTRWFTLHRNELKYFK
DQMSPEPIRILDLTECSAVQFDYSQERVNCFCLVFPFRTFYLCAKTGVEADE
WIKILRWKLSQIRKQLNQGEGTIR
Pleckstrin homology domain of Homo sapiens GRP1 (CYTH3)
(SEQ ID NO: 48)
PFKIPEDDGNDLTHTFFNPDREGWLLKLGGRVKTWKRRWFILTDNCLYYFE
YTTDKEPRGIIPLENLSIREVEDPRKPNCFELYNPSHKGQVIKACKTEADGRV
VEGNHVVYRISAPSPEEKEEWMKSIKASISRDPFYDMLATRKRRIANKK
Pleckstrin homology domain of Homo sapiens GRP1 (CYTH3) R284C
(SEQ ID NO: 49)
MPFKIPEDDGNDLTHTFFNPDREGWLLKLGGRVKTWKCRWFILTDNCLYYF
EYTTDKEPRGIIPLENLSIREVEDPRKPNCFELYNPSHKGQVIKACKTEADGR
WVEGNHVVYRISAPSPEEKEEWMKSIKASISRDPFYDMLATRKRRIANKK
Pleckstrin homology domain of Human OSBP1
(SEQ ID NO: 50)
MGSGSAREGWLFKWTNYIKGYQRRWFVLSNGLLSYYRSKAEMRHTCRGTINLATA
NITVEDSCNFIISNGGAQTYHLKASSEVERQRWVTALELAKAKAVK
Pleckstrin homology domain of Human OSBP1 R108E
(SEQ ID NO: 51)
MGSGSAREGWLFKWTNYIKGYQERWFVLSNGLLSYYRSKAEMRHTCRGTINLATA
NITVEDSCNFIISNGGAQTYHLKASSEVERQRWVTALELAKAKAVK
Pleckstrin homology domain of Human Btk1
(SEQ ID NO: 52)
MAAVILESIFLKRSQQKKKTSPLNFKKRLFLLTVHKLSYYEYDFERGRRGSKKGSIDV
EKITCVETVVPEKNPPPERQIPRRGEESSEMEQISIIERFPYPFQVVYDEGPLYVFSP
TEELRKRWIHQLKNVIRYNSDLVQKYHPCFWIDGQYLCCSQTAKNAMGCQILENRN
GSLKP
Pleckstrin homology domain of Human Btk1 R28C
(SEQ ID NO: 53)
MAAVILESIFLKRSQQKKKTSPLNFKKCLFLLTVHKLSYYEYDFERGRRGSKKGSIDV
EKITCVETVVPEKNPPPERQIPRRGEESSEMEQISIIERFPYPFQVVYDEGPLYVFSP
TEELRKRWIHQLKNVIRYNSDLVQKYHPCFWIDGQYLCCSQTAKNAMGCQILENRN
GSLKP
Pleckstrin homology domain of Human FAPP1
(SEQ ID NO: 54)
MEGVLYKWTNYLTGWQPRWFVLDNGILSYYDSQDDVCKGSKGSIKMAVCEIKVHS
ADNTRMELIIPGEQHFYMKAVNAAERQRWLVALGSSKACLTDT
Pleckstrin homology domain of Human CERT
(SEQ ID NO: 55)
PVERCGVLSKWTNYIHGWQDRWVVLKNNALSYYKSEDETEYGCRGSICLSKAVITP
HDFDECRFDISVNDSVWYLRAQDPDHRQQWIDAIEQHKT
Pleckstrin homology domain of Human PHLPP1
(SEQ ID NO: 56)
MRIQLSGMYNVRKGKMQLPVNRWTRRQVILCGTCLIVSSVKDSLTGKMHVLPLIGG
KVEEVKKHQHCLAFSSSGPQSQTYYICFDTFTEYLRWLRQVSKVAS
Pleckstrin homology domain of Human SWAP70
(SEQ ID NO: 57)
MDVLKQGYMMKKGHRRKNWTERWFVLKPNIISYYVSEDLKDKKGDILLDENCCVE
SLPDKDGKKCLFLVKCFDKTFEISASDKKKKQEWIQAIHSTIH
Pleckstrin homology domain of Human SWAP70 R223E, R224E
(SEQ ID NO: 58)
MDVLKQGYMMKKGHEEKNWTERWFVLKPNIISYYVSEDLKDKKGDILLDENCCVES
LPDKDGKKCLFLVKCFDKTFEISASDKKKKQEWIQAIHSTIH
Pleckstrin homology domain of Human MAPKAP1
(SEQ ID NO: 59)
MDMLSSHHYKSFKVSMIHRLRFTTDVQLGISGDKVEIDPVTNQKASTKFWIKQKPISI
DSDLLCACDLAEEKSPSHAIFKLTYLSNHDYKHLYFESDAATVNEIVLKVNYILES
Pleckstrin Homology Domain of Human PKD
(SEQ ID NO: 60)
MGTVMKEGWMVHYTSKDTLRKRHYWRLDSKCITLFQNDTGSRYYKEIPLSEILSLE
PVKTSALIPNGANPHCFEITTANVVYYVGENVVNPSSPSPNNSVLTSGVGADVARM
WEIAIQHALM
Pleckstrin homology domain of Human Son Of Sevenless Homolog 2
(SEQ ID NO: 61)
FIMEGPLTRIGAKHERHIFLFDGLMISCKPNHGQTRLPGYSSAEYRLKEKFVMRKIQI
CDKEDTCEHKHAFELVSKDENSIIFAAKSAEEKNNWMAALISLHYRS
Pleckstrin homology domain of Human Dynamin
(SEQ ID NO: 62)
QGTNLPPSRQIVIRKGWLTISNIGIMKGGSKGYWFVLTAESLSWYKDDEEKEKKYML
PLDNLKVRDVEKSFMSSKHIFALFNTEQRNVYKDYRFLELACDSQEDVDS
Pleckstrin homology domain of Human BCR
(SEQ ID NO: 63)
QLLKDSFMVELVEGARKLRHVFLFTDLLLCTKLKKQSGGKTQQYDCKWYIPLTDLSF
QMVDELEAVPNIPLVPDEELDALKIKISQIKNDIQREKRANKGSKATERLKKKLSEQE
SLLLLMSPSMAFRVHSRNGKSYTFLISSDYERAEWRENIREQQK
Pleckstrin homology domain of Human DBS
(SEQ ID NO: 64)
KLLMQGSFSVWTDHKRGHTKVKELARFKPMQRHLFLHEKAVLFCKKREEN
GEGYEKAPSYSYKQSLNMAAVGITENVKGDAKKFEIWYNAREEVYIVQAPT
PEIKAAWVNEIRKVLT
Pleckstrin homology domain of Homo sapiens phospholipase C81
(hPLC81)
(SEQ ID NO: 65)
MDSGRDFLTLHGLQDDEDLQALLKGSQLLKVKSSSWRRERFYKLQEDCKTI
WQESRKVMRTPESQLFSIEDIQEVRMGHRTEGLEKFARDVPEDRCFSIVFK
DQRNTLDLIAPSPADAQHWVLGLHKIIHHSGSMDQRQKLQHWIHSCLRKAD
KNKDNKMSFKELQNFLKELNIQ
Pleckstrin homology domain of Homo sapiens phospholipase C81
(hPLC81) R40L
(SEQ ID NO: 66)
MDSGRDFLTLHGLQDDEDLQALLKGSQLLKVKSTSWRRELFYKLQEDCKTI
WQESRKVMRTPESQLFSIEDIQEVRMGHRTEGLEKFARDVPEDRCFSIVFK
DQRNTLDLIAPSPADAQHWVLGLHKIIHHSGSMDQRQKLQHWIHSCLRKAD
KNKDNKMSFKELQNFLK
Pleckstrin homology domain of Homo sapiens Akt1 (hAkt)
(SEQ ID NO: 67)
MSDVAIVKEGWLHKRGEYIKTWRPRYFLLKNDGTFIGYKERPQDVDQREAP
LNNFSVAQCQLMKTERPRPNTFIIRCLQWTTVIERTFHVETPEEREEWTTAI
QTVADGLKKQEEEEMDFRSGSPSDNSGAEEMEVSLAKPKHRVTMNEFEYL
KLLGKGTFGKVDPPV
Pleckstrin homology domain of Homo sapiens Akt1 (hAkt) E17K
(SEQ ID NO: 68)
MSDVAIVKEGWLHKRGKYIKTWRPRYFLLKNDGTFIGYKERPQDVDQREAPLNNFS
VAQCQLMKTERPRPNTFIIRCLQWTTVIERTFHVETPEEREEWTTAIQTVADGLKKQ
EEEEMDFRSGSPSDNSGAEEMEVSLAKPKHRVTMNEFEYLKLLGKGTFGKVDPPV
Pleckstrin homology domain of Homo sapiens Akt1 (hAkt) K14R
(SEQ ID NO: 69)
MSDVAIVKEGWLHRRGEYIKTWRPRYFLLKNDGTFIGYKERPQDVDQREAPLNNFS
VAQCQLMKTERPRPNTFIIRCLQWTTVIERTFHVETPEEREEWTTAIQTVADGLKKQ
EEEEMDFRSGSPSDNSGAEEMEVSLAKPKHRVTMNEFEYLKLLGKGTFGKVDPPV
Pleckstrin homology domain of Homo sapiens Akt1 (hAkt) K8R
(SEQ ID NO: 70)
MSDVAIVREGWLHKRGEYIKTWRPRYFLLKNDGTFIGYKERPQDVDQREAPLNNFS
VAQCQLMKTERPRPNTFIIRCLQWTTVIERTFHVETPEEREEWTTAIQTVADGLKKQ
EEEEMDFRSGSPSDNSGAEEMEVSLAKPKHRVTMNEFEYLKLLGKGTFGKVDPPV
Pleckstrin homology domain of Homo sapiens Akt1 (hAkt) T72A
(SEQ ID NO: 71)
MSDVAIVKEGWLHKRGEYIKTWRPRYFLLKNDGTFIGYKERPQDVDQREAPLNNFS
VAQCQLMKTERPRPNAFIIRCLQWTTVIERTFHVETPEEREEWTTAIQTVADGLKKQ
EEEEMDFRSGSPSDNSGAEEMEVSLAKPKHRVTMNEFEYLKLLGKGTFGKVDPPV
Pleckstrin homology domain of Homo sapiens Akt1 (hAkt) T92A
(SEQ ID NO: 72)
MSDVAIVKEGWLHKRGEYIKTWRPRYFLLKNDGTFIGYKERPQDVDQREAPLNNFS
VAQCQLMKTERPRPNTFIIRCLQWTTVIERTFHVEAPEEREEWTTAIQTVADGLKKQ
EEEEMDFRSGSPSDNSGAEEMEVSLAKPKHRVTMNEFEYLKLLGKGTFGKVDPPV
Pleckstrin homology domain of Homo sapiens Akt1 (hAkt) R25C
(SEQ ID NO: 73)
MSDVAIVKEGWLHKRGEYIKTWRPCYFLLKNDGTFIGYKERPQDVDQREAP
LNNFSVAQCQLMKTERPRPNTFIIRCLQWTTVIERTFHVETPEEREEWTTAI
QTVADGLKKQEEEEMDFRSGSPSDNSGAEEMEVSLAKPKHRVTMNEFEYL
KLLGKGTFGKVDPPV
Pleckstrin homology domain of Homo sapiens Akt1 (hAkt) T34D
(SEQ ID NO: 74)
MSDVAIVKEGWLHKRGEYIKTWRPRYFLLKNDGDFIGYKERPQDVDQREAPLNNFS
VAQCQLMKTERPRPNTFIIRCLQWTTVIERTFHVETPEEREEWTTAIQTVADGLKKQ
EEEEMDFRSGSPSDNSGAEEMEVSLAKPKHRVTMNEFEYLKLLGKGTFGKVDPPV
Pleckstrin homology domain of Homo sapiens Akt1 (hAkt) T34F
(SEQ ID NO: 75)
MSDVAIVKEGWLHKRGEYIKTWRPRYFLLKNDGFFIGYKERPQDVDQREAP
LNNFSVAQCQLMKTERPRPNTFIIRCLQWTTVIERTFHVETPEEREEWTTAI
QTVADGLKKQEEEEMDFRSGSPSDNSGAEEMEVSLAKPKHRVTMNEFEYL
KLLGKGTFGKVDPPV
Pleckstrin homology domain of Homo sapiens Akt1 (hAkt) T34L
(SEQ ID NO: 76)
MSDVAIVKEGWLHKRGEYIKTWRPRYFLLKNDGLFIGYKERPQDVDQREAPLNNFS
VAQCQLMKTERPRPNTFIIRCLQWTTVIERTFHVETPEEREEWTTAIQTVADGLKKQ
EEEEMDFRSGSPSDNSGAEEMEVSLAKPKHRVTMNEFEYLKLLGKGTFGKVDPPV
Pleckstrin homology domain of Homo sapiens Akt1 (hAkt) T81Y
(SEQ ID NO: 77)
MSDVAIVKEGWLHKRGEYIKTWRPRYFLLKNDGTFIGYKERPQDVDQREAPLNNFS
VAQCQLMKTERPRPNTFIIRCLQWYTVIERTFHVETPEEREEWTTAIQTVADGLKKQ
EEEEMDFRSGSPSDNSGAEEMEVSLAKPKHRVTMNEFEYLKLLGKGTFGKVDPPV
Pleckstrin homology domain of Homo sapiens Akt1 (hAkt) K142A, H143A,
R144A
(SEQ ID NO: 78)
MSDVAIVKEGWLHKRGEYIKTWRPRYFLLKNDGTFIGYKERPQDVDQREAPLNNFS
VAQCQLMKTERPRPNTFIIRCLQWTTVIERTFHVETPEEREEWTTAIQTVADGLKKQ
EEEEMDFRSGSPSDNSGAEEMEVSLAKPAAAVTMNEFEYLKLLGKGTFGKVDPPV
Pleckstrin homology domain of Homo sapiens Akt1 (hAkt) T101C
(SEQ ID NO: 79)
MSDVAIVKEGWLHKRGEYIKTWRPRYFLLKNDGTFIGYKERPQDVDQREAPLNNFS
VAQCQLMKTERPRPNTFIIRCLQWTTVIERTFHVETPEEREEWTCAIQTVADGLKKQ
EEEEMDFRSGSPSDNSGAEEMEVSLAKPKHRVTMNEFEYLKLLGKGTFGKVDPPV
Pleckstrin homology domain of Homo sapiens PDPK1 (hPDPK1)
(SEQ ID NO: 80)
KMGPVDKRKGLFARRRQLLLTEGPHLYYVDPVNKVLKGEIPWSQELRPEAK
NFKTFFVHTPNRTYYLMDPSGNAHKWCRKIQEVWRQRYQSH
MS2 (RNA Binding protein)
(SEQ ID NO: 81)
MASNFTQFVLVDNGGTGDVTVAPSNFANGIAEWISSNSRSQAYKVTCSVRQ
SSAQKRKYTIKVEVPKVATQTVGGVELPVAAWRSYLNMELTIPIFATNSDCE
LIVKAMQGLLKDGNPIPSAIAANSGIY
COM (RNA Binding protein)
(SEQ ID NO: 82)
MKSIRCKNONKLLFKADSFDHIEIRCPRCKRHIIMLNACEHPTEKHCGKREKI
THSDETVRY
PP7 (RNA Binding protein)
(SEQ ID NO: 83)
MAKTIVLAVGEATRTLTEIQSTADRQIFEEKVGPLVGRLRLTASLRQNGAKTA
YRVNLKLDQADVVDASTSVAGELPKVRYTQVWSHDVTIVANSTEASRKSLY
DLTKSLVATSQVEDLVVNLVPLGRSLE
TBP (RNA Binding protein)
(SEQ ID NO: 84)
MAVPETRPNHTIYINNLNSKIKKDELKKSLYAIFSQFGQILDILVPRQRTPRGQ
AFVIFKEVSSATNALRSMQGFPFYDKPMRIQYAKTDKRIPAKMKGTFV
Human SLBP (RNA Binding protein)
(SEQ ID NO: 85)
MADFETDESVLMRRQKQINYGKNTIAYDRYIKEVPRHLRQPGIHPKTPNKFK
KYSRRSWDQQIKLWKVALHFWD
Herpes simplex virus (HSV) type 1 VP16 Transcription Activation
Domain
(SEQ ID NO: 86)
PTDALDDFDLDMLPADALDDFDLDMLPADALDDFDLDM
Herpes simplex virus (HSV) type 1 & Synthetic VP64
(SEQ ID NO: 87)
GRADALDDFDLDMLGSDALDDFDLDMLGSDALDDFDLDMLGSDALDDFDL
DML
Homo sapiens P65
(SEQ ID NO: 88)
SQYLPDTDDRHRIEEKRKRTYETFKSIMKKSPFSGPTDPRPPPRRIAVPSRS
SASVPKPAPQPYPFTSSLSTINYDEFPTMVFPSGQISQASALAPAPPQVLPQ
APAPAPAPAMVSALAQAPAPVPVLAPGPPQAVAPPAPKPTQAGEGTLSEAL
LQLQFDDEDLGALLGNSTDPAVFTDLASVDNSEFQQLLNQGIPVAPHTTEP
MLMEYPEAITRLVTGAQRPPDPAPAPLGAPGLPNGLLSGDEDFSSIADMDF
SALL
Kaposi's Sarcoma-Associated Herpesvirus Transactivator RTA
(SEQ ID NO: 89)
RDSREGMFLPKPEAGSAISDVFEGREVCQPKRIRPFHPPGSPWANRPLPAS
LAPTPTGPVHEPVGSLTPAPVPQPLDPAPAVTPEASHLLEDPDEETSQAVKA
LREMADTVIPQKEEAAICGQMDLSHPPPRGHLDELTTTLESMTEDLNLDSPL
TPELNEILDTFLNDECLLHAMHISTGLSIFDTSLF
Homo sapiens KRAB
(SEQ ID NO: 90)
MDAKSLTAWSRTLVTFKDVFVDFTREEWKLLDTAQQIVYRNVMLENYKNLV
SLGYQLTKPDVILRLEKGEEP
Homo sapiens MeCP2
(SEQ ID NO: 91)
EASVQVKRVLEKSPGKLLVKMPFQASPGGKGEGGGATTSAQVMVIKRPGR
KRKAEADPQAIPKKRGRKPGSVVAAAAAEAKKKAVKESSIRSVQETVLPIKK
RKTRETVSIEVKEVVKPLLVSTLGEKSGKGLKTCKSPGRKSKESSPKGRSSS
ASSPPKKEHHHHHHHAESPKAPMPLLPPPPPPEPQSSEDPISPPEPQDLSS
SICKEEKMPRAGSLESDGCPKEPAKTQPMVAAAATTTTTTTTTVAEKYKHR
GEGERKDIVSSSMPRPNREEPVDSRTPVTERVS
Homo sapiens Tet1
(SEQ ID NO: 92)
LPTCSCLDRVIQKDKGPYYTHLGAGPSVAAVREIMENRYGQKGNAIRIEIVVY
TGKEGKSSHGCPIAKWVLRRSSDEEKVLCLVRQRTGHHCPTAVMVVLIMV
WDGIPLPMADRLYTELTENLKSYNGHPTDRRCTLNENRTCTCQGIDPETCG
ASFSFGCSWSMYFNGCKFGRSPSPRRFRIDPSSPLHEKNLEDNLQSLATRL
APIYKQYAPVAYQNQVEYENVARECRLGSKEGRPFSGVTACLDFCAHPHR
DIHNMNNGSTVVCTLTREDNRSLGVIPQDEQLHVLPLYKLSDTDEFGSKEG
MEAKIKSGAIEVLAPRRKKRTCFTQPVPRSGKKRAAMMTEVLAHKIRAVEKK
PIPRIKRKNNSTTTNNSKPSSLPTLGSNTETVQPEVKSETEPHFILKSSDNTK
TYSLMPSAPHPVKEASPGFSWSPKTASATPAPLKNDATASCGFSERSSTPH
CTMPSGRLSGANAAAADGPGISQLGEVAPLPTLSAPVMEPLINSEPSTGVTE
PLTPHQPNHQPSFLTSPQDLASSPMEEDEQHSEADEPPSDEPLSDDPLSPA
EEKLPHIDEYWSDSEHIFLDANIGGVAIAPAHGSVLIECARRELHATTPVEHP
NRNHPTRLSLVFYQHKNLNKPQHGFELNKIKFEAKEAKNKKMKASEQKDQA
ANEGPEQSSEVNELNQIPSHKALTLTHDNVVTVSPYALTHVAGPYNHWV
Homo sapiens Dnmt3a
(SEQ ID NO: 93)
MPAMPSSGPGDTSSSAAEREEDRKDGEEQEEPRGKEERQEPSTTARKVG
RPGRKRKHPPVESGDTPKDPAVISKSPSMAQDSGASELLPNGDLEKRSEP
QPEEGSPAGGQKGGAPAEGEGAAETLPEASRAVENGCCTPKEGRGAPAE
AGKEQKETNIESMKMEGSRGRLRGGLGWESSLRQRPMPRLTFQAGDPYYI
SKRKRDEWLARWKREAEKKAKVIAGMNAVEENQGPGESQKVEEASPPAV
QQPTDPASPTVATTPEPVGSDAGDKNATKAGDDEPEYEDGRGFGIGELVW
GKLRGFSWWPGRIVSWWMTGRSRAAEGTRWVMWFGDGKFSVVCVEKLM
PLSSFCSAFHQATYNKQPMYRKAIYEVLQVASSRAGKLFPVCHDSDESDTA
KAVEVQNKPMIEWALGGFQPSGPKGLEPPEEEKNPYKEVYTDMWVEPEAA
AYAPPPPAKKPRKSTAEKPKVKEIIDERTRERLVYEVRQKCRNIEDICISCGS
LNVTLEHPLFVGGMCQNCKNCFLECAYQYDDDGYQSYCTICCGGREVLMC
GNNNCCRCFCVECVDLLVGPGAAQAAIKEDPWNCYMCGHKGTYGLLRRR
EDWPSRLQMFFANNHDQEFDPPKVYPPVPAEKRKPIRVLSLFDGIATGLLVL
KDLGIQVDRYIASEVCEDSITVGMVRHQGKIMYVGDVRSVTQKHIQEWGPF
DLVIGGSPCNDLSIVNPARKGLYEGTGRLFFEFYRLLHDARPKEGDDRPFF
WLFENVVAMGVSDKRDISRFLESNPVMIDAKEVSAAHRARYFWGNLPGMN
RPLASTVNDKLELQECLEHGRIAKFSKVRTITTRSNSIKQGKDQHFPVFMNE
KEDILWCTEMERVFGFPVHYTDVSNMSRLARQRLLGRSWSVPVIRHLFAPL
KEYFACV
Vesicular stomatitis virus Glycoprotein (VSVG) WT
(SEQ ID NO: 94)
MKCLLYLAFLFIGVNCKFTIVFPHNQKGNWKNVPSNYHYCPSSSDLNWHND
LIGTALQVKMPKSHKAIQADGWMCHASKWVTTCDFRWYGPKYITHSIRSFT
PSVEQCKESIEQTKQGTWLNPGFPPQSCGYATVTDAEAVIVQVTPHHVLVD
EYTGEWVDSQFINGKCSNYICPTVHNSTTWHSDYKVKGLCDSNLISMDITFF
SEDGELSSLGKEGTGFRSNYFAYETGGKACKMQYCKHWGVRLPSGVWFE
MADKDLFAAARFPECPEGSSISAPSQTSVDVSLIQDVERILDYSLCQETWSKI
RAGLPISPVDLSYLAPKNPGTGPAFTIINGTLKYFETRYIRVDIAAPILSRMVG
MISGTTTERELWDDWAPYEDVEIGPNGVLRTSSGYKFPLYMIGHGMLDSDL
HLSSKAQVFEHPHIQDAASQLPDDESLFFGDTGLSKNPIELVEGWFSSWKS
SIASFFFIIGLIIGLFLVLRVGIHLCIKLKHTKKRQIYTDIEMNRLGK
Vesicular stomatitis virus Glycoprotein (VSVG) (K47A)
(SEQ ID NO: 95)
MKCLLYLAFLFIGVNCKFTIVFPHNQKGNWKNVPSNYHYCPSSSDLNWHND
LIGTALQVKMPASHKAIQADGWMCHASKWVTTCDFRWYGPKYITHSIRSFTP
SVEQCKESIEQTKQGTWLNPGFPPQSCGYATVTDAEAVIVQVTPHHVLVDEY
TGEWVDSQFINGKCSNYICPTVHNSTTWHSDYKVKGLCDSNLISMDITFFSED
GELSSLGKEGTGFRSNYFAYETGGKACKMQYCKHWGVRLPSGVWFEMADK
DLFAAARFPECPEGSSISAPSQTSVDVSLIQDVERILDYSLCQETWSKIRAGLPI
SPVDLSYLAPKNPGTGPAFTIINGTLKYFETRYIRVDIAAPILSRMVGMISGTTT
ERELWDDWAPYEDVEIGPNGVLRTSSGYKFPLYMIGHGMLDSDLHLSSKAQ
VFEHPHIQDAASQLPDDESLFFGDTGLSKNPIELVEGWFSSWKSSIASFFFIIGLI
IGLFLVLRVGIHLCIKLKHTKKRQIYTDIEMNRLGK
Vesicular stomatitis virus Glycoprotein (VSVG) (K47E)
(SEQ ID NO: 96)
MKCLLYLAFLFIGVNCKFTIVFPHNQKGNWKNVPSNYHYCPSSSDLNWHND
LIGTALQVKMPESHKAIQADGWMCHASKWVTTCDFRWYGPKYITHSIRSFTP
SVEQCKESIEQTKQGTWLNPGFPPQSCGYATVTDAEAVIVQVTPHHVLVDEY
TGEWVDSQFINGKCSNYICPTVHNSTTWHSDYKVKGLCDSNLISMDITFFSED
GELSSLGKEGTGFRSNYFAYETGGKACKMQYCKHWGVRLPSGVWFEMADK
DLFAAARFPECPEGSSISAPSQTSVDVSLIQDVERILDYSLCQETWSKIRAGLPI
SPVDLSYLAPKNPGTGPAFTIINGTLKYFETRYIRVDIAAPILSRMVGMISGTTT
ERELWDDWAPYEDVEIGPNGVLRTSSGYKFPLYMIGHGMLDSDLHLSSKAQ
VFEHPHIQDAASQLPDDESLFFGDTGLSKNPIELVEGWFSSWKSSIASFFFIIGLI
IGLFLVLRVGIHLCIKLKHTKKRQIYTDIEMNRLGK
Vesicular stomatitis virus Glycoprotein (VSVG) (K47G)
(SEQ ID NO: 97)
MKCLLYLAFLFIGVNCKFTIVFPHNQKGNWKNVPSNYHYCPSSSDLNWHND
LIGTALQVKMPGSHKAIQADGWMCHASKWVTTCDFRWYGPKYITHSIRSFTP
SVEQCKESIEQTKQGTWLNPGFPPQSCGYATVTDAEAVIVQVTPHHVLVDEY
TGEWVDSQFINGKCSNYICPTVHNSTTWHSDYKVKGLCDSNLISMDITFFSED
GELSSLGKEGTGFRSNYFAYETGGKACKMQYCKHWGVRLPSGVWFEMADK
DLFAAARFPECPEGSSISAPSQTSVDVSLIQDVERILDYSLCQETWSKIRAGLPI
SPVDLSYLAPKNPGTGPAFTIINGTLKYFETRYIRVDIAAPILSRMVGMISGTTT
ERELWDDWAPYEDVEIGPNGVLRTSSGYKFPLYMIGHGMLDSDLHLSSKAQ
VFEHPHIQDAASQLPDDESLFFGDTGLSKNPIELVEGWFSSWKSSIASFFFIIGLI
IGLFLVLRVGIHLCIKLKHTKKRQIYTDIEMNRLGK
Vesicular stomatitis virus Glycoprotein (VSVG) (K47Q)
(SEQ ID NO: 98)
MKCLLYLAFLFIGVNCKFTIVFPHNQKGNWKNVPSNYHYCPSSSDLNWHND
LIGTALQVKMPQSHKAIQADGWMCHASKWVTTCDFRWYGPKYITHSIRSFTP
SVEQCKESIEQTKQGTWLNPGFPPQSCGYATVTDAEAVIVQVTPHHVLVDEY
TGEWVDSQFINGKCSNYICPTVHNSTTWHSDYKVKGLCDSNLISMDITFFSED
GELSSLGKEGTGFRSNYFAYETGGKACKMQYCKHWGVRLPSGVWFEMADK
DLFAAARFPECPEGSSISAPSQTSVDVSLIQDVERILDYSLCQETWSKIRAGLPI
SPVDLSYLAPKNPGTGPAFTIINGTLKYFETRYIRVDIAAPILSRMVGMISGTTT
ERELWDDWAPYEDVEIGPNGVLRTSSGYKFPLYMIGHGMLDSDLHLSSKAQ
VFEHPHIQDAASQLPDDESLFFGDTGLSKNPIELVEGWFSSWKSSIASFFFIIGLI
IGLFLVLRVGIHLCIKLKHTKKRQIYTDIEMNRLGK
Vesicular stomatitis virus Glycoprotein (VSVG) (K47W)
(SEQ ID NO: 99)
MKCLLYLAFLFIGVNCKFTIVFPHNQKGNWKNVPSNYHYCPSSSDLNWHND
LIGTALQVKMPWSHKAIQADGWMCHASKWVTTCDFRWYGPKYITHSIRSFT
PSVEQCKESIEQTKQGTWLNPGFPPQSCGYATVTDAEAVIVQVTPHHVLVDE
YTGEWVDSQFINGKCSNYICPTVHNSTTWHSDYKVKGLCDSNLISMDITFFSE
DGELSSLGKEGTGFRSNYFAYETGGKACKMQYCKHWGVRLPSGVWFEMAD
KDLFAAARFPECPEGSSISAPSQTSVDVSLIQDVERILDYSLCQETWSKIRAGLP
ISPVDLSYLAPKNPGTGPAFTIINGTLKYFETRYIRVDIAAPILSRMVGMISGTT
TERELWDDWAPYEDVEIGPNGVLRTSSGYKFPLYMIGHGMLDSDLHLSSKA
QVFEHPHIQDAASQLPDDESLFFGDTGLSKNPIELVEGWFSSWKSSIASFFFIIG
LIIGLFLVLRVGIHLCIKLKHTKKRQIYTDIEMNRLGK
Vesicular stomatitis virus Glycoprotein (VSVG) (K47A)(R354A)
(SEQ ID NO: 100)
MKCLLYLAFLFIGVNCKFTIVFPHNQKGNWKNVPSNYHYCPSSSDLNWHND
LIGTALQVKMPASHKAIQADGWMCHASKWVTTCDFRWYGPKYITHSIRSFTP
SVEQCKESIEQTKQGTWLNPGFPPQSCGYATVTDAEAVIVQVTPHHVLVDEY
TGEWVDSQFINGKCSNYICPTVHNSTTWHSDYKVKGLCDSNLISMDITFFSED
GELSSLGKEGTGFRSNYFAYETGGKACKMQYCKHWGVRLPSGVWFEMADK
DLFAAARFPECPEGSSISAPSQTSVDVSLIQDVERILDYSLCQETWSKIRAGLPI
SPVDLSYLAPKNPGTGPAFTIINGTLKYFETRYIRVDIAAPILSRMVGMISGTTT
EAELWDDWAPYEDVEIGPNGVLRTSSGYKFPLYMIGHGMLDSDLHLSSKAQ
VFEHPHIQDAASQLPDDESLFFGDTGLSKNPIELVEGWFSSWKSSIASFFFIIGLI
IGLFLVLRVGIHLCIKLKHTKKRQIYTDIEMNRLGK
Vesicular stomatitis virus Glycoprotein (VSVG) (K47E) (R354A)
(SEQ ID NO: 101)
MKCLLYLAFLFIGVNCKFTIVFPHNQKGNWKNVPSNYHYCPSSSDLNWHND
LIGTALQVKMPESHKAIQADGWMCHASKWVTTCDFRWYGPKYITHSIRSFTP
SVEQCKESIEQTKQGTWLNPGFPPQSCGYATVTDAEAVIVQVTPHHVLVDEY
TGEWVDSQFINGKCSNYICPTVHNSTTWHSDYKVKGLCDSNLISMDITFFSED
GELSSLGKEGTGFRSNYFAYETGGKACKMQYCKHWGVRLPSGVWFEMADK
DLFAAARFPECPEGSSISAPSQTSVDVSLIQDVERILDYSLCQETWSKIRAGLPI
SPVDLSYLAPKNPGTGPAFTIINGTLKYFETRYIRVDIAAPILSRMVGMISGTTT
EAELWDDWAPYEDVEIGPNGVLRTSSGYKFPLYMIGHGMLDSDLHLSSKAQ
VFEHPHIQDAASQLPDDESLFFGDTGLSKNPIELVEGWFSSWKSSIASFFFIIGLI
IGLFLVLRVGIHLCIKLKHTKKRQIYTDIEMNRLGK
Vesicular stomatitis virus Glycoprotein (VSVG) (K47G) (R354A)
(SEQ ID NO: 102)
MKCLLYLAFLFIGVNCKFTIVFPHNQKGNWKNVPSNYHYCPSSSDLNWHND
LIGTALQVKMPGSHKAIQADGWMCHASKWVTTCDFRWYGPKYITHSIRSFTP
SVEQCKESIEQTKQGTWLNPGFPPQSCGYATVTDAEAVIVQVTPHHVLVDEY
TGEWVDSQFINGKCSNYICPTVHNSTTWHSDYKVKGLCDSNLISMDITFFSED
GELSSLGKEGTGFRSNYFAYETGGKACKMQYCKHWGVRLPSGVWFEMADK
DLFAAARFPECPEGSSISAPSQTSVDVSLIQDVERILDYSLCQETWSKIRAGLPI
SPVDLSYLAPKNPGTGPAFTIINGTLKYFETRYIRVDIAAPILSRMVGMISGTTT
EAELWDDWAPYEDVEIGPNGVLRTSSGYKFPLYMIGHGMLDSDLHLSSKAQ
VFEHPHIQDAASQLPDDESLFFGDTGLSKNPIELVEGWFSSWKSSIASFFFIIGLI
IGLFLVLRVGIHLCIKLKHTKKRQIYTDIEMNRLGK
Vesicular stomatitis virus Glycoprotein (VSVG) (K47Q) (R354A)
(SEQ ID NO: 103)
MKCLLYLAFLFIGVNCKFTIVFPHNQKGNWKNVPSNYHYCPSSSDLNWHND
LIGTALQVKMPQSHKAIQADGWMCHASKWVTTCDFRWYGPKYITHSIRSFTP
SVEQCKESIEQTKQGTWLNPGFPPQSCGYATVTDAEAVIVQVTPHHVLVDEY
TGEWVDSQFINGKCSNYICPTVHNSTTWHSDYKVKGLCDSNLISMDITFFSED
GELSSLGKEGTGFRSNYFAYETGGKACKMQYCKHWGVRLPSGVWFEMADK
DLFAAARFPECPEGSSISAPSQTSVDVSLIQDVERILDYSLCQETWSKIRAGLPI
SPVDLSYLAPKNPGTGPAFTIINGTLKYFETRYIRVDIAAPILSRMVGMISGTTT
EAELWDDWAPYEDVEIGPNGVLRTSSGYKFPLYMIGHGMLDSDLHLSSKAQ
VFEHPHIQDAASQLPDDESLFFGDTGLSKNPIELVEGWFSSWKSSIASFFFIIGLI
IGLFLVLRVGIHLCIKLKHTKKRQIYTDIEMNRLGK
Vesicular stomatitis virus Glycoprotein (VSVG) (K47W) (R354A)
(SEQ ID NO: 104)
MKCLLYLAFLFIGVNCKFTIVFPHNQKGNWKNVPSNYHYCPSSSDLNWHND
LIGTALQVKMPWSHKAIQADGWMCHASKWVTTCDFRWYGPKYITHSIRSFT
PSVEQCKESIEQTKQGTWLNPGFPPQSCGYATVTDAEAVIVQVTPHHVLVDE
YTGEWVDSQFINGKCSNYICPTVHNSTTWHSDYKVKGLCDSNLISMDITFFSE
DGELSSLGKEGTGFRSNYFAYETGGKACKMQYCKHWGVRLPSGVWFEMAD
KDLFAAARFPECPEGSSISAPSQTSVDVSLIQDVERILDYSLCQETWSKIRAGLP
ISPVDLSYLAPKNPGTGPAFTIINGTLKYFETRYIRVDIAAPILSRMVGMISGTT
TEAELWDDWAPYEDVEIGPNGVLRTSSGYKFPLYMIGHGMLDSDLHLSSKA
QVFEHPHIQDAASQLPDDESLFFGDTGLSKNPIELVEGWFSSWKSSIASFFFIIG
LIIGLFLVLRVGIHLCIKLKHTKKRQIYTDIEMNRLGK
Targeting Domain-VSVG fusion site
MKCLLYLAFLFIGVNC- X, wherein X is a Targeting Domain-
(SEQ ID NO: 105)
KFTIVFPHNQKGNWKNVPSNYHYCPSSSDLNWHNDLIGTALQVKMPKSHKAI
QADGWMCHASKWVTTCDFRWYGPKYITHSIRSFTPSVEQCKESIEQTKQGT
WLNPGFPPQSCGYATVTDAEAVIVQVTPHHVLVDEYTGEWVDSQFINGKCS
NYICPTVHNSTTWHSDYKVKGLCDSNLISMDITFFSEDGELSSLGKEGTGFRS
NYFAYETGGKACKMQYCKHWGVRLPSGVWFEMADKDLFAAARFPECPEGS
SISAPSQTSVDVSLIQDVERILDYSLCQETWSKIRAGLPISPVDLSYLAPKNPGT
GPAFTIINGTLKYFETRYIRVDIAAPILSRMVGMISGTTTERELWDDWAPYED
VEIGPNGVLRTSSGYKFPLYMIGHGMLDSDLHLSSKAQVFEHPHIQDAASQLP
DDESLFFGDTGLSKNPIELVEGWFSSWKSSIASFFFIIGLIIGLFLVLRVGIHLCIK
LKHTKKRQIYTDIEMNRLGK
Vesicular stomatitis virus Glycoprotein (VSVG truncation) (F421 Truncation)
(SEQ ID NO: 106)
MKCLLYLAFLFIGVNCKFEHPHIQDAASQLPDDESLFFGDTGLSKNPIELVEG
WFSSWKSSIASFFFIIGLIIGLFLVLRVGIHLCIKLKHTKKRQIYTDIEMNRLGK
Vesicular stomatitis virus Glycoprotein (VSVG truncation) (F440 Truncation)
(SEQ ID NO: 107)
MKCLLYLAFLFIGVNCKFFGDTGLSKNPIELVEGWFSSWKSSIASFFFIIGLIIGL
FLVLRVGIHLCIKLKHTKKRQIYTDIEMNRLGK
Vesicular stomatitis virus Glycoprotein (VSVG truncation) (F448 Truncation)
(SEQ ID NO: 108)
MKCLLYLAFLFIGVNCKKNPIELVEGWFSSWKSSIASFFFIIGLIIGLFLVLRVGI
HLCIKLKHTKKRQIYTDIEMNRLGK
Targeting Domain-VSVG Truncation fusion 421
(SEQ ID NO: 109)
MKCLLYLAFLFIGVNCK-X, wherein X is a Targeting Domain-
FEHPHIQDAASQLPDDESLFFGDTGLSKNPIELVEGWFSSWKSSIASFFFIIGLII
GLFLVLRVGIHLCIKLKHTKKRQIYTDIEMNRLGK
Targeting Domain-VSVG Truncation fusion 440
(SEQ ID NO: 110)
MKCLLYLAFLFIGVNCK-X, wherein X is a targeting Domain-
FFGDTGLSKNPIELVEGWFSSWKSSIASFFFIIGLIIGLFLVLRVGIHLCIKLKHT
KKRQIYTDIEMNRLGK
Targeting Domain-VSVG Truncation fusion 448
(SEQ ID NO: 111)
MKCLLYLAFLFIGVNCK-X, wherein X is a Targeting Domain-
KNPIELVEGWFSSWKSSIASFFFIIGLIIGLFLVLRVGIHLCIKLKHTKKRQIYTDI
EMNRLGK
Amphotropic Murine leukemia virus Glycoprotein (AMLVG) WT
(SEQ ID NO: 112)
MARSTLSKPPQDKINPWKPLIVMGVLLGVGMAESPHQVFNVTWRVTNLMTG
RTANATSLLGTVQDAFPKLYFDLCDLVGEEWDPSDQEPYVGYGCKYPAGRQ
RTRTFDFYVCPGHTVKSGCGGPGEGYCGKWGCETTGQAYWKPTSSWDLISL
KRGNTPWDTGCSKVACGPCYDLSKVSNSFQGATRGGRCNPLVLEFTDAGKK
ANWDGPKSWGLRLYRTGTDPITMFSLTRQVLNVGPRVPIGPNPVLPDQRLPSS
PIEIVPAPQPPSPLNTSYPPSTTSTPSTSPTSPSVPQPPPGTGDRLLALVKGAYQA
LNLTNPDKTQECWLCLVSGPPYYEGVAVVGTYTNHSTAPANCTATSQHKLT
LSEVTGQGLCMGAVPKTHQALCNTTQSAGSGSYYLAAPAGTMWACSTGLTP
CLSTTVLNLTTDYCVLVELWPRVIYHSPDYMYGQLEQRTKYKREPVSLTLAL
LLGGLTMGGIAAGIGTGTTALIKTQQFEQLHAAIQTDLNEVEKSITNLEKSLTS
LSEVVLQNRRGLDLLFLKEGGLCAALKEECCFYADHTGLVRDSMAKLRERL
NQRQKLFETGQGWFEGLFNRSPWFTTLISTIMGPLIVLLLILLFGPCILNRLVQF
VKDRISVVQALVLTQQYHQLKPIEYEP
Amphotropic Murine leukemia virus Glycoprotein (AMLVG) (D86K) Receptor
binding domain mutant
(SEQ ID NO: 113)
MARSTLSKPPQDKINPWKPLIVMGVLLGVGMAESPHQVFNVTWRVTNLMTG
RTANATSLLGTVQDAFPKLYFDLCDLVGEEWDPSKQEPYVGYGCKYPAGRQ
RTRTFDFYVCPGHTVKSGCGGPGEGYCGKWGCETTGQAYWKPTSSWDLISL
KRGNTPWDTGCSKVACGPCYDLSKVSNSFQGATRGGRCNPLVLEFTDAGKK
ANWDGPKSWGLRLYRTGTDPITMFSLTRQVLNVGPRVPIGPNPVLPDQRLPSS
PIEIVPAPQPPSPLNTSYPPSTTSTPSTSPTSPSVPQPPPGTGDRLLALVKGAYQA
LNLTNPDKTQECWLCLVSGPPYYEGVAVVGTYTNHSTAPANCTATSQHKLT
LSEVTGQGLCMGAVPKTHQALCNTTQSAGSGSYYLAAPAGTMWACSTGLTP
CLSTTVLNLTTDYCVLVELWPRVIYHSPDYMYGQLEQRTKYKREPVSLTLAL
LLGGLTMGGIAAGIGTGTTALIKTQQFEQLHAAIQTDLNEVEKSITNLEKSLTS
LSEVVLQNRRGLDLLFLKEGGLCAALKEECCFYADHTGLVRDSMAKLRERL
NQRQKLFETGQGWFEGLFNRSPWFTTLISTIMGPLIVLLLILLFGPCILNRLVQF
VKDRISVVQALVLTQQYHQLKPIEYEP
Amphotropic Murine leukemia virus Glycoprotein (AMLVG) (R-domain deletion)
(SEQ ID NO: 114)
MARSTLSKPPQDKINPWKPLIVMGVLLGVGMAESPHQVFNVTWRVTNLMTG
RTANATSLLGTVQDAFPKLYFDLCDLVGEEWDPSDQEPYVGYGCKYPAGRQ
RTRTFDFYVCPGHTVKSGCGGPGEGYCGKWGCETTGQAYWKPTSSWDLISL
KRGNTPWDTGCSKVACGPCYDLSKVSNSFQGATRGGRCNPLVLEFTDAGKK
ANWDGPKSWGLRLYRTGTDPITMFSLTRQVLNVGPRVPIGPNPVLPDQRLPSS
PIEIVPAPQPPSPLNTSYPPSTTSTPSTSPTSPSVPQPPPGTGDRLLALVKGAYQA
LNLTNPDKTQECWLCLVSGPPYYEGVAVVGTYTNHSTAPANCTATSQHKLT
LSEVTGQGLCMGAVPKTHQALCNTTQSAGSGSYYLAAPAGTMWACSTGLTP
CLSTTVLNLTTDYCVLVELWPRVIYHSPDYMYGQLEQRTKYKREPVSLTLAL
LLGGLTMGGIAAGIGTGTTALIKTQQFEQLHAAIQTDLNEVEKSITNLEKSLTS
LSEVVLQNRRGLDLLFLKEGGLCAALKEECCFYADHTGLVRDSMAKLRERL
NQRQKLFETGQGWFEGLFNRSPWFTTLISTIMGPLIVLLLILLFGPCILNRLVQF
VKDRISVVQA
Amphotropic Murine leukemia virus Glycoprotein (AMLVG) (Furin-cleavage
mutant)
(SEQ ID NO: 115)
MARSTLSKPPQDKINPWKPLIVMGVLLGVGMAESPHQVFNVTWRVTNLMTG
RTANATSLLGTVQDAFPKLYFDLCDLVGEEWDPSDQEPYVGYGCKYPAGRQ
RTRTFDFYVCPGHTVKSGCGGPGEGYCGKWGCETTGQAYWKPTSSWDLISL
KRGNTPWDTGCSKVACGPCYDLSKVSNSFQGATRGGRCNPLVLEFTDAGKK
ANWDGPKSWGLRLYRTGTDPITMFSLTRQVLNVGPRVPIGPNPVLPDQRLPSS
PIEIVPAPQPPSPLNTSYPPSTTSTPSTSPTSPSVPQPPPGTGDRLLALVKGAYQA
LNLTNPDKTQECWLCLVSGPPYYEGVAVVGTYTNHSTAPANCTATSQHKLT
LSEVTGQGLCMGAVPKTHQALCNTTQSAGSGSYYLAAPAGTMWACSTGLTP
CLSTTVLNLTTDYCVLVELWPRVIYHSPDYMYGQLEQRTIEGREPVSLTLALL
LGGLTMGGIAAGIGTGTTALIKTQQFEQLHAAIQTDLNEVEKSITNLEKSLTSL
SEVVLQNRRGLDLLFLKEGGLCAALKEECCFYADHTGLVRDSMAKLRERLN
QRQKLFETGQGWFEGLFNRSPWFTTLISTIMGPLIVLLLILLFGPCILNRLVQFV
KDRISVVQALVLTQQYHQLKPIEYEP
Amphotropic Murine leukemia virus Glycoprotein (AMLVG) (L640A)
(SEQ ID NO: 116)
MARSTLSKPPQDKINPWKPLIVMGVLLGVGMAESPHQVFNVTWRVTNLMTG
RTANATSLLGTVQDAFPKLYFDLCDLVGEEWDPSDQEPYVGYGCKYPAGRQ
RTRTFDFYVCPGHTVKSGCGGPGEGYCGKWGCETTGQAYWKPTSSWDLISL
KRGNTPWDTGCSKVACGPCYDLSKVSNSFQGATRGGRCNPLVLEFTDAGKK
ANWDGPKSWGLRLYRTGTDPITMFSLTRQVLNVGPRVPIGPNPVLPDQRLPSS
PIEIVPAPQPPSPLNTSYPPSTTSTPSTSPTSPSVPQPPPGTGDRLLALVKGAYQA
LNLTNPDKTQECWLCLVSGPPYYEGVAVVGTYTNHSTAPANCTATSQHKLT
LSEVTGQGLCMGAVPKTHQALCNTTQSAGSGSYYLAAPAGTMWACSTGLTP
CLSTTVLNLTTDYCVLVELWPRVIYHSPDYMYGQLEQRTKYKREPVSLTLAL
LLGGLTMGGIAAGIGTGTTALIKTQQFEQLHAAIQTDLNEVEKSITNLEKSLTS
LSEVVLQNRRGLDLLFLKEGGLCAALKEECCFYADHTGLVRDSMAKLRERL
NQRQKLFETGQGWFEGLFNRSPWFTTLISTIMGPLIVLLLILLFGPCILNRLVQF
VKDRISVVQALVATQQYHQLKPIEYEP
Amphotropic Murine leukemia virus Glycoprotein (AMLVG) (Y644A)
Endocytosis signal mutant
(SEQ ID NO: 117)
MARSTLSKPPQDKINPWKPLIVMGVLLGVGMAESPHQVFNVTWRVTNLMTG
RTANATSLLGTVQDAFPKLYFDLCDLVGEEWDPSDQEPYVGYGCKYPAGRQ
RTRTFDFYVCPGHTVKSGCGGPGEGYCGKWGCETTGQAYWKPTSSWDLISL
KRGNTPWDTGCSKVACGPCYDLSKVSNSFQGATRGGRCNPLVLEFTDAGKK
ANWDGPKSWGLRLYRTGTDPITMFSLTRQVLNVGPRVPIGPNPVLPDQRLPSS
PIEIVPAPQPPSPLNTSYPPSTTSTPSTSPTSPSVPQPPPGTGDRLLALVKGAYQA
LNLTNPDKTQECWLCLVSGPPYYEGVAVVGTYTNHSTAPANCTATSQHKLT
LSEVTGQGLCMGAVPKTHQALCNTTQSAGSGSYYLAAPAGTMWACSTGLTP
CLSTTVLNLTTDYCVLVELWPRVIYHSPDYMYGQLEQRTKYKREPVSLTLAL
LLGGLTMGGIAAGIGTGTTALIKTQQFEQLHAAIQTDLNEVEKSITNLEKSLTS
LSEVVLQNRRGLDLLFLKEGGLCAALKEECCFYADHTGLVRDSMAKLRERL
NQRQKLFETGQGWFEGLFNRSPWFTTLISTIMGPLIVLLLILLFGPCILNRLVQF
VKDRISVVQALVLTQQAHQLKPIEYEP
Targeting Domain-AMLVG fusion site
(SEQ ID NO: 118)
MARSTLSKPPQDKINPWKPLIVMGVLLGVG-X, wherein X is a Targeting
Domain-
MAESPHQVFNVTWRVTNLMTGRTANATSLLGTVQDAFPKLYFDLCDLVGEE
WDPSDQEPYVGYGCKYPAGRQRTRTFDFYVCPGHTVKSGCGGPGEGYCGK
WGCETTGQAYWKPTSSWDLISLKRGNTPWDTGCSKVACGPCYDLSKVSNSF
QGATRGGRCNPLVLEFTDAGKKANWDGPKSWGLRLYRTGTDPITMFSLTRQ
VLNVGPRVPIGPNPVLPDQRLPSSPIEIVPAPQPPSPLNTSYPPSTTSTPSTSPTSP
SVPQPPPGTGDRLLALVKGAYQALNLTNPDKTQECWLCLVSGPPYYEGVAV
VGTYTNHSTAPANCTATSQHKLTLSEVTGQGLCMGAVPKTHQALCNTTQSA
GSGSYYLAAPAGTMWACSTGLTPCLSTTVLNLTTDYCVLVELWPRVIYHSPD
YMYGQLEQRTKYKREPVSLTLALLLGGLTMGGIAAGIGTGTTALIKTQQFEQ
LHAAIQTDLNEVEKSITNLEKSLTSLSEVVLQNRRGLDLLFLKEGGLCAALKE
ECCFY ADHTGLVRDSMAKLRERLNQRQKLFETGQGWFEGLFNRSPWFTTLIS
TIMGPLIVLLLILLFGPCILNRLVQFVKDRISVVQALVLTQQYHQLKPIEYEP
10A1 Murine leukemia virus Glycoprotein (10A1MLVG) WT
(SEQ ID NO: 119)
MARSTLSKPLKDKINPWKSLMVMGVLLRVGMAESPHQVFNVTWRVTNLMT
GRTANATSLLGTVQDAFPRLYFDLCDLVGEEWDPSDQEPYVGYGCKYPGGR
KRTRTFDFYVCPGHTVKSGCGGPREGYCGEWGCETTGQAYWKPTSSWDLIS
LKRGNTPWDTGCSKMACGPCYDLSKVSNSFQGATRGGRCNPLVLEFTDAGK
KANWDGPKSWGLRLYRTGTDPITMFSLTRQVLNIGPRIPIGPNPVITGQLPPSR
PVQIRLPRPPQPPPTGAASIVPETAPPSQQPGTGDRLLNLVEGAYRALNLTNPD
KTQECWLCLVSGPPYYEGVAVVGTYTNHSTAPASCTATSQHKLTLSEVTGQ
GLCMGAVPKTHQALCNTTQSAGSGSYYLAAPAGTMWACSTGLTPCLSTTML
NLTTDYCVLVELWPRIIYHSPDYMYGQLEQRTKYKREPVSLTLALLLGGLTM
GGIAAGIGTGTTALIKTQQFEQLHAAIQTDLNEVEKSITNLEKSLTSLSEVVLQ
NRRGLDLLFLKEGGLCAALKEECCFYADHTGLVRDSMAKLRERLNQRQKLF
ESGQGWFEGLFNRSPWFTTLISTIMGPLIVLLLILLFGPCILNRLVQFVKDRISV
VQALVLTQQYHQLKPIEYEP
10A1 Murine leukemia virus Glycoprotein (10A1MLVG) (D86K) Receptor
binding domain mutant
(SEQ ID NO: 120)
MARSTLSKPLKDKINPWKSLMVMGVLLRVGMAESPHQVFNVTWRVTNLMT
GRTANATSLLGTVQDAFPRLYFDLCDLVGEEWDPSKQEPYVGYGCKYPGGR
KRTRTFDFYVCPGHTVKSGCGGPREGYCGEWGCETTGQAYWKPTSSWDLIS
LKRGNTPWDTGCSKMACGPCYDLSKVSNSFQGATRGGRCNPLVLEFTDAGK
KANWDGPKSWGLRLYRTGTDPITMFSLTRQVLNIGPRIPIGPNPVITGQLPPSR
PVQIRLPRPPQPPPTGAASIVPETAPPSQQPGTGDRLLNLVEGAYRALNLTNPD
KTQECWLCLVSGPPYYEGVAVVGTYTNHSTAPASCTATSQHKLTLSEVTGQ
GLCMGAVPKTHQALCNTTQSAGSGSYYLAAPAGTMWACSTGLTPCLSTTML
NLTTDYCVLVELWPRIIYHSPDYMYGQLEQRTKYKREPVSLTLALLLGGLTM
GGIAAGIGTGTTALIKTQQFEQLHAAIQTDLNEVEKSITNLEKSLTSLSEVVLQ
NRRGLDLLFLKEGGLCAALKEECCFYADHTGLVRDSMAKLRERLNQRQKLF
ESGQGWFEGLFNRSPWFTTLISTIMGPLIVLLLILLFGPCILNRLVQFVKDRISV
VQALVLTQQYHQLKPIEYEP
10A1 Murine leukemia virus Glycoprotein (10A1MLVG) (R-domain deletion)
(SEQ ID NO: 121)
MARSTLSKPLKDKINPWKSLMVMGVLLRVGMAESPHQVFNVTWRVTNLMT
GRTANATSLLGTVQDAFPRLYFDLCDLVGEEWDPSDQEPYVGYGCKYPGGR
KRTRTFDFYVCPGHTVKSGCGGPREGYCGEWGCETTGQAYWKPTSSWDLIS
LKRGNTPWDTGCSKMACGPCYDLSKVSNSFQGATRGGRCNPLVLEFTDAGK
KANWDGPKSWGLRLYRTGTDPITMFSLTRQVLNIGPRIPIGPNPVITGQLPPSR
PVQIRLPRPPQPPPTGAASIVPETAPPSQQPGTGDRLLNLVEGAYRALNLTNPD
KTQECWLCLVSGPPYYEGVAVVGTYTNHSTAPASCTATSQHKLTLSEVTGQ
GLCMGAVPKTHQALCNTTQSAGSGSYYLAAPAGTMWACSTGLTPCLSTTML
NLTTDYCVLVELWPRIIYHSPDYMYGQLEQRTKYKREPVSLTLALLLGGLTM
GGIAAGIGTGTTALIKTQQFEQLHAAIQTDLNEVEKSITNLEKSLTSLSEVVLQ
NRRGLDLLFLKEGGLCAALKEECCFYADHTGLVRDSMAKLRERLNQRQKLF
ESGQGWFEGLFNRSPWFTTLISTIMGPLIVLLLILLFGPCILNRLVQFVKDRISV
VQA
10A1 Murine leukemia virus Glycoprotein (10A1MLVG) (Furin-cleavage mutant)
(SEQ ID NO: 122)
MARSTLSKPLKDKINPWKSLMVMGVLLRVGMAESPHQVFNVTWRVTNLMT
GRTANATSLLGTVQDAFPRLYFDLCDLVGEEWDPSDQEPYVGYGCKYPGGR
KRTRTFDFYVCPGHTVKSGCGGPREGYCGEWGCETTGQAYWKPTSSWDLIS
LKRGNTPWDTGCSKMACGPCYDLSKVSNSFQGATRGGRCNPLVLEFTDAGK
KANWDGPKSWGLRLYRTGTDPITMFSLTRQVLNIGPRIPIGPNPVITGQLPPSR
PVQIRLPRPPQPPPTGAASIVPETAPPSQQPGTGDRLLNLVEGAYRALNLTNPD
KTQECWLCLVSGPPYYEGVAVVGTYTNHSTAPASCTATSQHKLTLSEVTGQ
GLCMGAVPKTHQALCNTTQSAGSGSYYLAAPAGTMWACSTGLTPCLSTTML
NLTTDYCVLVELWPRIIYHSPDYMYGQLEQRTIEGREPVSLTLALLLGGLTMG
GIAAGIGTGTTALIKTQQFEQLHAAIQTDLNEVEKSITNLEKSLTSLSEVVLQN
RRGLDLLFLKEGGLCAALKEECCFYADHTGLVRDSMAKLRERLNQRQKLFE
SGQGWFEGLFNRSPWFTTLISTIMGPLIVLLLILLFGPCILNRLVQFVKDRISVV
QALVLTQQYHQLKPIEYEP
10A1 Murine leukemia virus Glycoprotein (10A1MLVG) (L631A)
(SEQ ID NO: 123)
MARSTLSKPLKDKINPWKSLMVMGVLLRVGMAESPHQVFNVTWRVTNLMT
GRTANATSLLGTVQDAFPRLYFDLCDLVGEEWDPSDQEPYVGYGCKYPGGR
KRTRTFDFYVCPGHTVKSGCGGPREGYCGEWGCETTGQAYWKPTSSWDLIS
LKRGNTPWDTGCSKMACGPCYDLSKVSNSFQGATRGGRCNPLVLEFTDAGK
KANWDGPKSWGLRLYRTGTDPITMFSLTRQVLNIGPRIPIGPNPVITGQLPPSR
PVQIRLPRPPQPPPTGAASIVPETAPPSQQPGTGDRLLNLVEGAYRALNLTNPD
KTQECWLCLVSGPPYYEGVAVVGTYTNHSTAPASCTATSQHKLTLSEVTGQ
GLCMGAVPKTHQALCNTTQSAGSGSYYLAAPAGTMWACSTGLTPCLSTTML
NLTTDYCVLVELWPRIIYHSPDYMYGQLEQRTKYKREPVSLTLALLLGGLTM
GGIAAGIGTGTTALIKTQQFEQLHAAIQTDLNEVEKSITNLEKSLTSLSEVVLQ
NRRGLDLLFLKEGGLCAALKEECCFYADHTGLVRDSMAKLRERLNQRQKLF
ESGQGWFEGLFNRSPWFTTLISTIMGPLIVLLLILLFGPCILNRLVQFVKDRISV
VQALVATQQYHQLKPIEYEP
10A1 Murine leukemia virus Glycoprotein (10A1MLVG) (Y635A) Endocytosis
signal mutant
(SEQ ID NO: 124)
MARSTLSKPLKDKINPWKSLMVMGVLLRVGMAESPHQVFNVTWRVTNLMT
GRTANATSLLGTVQDAFPRLYFDLCDLVGEEWDPSDQEPYVGYGCKYPGGR
KRTRTFDFYVCPGHTVKSGCGGPREGYCGEWGCETTGQAYWKPTSSWDLIS
LKRGNTPWDTGCSKMACGPCYDLSKVSNSFQGATRGGRCNPLVLEFTDAGK
KANWDGPKSWGLRLYRTGTDPITMFSLTRQVLNIGPRIPIGPNPVITGQLPPSR
PVQIRLPRPPQPPPTGAASIVPETAPPSQQPGTGDRLLNLVEGAYRALNLTNPD
KTQECWLCLVSGPPYYEGVAVVGTYTNHSTAPASCTATSQHKLTLSEVTGQ
GLCMGAVPKTHQALCNTTQSAGSGSYYLAAPAGTMWACSTGLTPCLSTTML
NLTTDYCVLVELWPRIIYHSPDYMYGQLEQRTKYKREPVSLTLALLLGGLTM
GGIAAGIGTGTTALIKTQQFEQLHAAIQTDLNEVEKSITNLEKSLTSLSEVVLQ
NRRGLDLLFLKEGGLCAALKEECCFYADHTGLVRDSMAKLRERLNQRQKLF
ESGQGWFEGLFNRSPWFTTLISTIMGPLIVLLLILLFGPCILNRLVQFVKDRISV
VQALVLTQQAHQLKPIEYEP
Targeting Domain-10A1MLVG fusion site
(SEQ ID NO: 125)
MARSTLSKPLKDKINPWKSLMVMGVLLRVG-X, wherein X is a targeting Domain-
MAESPHQV
FNVTWRVTNLMTGRTANATSLLGTVQDAFPRLYFDLCDLVGEEWDPSDQEP
YVGYGCKYPGGRKRTRTFDFYVCPGHTVKSGCGGPREGYCGEWGCETTGQ
AYWKPTSSWDLISLKRGNTPWDTGCSKMACGPCYDLSKVSNSFQGATRGGR
CNPLVLEFTDAGKKANWDGPKSWGLRLYRTGTDPITMFSLTRQVLNIGPRIPI
GPNPVITGQLPPSRPVQIRLPRPPQPPPTGAASIVPETAPPSQQPGTGDRLLNLV
EGAYRALNLTNPDKTQECWLCLVSGPPYYEGVAVVGTYTNHSTAPASCTAT
SQHKLTLSEVTGQGLCMGAVPKTHQALCNTTQSAGSGSYYLAAPAGTMWA
CSTGLTPCLSTTMLNLTTDYCVLVELWPRIIYHSPDYMYGQLEQRTKYKREP
VSLTLALLLGGLTMGGIAAGIGTGTTALIKTQQFEQLHAAIQTDLNEVEKSITN
LEKSLTSLSEVVLQNRRGLDLLFLKEGGLCAALKEECCFYADHTGLVRDSMA
KLRERLNQRQKLFESGQGWFEGLFNRSPWFTTLISTIMGPLIVLLLILLFGPCIL
NRLVQFVKDRISVVQALVLTQQYHQLKPIEYEP
Influenza A (FPV)/Rostock/1934, subtype H7 virus Hemagglutinin WT
(SEQ ID NO: 126)
MNTQILVFALVAVIPTNADKICLGHHAVSNGTKVNTLTERGVEVVNATETVERTNIPKI
CSKGKRTTDLGQCGLLGTITGPPQCDQFLEFSADLIIERREGNDVCYPGKFVNEEAL
RQILRGSGGIDKETMGFTYSGIRTNGTTSACRRSGSSFYAEMEWLLSNTDNASFPQ
MTKSYKNTRRESALIVWGIHHSGSTTEQTKLYGSGNKLITVGSSKYHQSFVPSPGT
RPQINGQSGRIDFHWLILDPNDTVTFSFNGAFIAPNRASFLRGKSMGIQSDVQVDAN
CEGECYHSGGTITSRLPFQNINSRAVGKCPRYVKQESLLLATGMKNVPEPSKKRKK
RGLFGAIAGFIENGWEGLVDGWYGFRHQNAQGEGTAADYKSTQSAIDQITGKLNRL
IEKTNQQFELIDNEFTEVEKQIGNLINWTKDSITEVWSYNAELIVAMENQHTIDLADSE
MNRLYERVRKQLRENAEEDGTGCFEIFHKCDDDCMASIRNNTYDHSKYREEAMQN
RIQIDPVKLSSGYKDVILWFSFGASCFLLLAIAMGLVFICVKNGNMRCTICI
Influenza A (FPV)/Rostock/1934, subtype H7 virus Hemagglutinin
(Y106F) (E199Q) (G237K)
(SEQ ID NO: 127)
MNTQILVFALVAVIPTNADKICLGHHAVSNGTKVNTLTERGVEVVNATETVERTNIPKI
CSKGKRTTDLGQCGLLGTITGPPQCDQFLEFSADLIIERREGNDVCFPGKFVNEEAL
RQILRGSGGIDKETMGFTYSGIRTNGTTSACRRSGSSFYAEMEWLLSNTDNASFPQ
MTKSYKNTRRESALIVWGIHHSGSTTQQTKLYGSGNKLITVGSSKYHQSFVPSPGT
RPQINGQSKRIDFHWLILDPNDTVTFSFNGAFIAPNRASFLRGKSMGIQSDVQVDAN
CEGECYHSGGTITSRLPFQNINSRAVGKCPRYVKQESLLLATGMKNVPEPSKKRKK
RGLFGAIAGFIENGWEGLVDGWYGFRHQNAQGEGTAADYKSTQSAIDQITGKLNRL
IEKTNQQFELIDNEFTEVEKQIGNLINWTKDSITEVWSYNAELIVAMENQHTIDLADSE
MNRLYERVRKQLRENAEEDGTGCFEIFHKCDDDCMASIRNNTYDHSKYREEAMQN
RIQIDPVKLSSGYKDVILWFSFGASCFLLLAIAMGLVFICVKNGNMRCTICI
Influenza A (FPV)/Rostock/1934, subtype H7 virus Hemagglutinin (Furin
cleavage mutation)
(SEQ ID NO: 128)
MNTQILVFALVAVIPTNADKICLGHHAVSNGTKVNTLTERGVEVVNATETVE
RTNIPKICSKGKRTTDLGQCGLLGTITGPPQCDQFLEFSADLIIERREGNDVCYP
GKFVNEEALRQILRGSGGIDKETMGFTYSGIRTNGTTSACRRSGSSFYAEMEW
LLSNTDNASFPQMTKSYKNTRRESALIVWGIHHSGSTTEQTKLYGSGNKLITV
GSSKYHQSFVPSPGTRPQINGQSGRIDFHWLILDPNDTVTFSFNGAFIAPNRAS
FLRGKSMGIQSDVQVDANCEGECYHSGGTITSRLPFQNINSRAVGKCPRYVK
QESLLLATGMKNVPEPSKKRKGKRGLFGAIAGFIENGWEGLVDGWYGFRHQ
NAQGEGTAADYKSTQSAIDQITGKLNRLIEKTNQQFELIDNEFTEVEKQIGNLI
NWTKDSITEVWSYNAELIVAMENQHTIDLADSEMNRLYERVRKQLRENAEE
DGTGCFEIFHKCDDDCMASIRNNTYDHSKYREEAMQNRIQIDPVKLSSGYKD
VILWFSFGASCFLLLAIAMGLVFICVKNGNMRCTICI
Influenza A/Puerto Rico/8/34, subtype N1 Neuraminidase WT
(SEQ ID NO: 129)
MNPNQKIITIGSICLVVGLISLILQIGNIISIWISHSIQTGSQNHTGICNQNIITYKN
STWVKDTTSVILTGNSSLCPIRGWAIYSKDNSIRIGSKGDVFVIREPFISCSHLE
CRTFFLTQGALLNDKHSSGTVKDRSPYRALMSCPVGEAPSPYNSRFESVAWS
ASACHDGMGWLTIGISGPDNGAVAVLKYNGIITETIKSWRKKILRTQESECAC
VNGSCFTIMTDGPSDGLASYKIFKIEKGKVTKSIELNAPNSHYEECSCYPDTGK
VMCVCRDNWHGSNRPWVSFDQNLDYQIGYICSGVFGDNPRPEDGTGSCGPV
YVDGANGVKGFSYRYGNGVWIGRTKSHSSRHGFEMIWDPNGWTETDSKFSV
RQDVVAMTDWSGYSGSFVQHPELTGLDCMRPCFWVELIRGRPKEKTIWTSA
SSISFCGVNSDTVDWSWPDGAELPFSIDK
Influenza A/Puerto Rico/8/34, subtype N1 Neuraminidase (T55A)
(SEQ ID NO: 130)
MNPNQKIITIGSICLVVGLISLILQIGNIISIWISHSIQTGSQNHTGICNQNIIAYKN
STWVKDTTSVILTGNSSLCPIRGWAIYSKDNSIRIGSKGDVFVIREPFISCSHLE
CRTFFLTQGALLNDKHSSGTVKDRSPYRALMSCPVGEAPSPYNSRFESVAWS
ASACHDGMGWLTIGISGPDNGAVAVLKYNGIITETIKSWRKKILRTQESECAC
VNGSCFTIMTDGPSDGLASYKIFKIEKGKVTKSIELNAPNSHYEECSCYPDTGK
VMCVCRDNWHGSNRPWVSFDQNLDYQIGYICSGVFGDNPRPEDGTGSCGPV
YVDGANGVKGFSYRYGNGVWIGRTKSHSSRHGFEMIWDPNGWTETDSKFSV
RQDVVAMTDWSGYSGSFVQHPELTGLDCMRPCFWVELIRGRPKEKTIWTSA
SSISFCGVNSDTVDWSWPDGAELPFSIDK
Sindbis Virus Glycoprotein (SINVG) WT
(SEQ ID NO: 131)
SAAPLVTAMCLLGNVSFPCNRPPTCYTREPSRALDILEENVNHEAYDTLLNAI
LRCGSSGRSKRSVTDDFTLTSPYLGTCSYCHHTEPCFSPIKIEQVWDEADDNTI
RIQTSAQFGYDQSGAASSNKYRYMSLEQDHTVKEGTMDDIKISTSGPCRRLS
YKGYFLLAKCPPGDSVTVSIASSNSATSCTMARKIKPKFVGREKYDLPPVHGK
KIPCTVYDRLKETTAGYITMHRPGPHAYTSYLEESSGKVYAKPPSGKNITYEC
KCGDYKTGTVTTRTEITGCTAIKQCVAYKSDQTKWVFNSPDLIRHADHTAQG
KLHLPFKLIPSTCMVPVAHAPNVVHGFKHISLQLDTDHLTLLTTRRLGANPEP
TTEWIIGKTVRNFTVDRDGLEYIWGNHEPVRVYAQESAPGDPHGWPHEIVQH
YYHRHPVYTILAVASAAVAMMIGVTVAALCACKARRECLTPYALAPNAVIPT
SLALLCCVRSANAETFTETMSYLWSNSQPFFWVQLCIPLAAVIVLMRCCSCCL
PFLVVAGAYLAKVDAYEHATTVPNVPQIPYKALVERAGYAPLNLEITVMSSE
VLPSTNQEYITCKFTTVVPSPKVKCCGSLECQPAAHADYTCKVFGGVYPFMW
GGAQCFCDSENSQMSEAYVELSADCATDHAQAIKVHTAAMKVGLRIVYGNT
TSFLDVYVNGVTPGTSKDLKVIAGPISASFTPFDHKVVIHRGLVYNYDFPEYG
AMKPGAFGDIQATSLTSKDLIASTDIRLLKPSAKNVHVPYTQAASGFEMWKN
NSGRPLQETAPFGCKIAVNPLRAVDCSYGNIPISIDIPNAAFIRTSDAPLVSTVK
CDVSECTYSADFGGMATLQYVSDREGQCPVHSHSSTATLQESTVHVLEKGA
VTVHFSTASPQANFIVSLCGKKTTCNAECKPPADHIVSTPHKNDQEFQAAISK
TSWSWLFALFGGASSLLIIGLMIFACSMMLTSTRR
Sindbis Virus Glycoprotein (SINVG) triple MUT, E3(61-64del)
E2(68SLEQ71 to 68AAAA71) E2(159KE160 to 159AA160)
(SEQ ID NO: 132)
SAAPLVTAMCLLGNVSFPCNRPPTCYTREPSRALDILEENVNHEAYDTLLNAI
LRCGSSGSVTDDFTLTSPYLGTCSYCHHTEPCFSPIKIEQVWDEADDNTIRIQT
SAQFGYDQSGAASSNKYRYMAAAADHTVKEGTMDDIKISTSGPCRRLSYKG
YFLLAKCPPGDSVTVSIASSNSATSCTMARKIKPKFVGREKYDLPPVHGKKIP
CTVYDRLAATTAGYITMHRPGPHAYTSYLEESSGKVYAKPPSGKNITYECKC
GDYKTGTVTTRTEITGCTAIKQCVAYKSDQTKWVFNSPDLIRHADHTAQGKL
HLPFKLIPSTCMVPVAHAPNVVHGFKHISLQLDTDHLTLLTTRRLGANPEPTT
EWIIGKTVRNFTVDRDGLEYIWGNHEPVRVYAQESAPGDPHGWPHEIVQHY
YHRHPVYTILAVASAAVAMMIGVTVAALCACKARRECLTPYALAPNAVIPTS
LALLCCVRSANAETFTETMSYLWSNSQPFFWVQLCIPLAAVIVLMRCCSCCLP
FLVVAGAYLAKVDAYEHATTVPNVPQIPYKALVERAGYAPLNLEITVMSSEV
LPSTNQEYITCKFTTVVPSPKVKCCGSLECQPAAHADYTCKVFGGVYPFMWG
GAQCFCDSENSQMSEAYVELSADCATDHAQAIKVHTAAMKVGLRIVYGNTT
SFLDVYVNGVTPGTSKDLKVIAGPISASFTPFDHKVVIHRGLVYNYDFPEYGA
MKPGAFGDIQATSLTSKDLIASTDIRLLKPSAKNVHVPYTQAASGFEMWKNN
SGRPLQETAPFGCKIAVNPLRAVDCSYGNIPISIDIPNAAFIRTSDAPLVSTVKC
DVSECTYSADFGGMATLQYVSDREGQCPVHSHSSTATLQESTVHVLEKGAV
TVHFSTASPQANFIVSLCGKKTTCNAECKPPADHIVSTPHKNDQEFQAAISKTS
WSWLFALFGGASSLLIIGLMIFACSMMLTSTRR
Sindbis Virus Glycoprotein (SINVG) triple MUT, E3(61-64del)
E2(68SLEQ71 to 68AAAA71) E2(159KE160 to 159AA160) with HA TAG
(SEQ ID NO: 133)
SAAPLVTAMCLLGNVSFPCNRPPTCYTREPSRALDILEENVNHEAYDTLLNAI
LRCGSSGSVTDDFTLTSPYLGTCSYCHHTEPCFSPIKIEQVWDEADDNTIRIQT
SAQFGYDQSGAASSNKYRYMAAAAMYPYDVPDYATVKEGTMDDIKISTSGP
CRRLSYKGYFLLAKCPPGDSVTVSIASSNSATSCTMARKIKPKFVGREKYDLP
PVHGKKIPCTVYDRLAATTAGYITMHRPGPHAYTSYLEESSGKVYAKPPSGK
NITYECKCGDYKTGTVTTRTEITGCTAIKQCVAYKSDQTKWVFNSPDLIRHA
DHTAQGKLHLPFKLIPSTCMVPVAHAPNVVHGFKHISLQLDTDHLTLLTTRRL
GANPEPTTEWIIGKTVRNFTVDRDGLEYIWGNHEPVRVYAQESAPGDPHGWP
HEIVQHYYHRHPVYTILAVASAAVAMMIGVTVAALCACKARRECLTPYALA
PNAVIPTSLALLCCVRSANAETFTETMSYLWSNSQPFFWVQLCIPLAAVIVLM
RCCSCCLPFLVVAGAYLAKVDAYEHATTVPNVPQIPYKALVERAGYAPLNLE
ITVMSSEVLPSTNQEYITCKFTTVVPSPKVKCCGSLECQPAAHADYTCKVFGG
VYPFMWGGAQCFCDSENSQMSEAYVELSADCATDHAQAIKVHTAAMKVGL
RIVYGNTTSFLDVYVNGVTPGTSKDLKVIAGPISASFTPFDHKVVIHRGLVYN
YDFPEYGAMKPGAFGDIQATSLTSKDLIASTDIRLLKPSAKNVHVPYTQAASG
FEMWKNNSGRPLQETAPFGCKIAVNPLRAVDCSYGNIPISIDIPNAAFIRTSDA
PLVSTVKCDVSECTYSADFGGMATLQYVSDREGQCPVHSHSSTATLQESTVH
VLEKGAVTVHFSTASPQANFIVSLCGKKTTCNAECKPPADHIVSTPHKNDQEF
QAAISKTSWSWLFALFGGASSLLIIGLMIFACSMMLTSTRR
Sindbis Virus Glycoprotein (SINVG) triple MUT, E3(61-64del)
E2(68SLEQ71 to 68AAAA71) E2(159KE160 to 159AA160) with targeting
domain fusion site
(SEQ ID NO: 134)
SAAPLVTAMCLLGNVSFPCNRPPTCYTREPSRALDILEENVNHEAYDTLLNAI
LRCGSSGSVTDDFTLTSPYLGTCSYCHHTEPCFSPIKIEQVWDEADDNTIRIQT
SAQFGYDQSGAASSNKYRYMAAAA-(X, WHEREIN X IS A TARGETING
DOMAIN)-
TVKEGTMDDIKISTSGPCRRLSYKGYFLLAKCPPGDSVTVSIASSNSATSCTM
ARKIKPKFVGREKYDLPPVHGKKIPCTVYDRLAATTAGYITMHRPGPHAYTS
YLEESSGKVYAKPPSGKNITYECKCGDYKTGTVTTRTEITGCTAIKQCVAYKS
DQTKWVFNSPDLIRHADHTAQGKLHLPFKLIPSTCMVPVAHAPNVVHGFKHI
SLQLDTDHLTLLTTRRLGANPEPTTEWIIGKTVRNFTVDRDGLEYIWGNHEPV
RVYAQESAPGDPHGWPHEIVQHYYHRHPVYTILAVASAAVAMMIGVTVAAL
CACKARRECLTPYALAPNAVIPTSLALLCCVRSANAETFTETMSYLWSNSQPF
FWVQLCIPLAAVIVLMRCCSCCLPFLVVAGAYLAKVDAYEHATTVPNVPQIP
YKALVERAGYAPLNLEITVMSSEVLPSTNQEYITCKFTTVVPSPKVKCCGSLE
CQPAAHADYTCKVFGGVYPFMWGGAQCFCDSENSQMSEAYVELSADCATD
HAQAIKVHTAAMKVGLRIVYGNTTSFLDVYVNGVTPGTSKDLKVIAGPISAS
FTPFDHKVVIHRGLVYNYDFPEYGAMKPGAFGDIQATSLTSKDLIASTDIRLL
KPSAKNVHVPYTQAASGFEMWKNNSGRPLQETAPFGCKIAVNPLRAVDCSY
GNIPISIDIPNAAFIRTSDAPLVSTVKCDVSECTYSADFGGMATLQYVSDREGQ
CPVHSHSSTATLQESTVHVLEKGAVTVHFSTASPQANFIVSLCGKKTTCNAEC
KPPADHIVSTPHKNDQEFQAAISKTSWSWLFALFGGASSLLIIGLMIFACSMM
LTSTRR
Sindbis Virus Glycoprotein (SINVG) triple MUT version 2, E3(61-64del)
E2(68SLEQ71 to 68AAAA71) E2(159KE160 to 159AA160)
(SEQ ID NO: 135)
SAAPLVTAMCLLGNVSFPCDRPPTCYTREPSRALDILEENVNHEAYDTLLNAI
LRCGSSGSVIDDFTLTSPYLGTCSYCHHTEPCFSPVKIEQVWDEADDNTIRIQT
SAQFGYDQSGAASANKYRYMAAAADHTVKEGTMDDIKISTSGPCRRLSYKG
YFLLAKCPPGDSVTVSIVSSNSATSCTLARKIKPKFVGREKYDLPPVHGKKIPC
TVYDRLAATTAGYITMHRPGPHAYTSYLEESSGKVYAKPPSGKNITYECKCG
DYKTGTVSTRTEITGCTAIKQCVAYKSDQTKWVFNSPDLIRHDDHTVQGKLH
LPFKLIPSTCMVPVAHAPNVIHGFKHISLQLDTDHLTLLTTRRLGANPEPTTEW
IVGKTVRNFTVDRDGLEYIWGNHEPVRVYAQESAPGDPHGWPHEIVQHYYH
RHPVYTILAVASATVAMMIGVTVAVLCACKARRECLTPYALAPNAVIPTSLA
LLCCVRSANAETFTETMSYLWSNSQPFFWVQLCIPLAAFIVLMRCCSCCLPFL
VVAGAYLAKVDAYEHATTVPNVPQIPYKALVERAGYAPLNLEITVMSSEVLP
STNQEYITCKFTTVVPSPKIKCCGSLECQPAAHAGYTCKVFGGVYPFMWGGA
QCFCDSENSQMSEAYVELSADCASDHAQAIKVHTAAMKVGLRIVYGNTTSFL
DVYVNGVTPGTSKDLKVIAGPISASFTPFDHKVVIHRGLVYNYDFPEYGAMK
PGAFGDIQATSLTSKDLIASTDIRLLKPSAKNVHVPYTQASSGFEMWKNNSGR
PLQETAPFGCKIAVNPLRAVDCSYGNIPISIDIPNAAFIRTSDAPLVSTVKCEVS
ECTYSADFGGMATLQYVSDREGQCPVHSHSSTATLQESTVHVLEKGAVTVH
FSTASPQANFIVSLCGKKTTCNAECKPPADHIVSTPHKNDQEFQAAISKTSWS
WLFALFGGASSLLIIGLMIFACSMMLTSTRR
Sindbis Virus Glycoprotein (SINVG) triple MUT version 2, E3(61-64del)
E2(68SLEQ71 to 68AAAA71) E2(159KE160 to 159AA160) with HA TAG
(SEQ ID NO: 136)
SAAPLVTAMCLLGNVSFPCDRPPTCYTREPSRALDILEENVNHEAYDTLLNAI
LRCGSSGSVIDDFTLTSPYLGTCSYCHHTEPCFSPVKIEQVWDEADDNTIRIQT
SAQFGYDQSGAASANKYRYMAAAAMYPYDVPDYATVKEGTMDDIKISTSGP
CRRLSYKGYFLLAKCPPGDSVTVSIVSSNSATSCTLARKIKPKFVGREKYDLPP
VHGKKIPCTVYDRLAATTAGYITMHRPGPHAYTSYLEESSGKVYAKPPSGKN
ITYECKCGDYKTGTVSTRTEITGCTAIKQCVAYKSDQTKWVFNSPDLIRHDDH
TVQGKLHLPFKLIPSTCMVPVAHAPNVIHGFKHISLQLDTDHLTLLTTRRLGA
NPEPTTEWIVGKTVRNFTVDRDGLEYIWGNHEPVRVYAQESAPGDPHGWPH
EIVQHYYHRHPVYTILAVASATVAMMIGVTVAVLCACKARRECLTPYALAP
NAVIPTSLALLCCVRSANAETFTETMSYLWSNSQPFFWVQLCIPLAAFIVLMR
CCSCCLPFLVVAGAYLAKVDAYEHATTVPNVPQIPYKALVERAGYAPLNLEI
TVMSSEVLPSTNQEYITCKFTTVVPSPKIKCCGSLECQPAAHAGYTCKVFGGV
YPFMWGGAQCFCDSENSQMSEAYVELSADCASDHAQAIKVHTAAMKVGLRI
VYGNTTSFLDVYVNGVTPGTSKDLKVIAGPISASFTPFDHKVVIHRGLVYNYD
FPEYGAMKPGAFGDIQATSLTSKDLIASTDIRLLKPSAKNVHVPYTQASSGFE
MWKNNSGRPLQETAPFGCKIAVNPLRAVDCSYGNIPISIDIPNAAFIRTSDAPL
VSTVKCEVSECTYSADFGGMATLQYVSDREGQCPVHSHSSTATLQESTVHVL
EKGAVTVHFSTASPQANFIVSLCGKKTTCNAECKPPADHIVSTPHKNDQEFQA
AISKTSWSWLFALFGGASSLLIIGLMIFACSMMLTSTRR
Sindbis Virus Glycoprotein (SINVG) triple MUT version 2, E3(61-64del)
E2(68SLEQ71 to 68AAAA71) E2(159KE160 to 159AA160) with targeting
domain fusion site
(SEQ ID NO: 137)
SAAPLVTAMCLLGNVSFPCDRPPTCYTREPSRALDILEENVNHEAYDTLLNAI
LRCGSSGSVIDDFTLTSPYLGTCSYCHHTEPCFSPVKIEQVWDEADDNTIRIQT
SAQFGYDQSGAASANKYRYMAAAA-(X, WHEREIN X IS A TARGETING
DOMAIN)-
TVKEGTMDDIKISTSGPCRRLSYKGYFLLAKCPPGDSVTVSIVSSNSATSCTLA
RKIKPKFVGREKYDLPPVHGKKIPCTVYDRLAATTAGYITMHRPGPHAYTSY
LEESSGKVYAKPPSGKNITYECKCGDYKTGTVSTRTEITGCTAIKQCVAYKSD
QTKWVFNSPDLIRHDDHTVQGKLHLPFKLIPSTCMVPVAHAPNVIHGFKHISL
QLDTDHLTLLTTRRLGANPEPTTEWIVGKTVRNFTVDRDGLEYIWGNHEPVR
VYAQESAPGDPHGWPHEIVQHYYHRHPVYTILAVASATVAMMIGVTVAVLC
ACKARRECLTPYALAPNAVIPTSLALLCCVRSANAETFTETMSYLWSNSQPFF
WVQLCIPLAAFIVLMRCCSCCLPFLVVAGAYLAKVDAYEHATTVPNVPQIPY
KALVERAGYAPLNLEITVMSSEVLPSTNQEYITCKFTTVVPSPKIKCCGSLECQ
PAAHAGYTCKVFGGVYPFMWGGAQCFCDSENSQMSEAYVELSADCASDHA
QAIKVHTAAMKVGLRIVYGNTTSFLDVYVNGVTPGTSKDLKVIAGPISASFTP
FDHKVVIHRGLVYNYDFPEYGAMKPGAFGDIQATSLTSKDLIASTDIRLLKPS
AKNVHVPYTQASSGFEMWKNNSGRPLQETAPFGCKIAVNPLRAVDCSYGNIP
ISIDIPNAAFIRTSDAPLVSTVKCEVSECTYSADFGGMATLQYVSDREGQCPVH
SHSSTATLQESTVHVLEKGAVTVHFSTASPQANFIVSLCGKKTTCNAECKPPA
DHIVSTPHKNDQEFQAAISKTSWSWLFALFGGASSLLIIGLMIFACSMMLTSTR
R
Sindbis Virus Glycoprotein (SINVG) quad MUT, E3(61-64del)
E2(68SLEQ71 to 68AAAA71) E2(159KE160 to 159AA160) E1(250AK251 to
250SG251)
(SEQ ID NO: 138)
SAAPLVTAMCLLGNVSFPCNRPPTCYTREPSRALDILEENVNHEAYDTLLNAI
LRCGSSGSVTDDFTLTSPYLGTCSYCHHTEPCFSPIKIEQVWDEADDNTIRIQT
SAQFGYDQSGAASSNKYRYMAAAADHTVKEGTMDDIKISTSGPCRRLSYKG
YFLLAKCPPGDSVTVSIASSNSATSCTMARKIKPKFVGREKYDLPPVHGKKIP
CTVYDRLAATTAGYITMHRPGPHAYTSYLEESSGKVYAKPPSGKNITYECKC
GDYKTGTVTTRTEITGCTAIKQCVAYKSDQTKWVFNSPDLIRHADHTAQGKL
HLPFKLIPSTCMVPVAHAPNVVHGFKHISLQLDTDHLTLLTTRRLGANPEPTT
EWIIGKTVRNFTVDRDGLEYIWGNHEPVRVYAQESAPGDPHGWPHEIVQHY
YHRHPVYTILAVASAAVAMMIGVTVAALCACKARRECLTPYALAPNAVIPTS
LALLCCVRSANAETFTETMSYLWSNSQPFFWVQLCIPLAAVIVLMRCCSCCLP
FLVVAGAYLAKVDAYEHATTVPNVPQIPYKALVERAGYAPLNLEITVMSSEV
LPSTNQEYITCKFTTVVPSPKVKCCGSLECQPAAHADYTCKVFGGVYPFMWG
GAQCFCDSENSQMSEAYVELSADCATDHAQAIKVHTAAMKVGLRIVYGNTT
SFLDVYVNGVTPGTSKDLKVIAGPISASFTPFDHKVVIHRGLVYNYDFPEYGA
MKPGAFGDIQATSLTSKDLIASTDIRLLKPSSGNVHVPYTQAASGFEMWKNN
SGRPLQETAPFGCKIAVNPLRAVDCSYGNIPISIDIPNAAFIRTSDAPLVSTVKC
DVSECTYSADFGGMATLQYVSDREGQCPVHSHSSTATLQESTVHVLEKGAV
TVHFSTASPQANFIVSLCGKKTTCNAECKPPADHIVSTPHKNDQEFQAAISKTS
WSWLFALFGGASSLLIIGLMIFACSMMLTSTRR
Sindbis Virus Glycoprotein (SINVG) quad MUT, E3(61-64del)
E2(68SLEQ71 to 68AAAA71) E2(159KE160 to 159AA160) E1(250AK251 to
250SG251) with HA TAG
(SEQ ID NO: 139)
SAAPLVTAMCLLGNVSFPCNRPPTCYTREPSRALDILEENVNHEAYDTLLNAI
LRCGSSGSVTDDFTLTSPYLGTCSYCHHTEPCFSPIKIEQVWDEADDNTIRIQT
SAQFGYDQSGAASSNKYRYMAAAAMYPYDVPDYATVKEGTMDDIKISTSGP
CRRLSYKGYFLLAKCPPGDSVTVSIASSNSATSCTMARKIKPKFVGREKYDLP
PVHGKKIPCTVYDRLAATTAGYITMHRPGPHAYTSYLEESSGKVYAKPPSGK
NITYECKCGDYKTGTVTTRTEITGCTAIKQCVAYKSDQTKWVFNSPDLIRHA
DHTAQGKLHLPFKLIPSTCMVPVAHAPNVVHGFKHISLQLDTDHLTLLTTRRL
GANPEPTTEWIIGKTVRNFTVDRDGLEYIWGNHEPVRVYAQESAPGDPHGWP
HEIVQHYYHRHPVYTILAVASAAVAMMIGVTVAALCACKARRECLTPYALA
PNAVIPTSLALLCCVRSANAETFTETMSYLWSNSQPFFWVQLCIPLAAVIVLM
RCCSCCLPFLVVAGAYLAKVDAYEHATTVPNVPQIPYKALVERAGYAPLNLE
ITVMSSEVLPSTNQEYITCKFTTVVPSPKVKCCGSLECQPAAHADYTCKVFGG
VYPFMWGGAQCFCDSENSQMSEAYVELSADCATDHAQAIKVHTAAMKVGL
RIVYGNTTSFLDVYVNGVTPGTSKDLKVIAGPISASFTPFDHKVVIHRGLVYN
YDFPEYGAMKPGAFGDIQATSLTSKDLIASTDIRLLKPSSGNVHVPYTQAASG
FEMWKNNSGRPLQETAPFGCKIAVNPLRAVDCSYGNIPISIDIPNAAFIRTSDA
PLVSTVKCDVSECTYSADFGGMATLQYVSDREGQCPVHSHSSTATLQESTVH
VLEKGAVTVHFSTASPQANFIVSLCGKKTTCNAECKPPADHIVSTPHKNDQEF
QAAISKTSWSWLFALFGGASSLLIIGLMIFACSMMLTSTRR
Sindbis Virus Glycoprotein (SINVG) quad MUT, E3(61-64del)
E2(68SLEQ71 to 68AAAA71) E2(159KE160 to 159AA160) E1(250AK251 to
250SG251) with targeting domain fusion site
(SEQ ID NO: 140)
SAAPLVTAMCLLGNVSFPCNRPPTCYTREPSRALDILEENVNHEAYDTLLNAI
LRCGSSGSVTDDFTLTSPYLGTCSYCHHTEPCFSPIKIEQVWDEADDNTIRIQT
SAQFGYDQSGAASSNKYRYMAAAA-(X, WHEREIN X IS A TARGETING
DOMAIN)-
TVKEGTMDDIKISTSGPCRRLSYKGYFLLAKCPPGDSVTVSIASSNSATSCTM
ARKIKPKFVGREKYDLPPVHGKKIPCTVYDRLAATTAGYITMHRPGPHAYTS
YLEESSGKVYAKPPSGKNITYECKCGDYKTGTVTTRTEITGCTAIKQCVAYKS
DQTKWVFNSPDLIRHADHTAQGKLHLPFKLIPSTCMVPVAHAPNVVHGFKHI
SLQLDTDHLTLLTTRRLGANPEPTTEWIIGKTVRNFTVDRDGLEYIWGNHEPV
RVYAQESAPGDPHGWPHEIVQHYYHRHPVYTILAVASAAVAMMIGVTVAAL
CACKARRECLTPYALAPNAVIPTSLALLCCVRSANAETFTETMSYLWSNSQPF
FWVQLCIPLAAVIVLMRCCSCCLPFLVVAGAYLAKVDAYEHATTVPNVPQIP
YKALVERAGYAPLNLEITVMSSEVLPSTNQEYITCKFTTVVPSPKVKCCGSLE
CQPAAHADYTCKVFGGVYPFMWGGAQCFCDSENSQMSEAYVELSADCATD
HAQAIKVHTAAMKVGLRIVYGNTTSFLDVYVNGVTPGTSKDLKVIAGPISAS
FTPFDHKVVIHRGLVYNYDFPEYGAMKPGAFGDIQATSLTSKDLIASTDIRLL
KPSSGNVHVPYTQAASGFEMWKNNSGRPLQETAPFGCKIAVNPLRAVDCSY
GNIPISIDIPNAAFIRTSDAPLVSTVKCDVSECTYSADFGGMATLQYVSDREGQ
CPVHSHSSTATLQESTVHVLEKGAVTVHFSTASPQANFIVSLCGKKTTCNAEC
KPPADHIVSTPHKNDQEFQAAISKTSWSWLFALFGGASSLLIIGLMIFACSMM
LTSTRR
Sindbis Virus Glycoprotein (SINVG) quad MUT version 2, E3(61-64del)
E2(68SLEQ71 to 68AAAA71) E2(159KE160 to 159AA160) E1(250AK251 to
250SG251)
(SEQ ID NO: 141)
SAAPLVTAMCLLGNVSFPCDRPPTCYTREPSRALDILEENVNHEAYDTLLNAI
LRCGSSGSVIDDFTLTSPYLGTCSYCHHTEPCFSPVKIEQVWDEADDNTIRIQT
SAQFGYDQSGAASANKYRYMAAAADHTVKEGTMDDIKISTSGPCRRLSYKG
YFLLAKCPPGDSVTVSIVSSNSATSCTLARKIKPKFVGREKYDLPPVHGKKIPC
TVYDRLAATTAGYITMHRPGPHAYTSYLEESSGKVYAKPPSGKNITYECKCG
DYKTGTVSTRTEITGCTAIKQCVAYKSDQTKWVFNSPDLIRHDDHTVQGKLH
LPFKLIPSTCMVPVAHAPNVIHGFKHISLQLDTDHLTLLTTRRLGANPEPTTEW
IVGKTVRNFTVDRDGLEYIWGNHEPVRVYAQESAPGDPHGWPHEIVQHYYH
RHPVYTILAVASATVAMMIGVTVAVLCACKARRECLTPYALAPNAVIPTSLA
LLCCVRSANAETFTETMSYLWSNSQPFFWVQLCIPLAAFIVLMRCCSCCLPFL
VVAGAYLAKVDAYEHATTVPNVPQIPYKALVERAGYAPLNLEITVMSSEVLP
STNQEYITCKFTTVVPSPKIKCCGSLECQPAAHAGYTCKVFGGVYPFMWGGA
QCFCDSENSQMSEAYVELSADCASDHAQAIKVHTAAMKVGLRIVYGNTTSFL
DVYVNGVTPGTSKDLKVIAGPISASFTPFDHKVVIHRGLVYNYDFPEYGAMK
PGAFGDIQATSLTSKDLIASTDIRLLKPSSGNVHVPYTQASSGFEMWKNNSGR
PLQETAPFGCKIAVNPLRAVDCSYGNIPISIDIPNAAFIRTSDAPLVSTVKCEVS
ECTYSADFGGMATLQYVSDREGQCPVHSHSSTATLQESTVHVLEKGAVTVH
FSTASPQANFIVSLCGKKTTCNAECKPPADHIVSTPHKNDQEFQAAISKTSWS
WLFALFGGASSLLIIGLMIFACSMMLTSTRR
Sindbis Virus Glycoprotein (SINVG) quad MUT version 2, E3(61-64del)
E2(68SLEQ71 to 68AAAA71) E2(159KE160 to 159AA160) E1(250AK251 to
250SG251) with HA TAG
(SEQ ID NO: 142)
SAAPLVTAMCLLGNVSFPCDRPPTCYTREPSRALDILEENVNHEAYDTLLNAI
LRCGSSGSVIDDFTLTSPYLGTCSYCHHTEPCFSPVKIEQVWDEADDNTIRIQT
SAQFGYDQSGAASANKYRYMAAAAMYPYDVPDYATVKEGTMDDIKISTSGP
CRRLSYKGYFLLAKCPPGDSVTVSIVSSNSATSCTLARKIKPKFVGREKYDLPP
VHGKKIPCTVYDRLAATTAGYITMHRPGPHAYTSYLEESSGKVYAKPPSGKN
ITYECKCGDYKTGTVSTRTEITGCTAIKQCVAYKSDQTKWVFNSPDLIRHDDH
TVQGKLHLPFKLIPSTCMVPVAHAPNVIHGFKHISLQLDTDHLTLLTTRRLGA
NPEPTTEWIVGKTVRNFTVDRDGLEYIWGNHEPVRVYAQESAPGDPHGWPH
EIVQHYYHRHPVYTILAVASATVAMMIGVTVAVLCACKARRECLTPYALAP
NAVIPTSLALLCCVRSANAETFTETMSYLWSNSQPFFWVQLCIPLAAFIVLMR
CCSCCLPFLVVAGAYLAKVDAYEHATTVPNVPQIPYKALVERAGYAPLNLEI
TVMSSEVLPSTNQEYITCKFTTVVPSPKIKCCGSLECQPAAHAGYTCKVFGGV
YPFMWGGAQCFCDSENSQMSEAYVELSADCASDHAQAIKVHTAAMKVGLRI
VYGNTTSFLDVYVNGVTPGTSKDLKVIAGPISASFTPFDHKVVIHRGLVYNYD
FPEYGAMKPGAFGDIQATSLTSKDLIASTDIRLLKPSSGNVHVPYTQASSGFE
MWKNNSGRPLQETAPFGCKIAVNPLRAVDCSYGNIPISIDIPNAAFIRTSDAPL
VSTVKCEVSECTYSADFGGMATLQYVSDREGQCPVHSHSSTATLQESTVHVL
EKGAVTVHFSTASPQANFIVSLCGKKTTCNAECKPPADHIVSTPHKNDQEFQA
AISKTSWSWLFALFGGASSLLIIGLMIFACSMMLTSTRR
Sindbis Virus Glycoprotein (SINVG) quad MUT version 2, E3(61-64del)
E2(68SLEQ71 to 68AAAA71) E2(159KE160 to 159AA160) E1(250AK251 to
250SG251) with targeting domain fusion site
(SEQ ID NO: 143)
SAAPLVTAMCLLGNVSFPCDRPPTCYTREPSRALDILEENVNHEAYDTLLNAI
LRCGSSGSVIDDFTLTSPYLGTCSYCHHTEPCFSPVKIEQVWDEADDNTIRIQT
SAQFGYDQSGAASANKYRYMAAAA-(X, WHEREIN X IS A TARGETING
DOMAIN)-
TVKEGTMDDIKISTSGPCRRLSYKGYFLLAKCPPGDSVTVSIVSSNSATSCTLA
RKIKPKFVGREKYDLPPVHGKKIPCTVYDRLAATTAGYITMHRPGPHAYTSY
LEESSGKVYAKPPSGKNITYECKCGDYKTGTVSTRTEITGCTAIKQCVAYKSD
QTKWVFNSPDLIRHDDHTVQGKLHLPFKLIPSTCMVPVAHAPNVIHGFKHISL
QLDTDHLTLLTTRRLGANPEPTTEWIVGKTVRNFTVDRDGLEYIWGNHEPVR
VYAQESAPGDPHGWPHEIVQHYYHRHPVYTILAVASATVAMMIGVTVAVLC
ACKARRECLTPYALAPNAVIPTSLALLCCVRSANAETFTETMSYLWSNSQPFF
WVQLCIPLAAFIVLMRCCSCCLPFLVVAGAYLAKVDAYEHATTVPNVPQIPY
KALVERAGYAPLNLEITVMSSEVLPSTNQEYITCKFTTVVPSPKIKCCGSLECQ
PAAHAGYTCKVFGGVYPFMWGGAQCFCDSENSQMSEAYVELSADCASDHA
QAIKVHTAAMKVGLRIVYGNTTSFLDVYVNGVTPGTSKDLKVIAGPISASFTP
FDHKVVIHRGLVYNYDFPEYGAMKPGAFGDIQATSLTSKDLIASTDIRLLKPS
SGNVHVPYTQASSGFEMWKNNSGRPLQETAPFGCKIAVNPLRAVDCSYGNIP
ISIDIPNAAFIRTSDAPLVSTVKCEVSECTYSADFGGMATLQYVSDREGQCPVH
SHSSTATLQESTVHVLEKGAVTVHFSTASPQANFIVSLCGKKTTCNAECKPPA
DHIVSTPHKNDQEFQAAISKTSWSWLFALFGGASSLLIIGLMIFACSMMLTSTR
R
Measles Virus Hemagglutinin (MeV H) WT
(SEQ ID NO: 144)
MSPQRDRINAFYKDNPHPKGSRIVINREHLMIDRPYVLLAVLFVMFLSLIGLL
AIAGIRLHRAAIYTAEIHKSLSTNLDVTNSIEHQVKDVLTPLFKIIGDEVGLRTP
QRFTDLVKFISDKIKFLNPDREYDFRDLTWCINPPERIKLDYDQYCADVAAEE
LMNALVNSTLLETRTTNQFLAVSKGNCSGPTTIRGQFSNMSLSLLDLYLGRG
YNVSSIVTMTSQGMYGGTYLVEKPNLSSKRSELSQLSMYRVFEVGVIRNPGL
GAPVFHMTNYLEQPVSNDLSNCMVALGELKLAALCHGEDSITIPYQGSGKGV
SFQLVKLGVWKSPTDMQSWVPLSTDDPVIDRLYLSSHRGVIADNQAKWAVP
TTRTDDKLRMETCFQQACKGKIQALCENPEWAPLKDNRIPSYGVLSVDLSLT
VELKIKIASGFGPLITHGSGMDLYKSNHNNVYWLTIPPMKNLALGVINTLEWI
PRFKVSPYLFNVPIKEAGEDCHAPTYLPAEVDGDVKLSSNLVILPGQDLQYVL
ATYDTSRVEHAVVYYVYSPSRSFSYFYPFRLPIKGVPIELQVECFTWDQKLWC
RHFCVLADSESGGHITHSGMEGMGVSCTVTREDGTNRR
Measles Virus Hemagglutinin (MeV H) delta 18
(SEQ ID NO: 145)
MGSRIVINREHLMIDRPYVLLAVLFVMFLSLIGLLAIAGIRLHRAAIYTAEIHKS
LSTNLDVTNSIEHQVKDVLTPLFKIIGDEVGLRTPQRFTDLVKFISDKIKFLNPD
REYDFRDLTWCINPPERIKLDYDQYCADVAAEELMNALVNSTLLETRTTNQF
LAVSKGNCSGPTTIRGQFSNMSLSLLDLYLGRGYNVSSIVTMTSQGMYGGTY
LVEKPNLSSKRSELSQLSMYRVFEVGVIRNPGLGAPVFHMTNYLEQPVSNDLS
NCMVALGELKLAALCHGEDSITIPYQGSGKGVSFQLVKLGVWKSPTDMQSW
VPLSTDDPVIDRLYLSSHRGVIADNQAKWAVPTTRTDDKLRMETCFQQACKG
KIQALCENPEWAPLKDNRIPSYGVLSVDLSLTVELKIKIASGFGPLITHGSGMD
LYKSNHNNVYWLTIPPMKNLALGVINTLEWIPRFKVSPYLFTVPIKEAGGDCH
APTYLPAEVDGDVKLSSNLVILPGQDLQYVLATYDTSRVEHAVVYYVYSPSR
SFSYFYPFRLPIKGVPIELQVECFTWDQKLWCRHFCVLADSESGGHITHSGMV
GMGVSCTVTREDGTNRR
Measles Virus Hemagglutinin (MeV H) delta 18 double mut
(Y463A)(R515A)
(SEQ ID NO: 146)
MGSRIVINREHLMIDRPYVLLAVLFVMFLSLIGLLAIAGIRLHRAAIYTAEIHKSL
STNLDVTNSIEHQVKDVLTPLFKIIGDEVGLRTPQRFTDLVKFISDKIKFLNPD
REYDFRDLTWCINPPERIKLDYDQYCADVAAEELMNALVNSTLLETRTTNQF
LAVSKGNCSGPTTIRGQFSNMSLSLLDLYLGRGYNVSSIVTMTSQGMYGGT
YLVEKPNLSSKRSELSQLSMYRVFEVGVIRNPGLGAPVFHMTNYLEQPVSN
DLSNCMVALGELKLAALCHGEDSITIPYQGSGKGVSFQLVKLGVWKSPTDM
QSWVPLSTDDPVIDRLYLSSHRGVIADNQAKWAVPTTRTDDKLRMETCFQQ
ACKGKIQALCENPEWAPLKDNRIPSYGVLSVDLSLTVELKIKIASGFGPLITHG
SGMDLYKSNHNNVYWLTIPPMKNLALGVINTLEWIPRFKVSPALFNVPIKEA
GGDCHAPTYLPAEVDGDVKLSSNLVILPGQDLQYVLATYDTSAVEHAVVYY
VYSPSRSFSYFYPFRLPIKGVPIELQVECFTWDQKLWCRHFCVLADSESGG
HITHSGMVGMGVSCTVTREAAARGS
Measles Virus Hemagglutinin (MeV H) delta 18 double mut
(Y463A)(R515A) with targeting domain fusion site
(SEQ ID NO: 147)
MGSRIVINREHLMIDRPYVLLAVLFVMFLSLIGLLAIAGIRLHRAAIYTAEIHKSL
STNLDVTNSIEHQVKDVLTPLFKIIGDEVGLRTPQRFTDLVKFISDKIKFLNPD
REYDFRDLTWCINPPERIKLDYDQYCADVAAEELMNALVNSTLLETRTTNQF
LAVSKGNCSGPTTIRGQFSNMSLSLLDLYLGRGYNVSSIVTMTSQGMYGGT
YLVEKPNLSSKRSELSQLSMYRVFEVGVIRNPGLGAPVFHMTNYLEQPVSN
DLSNCMVALGELKLAALCHGEDSITIPYQGSGKGVSFQLVKLGVWKSPTDM
QSWVPLSTDDPVIDRLYLSSHRGVIADNQAKWAVPTTRTDDKLRMETCFQQ
ACKGKIQALCENPEWAPLKDNRIPSYGVLSVDLSLTVELKIKIASGFGPLITHG
SGMDLYKSNHNNVYWLTIPPMKNLALGVINTLEWIPRFKVSPALFNVPIKEA
GGDCHAPTYLPAEVDGDVKLSSNLVILPGQDLQYVLATYDTSAVEHAVVYY
VYSPSRSFSYFYPFRLPIKGVPIELQVECFTWDQKLWCRHFCVLADSESGG
HITHSGMVGMGVSCTVTRE-(X, WHEREIN X IS A TARGETING DOMAIN)-
AAARGS
Measles Virus Hemagglutinin (MeV H) delta 18 quad mut (Y463A)(R515A)
(530SF531 to 530LS531)
(SEQ ID NO: 148)
MGSRIVINREHLMIDRPYVLLAVLFVMFLSLIGLLAIAGIRLHRAAIYTAEIHKS
LSTNLDVTNSIEHQVKDVLTPLFKIIGDEVGLRTPQRFTDLVKFISDKIKFLNPD
REYDFRDLTWCINPPERIKLDYDQYCADVAAEELMNALVNSTLLETRTTNQF
LAVSKGNCSGPTTIRGQFSNMSLSLLDLYLGRGYNVSSIVTMTSQGMYGGTY
LVEKPNLSSKRSELSQLSMYRVFEVGVIRNPGLGAPVFHMTNYLEQPVSNDLS
NCMVALGELKLAALCHGEDSITIPYQGSGKGVSFQLVKLGVWKSPTDMQSW
VPLSTDDPVIDRLYLSSHRGVIADNQAKWAVPTTRTDDKLRMETCFQQACKG
KIQALCENPEWAPLKDNRIPSYGVLSVDLSLTVELKIKIASGFGPLITHGSGMD
LYKSNHNNVYWLTIPPMKNLALGVINTLEWIPRFKVSPALFNVPIKEAGGDC
HAPTYLPAEVDGDVKLSSNLVILPGQDLQYVLATYDTSAVEHAVVYYVYSPS
RLSSYFYPFRLPIKGVPIELQVECFTWDQKLWCRHFCVLADSESGGHITHSGM
VGMGVSCTVTREAAARGS
Measles Virus Hemagglutinin (MeV H) delta 18 quad mut (Y463A)(R515A)
(530SF531 to 530LS531) with targeting domain fusion site
(SEQ ID NO: 149)
MGSRIVINREHLMIDRPYVLLAVLFVMFLSLIGLLAIAGIRLHRAAIYTAEIHKS
LSTNLDVTNSIEHQVKDVLTPLFKIIGDEVGLRTPQRFTDLVKFISDKIKFLNPD
REYDFRDLTWCINPPERIKLDYDQYCADVAAEELMNALVNSTLLETRTTNQF
LAVSKGNCSGPTTIRGQFSNMSLSLLDLYLGRGYNVSSIVTMTSQGMYGGTY
LVEKPNLSSKRSELSQLSMYRVFEVGVIRNPGLGAPVFHMTNYLEQPVSNDLS
NCMVALGELKLAALCHGEDSITIPYQGSGKGVSFQLVKLGVWKSPTDMQSW
VPLSTDDPVIDRLYLSSHRGVIADNQAKWAVPTTRTDDKLRMETCFQQACKG
KIQALCENPEWAPLKDNRIPSYGVLSVDLSLTVELKIKIASGFGPLITHGSGMD
LYKSNHNNVYWLTIPPMKNLALGVINTLEWIPRFKVSPALFNVPIKEAGGDC
HAPTYLPAEVDGDVKLSSNLVILPGQDLQYVLATYDTSAVEHAVVYYVYSPS
RLSSYFYPFRLPIKGVPIELQVECFTWDQKLWCRHFCVLADSESGGHITHSGM
VGMGVSCTVTRE-(X, WHEREIN X IS A TARGETING DOMAIN)-AAARGS
Measles Virus Hemagglutinin (MeV H) delta 19
(SEQ ID NO: 150)
MSRIVINREHLMIDRPYVLLAVLFVMFLSLIGLLAIAGIRLHRAAIYTAEIHKSL
STNLDVTNSIEHQVKDVLTPLFKIIGDEVGLRTPQRFTDLVKFISDKIKFLNPDR
EYDFRDLTWCINPPERIKLDYDQYCADVAAEELMNALVNSTLLETRTTNQFL
AVSKGNCSGPTTIRGQFSNMSLSLLDLYLGRGYNVSSIVTMTSQGMYGGTYL
VEKPNLSSKRSELSQLSMYRVFEVGVIRNPGLGAPVFHMTNYLEQPVSNDLS
NCMVALGELKLAALCHGEDSITIPYQGSGKGVSFQLVKLGVWKSPTDMQSW
VPLSTDDPVIDRLYLSSHRGVIADNQAKWAVPTTRTDDKLRMETCFQQACKG
KIQALCENPEWAPLKDNRIPSYGVLSVDLSLTVELKIKIASGFGPLITHGSGMD
LYKSNHNNVYWLTIPPMKNLALGVINTLEWIPRFKVSPYLFTVPIKEAGGDCH
APTYLPAEVDGDVKLSSNLVILPGQDLQYVLATYDTSRVEHAVVYYVYSPSR
SFSYFYPFRLPIKGVPIELQVECFTWDQKLWCRHFCVLADSESGGHITHSGMV
GMGVSCTVTREDGTNRR
Measles Virus Hemagglutinin (MeV H) delta 19 double mut
(Y463A)(R515A)
(SEQ ID NO: 151)
MSRIVINREHLMIDRPYVLLAVLFVMFLSLIGLLAIAGIRLHRAAIYTAEIHKSLS
TNLDVTNSIEHQVKDVLTPLFKIIGDEVGLRTPQRFTDLVKFISDKIKFLNPDR
EYDFRDLTWCINPPERIKLDYDQYCADVAAEELMNALVNSTLLETRTTNQFL
AVSKGNCSGPTTIRGQFSNMSLSLLDLYLGRGYNVSSIVTMTSQGMYGGTY
LVEKPNLSSKRSELSQLSMYRVFEVGVIRNPGLGAPVFHMTNYLEQPVSND
LSNCMVALGELKLAALCHGEDSITIPYQGSGKGVSFQLVKLGVWKSPTDMQ
SWVPLSTDDPVIDRLYLSSHRGVIADNQAKWAVPTTRTDDKLRMETCFQQA
CKGKIQALCENPEWAPLKDNRIPSYGVLSVDLSLTVELKIKIASGFGPLITHGS
GMDLYKSNHNNVYWLTIPPMKNLALGVINTLEWIPRFKVSPALFNVPIKEAG
GDCHAPTYLPAEVDGDVKLSSNLVILPGQDLQYVLATYDTSAVEHAVVYYVY
SPSRSFSYFYPFRLPIKGVPIELQVECFTWDQKLWCRHFCVLADSESGGHIT
HSGMVGMGVSCTVTREAAARGS
Measles Virus Hemagglutinin (MeV H) delta 19 double mut
(Y463A)(R515A) with targeting domain fusion site
(SEQ ID NO: 152)
MSRIVINREHLMIDRPYVLLAVLFVMFLSLIGLLAIAGIRLHRAAIYTAEIHKSLS
TNLDVTNSIEHQVKDVLTPLFKIIGDEVGLRTPQRFTDLVKFISDKIKFLNPDR
EYDFRDLTWCINPPERIKLDYDQYCADVAAEELMNALVNSTLLETRTTNQFL
AVSKGNCSGPTTIRGQFSNMSLSLLDLYLGRGYNVSSIVTMTSQGMYGGTY
LVEKPNLSSKRSELSQLSMYRVFEVGVIRNPGLGAPVFHMTNYLEQPVSND
LSNCMVALGELKLAALCHGEDSITIPYQGSGKGVSFQLVKLGVWKSPTDMQ
SWVPLSTDDPVIDRLYLSSHRGVIADNQAKWAVPTTRTDDKLRMETCFQQA
CKGKIQALCENPEWAPLKDNRIPSYGVLSVDLSLTVELKIKIASGFGPLITHGS
GMDLYKSNHNNVYWLTIPPMKNLALGVINTLEWIPRFKVSPALFNVPIKEAG
GDCHAPTYLPAEVDGDVKLSSNLVILPGQDLQYVLATYDTSAVEHAVVYYVY
SPSRSFSYFYPFRLPIKGVPIELQVECFTWDQKLWCRHFCVLADSESGGHIT
HSGMVGMGVSCTVTRE-(X, WHEREIN X IS A TARGETING DOMAIN)-
AAARGS
Measles Virus Hemagglutinin (MeV H) delta 19 quad mut (Y463A)(R515A)
(530SF531 to 530LS531)
(SEQ ID NO: 153)
MSRIVINREHLMIDRPYVLLAVLFVMFLSLIGLLAIAGIRLHRAAIYTAEIHKSL
STNLDVTNSIEHQVKDVLTPLFKIIGDEVGLRTPQRFTDLVKFISDKIKFLNPDR
EYDFRDLTWCINPPERIKLDYDQYCADVAAEELMNALVNSTLLETRTTNQFL
AVSKGNCSGPTTIRGQFSNMSLSLLDLYLGRGYNVSSIVTMTSQGMYGGTYL
VEKPNLSSKRSELSQLSMYRVFEVGVIRNPGLGAPVFHMTNYLEQPVSNDLS
NCMVALGELKLAALCHGEDSITIPYQGSGKGVSFQLVKLGVWKSPTDMQSW
VPLSTDDPVIDRLYLSSHRGVIADNQAKWAVPTTRTDDKLRMETCFQQACKG
KIQALCENPEWAPLKDNRIPSYGVLSVDLSLTVELKIKIASGFGPLITHGSGMD
LYKSNHNNVYWLTIPPMKNLALGVINTLEWIPRFKVSPALFNVPIKEAGGDC
HAPTYLPAEVDGDVKLSSNLVILPGQDLQYVLATYDTSAVEHAVVYYVYSPS
RLSSYFYPFRLPIKGVPIELQVECFTWDQKLWCRHFCVLADSESGGHITHSGM
VGMGVSCTVTREAAARGS
Measles Virus Hemagglutinin (MeV H) delta 19 quad mut (Y463A)(R515A)
(530SF531 to 530LS531) with targeting domain fusion site
(SEQ ID NO: 154)
MSRIVINREHLMIDRPYVLLAVLFVMFLSLIGLLAIAGIRLHRAAIYTAEIHKSL
STNLDVTNSIEHQVKDVLTPLFKIIGDEVGLRTPQRFTDLVKFISDKIKFLNPDR
EYDFRDLTWCINPPERIKLDYDQYCADVAAEELMNALVNSTLLETRTTNQFL
AVSKGNCSGPTTIRGQFSNMSLSLLDLYLGRGYNVSSIVTMTSQGMYGGTYL
VEKPNLSSKRSELSQLSMYRVFEVGVIRNPGLGAPVFHMTNYLEQPVSNDLS
NCMVALGELKLAALCHGEDSITIPYQGSGKGVSFQLVKLGVWKSPTDMQSW
VPLSTDDPVIDRLYLSSHRGVIADNQAKWAVPTTRTDDKLRMETCFQQACKG
KIQALCENPEWAPLKDNRIPSYGVLSVDLSLTVELKIKIASGFGPLITHGSGMD
LYKSNHNNVYWLTIPPMKNLALGVINTLEWIPRFKVSPALFNVPIKEAGGDC
HAPTYLPAEVDGDVKLSSNLVILPGQDLQYVLATYDTSAVEHAVVYYVYSPS
RLSSYFYPFRLPIKGVPIELQVECFTWDQKLWCRHFCVLADSESGGHITHSGM
VGMGVSCTVTRE-(X, WHEREIN X IS A TARGETING DOMAIN)-AAARGS
Measles Virus Hemagglutinin (MeV H) delta 24AAAA
(SEQ ID NO: 155)
MAAAANREHLMIDRPYVLLAVLFVMFLSLIGLLAIAGIRLHRAAIYTAEIHKS
LSTNLDVTNSIEHQVKDVLTPLFKIIGDEVGLRTPQRFTDLVKFISDKIKFLNPD
REYDFRDLTWCINPPERIKLDYDQYCADVAAEELMNALVNSTLLETRTTNQF
LAVSKGNCSGPTTIRGQFSNMSLSLLDLYLGRGYNVSSIVTMTSQGMYGGTY
LVEKPNLSSKRSELSQLSMYRVFEVGVIRNPGLGAPVFHMTNYLEQPVSNDLS
NCMVALGELKLAALCHGEDSITIPYQGSGKGVSFQLVKLGVWKSPTDMQSW
VPLSTDDPVIDRLYLSSHRGVIADNQAKWAVPTTRTDDKLRMETCFQQACKG
KIQALCENPEWAPLKDNRIPSYGVLSVDLSLTVELKIKIASGFGPLITHGSGMD
LYKSNHNNVYWLTIPPMKNLALGVINTLEWIPRFKVSPYLFTVPIKEAGGDCH
APTYLPAEVDGDVKLSSNLVILPGQDLQYVLATYDTSRVEHAVVYYVYSPSR
SFSYFYPFRLPIKGVPIELQVECFTWDQKLWCRHFCVLADSESGGHITHSGMV
GMGVSCTVTREDGTNRR
Measles Virus Hemagglutinin (MeV H) delta 24AAAA double mut
(Y463A)(R515A)
(SEQ ID NO: 156)
MAAAANREHLMIDRPYVLLAVLFVMFLSLIGLLAIAGIRLHRAAIYTAEIHKSLS
TNLDVTNSIEHQVKDVLTPLFKIIGDEVGLRTPQRFTDLVKFISDKIKFLNPDR
EYDFRDLTWCINPPERIKLDYDQYCADVAAEELMNALVNSTLLETRTTNQFL
AVSKGNCSGPTTIRGQFSNMSLSLLDLYLGRGYNVSSIVTMTSQGMYGGTY
LVEKPNLSSKRSELSQLSMYRVFEVGVIRNPGLGAPVFHMTNYLEQPVSND
LSNCMVALGELKLAALCHGEDSITIPYQGSGKGVSFQLVKLGVWKSPTDMQ
SWVPLSTDDPVIDRLYLSSHRGVIADNQAKWAVPTTRTDDKLRMETCFQQA
CKGKIQALCENPEWAPLKDNRIPSYGVLSVDLSLTVELKIKIASGFGPLITHGS
GMDLYKSNHNNVYWLTIPPMKNLALGVINTLEWIPRFKVSPALFNVPIKEAG
GDCHAPTYLPAEVDGDVKLSSNLVILPGQDLQYVLATYDTSAVEHAVVYYVY
SPSRSFSYFYPFRLPIKGVPIELQVECFTWDQKLWCRHFCVLADSESGGHIT
HSGMVGMGVSCTVTREAAARGS
Measles Virus Hemagglutinin (MeV H) delta 24AAAA double mut
(Y463A)(R515A) with targeting domain fusion site
(SEQ ID NO: 157)
MAAAANREHLMIDRPYVLLAVLFVMFLSLIGLLAIAGIRLHRAAIYTAEIHKSLS
TNLDVTNSIEHQVKDVLTPLFKIIGDEVGLRTPQRFTDLVKFISDKIKFLNPDR
EYDFRDLTWCINPPERIKLDYDQYCADVAAEELMNALVNSTLLETRTTNQFL
AVSKGNCSGPTTIRGQFSNMSLSLLDLYLGRGYNVSSIVTMTSQGMYGGTY
LVEKPNLSSKRSELSQLSMYRVFEVGVIRNPGLGAPVFHMTNYLEQPVSND
LSNCMVALGELKLAALCHGEDSITIPYQGSGKGVSFQLVKLGVWKSPTDMQ
SWVPLSTDDPVIDRLYLSSHRGVIADNQAKWAVPTTRTDDKLRMETCFQQA
CKGKIQALCENPEWAPLKDNRIPSYGVLSVDLSLTVELKIKIASGFGPLITHGS
GMDLYKSNHNNVYWLTIPPMKNLALGVINTLEWIPRFKVSPALFNVPIKEAG
GDCHAPTYLPAEVDGDVKLSSNLVILPGQDLQYVLATYDTSAVEHAVVYYVY
SPSRSFSYFYPFRLPIKGVPIELQVECFTWDQKLWCRHFCVLADSESGGHIT
HSGMVGMGVSCTVTRE-(X, WHEREIN X IS A TARGETING DOMAIN)-
AAARGS
Measles Virus Hemagglutinin (MeV H) delta 24AAAA quad mut
(Y463A)(R515A) (530SF531 to 530LS531)
(SEQ ID NO: 158)
MAAAANREHLMIDRPYVLLAVLFVMFLSLIGLLAIAGIRLHRAAIYTAEIHKS
LSTNLDVTNSIEHQVKDVLTPLFKIIGDEVGLRTPQRFTDLVKFISDKIKFLNPD
REYDFRDLTWCINPPERIKLDYDQYCADVAAEELMNALVNSTLLETRTTNQF
LAVSKGNCSGPTTIRGQFSNMSLSLLDLYLGRGYNVSSIVTMTSQGMYGGTY
LVEKPNLSSKRSELSQLSMYRVFEVGVIRNPGLGAPVFHMTNYLEQPVSNDLS
NCMVALGELKLAALCHGEDSITIPYQGSGKGVSFQLVKLGVWKSPTDMQSW
VPLSTDDPVIDRLYLSSHRGVIADNQAKWAVPTTRTDDKLRMETCFQQACKG
KIQALCENPEWAPLKDNRIPSYGVLSVDLSLTVELKIKIASGFGPLITHGSGMD
LYKSNHNNVYWLTIPPMKNLALGVINTLEWIPRFKVSPALFNVPIKEAGGDC
HAPTYLPAEVDGDVKLSSNLVILPGQDLQYVLATYDTSAVEHAVVYYVYSPS
RLSSYFYPFRLPIKGVPIELQVECFTWDQKLWCRHFCVLADSESGGHITHSGM
VGMGVSCTVTREAAARGS
Measles Virus Hemagglutinin (MeV H) delta 24AAAA quad mut
(Y463A)(R515A) (530SF531 to 530LS531) with targeting domain fusion site
(SEQ ID NO: 159)
MAAAANREHLMIDRPYVLLAVLFVMFLSLIGLLAIAGIRLHRAAIYTAEIHKS
LSTNLDVTNSIEHQVKDVLTPLFKIIGDEVGLRTPQRFTDLVKFISDKIKFLNPD
REYDFRDLTWCINPPERIKLDYDQYCADVAAEELMNALVNSTLLETRTTNQF
LAVSKGNCSGPTTIRGQFSNMSLSLLDLYLGRGYNVSSIVTMTSQGMYGGTY
LVEKPNLSSKRSELSQLSMYRVFEVGVIRNPGLGAPVFHMTNYLEQPVSNDLS
NCMVALGELKLAALCHGEDSITIPYQGSGKGVSFQLVKLGVWKSPTDMQSW
VPLSTDDPVIDRLYLSSHRGVIADNQAKWAVPTTRTDDKLRMETCFQQACKG
KIQALCENPEWAPLKDNRIPSYGVLSVDLSLTVELKIKIASGFGPLITHGSGMD
LYKSNHNNVYWLTIPPMKNLALGVINTLEWIPRFKVSPALFNVPIKEAGGDC
HAPTYLPAEVDGDVKLSSNLVILPGQDLQYVLATYDTSAVEHAVVYYVYSPS
RLSSYFYPFRLPIKGVPIELQVECFTWDQKLWCRHFCVLADSESGGHITHSGM
VGMGVSCTVTRE-(X, WHEREIN X IS A TARGETING DOMAIN)-AAARGS
Measles Virus Fusion (MeV F) delta 24
(SEQ ID NO: 160)
MGLKVNVSAIFMAVLLTLQTPTGQIHWGNLSKIGVVGIGSASYKVMTRSSHQ
SLVIKLMPNITLLNNCTRVEIAEYRRLLRTVLEPIRDALNAMTQNIRPVQSVAS
SRRHKRFAGVVLAGAALGVATAAQITAGIALHQSMLNSQAIDNLRASLETTN
QAIEAIRQAGQEMILAVQGVQDYINNELIPSMNQLSCDLIGQKLGLKLLRYYT
EILSLFGPSLRDPISAEISIQALSYALGGDINKVLEKLGYSGGDLLGILESRGIKA
RITHVDTESYFIVLSIAYPTLSEIKGVIVHRLEGVSYNIGSQEWYTTVPKYVAT
QGYLISNFDESSCTFMPEGTVCSQNALYPMSPLLQECLRGSTKSCARTLVSGS
FGNRFILSQGNLIANCASILCKCYTTGTIINQDPDKILTYIAADHCPVVEVNGV
TIQVGSRRYPDAVYLHRIDLGPPISLERLDVGTNLGNAIAKLEDAKELLESSDQ
ILRSMKGLSSTSIVYILIAVCLGGLIGIPALICCCRGRCNKKGE
Measles Virus Fusion (MeV F) delta 24 (T461l) hyperfusogenic mut
(SEQ ID NO: 161)
MGLKVNVSAIFMAVLLTLQTPTGQIHWGNLSKIGVVGIGSASYKVMTRSSHQ
SLVIKLMPNITLLNNCTRVEIAEYRRLLRTVLEPIRDALNAMTQNIRPVQSVAS
SRRHKRFAGVVLAGAALGVATAAQITAGIALHQSMLNSQAIDNLRASLETTN
QAIEAIRQAGQEMILAVQGVQDYINNELIPSMNQLSCDLIGQKLGLKLLRYYT
EILSLFGPSLRDPISAEISIQALSYALGGDINKVLEKLGYSGGDLLGILESRGIKA
RITHVDTESYFIVLSIAYPTLSEIKGVIVHRLEGVSYNIGSQEWYTTVPKYVAT
QGYLISNFDESSCTFMPEGTVCSQNALYPMSPLLQECLRGSTKSCARTLVSGS
FGNRFILSQGNLIANCASILCKCYTTGTIINQDPDKILTYIAADHCPVVEVNGV
TIQVGSRRYPDAVYLHRIDLGPPISLERLDVGINLGNAIAKLEDAKELLESSDQI
LRSMKGLSSTSIVYILIAVCLGGLIGIPALICCCRGRCNKKGE
Measles Virus Fusion (MeV F) delta 30
(SEQ ID NO: 162)
MGLKVNVSAIFMAVLLTLQTPTGQIHWGNLSKIGVVGIGSASYKVMTRSSHQ
SLVIKLMPNITLLNNCTRVEIAEYRRLLRTVLEPIRDALNAMTQNIRPVQSVAS
SRRHKRFAGVVLAGAALGVATAAQITAGIALHQSMLNSQAIDNLRASLETTN
QAIEAIRQAGQEMILAVQGVQDYINNELIPSMNQLSCDLIGQKLGLKLLRYYT
EILSLFGPSLRDPISAEISIQALSYALGGDINKVLEKLGYSGGDLLGILESRGIKA
RITHVDTESYFIVLSIAYPTLSEIKGVIVHRLEGVSYNIGSQEWYTTVPKYVAT
QGYLISNFDESSCTFMPEGTVCSQNALYPMSPLLQECLRGSTKSCARTLVSGS
FGNRFILSQGNLIANCASILCKCYTTGTIINQDPDKILTYIAADHCPVVEVNGV
TIQVGSRRYPDAVYLHRIDLGPPISLERLDVGTNLGNAIAKLEDAKELLESSDQ
ILRSMKGLSSTSIVYILIAVCLGGLIGIPALICCCRGR
Measles Virus Fusion (MeV F) delta 30 (T461l) hyperfusogenic mut
(SEQ ID NO: 163)
MGLKVNVSAIFMAVLLTLQTPTGQIHWGNLSKIGVVGIGSASYKVMTRSSHQ
SLVIKLMPNITLLNNCTRVEIAEYRRLLRTVLEPIRDALNAMTQNIRPVQSVAS
SRRHKRFAGVVLAGAALGVATAAQITAGIALHQSMLNSQAIDNLRASLETTN
QAIEAIRQAGQEMILAVQGVQDYINNELIPSMNQLSCDLIGQKLGLKLLRYYT
EILSLFGPSLRDPISAEISIQALSYALGGDINKVLEKLGYSGGDLLGILESRGIKA
RITHVDTESYFIVLSIAYPTLSEIKGVIVHRLEGVSYNIGSQEWYTTVPKYVAT
QGYLISNFDESSCTFMPEGTVCSQNALYPMSPLLQECLRGSTKSCARTLVSGS
FGNRFILSQGNLIANCASILCKCYTTGTIINQDPDKILTYIAADHCPVVEVNGV
TIQVGSRRYPDAVYLHRIDLGPPISLERLDVGINLGNAIAKLEDAKELLESSDQI
LRSMKGLSSTSIVYILIAVCLGGLIGIPALICCCRGR
Tupaia Paramyxovirus Hemagglutinin (TPMV H) WT
(SEQ ID NO: 164)
MDYHSHTTQTGSNETLYQDPLQSQSGSRDTLDGPPSTLQHYSNPPPYSEEDQ
GIDGPQRSQPLSTPHQYDRYYGVNIQHTRVYNHLGTIYKGLKLAFQILGWVS
VIITMIITVTTLKKMSDGNSQDSAMLKSLDENFDAIQEVANLLDNEVRPKLGV
TMTQTTFQLPKELSEIKRYLLRLERNCPVCGTEATPQGSKGNASGDTAFCPPC
LTRQCSEDSTHDQGPGVEGTSRNHKGKINFPHILQSDDCGRSDNLIVYSINLVP
GLSFIQLPSGTKHCIIDVSYTFSDTLAGYLIVGGVDGCQLHNKAIIYLSLGYYK
TKMIYPPDYIAIATYTYDLVPNLRDCSIAVNQTSLAAICTSKKTKENQDFSTSG
VHPFYIFTLNTDGIFTVTVIEQSQLKLDYQYAALYPATGPGIFIGDHLVFLMW
GGLMTKAEGDAYCQASGCNDAHRTSCNIAQMPSAYGHRQLVNGLLMLPIKE
LGSHLIQPSLETISPKINWAGGHGRLYYNWEINTTYIYIEGKTWRSRPNLGIIS
WSKPLSIRWIDHSVARRPGARPCDSANDCPEDCLVGGYYDMFPMSSDYKTAI
TIIPTHHQWPSSPALKLFNTNREVRVVMILRPPNNVKKTTISCIRIMQTNWCLG
FIIFKEGNNAWGQIYSYIYQVESTCPNTK
Tupaia Paramyxovirus Hemagglutinin (TPMV H) delta 32
(SEQ ID NO: 165)
MGPPSTLQHYSNPPPYSEEDQGIDGPQRSQPLSTPHQYDRYYGVNIQHTRVYN
HLGTIYSGLKLAFQILGWVSVIITMIITVTTLKKMSDGNSQDSAMLKSLDENF
DAIQEVANLLDNEVRPKLGVTMTQTTFQLPKELSEIKRYLLRLERNCPVCGTE
ATPQGSKGNASGDTAFCPPCLTRQCSEDSTHDQGPGVEGTSRNHKGKINFPHI
LQSDDCGRSDNLIVYSINLVPGLSFIQLPSGTKHCIIDVSYTFSDTLAGYLIVGG
VDGCQLHNKAIIYLSLGYYKTKMIYPPDYIAIATYTYDLVPNLRDCSIAVNQT
SLAAICTSKKTKENQDFSTSGVHPFYIFTLNTDGIFTVTVIEQSQLKLDYQYAA
LYPATGPGIFIGDHLVFLMWGGLMTKAEGDAYCQASGCNDAHRTSCNIAQM
PSAYGHRQLVNGLLMLPIKELGSHLIQPSLETISPKINWAGGHGRLYYNWEIN
TTYIYIEGKTWRSRPNLGIISWSKPLSIRWIDHSVARRPGARPCDSANDCPEDC
LVGGYYDMFPMSSDYKTAITIIPTHHQWPSSPALKLFNTNREVRVVMILRPPN
NVKKTTISCIRIMQTNWCLGFIIFKEGNNAWGQIYSYIYQVESTCPNTKTAAR
GTGS
Tupaia Paramyxovirus Hemagglutinin (TPMV H) delta 32 with targeting
domain fusion site
(SEQ ID NO: 166)
MGPPSTLQHYSNPPPYSEEDQGIDGPQRSQPLSTPHQYDRYYGVNIQHTRVYN
HLGTIYSGLKLAFQILGWVSVIITMIITVTTLKKMSDGNSQDSAMLKSLDENF
DAIQEVANLLDNEVRPKLGVTMTQTTFQLPKELSEIKRYLLRLERNCPVCGTE
ATPQGSKGNASGDTAFCPPCLTRQCSEDSTHDQGPGVEGTSRNHKGKINFPHI
LQSDDCGRSDNLIVYSINLVPGLSFIQLPSGTKHCIIDVSYTFSDTLAGYLIVGG
VDGCQLHNKAIIYLSLGYYKTKMIYPPDYIAIATYTYDLVPNLRDCSIAVNQT
SLAAICTSKKTKENQDFSTSGVHPFYIFTLNTDGIFTVTVIEQSQLKLDYQYAA
LYPATGPGIFIGDHLVFLMWGGLMTKAEGDAYCQASGCNDAHRTSCNIAQM
PSAYGHRQLVNGLLMLPIKELGSHLIQPSLETISPKINWAGGHGRLYYNWEIN
TTYIYIEGKTWRSRPNLGIISWSKPLSIRWIDHSVARRPGARPCDSANDCPEDC
LVGGYYDMFPMSSDYKTAITIIPTHHQWPSSPALKLFNTNREVRVVMILRPPN
NVKKTTISCIRIMQTNWCLGFIIFKEGNNAWGQIYSYIYQVESTCPNTKT-(X,
WHEREIN X IS A TARGETING DOMAIN)-AARGTGS
Tupaia Paramyxovirus Hemagglutinin (TPMV H) delta 80
(SEQ ID NO: 167)
MRVYNHLGTIYKGLKLAFQILGWVSVIITMIITVTTLKKMSDGNSQDSAMLKS
LDENFDAIQEVANLLDNEVRPKLGVTMTQTTFQLPKELSEIKRYLLRLERNCP
VCGTEATPQGSKGNASGDTAFCPPCLTRQCSEDSTHDQGPGVEGTSRNHKGK
INFPHILQSDDCGRSDNLIVYSINLVPGLSFIQLPSGTKHCIIDVSYTFSDTLAGY
LIVGGVDGCQLHNKAIIYLSLGYYKTKMIYPPDYIAIATYTYDLVPNLRDCSIA
VNQTSLAAICTSKKTKENQDFSTSGVHPFYIFTLNTDGIFTVTVIEQSQLKLDY
QYAALYPATGPGIFIGDHLVFLMWGGLMTKAEGDAYCQASGCNDAHRTSCN
IAQMPSAYGHRQLVNGLLMLPIKELGSHLIQPSLETISPKINWAGGHGRLYYN
WEINTTYIYIEGKTWRSRPNLGIISWSKPLSIRWIDHSVARRPGARPCDSANDC
PEDCLVGGYYDMFPMSSDYKTAITIIPTHHQWPSSPALKLFNTNREVRVVMIL
RPPNNVKKTTISCIRIMQTNWCLGFIIFKEGNNAWGQIYSYIYQVESTCPNTK
Tupaia Paramyxovirus Hemagglutinin (TPMV H) delta 80 with targeting
domain fusion site
(SEQ ID NO: 168)
MRVYNHLGTIYKGLKLAFQILGWVSVIITMIITVTTLKKMSDGNSQDSAMLKS
LDENFDAIQEVANLLDNEVRPKLGVTMTQTTFQLPKELSEIKRYLLRLERNCP
VCGTEATPQGSKGNASGDTAFCPPCLTRQCSEDSTHDQGPGVEGTSRNHKGK
INFPHILQSDDCGRSDNLIVYSINLVPGLSFIQLPSGTKHCIIDVSYTFSDTLAGY
LIVGGVDGCQLHNKAIIYLSLGYYKTKMIYPPDYIAIATYTYDLVPNLRDCSIA
VNQTSLAAICTSKKTKENQDFSTSGVHPFYIFTLNTDGIFTVTVIEQSQLKLDY
QYAALYPATGPGIFIGDHLVFLMWGGLMTKAEGDAYCQASGCNDAHRTSCN
IAQMPSAYGHRQLVNGLLMLPIKELGSHLIQPSLETISPKINWAGGHGRLYYN
WEINTTYIYIEGKTWRSRPNLGIISWSKPLSIRWIDHSVARRPGARPCDSANDC
PEDCLVGGYYDMFPMSSDYKTAITIIPTHHQWPSSPALKLFNTNREVRVVMIL
RPPNNVKKTTISCIRIMQTNWCLGFIIFKEGNNAWGQIYSYIYQVESTCPNTKT-
(X, WHEREIN X IS A TARGETING DOMAIN)-AARGTGS
Tupaia Paramyxovirus Fusion (TPMV F) WT
(SEQ ID NO: 169)
MASLLKTICYIYLITYAKLEPTPKSQLDLDSLASIGVVDAGKYNYKLMTTGSE
KLMVIKLVPNITYATNCNLTAHTAYTKMIERLLTPINQSLYEMRSVITERDGG
TIFWGAIIAGAALGVATAAAITAGVALHRAEQNARNIAALKDALRNSNEAIQ
HLKDAQGHTVLAIQGLQEQINNNIIPKLKESHCLGVNNQLGLLLNQYYSEILT
VFGPNLQNPVSASLTIQAIAKAFNGDFNSLMTNLNYDPTDLLDILESNSINGRII
DVNLNEKYIALSIEIPNFITLTDAKIQTFNRITYGYGSNEWLTLIPDNILEYGNLI
SNVDLTSCVKTKSSYICNQDTSYPISSELTRCLRGDTSSCPRTPVVNSRAPTFA
LSGGHIYANCAKAACRCEKPPMAIVQPATSTLTFLTEKECQEVVIDQINIQLAP
NRLNKTIITDGIDLGPEVIINPIDVSAELGNIELEMDKTQKALDRSNKILDSMIT
EVTPDKLLIAMIVVFGILLLWLFGVSYYAFKIWSKLHFLDSYVYSLRNPSHHR
SNGHQNHSFSTDISG
Tupaia Paramyxovirus Fusion (TPMV F) delta 32
(SEQ ID NO: 170)
MASLLKTICYIYLITYAKLEPTPKSQLDLDSLASIGVVDAGKYNYKLMTTGSE
KLMVIKLVPNITYATNCNLTAHTAYTKMIERLLTPINQSLYEMRSVITERDGG
TIFWGAIIAGAALGVATAAAITAGVALHRAEQNARNIAALKDALRNSNEAIQ
HLKDAQGHTVLAIQGLQEQINNNIIPKLKESHCLGVNNQLGLLLNQYYSEILT
VFGPNLQNPVSASLTIQAIAKAFNGDFNSLMTNLNYDPTDLLDILESNSINGRII
DVNLNEKYIALSIEIPNFITLTDAKIQTFNRITYGYGSNEWLTLIPDNILEYGNLI
SNVDLTSCVKTKSSYICNQDTSYPISSELTRCLRGDTSSCPRTPVVNSRAPTFA
LSGGHIYANCAKAACRCEKPPMAIVQPATSTLTFLTEKECQEVVIDQINIQLAP
NRLNKTIITDGIDLGPEVIINPIDVSAELGNIELEMDKTQKALDRSNKILDSMIT
EVTPDKLLIAMIVVFGILLLWLFGVSYYAFKIWSKL
Canine distemper virus Hemagglutinin (CDV H) WT
(SEQ ID NO: 171)
MLPYQDKVGAFYKDNARANSTKLSLVTEGHGGRRPPYLLFVLLILLVGILAL
LAITGVRFHQVSTSNMEFSRLLKEDMEKSEAVHHQVIDVLTPLFKIIGDEIGLR
LPQKLNEIKQFILQKTNFFNPNREFDFRDLHWCINPPSTVKVNFTNYCESIGIR
KAIASAANPILLSALSGGRGDIFPPHRCSGATTSVGKVFPLSVSLSMSLISRTSE
VINMLTAISDGVYGKTYLLVPDDIEREFDTREIRVFEIGFIKRWLNDMPLLQTT
NYMVLPKNSKAKVCTIAVGELTLASLCVEESTVLLYHDSSGSQDGILVVTLGI
FWATPMDHIEEVIPVAHPSMKKIHITNHRGFIKDSIATWMVPALASEKQEEQK
GCLESACQRKTYPMCNQASWEPFGGRQLPSYGRLTLPLDASVDLQLNISFTY
GPVILNGDGMDYYESPLLNSGWLTIPPKDGTISGLINKAGRGDQFTVLPHVLT
FAPRESSGNCYLPIQTSQIRDRDVLIESNIVVLPTQSIRYVIATYDISRSDHAIVY
YVYDPIRTISYTHPFRLTTKGRPDFLRIECFVWDDNLWCHQFYRFEADIANST
TSVENLVRIRFSCNR
Canine distemper virus Hemagglutinin (CDV H) WT with targeting
domain fusion site
(SEQ ID NO: 172)
MLPYQDKVGAFYKDNARANSTKLSLVTEGHGGRRPPYLLFVLLILLVGILAL
LAITGVRFHQVSTSNMEFSRLLKEDMEKSEAVHHQVIDVLTPLFKIIGDEIGLR
LPQKLNEIKQFILQKTNFFNPNREFDFRDLHWCINPPSTVKVNFTNYCESIGIR
KAIASAANPILLSALSGGRGDIFPPHRCSGATTSVGKVFPLSVSLSMSLISRTSE
VINMLTAISDGVYGKTYLLVPDDIEREFDTREIRVFEIGFIKRWLNDMPLLQTT
NYMVLPKNSKAKVCTIAVGELTLASLCVEESTVLLYHDSSGSQDGILVVTLGI
FWATPMDHIEEVIPVAHPSMKKIHITNHRGFIKDSIATWMVPALASEKQEEQK
GCLESACQRKTYPMCNQASWEPFGGRQLPSYGRLTLPLDASVDLQLNISFTY
GPVILNGDGMDYYESPLLNSGWLTIPPKDGTISGLINKAGRGDQFTVLPHVLT
FAPRESSGNCYLPIQTSQIRDRDVLIESNIVVLPTQSIRYVIATYDISRSDHAIVY
YVYDPIRTISYTHPFRLTTKGRPDFLRIECFVWDDNLWCHQFYRFEADIANST
TSVENLVRIRFSCNR-(X, WHEREIN X IS A TARGETING DOMAIN)-GS
Canine distemper virus Hemagglutinin (CDV H) delta 18
(SEQ ID NO: 173)
MSTKLSLVTEGHGGRRPPYLLFVLLILLVGILALLAITGVRFHQVSTSNMEFS
RLLKEDMEKSEAVHHQVIDVLTPLFKIIGDEIGLRLPQKLNEIKQFILQKTNFFN
PNREFDFRDLHWCINPPSTVKVNFTNYCESIGIRKAIASAANPILLSALSGGR
GDIFPPHRCSGATTSVGKVFPLSVSLSMSLISRTSEVINMLTAISDGVYGKTY
LLVPDDIEREFDTREIRVFEIGFIKRWLNDMPLLQTTNYMVLPKNSKAKVCTIA
VGELTLASLCVEESTVLLYHDSSGSQDGILVVTLGIFWATPMDHIEEVIPVAH
PSMKKIHITNHRGFIKDSIATWMVPALASEKQEEQKGCLESACQRKTYPMCN
QASWEPFGGRQLPSYGRLTLPLDASVDLQLNISFTYGPVILNGDGMDYYES
PLLNSGWLTIPPKDGTISGLINKAGRGDQFTVLPHVLTFAPRESSGNCYLPIQ
TSQIRDRDVLIESNIVVLPTQSIRYVIATYDISRSDHAIVYYVYDPIRTISYTHPF
RLTTKGRPDFLRIECFVWDDNLWCHQFYRFEADIANSTTSVENLVRIRFSCN
R
Canine distemper virus Hemagglutinin (CDV H) delta 18 with targeting
domain fusion site
(SEQ ID NO: 174)
MSTKLSLVTEGHGGRRPPYLLFVLLILLVGILALLAITGVRFHQVSTSNMEFS
RLLKEDMEKSEAVHHQVIDVLTPLFKIIGDEIGLRLPQKLNEIKQFILQKTNFFN
PNREFDFRDLHWCINPPSTVKVNFTNYCESIGIRKAIASAANPILLSALSGGR
GDIFPPHRCSGATTSVGKVFPLSVSLSMSLISRTSEVINMLTAISDGVYGKTY
LLVPDDIEREFDTREIRVFEIGFIKRWLNDMPLLQTTNYMVLPKNSKAKVCTIA
VGELTLASLCVEESTVLLYHDSSGSQDGILVVTLGIFWATPMDHIEEVIPVAH
PSMKKIHITNHRGFIKDSIATWMVPALASEKQEEQKGCLESACQRKTYPMCN
QASWEPFGGRQLPSYGRLTLPLDASVDLQLNISFTYGPVILNGDGMDYYES
PLLNSGWLTIPPKDGTISGLINKAGRGDQFTVLPHVLTFAPRESSGNCYLPIQ
TSQIRDRDVLIESNIVVLPTQSIRYVIATYDISRSDHAIVYYVYDPIRTISYTHPF
RLTTKGRPDFLRIECFVWDDNLWCHQFYRFEADIANSTTSVENLVRIRFSCN
R-(X, WHEREIN X IS A TARGETING DOMAIN)-GS
Canine distemper virus Hemagglutinin (CDV H) delta 19
(SEQ ID NO: 175)
MTKLSLVTEGHGGRRPPYLLFVLLILLVGILALLAITGVRFHQVSTSNMEFSRL
LKEDMEKSEAVHHQVIDVLTPLFKIIGDEIGLRLPQKLNEIKQFILQKTNFFNP
NREFDFRDLHWCINPPSTVKVNFTNYCESIGIRKAIASAANPILLSALSGGRG
DIFPPHRCSGATTSVGKVFPLSVSLSMSLISRTSEVINMLTAISDGVYGKTYLL
VPDDIEREFDTREIRVFEIGFIKRWLNDMPLLQTTNYMVLPKNSKAKVCTIAV
GELTLASLCVEESTVLLYHDSSGSQDGILVVTLGIFWATPMDHIEEVIPVAHP
SMKKIHITNHRGFIKDSIATWMVPALASEKQEEQKGCLESACQRKTYPMCN
QASWEPFGGRQLPSYGRLTLPLDASVDLQLNISFTYGPVILNGDGMDYYES
PLLNSGWLTIPPKDGTISGLINKAGRGDQFTVLPHVLTFAPRESSGNCYLPIQ
TSQIRDRDVLIESNIVVLPTQSIRYVIATYDISRSDHAIVYYVYDPIRTISYTHPF
RLTTKGRPDFLRIECFVWDDNLWCHQFYRFEADIANSTTSVENLVRIRFSCN
R
Canine distemper virus Hemagglutinin (CDV H) delta 19 with targeting
domain fusion site
(SEQ ID NO: 176)
MTKLSLVTEGHGGRRPPYLLFVLLILLVGILALLAITGVRFHQVSTSNMEFSRL
LKEDMEKSEAVHHQVIDVLTPLFKIIGDEIGLRLPQKLNEIKQFILQKTNFFNP
NREFDFRDLHWCINPPSTVKVNFTNYCESIGIRKAIASAANPILLSALSGGRG
DIFPPHRCSGATTSVGKVFPLSVSLSMSLISRTSEVINMLTAISDGVYGKTYLL
VPDDIEREFDTREIRVFEIGFIKRWLNDMPLLQTTNYMVLPKNSKAKVCTIAV
GELTLASLCVEESTVLLYHDSSGSQDGILVVTLGIFWATPMDHIEEVIPVAHP
SMKKIHITNHRGFIKDSIATWMVPALASEKQEEQKGCLESACQRKTYPMCN
QASWEPFGGRQLPSYGRLTLPLDASVDLQLNISFTYGPVILNGDGMDYYES
PLLNSGWLTIPPKDGTISGLINKAGRGDQFTVLPHVLTFAPRESSGNCYLPIQ
TSQIRDRDVLIESNIVVLPTQSIRYVIATYDISRSDHAIVYYVYDPIRTISYTHPF
RLTTKGRPDFLRIECFVWDDNLWCHQFYRFEADIANSTTSVENLVRIRFSCN
R-(X, WHEREIN X IS A TARGETING DOMAIN)-GS
Canine distemper virus Fusion (CDV F) WT
(SEQ ID NO: 177)
MHRGIPKSSKTQTHTQQDRPPQPSTELEETRTSRARHSTTSAQRSTHYDPRTS
DRPVSYTMNRTRSRKQTSHRLKNIPVHGNHEATIQHIPESVSKGARSQIERRQ
PNAINSGSHCTWLVLWCLGMASLFLCSKAQIHWDNLSTIGIIGTDNVHYKIMT
RPSHQYLVIKLIPNASLIENCTKAELGEYEKLLNSVLEPINQALTLMTKNVKPL
QSLGSGRRQRRFAGVVLAGVALGVATAAQITAGIALHQSNLNAQAIQSLRTS
LEQSNKAIEEIREATQETVIAVQGVQDYVNNELVPAMQHMSCELVGQRLGLR
LLRYYTELLSIFGPSLRDPISAEISIQALIYALGGEIHKILEKLGYSGSDMIAILES
RGIKTKITHVDLPGKFIILSISYPTLSEVKGVIVHRLEAVSYNIGSQEWYTTVPR
YIATNGYLISNFDESSCVFVSESAICSQNSLYPMSPLLQQCIRGDTSSCARTLVS
GTMGNKFILSKGNIVANCASILCKCYSTSTIINQSPDKLLTFIASDTCPLVEIDG
ATIQVGGRQYPDMVYEGKVALGPAISLDRLDVGTNLGNALKKLDDAKVLID
SSNQILETVRRSSFNFGSLLSVPILSCTALALLLLIYCCKRRYQQTLKQHTKVD
PAFKPDLTGTSKSYVRSL
Canine distemper virus Fusion (CDV F) T to I hyperfusogenic mutation
(SEQ ID NO: 178)
MHRGIPKSSKTQTHTQQDRPPQPSTELEETRTSRARHSTTSAQRSTHYDPRTS
DRPVSYTMNRTRSRKQTSHRLKNIPVHGNHEATIQHIPESVSKGARSQIERRQ
PNAINSGSHCTWLVLWCLGMASLFLCSKAQIHWDNLSTIGIIGTDNVHYKIMT
RPSHQYLVIKLIPNASLIENCTKAELGEYEKLLNSVLEPINQALTLMTKNVKPL
QSLGSGRRQRRFAGVVLAGVALGVATAAQITAGIALHQSNLNAQAIQSLRTS
LEQSNKAIEEIREATQETVIAVQGVQDYVNNELVPAMQHMSCELVGQRLGLR
LLRYYTELLSIFGPSLRDPISAEISIQALIYALGGEIHKILEKLGYSGSDMIAILES
RGIKTKITHVDLPGKFIILSISYPTLSEVKGVIVHRLEAVSYNIGSQEWYTTVPR
YIATNGYLISNFDESSCVFVSESAICSQNSLYPMSPLLQQCIRGDTSSCARTLVS
GTMGNKFILSKGNIVANCASILCKCYSTSTIINQSPDKLLTFIASDTCPLVEIDG
ATIQVGGRQYPDMVYEGKVALGPAISLDRLDVGINLGNALKKLDDAKVLIDS
SNQILETVRRSSFNFGSLLSVPILSCTALALLLLIYCCKRRYQQTLKQHTKVDP
AFKPDLTGTSKSYVRSL
Canine distemper virus Fusion (CDV F) delta 24
(SEQ ID NO: 179)
MHRGIPKSSKTQTHTQQDRPPQPSTELEETRTSRARHSTTSAQRSTHYDPR
TSDRPVSYTMNRTRSRKQTSHRLKNIPVHGNHEATIQHIPESVSKGARSQIE
RRQPNAINSGSHCTWLVLWCLGMASLFLCSKAQIHWDNLSTIGIIGTDNVHY
KIMTRPSHQYLVIKLIPNASLIENCTKAELGEYEKLLNSVLEPINQALTLMTKN
VKPLQSLGSGRRQRRFAGVVLAGVALGVATAAQITAGIALHQSNLNAQAIQS
LRTSLEQSNKAIEEIREATQETVIAVQGVQDYVNNELVPAMQHMSCELVGQ
RLGLRLLRYYTELLSIFGPSLRDPISAEISIQALIYALGGEIHKILEKLGYSGSD
MIAILESRGIKTKITHVDLPGKFIILSISYPTLSEVKGVIVHRLEAVSYNIGSQEW
YTTVPRYIATNGYLISNFDESSCVFVSESAICSQNSLYPMSPLLQQCIRGDTS
SCARTLVSGTMGNKFILSKGNIVANCASILCKCYSTSTIINQSPDKLLTFIASDT
CPLVEIDGATIQVGGRQYPDMVYEGKVALGPAISLDRLDVGTNLGNALKKLD
DAKVLIDSSNQILETVRRSSFNFGSLLSVPILSCTALALLLLIYCCKRRYQQTL
K
Canine distemper virus Fusion (CDV F) delta 24 T to I hyperfusogenic
mutation
(SEQ ID NO: 180)
MHRGIPKSSKTQTHTQQDRPPQPSTELEETRTSRARHSTTSAQRSTHYDPR
TSDRPVSYTMNRTRSRKQTSHRLKNIPVHGNHEATIQHIPESVSKGARSQIE
RRQPNAINSGSHCTWLVLWCLGMASLFLCSKAQIHWDNLSTIGIIGTDNVHY
KIMTRPSHQYLVIKLIPNASLIENCTKAELGEYEKLLNSVLEPINQALTLMTKN
VKPLQSLGSGRRQRRFAGVVLAGVALGVATAAQITAGIALHQSNLNAQAIQS
LRTSLEQSNKAIEEIREATQETVIAVQGVQDYVNNELVPAMQHMSCELVGQ
RLGLRLLRYYTELLSIFGPSLRDPISAEISIQALIYALGGEIHKILEKLGYSGSD
MIAILESRGIKTKITHVDLPGKFIILSISYPTLSEVKGVIVHRLEAVSYNIGSQEW
YTTVPRYIATNGYLISNFDESSCVFVSESAICSQNSLYPMSPLLQQCIRGDTS
SCARTLVSGTMGNKFILSKGNIVANCASILCKCYSTSTIINQSPDKLLTFIASDT
CPLVEIDGATIQVGGRQYPDMVYEGKVALGPAISLDRLDVGINLGNALKKLD
DAKVLIDSSNQILETVRRSSFNFGSLLSVPILSCTALALLLLIYCCKRRYQQTL
K
Canine distemper virus Fusion (CDV F) delta 30
(SEQ ID NO: 181)
MHRGIPKSSKTQTHTQQDRPPQPSTELEETRTSRARHSTTSAQRSTHYDPRTS
DRPVSYTMNRTRSRKQTSHRLKNIPVHGNHEATIQHIPESVSKGARSQIERRQ
PNAINSGSHCTWLVLWCLGMASLFLCSKAQIHWDNLSTIGIIGTDNVHYKIMT
RPSHQYLVIKLIPNASLIENCTKAELGEYEKLLNSVLEPINQALTLMTKNVKPL
QSLGSGRRQRRFAGVVLAGVALGVATAAQITAGIALHQSNLNAQAIQSLRTS
LEQSNKAIEEIREATQETVIAVQGVQDYVNNELVPAMQHMSCELVGQRLGLR
LLRYYTELLSIFGPSLRDPISAEISIQALIYALGGEIHKILEKLGYSGSDMIAILES
RGIKTKITHVDLPGKFIILSISYPTLSEVKGVIVHRLEAVSYNIGSQEWYTTVPR
YIATNGYLISNFDESSCVFVSESAICSQNSLYPMSPLLQQCIRGDTSSCARTLVS
GTMGNKFILSKGNIVANCASILCKCYSTSTIINQSPDKLLTFIASDTCPLVEIDG
ATIQVGGRQYPDMVYEGKVALGPAISLDRLDVGTNLGNALKKLDDAKVLID
SSNQILETVRRSSFNFGSLLSVPILSCTALALLLLIYCCKRR
Canine distemper virus Fusion (CDV F) delta 30 T to I hyperfusogenic
mutation
(SEQ ID NO: 182)
MHRGIPKSSKTQTHTQQDRPPQPSTELEETRTSRARHSTTSAQRSTHYDPRTS
DRPVSYTMNRTRSRKQTSHRLKNIPVHGNHEATIQHIPESVSKGARSQIERRQ
PNAINSGSHCTWLVLWCLGMASLFLCSKAQIHWDNLSTIGIIGTDNVHYKIMT
RPSHQYLVIKLIPNASLIENCTKAELGEYEKLLNSVLEPINQALTLMTKNVKPL
QSLGSGRRQRRFAGVVLAGVALGVATAAQITAGIALHQSNLNAQAIQSLRTS
LEQSNKAIEEIREATQETVIAVQGVQDYVNNELVPAMQHMSCELVGQRLGLR
LLRYYTELLSIFGPSLRDPISAEISIQALIYALGGEIHKILEKLGYSGSDMIAILES
RGIKTKITHVDLPGKFIILSISYPTLSEVKGVIVHRLEAVSYNIGSQEWYTTVPR
YIATNGYLISNFDESSCVFVSESAICSQNSLYPMSPLLQQCIRGDTSSCARTLVS
GTMGNKFILSKGNIVANCASILCKCYSTSTIINQSPDKLLTFIASDTCPLVEIDG
ATIQVGGRQYPDMVYEGKVALGPAISLDRLDVGINLGNALKKLDDAKVLIDS
SNQILETVRRSSFNFGSLLSVPILSCTALALLLLIYCCKRR
Canine distemper virus Fusion (CDV F) WT mini signal sequence d107
(SEQ ID NO: 183)
MNAINSGSHCTWLVLWCLGMASLFLCSKAQIHWDNLSTIGIIGTDNVHYKIM
TRPSHQYLVIKLIPNASLIENCTKAELGEYEKLLNSVLEPINQALTLMTKNVKP
LQSLGSGRRQRRFAGVVLAGVALGVATAAQITAGIALHQSNLNAQAIQSLRT
SLEQSNKAIEEIREATQETVIAVQGVQDYVNNELVPAMQHMSCELVGQRLGL
RLLRYYTELLSIFGPSLRDPISAEISIQALIYALGGEIHKILEKLGYSGSDMIAILE
SRGIKTKITHVDLPGKFIILSISYPTLSEVKGVIVHRLEAVSYNIGSQEWYTTVP
RYIATNGYLISNFDESSCVFVSESAICSQNSLYPMSPLLQQCIRGDTSSCARTLV
SGTMGNKFILSKGNIVANCASILCKCYSTSTIINQSPDKLLTFIASDTCPLVEID
GATIQVGGRQYPDMVYEGKVALGPAISLDRLDVGTNLGNALKKLDDAKVLI
DSSNQILETVRRSSFNFGSLLSVPILSCTALALLLLIYCCKRRYQQTLKQHTKV
DPAFKPDLTGTSKSYVRSL
Canine distemper virus Fusion (CDV F) WT mini signal sequence d107 T
to I hyperfusogenic mutation
(SEQ ID NO: 184)
MNAINSGSHCTWLVLWCLGMASLFLCSKAQIHWDNLSTIGIIGTDNVHYKIM
TRPSHQYLVIKLIPNASLIENCTKAELGEYEKLLNSVLEPINQALTLMTKNVKP
LQSLGSGRRQRRFAGVVLAGVALGVATAAQITAGIALHQSNLNAQAIQSLRT
SLEQSNKAIEEIREATQETVIAVQGVQDYVNNELVPAMQHMSCELVGQRLGL
RLLRYYTELLSIFGPSLRDPISAEISIQALIYALGGEIHKILEKLGYSGSDMIAILE
SRGIKTKITHVDLPGKFIILSISYPTLSEVKGVIVHRLEAVSYNIGSQEWYTTVP
RYIATNGYLISNFDESSCVFVSESAICSQNSLYPMSPLLQQCIRGDTSSCARTLV
SGTMGNKFILSKGNIVANCASILCKCYSTSTIINQSPDKLLTFIASDTCPLVEID
GATIQVGGRQYPDMVYEGKVALGPAISLDRLDVGINLGNALKKLDDAKVLID
SSNQILETVRRSSFNFGSLLSVPILSCTALALLLLIYCCKRRYQQTLKQHTKVD
PAFKPDLTGTSKSYVRSL
Canine distemper virus Fusion (CDV F) mini signal sequence d107 delta 24
(SEQ ID NO: 185)
MNAINSGSHCTWLVLWCLGMASLFLCSKAQIHWDNLSTIGIIGTDNVHYKIM
TRPSHQYLVIKLIPNASLIENCTKAELGEYEKLLNSVLEPINQALTLMTKNVKP
LQSLGSGRRQRRFAGVVLAGVALGVATAAQITAGIALHQSNLNAQAIQSLRT
SLEQSNKAIEEIREATQETVIAVQGVQDYVNNELVPAMQHMSCELVGQRLGL
RLLRYYTELLSIFGPSLRDPISAEISIQALIYALGGEIHKILEKLGYSGSDMIAILE
SRGIKTKITHVDLPGKFIILSISYPTLSEVKGVIVHRLEAVSYNIGSQEWYTTVP
RYIATNGYLISNFDESSCVFVSESAICSQNSLYPMSPLLQQCIRGDTSSCARTLV
SGTMGNKFILSKGNIVANCASILCKCYSTSTIINQSPDKLLTFIASDTCPLVEID
GATIQVGGRQYPDMVYEGKVALGPAISLDRLDVGTNLGNALKKLDDAKVLI
DSSNQILETVRRSSFNFGSLLSVPILSCTALALLLLIYCCKRRYQQTLK
Canine distemper virus Fusion (CDV F) mini signal sequence d107 delta
24 T to I hyperfusogenic mutation
(SEQ ID NO: 186)
MNAINSGSHCTWLVLWCLGMASLFLCSKAQIHWDNLSTIGIIGTDNVHYKIM
TRPSHQYLVIKLIPNASLIENCTKAELGEYEKLLNSVLEPINQALTLMTKNVKP
LQSLGSGRRQRRFAGVVLAGVALGVATAAQITAGIALHQSNLNAQAIQSLRT
SLEQSNKAIEEIREATQETVIAVQGVQDYVNNELVPAMQHMSCELVGQRLGL
RLLRYYTELLSIFGPSLRDPISAEISIQALIYALGGEIHKILEKLGYSGSDMIAILE
SRGIKTKITHVDLPGKFIILSISYPTLSEVKGVIVHRLEAVSYNIGSQEWYTTVP
RYIATNGYLISNFDESSCVFVSESAICSQNSLYPMSPLLQQCIRGDTSSCARTLV
SGTMGNKFILSKGNIVANCASILCKCYSTSTIINQSPDKLLTFIASDTCPLVEID
GATIQVGGRQYPDMVYEGKVALGPAISLDRLDVGINLGNALKKLDDAKVLID
SSNQILETVRRSSFNFGSLLSVPILSCTALALLLLIYCCKRRYQQTLK
Canine distemper virus Fusion (CDV F) mini signal sequence d107 delta 30
(SEQ ID NO: 187)
MNAINSGSHCTWLVLWCLGMASLFLCSKAQIHWDNLSTIGIIGTDNVHYKIM
TRPSHQYLVIKLIPNASLIENCTKAELGEYEKLLNSVLEPINQALTLMTKNVKP
LQSLGSGRRQRRFAGVVLAGVALGVATAAQITAGIALHQSNLNAQAIQSLRT
SLEQSNKAIEEIREATQETVIAVQGVQDYVNNELVPAMQHMSCELVGQRLGL
RLLRYYTELLSIFGPSLRDPISAEISIQALIYALGGEIHKILEKLGYSGSDMIAILE
SRGIKTKITHVDLPGKFIILSISYPTLSEVKGVIVHRLEAVSYNIGSQEWYTTVP
RYIATNGYLISNFDESSCVFVSESAICSQNSLYPMSPLLQQCIRGDTSSCARTLV
SGTMGNKFILSKGNIVANCASILCKCYSTSTIINQSPDKLLTFIASDTCPLVEID
GATIQVGGRQYPDMVYEGKVALGPAISLDRLDVGTNLGNALKKLDDAKVLI
DSSNQILETVRRSSFNFGSLLSVPILSCTALALLLLIYCCKRR
Canine distemper virus Fusion (CDV F) mini signal sequence d107 delta
30 T to I hyperfusogenic mutation
(SEQ ID NO: 188)
MNAINSGSHCTWLVLWCLGMASLFLCSKAQIHWDNLSTIGIIGTDNVHYKIM
TRPSHQYLVIKLIPNASLIENCTKAELGEYEKLLNSVLEPINQALTLMTKNVKP
LQSLGSGRRQRRFAGVVLAGVALGVATAAQITAGIALHQSNLNAQAIQSLRT
SLEQSNKAIEEIREATQETVIAVQGVQDYVNNELVPAMQHMSCELVGQRLGL
RLLRYYTELLSIFGPSLRDPISAEISIQALIYALGGEIHKILEKLGYSGSDMIAILE
SRGIKTKITHVDLPGKFIILSISYPTLSEVKGVIVHRLEAVSYNIGSQEWYTTVP
RYIATNGYLISNFDESSCVFVSESAICSQNSLYPMSPLLQQCIRGDTSSCARTLV
SGTMGNKFILSKGNIVANCASILCKCYSTSTIINQSPDKLLTFIASDTCPLVEID
GATIQVGGRQYPDMVYEGKVALGPAISLDRLDVGINLGNALKKLDDAKVLID
SSNQILETVRRSSFNFGSLLSVPILSCTALALLLLIYCCKRR
Nipah virus Glycoprotein (NiVG) WT
(SEQ ID NO: 189)
MPAENKKVRFENTTSDKGKIPSKVIKSYYGTMDIKKINEGLLDSKILSAFNTVI
ALLGSIVIIVMNIMIIQNYTRSTDNQAVIKDALQGIQQQIKGLADKIGTEIGPKV
SLIDTSSTITIPANIGLLGSKISQSTASINENVNEKCKFTLPPLKIHECNISCPNPLP
FREYRPQTEGVSNLVGLPNNICLQKTSNQILKPKLISYTLPVVGQSGTCITDPL
LAMDEGYFAYSHLERIGSCSRGVSKQRIIGVGEVLDRGDEVPSLFMTNVWTP
PNPNTVYHCSAVYNNEFYYVLCAVSTVGDPILNSTYWSGSLMMTRLAVKPK
SNGGGYNQHQLALRSIEKGRYDKVMPYGPSGIKQGDTLYFPAVGFLVRTEFK
YNDSNCPITKCQYSKPENCRLSMGIRPNSHYILRSGLLKYNLSDGENPKVVFIE
ISDQRLSIGSPSKIYDSLGQPVFYQASFSWDTMIKFGDVLTVNPLVVNWRNNT
VISRPGQSQCPRFNTCPEICWEGVYNDAFLIDRINWISAGVFLDSNQTAENPVF
TVFKDNEILYRAQLASEDTNAQKTITNCFLLKNKIWCISLVEIYDTGDNVIRPK
LFAVKIPEQCT
Nipah virus Glycoprotein (NiVG) WT with targeting domain fusion site
(SEQ ID NO: 199)
MPAENKKVRFENTTSDKGKIPSKVIKSYYGTMDIKKINEGLLDSKILSAFNTVI
ALLGSIVIIVMNIMIIQNYTRSTDNQAVIKDALQGIQQQIKGLADKIGTEIGPKV
SLIDTSSTITIPANIGLLGSKISQSTASINENVNEKCKFTLPPLKIHECNISCPNPLP
FREYRPQTEGVSNLVGLPNNICLQKTSNQILKPKLISYTLPVVGQSGTCITDPL
LAMDEGYFAYSHLERIGSCSRGVSKQRIIGVGEVLDRGDEVPSLFMTNVWTP
PNPNTVYHCSAVYNNEFYYVLCAVSTVGDPILNSTYWSGSLMMTRLAVKPK
SNGGGYNQHQLALRSIEKGRYDKVMPYGPSGIKQGDTLYFPAVGFLVRTEFK
YNDSNCPITKCQYSKPENCRLSMGIRPNSHYILRSGLLKYNLSDGENPKVVFIE
ISDQRLSIGSPSKIYDSLGQPVFYQASFSWDTMIKFGDVLTVNPLVVNWRNNT
VISRPGQSQCPRFNTCPEICWEGVYNDAFLIDRINWISAGVFLDSNQTAENPVF
TVFKDNEILYRAQLASEDTNAQKTITNCFLLKNKIWCISLVEIYDTGDNVIRPK
LFAVKIPEQCT-(X, WHEREIN X IS A TARGETING DOMAIN)-GS
Nipah virus Glycoprotein (NiVG) delta 33
(SEQ ID NO: 200)
MIKKINEGLLDSKILSAFNTVIALLGSIVIIVMNIMIIQNYTRSTDNQAVIKDAL
QGIQQQIKGLADKIGTEIGPKVSLIDTSSTITIPANIGLLGSKISQSTASINENVNE
KCKFTLPPLKIHECNISCPNPLPFREYRPQTEGVSNLVGLPNNICLQKTSNQILK
PKLISYTLPVVGQSGTCITDPLLAMDEGYFAYSHLERIGSCSRGVSKQRIIGVG
EVLDRGDEVPSLFMTNVWTPPNPNTVYHCSAVYNNEFYYVLCAVSTVGDPIL
NSTYWSGSLMMTRLAVKPKSNGGGYNQHQLALRSIEKGRYDKVMPYGPSGI
KQGDTLYFPAVGFLVRTEFKYNDSNCPITKCQYSKPENCRLSMGIRPNSHYIL
RSGLLKYNLSDGENPKVVFIEISDQRLSIGSPSKIYDSLGQPVFYQASFSWDTM
IKFGDVLTVNPLVVNWRNNTVISRPGQSQCPRFNTCPEICWEGVYNDAFLIDR
INWISAGVFLDSNQTAENPVFTVFKDNEILYRAQLASEDTNAQKTITNCFLLK
NKIWCISLVEIYDTGDNVIRPKLFAVKIPEQCT
Nipah virus Glycoprotein (NiVG) delta 33 with targeting domain fusion site
(SEQ ID NO: 201)
MIKKINEGLLDSKILSAFNTVIALLGSIVIIVMNIMIIQNYTRSTDNQAVIKDAL
QGIQQQIKGLADKIGTEIGPKVSLIDTSSTITIPANIGLLGSKISQSTASINENVNE
KCKFTLPPLKIHECNISCPNPLPFREYRPQTEGVSNLVGLPNNICLQKTSNQILK
PKLISYTLPVVGQSGTCITDPLLAMDEGYFAYSHLERIGSCSRGVSKQRIIGVG
EVLDRGDEVPSLFMTNVWTPPNPNTVYHCSAVYNNEFYYVLCAVSTVGDPIL
NSTYWSGSLMMTRLAVKPKSNGGGYNQHQLALRSIEKGRYDKVMPYGPSGI
KQGDTLYFPAVGFLVRTEFKYNDSNCPITKCQYSKPENCRLSMGIRPNSHYIL
RSGLLKYNLSDGENPKVVFIEISDQRLSIGSPSKIYDSLGQPVFYQASFSWDTM
IKFGDVLTVNPLVVNWRNNTVISRPGQSQCPRFNTCPEICWEGVYNDAFLIDR
INWISAGVFLDSNQTAENPVFTVFKDNEILYRAQLASEDTNAQKTITNCFLLK
NKIWCISLVEIYDTGDNVIRPKLFAVKIPEQCT-(X, WHEREIN X IS A
TARGETING DOMAIN)-GS
Nipah virus Glycoprotein (NiVG) delta 34
(SEQ ID NO: 202)
MKKINEGLLDSKILSAFNTVIALLGSIVIIVMNIMIIQNYTRSTDNQAVIKDALQ
GIQQQIKGLADKIGTEIGPKVSLIDTSSTITIPANIGLLGSKISQSTASINENVNEK
CKFTLPPLKIHECNISCPNPLPFREYRPQTEGVSNLVGLPNNICLQKTSNQILKP
KLISYTLPVVGQSGTCITDPLLAMDEGYFAYSHLERIGSCSRGVSKQRIIGVGE
VLDRGDEVPSLFMTNVWTPPNPNTVYHCSAVYNNEFYYVLCAVSTVGDPIL
NSTYWSGSLMMTRLAVKPKSNGGGYNQHQLALRSIEKGRYDKVMPYGPSGI
KQGDTLYFPAVGFLVRTEFKYNDSNCPITKCQYSKPENCRLSMGIRPNSHYIL
RSGLLKYNLSDGENPKVVFIEISDQRLSIGSPSKIYDSLGQPVFYQASFSWDTM
IKFGDVLTVNPLVVNWRNNTVISRPGQSQCPRFNTCPEICWEGVYNDAFLIDR
INWISAGVFLDSNQTAENPVFTVFKDNEILYRAQLASEDTNAQKTITNCFLLK
NKIWCISLVEIYDTGDNVIRPKLFAVKIPEQCT
Nipah virus Glycoprotein (NiVG) delta 34 with targeting domain fusion site
(SEQ ID NO: 203)
MKKINEGLLDSKILSAFNTVIALLGSIVIIVMNIMIIQNYTRSTDNQAVIKDALQ
GIQQQIKGLADKIGTEIGPKVSLIDTSSTITIPANIGLLGSKISQSTASINENVNEK
CKFTLPPLKIHECNISCPNPLPFREYRPQTEGVSNLVGLPNNICLQKTSNQILKP
KLISYTLPVVGQSGTCITDPLLAMDEGYFAYSHLERIGSCSRGVSKQRIIGVGE
VLDRGDEVPSLFMTNVWTPPNPNTVYHCSAVYNNEFYYVLCAVSTVGDPIL
NSTYWSGSLMMTRLAVKPKSNGGGYNQHQLALRSIEKGRYDKVMPYGPSGI
KQGDTLYFPAVGFLVRTEFKYNDSNCPITKCQYSKPENCRLSMGIRPNSHYIL
RSGLLKYNLSDGENPKVVFIEISDQRLSIGSPSKIYDSLGQPVFYQASFSWDTM
IKFGDVLTVNPLVVNWRNNTVISRPGQSQCPRFNTCPEICWEGVYNDAFLIDR
INWISAGVFLDSNQTAENPVFTVFKDNEILYRAQLASEDTNAQKTITNCFLLK
NKIWCISLVEIYDTGDNVIRPKLFAVKIPEQCT-(X, WHEREIN X IS A
TARGETING DOMAIN)-GS
Nipah virus Fusion WT
(SEQ ID NO: 204)
MVVILDKRCYCNLLILILMISECSVGILHYEKLSKIGLVKGVTRKYKIKSNPLT
KDIVIKMIPNVSNMSQCTGSVMENYKTRLNGILTPIKGALEIYKNNTHDLVGD
VRLAGVIMAGVAIGIATAAQITAGVALYEAMKNADNINKLKSSIESTNEAVV
KLQETAEKTVYVLTALQDYINTNLVPTIDKISCKQTELSLDLALSKYLSDLLFV
FGPNLQDPVSNSMTIQAISQAFGGNYETLLRTLGYATEDFDDLLESDSITGQII
YVDLSSYYIIVRVYFPILTEIQQAYIQELLPVSFNNDNSEWISIVPNFILVRNTLIS
NIEIGFCLITKRSVICNQDYATPMTNNMRECLTGSTEKCPRELVVSSHVPRFAL
SNGVLFANCISVTCQCQTTGRAISQSGEQTLLMIDNTTCPTAVLGNVIISLGKY
LGSVNYNSEGIAIGPPVFTDKVDISSQISSMNQSLQQSKDYIKEAQRLLDTVNP
SLISMLSMIILYVLSIASLCIGLITFISFIIVEKKRNTYSRLEDRRVRPTSSGDLYYI
GT
Nipah virus Fusion delta 22
(SEQ ID NO: 205)
MVVILDKRCYCNLLILILMISECSVGILHYEKLSKIGLVKGVTRKYKIKSNPLT
KDIVIKMIPNVSNMSQCTGSVMENYKTRLNGILTPIKGALEIYKNNTHDLVGD
VRLAGVIMAGVAIGIATAAQITAGVALYEAMKNADNINKLKSSIESTNEAVV
KLQETAEKTVYVLTALQDYINTNLVPTIDKISCKQTELSLDLALSKYLSDLLFV
FGPNLQDPVSNSMTIQAISQAFGGNYETLLRTLGYATEDFDDLLESDSITGQII
YVDLSSYYIIVRVYFPILTEIQQAYIQELLPVSFNNDNSEWISIVPNFILVRNTLIS
NIEIGFCLITKRSVICNQDYATPMTNNMRECLTGSTEKCPRELVVSSHVPRFAL
SNGVLFANCISVTCQCQTTGRAISQSGEQTLLMIDNTTCPTAVLGNVIISLGKY
LGSVNYNSEGIAIGPPVFTDKVDISSQISSMNQSLQQSKDYIKEAQRLLDTVNP
SLISMLSMIILYVLSIASLCIGLITFISFIIVEKKRNT
Nipah virus Fusion delta 25
(SEQ ID NO: 206)
MVVILDKRCYCNLLILILMISECSVGILHYEKLSKIGLVKGVTRKYKIKSNPLT
KDIVIKMIPNVSNMSQCTGSVMENYKTRLNGILTPIKGALEIYKNNTHDLVGD
VRLAGVIMAGVAIGIATAAQITAGVALYEAMKNADNINKLKSSIESTNEAVV
KLQETAEKTVYVLTALQDYINTNLVPTIDKISCKQTELSLDLALSKYLSDLLFV
FGPNLQDPVSNSMTIQAISQAFGGNYETLLRTLGYATEDFDDLLESDSITGQII
YVDLSSYYIIVRVYFPILTEIQQAYIQELLPVSFNNDNSEWISIVPNFILVRNTLIS
NIEIGFCLITKRSVICNQDYATPMTNNMRECLTGSTEKCPRELVVSSHVPRFAL
SNGVLFANCISVTCQCQTTGRAISQSGEQTLLMIDNTTCPTAVLGNVIISLGKY
LGSVNYNSEGIAIGPPVFTDKVDISSQISSMNQSLQQSKDYIKEAQRLLDTVNP
SLISMLSMIILYVLSIASLCIGLITFISFIIVEKK
Nipah Virus Glycoprotein (NIVG) (E501A)
(SEQ ID NO: 207)
MPAENKKVRFENTTSDKGKIPSKVIKSYYGTMDIKKINEGLLDSKILSAFNTVI
ALLGSIVIIVMNIMIIQNYTRSTDNQAVIKDALQGIQQQIKGLADKIGTEIGPKV
SLIDTSSTITIPANIGLLGSKISQSTASINENVNEKCKFTLPPLKIHECNISCPNPLP
FREYRPQTEGVSNLVGLPNNICLQKTSNQILKPKLISYTLPVVGQSGTCITDPL
LAMDEGYFAYSHLERIGSCSRGVSKQRIIGVGEVLDRGDEVPSLFMTNVWTP
PNPNTVYHCSAVYNNEFYYVLCAVSTVGDPILNSTYWSGSLMMTRLAVKPK
SNGGGYNQHQLALRSIEKGRYDKVMPYGPSGIKQGDTLYFPAVGFLVRTEFK
YNDSNCPITKCQYSKPENCRLSMGIRPNSHYILRSGLLKYNLSDGENPKVVFIE
ISDQRLSIGSPSKIYDSLGQPVFYQASFSWDTMIKFGDVLTVNPLVVNWRNNT
VISRPGQSQCPRFNTCPAICWEGVYNDAFLIDRINWISAGVFLDSNQTAENPVF
TVFKDNEILYRAQLASEDTNAQKTITNCFLLKNKIWCISLVEIYDTGDNVIRPK
LFAVKIPEQCT
Nipah Virus Glycoprotein (NIVG) (W504A)
(SEQ ID NO: 208)
MPAENKKVRFENTTSDKGKIPSKVIKSYYGTMDIKKINEGLLDSKILSAFNTVI
ALLGSIVIIVMNIMIIQNYTRSTDNQAVIKDALQGIQQQIKGLADKIGTEIGPKV
SLIDTSSTITIPANIGLLGSKISQSTASINENVNEKCKFTLPPLKIHECNISCPNPLP
FREYRPQTEGVSNLVGLPNNICLQKTSNQILKPKLISYTLPVVGQSGTCITDPL
LAMDEGYFAYSHLERIGSCSRGVSKQRIIGVGEVLDRGDEVPSLFMTNVWTP
PNPNTVYHCSAVYNNEFYYVLCAVSTVGDPILNSTYWSGSLMMTRLAVKPK
SNGGGYNQHQLALRSIEKGRYDKVMPYGPSGIKQGDTLYFPAVGFLVRTEFK
YNDSNCPITKCQYSKPENCRLSMGIRPNSHYILRSGLLKYNLSDGENPKVVFIE
ISDQRLSIGSPSKIYDSLGQPVFYQASFSWDTMIKFGDVLTVNPLVVNWRNNT
VISRPGQSQCPRFNTCPEICAEGVYNDAFLIDRINWISAGVFLDSNQTAENPVF
TVFKDNEILYRAQLASEDTNAQKTITNCFLLKNKIWCISLVEIYDTGDNVIRPK
LFAVKIPEQCT
Nipah Virus Glycoprotein (NIVG) (Q530A)
(SEQ ID NO: 209)
MPAENKKVRFENTTSDKGKIPSKVIKSYYGTMDIKKINEGLLDSKILSAFNTVI
ALLGSIVIIVMNIMIIQNYTRSTDNQAVIKDALQGIQQQIKGLADKIGTEIGPKV
SLIDTSSTITIPANIGLLGSKISQSTASINENVNEKCKFTLPPLKIHECNISCPNPLP
FREYRPQTEGVSNLVGLPNNICLQKTSNQILKPKLISYTLPVVGQSGTCITDPL
LAMDEGYFAYSHLERIGSCSRGVSKQRIIGVGEVLDRGDEVPSLFMTNVWTP
PNPNTVYHCSAVYNNEFYYVLCAVSTVGDPILNSTYWSGSLMMTRLAVKPK
SNGGGYNQHQLALRSIEKGRYDKVMPYGPSGIKQGDTLYFPAVGFLVRTEFK
YNDSNCPITKCQYSKPENCRLSMGIRPNSHYILRSGLLKYNLSDGENPKVVFIE
ISDQRLSIGSPSKIYDSLGQPVFYQASFSWDTMIKFGDVLTVNPLVVNWRNNT
VISRPGQSQCPRFNTCPEICWEGVYNDAFLIDRINWISAGVFLDSNATAENPVF
TVFKDNEILYRAQLASEDTNAQKTITNCFLLKNKIWCISLVEIYDTGDNVIRPK
LFAVKIPEQCT
Nipah Virus Glycoprotein (NIVG) (E533A)
(SEQ ID NO: 210)
MPAENKKVRFENTTSDKGKIPSKVIKSYYGTMDIKKINEGLLDSKILSAFNTVI
ALLGSIVIIVMNIMIIQNYTRSTDNQAVIKDALQGIQQQIKGLADKIGTEIGPKV
SLIDTSSTITIPANIGLLGSKISQSTASINENVNEKCKFTLPPLKIHECNISCPNPLP
FREYRPQTEGVSNLVGLPNNICLQKTSNQILKPKLISYTLPVVGQSGTCITDPL
LAMDEGYFAYSHLERIGSCSRGVSKQRIIGVGEVLDRGDEVPSLFMTNVWTP
PNPNTVYHCSAVYNNEFYYVLCAVSTVGDPILNSTYWSGSLMMTRLAVKPK
SNGGGYNQHQLALRSIEKGRYDKVMPYGPSGIKQGDTLYFPAVGFLVRTEFK
YNDSNCPITKCQYSKPENCRLSMGIRPNSHYILRSGLLKYNLSDGENPKVVFIE
ISDQRLSIGSPSKIYDSLGQPVFYQASFSWDTMIKFGDVLTVNPLVVNWRNNT
VISRPGQSQCPRFNTCPEICWEGVYNDAFLIDRINWISAGVFLDSNQTAANPVF
TVFKDNEILYRAQLASEDTNAQKTITNCFLLKNKIWCISLVEIYDTGDNVIRPK
LFAVKIPEQCT
Cocal virus glycoprotein (CVG) WT
(SEQ ID NO: 211)
MNFLLLTFIVLPLCSHAKFSIVFPQSQKGNWKNVPSSYHYCPSSSDQNWHNDL
LGITMKVKMPKTHKAIQADGWMCHAAKWITTCDFRWYGPKYITHSIHSIQPT
SEQCKESIKQTKQGTWMSPGFPPQNCGYATVTDSVAVVVQATPHHVLVDEY
TGEWIDSQFPNGKCETEECETVHNSTVWYSDYKVTGLCDATLVDTEITFFSED
GKKESIGKPNTGYRSNYFAYEKGDKVCKMNYCKHAGVRLPSGVWFEFVDQ
DVYAAAKLPECPVGATISAPTQTSVDVSLILDVERILDYSLCQETWSKIRSKQP
VSPVDLSYLAPKNPGTGPAFTIINGTLKYFETRYIRIDIDNPIISKMVGKISGSQT
ERELWTEWFPYEGVEIGPNGILKTPTGYKFPLFMIGHGMLDSDLHKTSQAEVF
EHPHLAEAPKQLPEEETLFFGDTGISKNPVELIEGWFSSWKSTVVTFFFAIGVFI
LLYVVARIVIAVRYRYQGSNNKRIYNDIEMSRFRK
Cocal virus glycoprotein (CVG) (K64Q)
(SEQ ID NO: 212)
MNFLLLTFIVLPLCSHAKFSIVFPQSQKGNWKNVPSSYHYCPSSSDQNWHNDLLGIT
MKVKMPQTHKAIQADGWMCHAAKWITTCDFRWYGPKYITHSIHSIQPTSEQCKESI
KQTKQGTWMSPGFPPQNCGYATVTDSVAVWVQATPHHVLVDEYTGEWIDSQFPN
GKCETEECETVHNSTVWYSDYKVTGLCDATLVDTEITFFSEDGKKESIGKPNTGYR
SNYFAYEKGDKVCKMNYCKHAGVRLPSGVWFEFVDQDVYAAAKLPECPVGATISA
PTQTSVDVSLILDVERILDYSLCQETWSKIRSKQPVSPVDLSYLAPKNPGTGPAFTIIN
GTLKYFETRYIRIDIDNPIISKMVGKISGSQTERELWTEWFPYEGVEIGPNGILKTPTG
YKFPLFMIGHGMLDSDLHKTSQAEVFEHPHLAEAPKQLPEEETLFFGDTGISKNPVE
LIEGWFSSWKSTVVTFFFAIGVFILLYVVARIVIAVRYRYQGSNNKRIYNDIEMSRFRK
Cocal virus glycoprotein (CVG) (R371A)
(SEQ ID NO: 213)
MNFLLLTFIVLPLCSHAKFSIVFPQSQKGNWKNVPSSYHYCPSSSDQNWHNDLLGIT
MKVKMPKTHKAIQADGWMCHAAKWITTCDFRWYGPKYITHSIHSIQPTSEQCKESI
KQTKQGTWMSPGFPPQNCGYATVTDSVAVVVQATPHHVLVDEYTGEWIDSQFPN
GKCETEECETVHNSTVWYSDYKVTGLCDATLVDTEITFFSEDGKKESIGKPNTGYR
SNYFAYEKGDKVCKMNYCKHAGVRLPSGVWFEFVDQDVYAAAKLPECPVGATISA
PTQTSVDVSLILDVERILDYSLCQETWSKIRSKQPVSPVDLSYLAPKNPGTGPAFTIIN
GTLKYFETRYIRIDIDNPIISKMVGKISGSQTEAELWTEWFPYEGVEIGPNGILKTPTG
YKFPLFMIGHGMLDSDLHKTSQAEVFEHPHLAEAPKQLPEEETLFFGDTGISKNPVE
LIEGWFSSWKSTVVTFFFAIGVFILLYVVARIVIAVRYRYQGSNNKRIYNDIEMSRFRK
Cocal virus glycoprotein (CVG) (K64Q) (R371A)
(SEQ ID NO: 214))
MNFLLLTFIVLPLCSHAKFSIVFPQSQKGNWKNVPSSYHYCPSSSDQNWHNDL
LGITMKVKMPQTHKAIQADGWMCHAAKWITTCDFRWYGPKYITHSIHSIQPT
SEQCKESIKQTKQGTWMSPGFPPQNCGYATVTDSVAVVVQATPHHVLVDEY
TGEWIDSQFPNGKCETEECETVHNSTVWYSDYKVTGLCDATLVDTEITFFSED
GKKESIGKPNTGYRSNYFAYEKGDKVCKMNYCKHAGVRLPSGVWFEFVDQ
DVYAAAKLPECPVGATISAPTQTSVDVSLILDVERILDYSLCQETWSKIRSKQP
VSPVDLSYLAPKNPGTGPAFTIINGTLKYFETRYIRIDIDNPIISKMVGKISGSQT
EAELWTEWFPYEGVEIGPNGILKTPTGYKFPLFMIGHGMLDSDLHKTSQAEVF
EHPHLAEAPKQLPEEETLFFGDTGISKNPVELIEGWFSSWKSTVVTFFFAIGVFI
LLYVVARIVIAVRYRYQGSNNKRIYNDIEMSRFRK
Targeting domain fusion site to transmembrane PDGFR anchor
(SEQ ID NO: 215)
MALPVTALLLPLALLLHAARPEQKLISEEDLGSSGSGSAVS-(X, WHEREIN X
IS A TARGETING DOMAIN)-
NAVGODTQEVIVVPHSLPFKVVVISAILALVVLTIISLIILIMLWQKKPR
Targeting domain fusion site to transmembrane CD9 anchor
(SEQ ID NO: 216)
MLTRTLAVRSFAATMSPVKGGTKCIKYLLFGFNFIFWLAGIAVLAIGLWLRFD
SQTKSIFEQETN-(X, WHEREIN X IS A TARGETING DOMAIN)-
NNNSSFYTGVYILIGAGALMMLVGFLGCCGAVQESQCMLGLFFGFLLVIFAIE
IAAAIWGYSHKDEVIKEVQEFYKDTYNKLKTKDEPQRETLKAIHYALNCCGL
AGGVEQFISDICPKKDVLETFTVKSCPDAIKEVFDNKFHIIGAVGIGIAVVMIFG
MIFSMILCCAIRRNREMV
Targeting domain fusion site to transmembrane CD28 anchor
(SEQ ID NO: 217)
MLTRTLAVRSFAATMALPVTALLLPLALLLHAARPEQKLISEEDL-(X,
WHEREIN X IS A TARGETING DOMAIN)-
TGKLFWALVVVAGVLFCYGLLVTVALCVIWVRSG
Targeting domain fusion site to transmembrane CD8 anchor
(SEQ ID NO: 218)
MLTRTLAVRSFAATMALPVTALLLPLALLLHAARPEQKLISEEDL-(X,
WHEREIN X IS A TARGETING DOMAIN)-
IYIWAPLAGTCGVLLLSLVITLYCNHRNRRRVCKCPRPVVKSGDKPSLSARYV
Targeting domain fusion site to transmembrane CD4 anchor
(SEQ ID NO: 219)
MLTRTLAVRSFAATMALPVTALLLPLALLLHAARPEQKLISEEDL-(X,
WHEREIN X IS A TARGETING DOMAIN)-MALIVLGGVAGLLLFIGLGIFFCV
RCRHRRRQAERMSQIKRLLSEKKTCQCPHRFQKTCSPI
Targeting domain fusion site to transmembrane CD63 anchor
(SEQ ID NO: 220)
MLTRTLAVRSFAATMAVEGGMKCVKFLLYVLLLAFCACAVGLIAVGVGAQ-
(X, WHEREIN X IS A TARGETING DOMAIN)-
LVLSQTIIQGATPGSLLPVVIIAVGVFLFLVAFVGCCGACKENYCLMITFAIFLS
LIMLVEVAAAIAGYVFRDKVMSEFNNNFRQQMENYPKNNHTASILDRMQAD
FKCCGAANYTDWEKIPSMSKNRVPDSCCINVTVGCGINFNEKAIHKEGCVEKI
GGWLRKNVLVVAAAALGIAFVEVLGIVFACCLVKSIRSGYEVM
Targeting domain fusion site to transmembrane CD81 anchor
(SEQ ID NO: 221)
MLTRTLAVRSFAATMGVEGCTKCIKYLLFVFNFVFWLAGGVILGVALWLRHD
PQTTNLLYLEL-(X, WHEREIN X IS A TARGETING DOMAIN)-
GDKPAPNTFYVGIYILIAVGAVMMFVGFLGCYGAIQESQCLLGTFFTCLVILF
ACEVAAGIWGFVNKDQIAKDVKQFYDQALQQAVVDDDANNAKAVVKTFHE
TLDCCGSSTLTALTTSVLKNNLCPSGSNIISNLFKEDCHQKIDDLFSGKLYLIGI
AAIVVAVIMIFEMILSMVLCCGIRNSSVY
Targeting domain fusion site to transmembrane CD86 anchor
(SEQ ID NO: 222)
MLTRTLAVRSFAATMALPVTALLLPLALLLHAARPEQKLISEEDL-(X,
WHEREIN X IS A TARGETING DOMAIN)-
PPDHIPWITAVLPTVIICVMVFCLILWKWKKKKRPRS
Targeting domain fusion site to transmembrane Notch anchor
(SEQ ID NO: 223)
MLTRTLAVRSFAATMALPVTALLLPLALLLHAARPEQKLISEEDL-(X,
WHEREIN X IS A TARGETING DOMAIN)-
ILDYSFTGGAGRDIPPPQIEEACELPECQVDAGNKVCNLQCNNHACGWDGGD
CSLNFNDPWKNCTQSLQCWKYFSDGHCDSQCNSAGCLFDGFDCQLTEGQCN
PLYDQYCKDHFSDGHCDQGCNSAECEWDGLDCAEHVPERLAAGTLVLVVLL
PPDQLRNNSFHFLRELSHVLHTNVVFKRDAQGQQMIFPYYGHEEELRKHPIK
RSTVGWATSSLLPGTSGGRQRRELDPMDIRGSIVYLEIDNRQCVQSSSQCFQS
ATDVAAFLGALASLGSLNIPYKIEAVKSEPVEPPLPSQLHLMYVAAAAFVLLF
FVGCGVLLSRKRRRQLCIQKL

### EXAMPLES

The invention is further described in the following examples, which do not limit the scope of the invention described in the claims.

### Example 1. Programmed Tropism Virus-Like Particles Deliver Gene Editing Cargo to Target Cells

### Methods

ptVLP particles were produced in HEK293T cells by using polyethylenimine (PEI) to transfect plasmids into these cells. PEI is Polyethylenimine 25 kD linear (Polysciences #23966-2). To make a stock ‘PEI MAX’ solution, Ig of PEI was added to 1 L endotoxin-free dH₂O that was previously heated to ˜80° C. and cooled to room temperature. This mixture was neutralized to pH 7.1 by addition of TON NaOH and filter sterilized with 0.22 μm polyethersulfone (PES). PEI MAX solution was stored at −20° C.

HEK293T cells were split to reach a confluency of 70%-90% at time of transfection and are cultured in 10% FBS DMEM media. Plasmid vectors encoding cargo, e.g., one encoding a CMV promoter driving expression of a fusion protein comprising hPLCδ1 PH domain linked to codon optimized Cas9, were co-transfected with plasmids encoding a U6 promoter driving expression of a Cas9 sgRNA, a membrane-anchored targeting moiety, and a mutated VSV-G envelope plasmid. Transfection reactions were assembled in reduced serum media (Opti-MEM; GIBCO #31985-070). For ptVLP particle production on 10 cm plates, 7.5 μg PH-Cas9 expressing plasmid, 7.5 μg sgRNA-expression plasmid and 5 μg programmed tropism ENV expressing plasmid were mixed in 1 mL Opti-MEM, followed by addition of 27.5 μl PEI MAX. After 20-30 min incubation at room temperature, the transfection reactions were dispersed dropwise over the HEK293T cells.

ptVLPs were harvested at 48-72 hours post-transfection. To do this, ptVLP supernatants were filtered using 0.45 μm PVDF or cellulose acetate or 0.8 μm PES membrane filters and transferred to polypropylene Beckman ultracentrifuge tubes that are used with the SW28 rotor (Beckman Coulter #326823). Each ultracentrifuge tube is filled with ptVLP-containing supernatant from three 10 cm plates to reach an approximate final volume of 35-37.5 ml. ptVLP supernatant underwent ultracentrifugation at approximately 100,000 xg, or 25,000 rpm, at 4° C. for 2 hours. After ultracentrifugation, supernatants were decanted and ptVLP pellets resuspended in DMEM 10% FBS media such that they were now approximately 1,000 times more concentrated than they were before ultracentrifugation. ptVLPs were added dropwise to cells that were seeded in a 24-well plate 24-hours prior to transduction. Polybrene (5-10 μg/mL in cell culture medium; Sigma-Aldrich #TR-1003-G) was supplemented to enhance transduction efficiency, if necessary. Vectofusin-1 (10 μg/mL in cell culture medium, Miltenyi Biotec #130-111-163) was supplemented to enhance transduction efficiency, if necessary. Immediately following the addition of ptVLPs, the 24-well plate was centrifuged at 1,150×g for 30 min at room temperature to enhance transduction efficiency, if necessary.

### Example 1.1

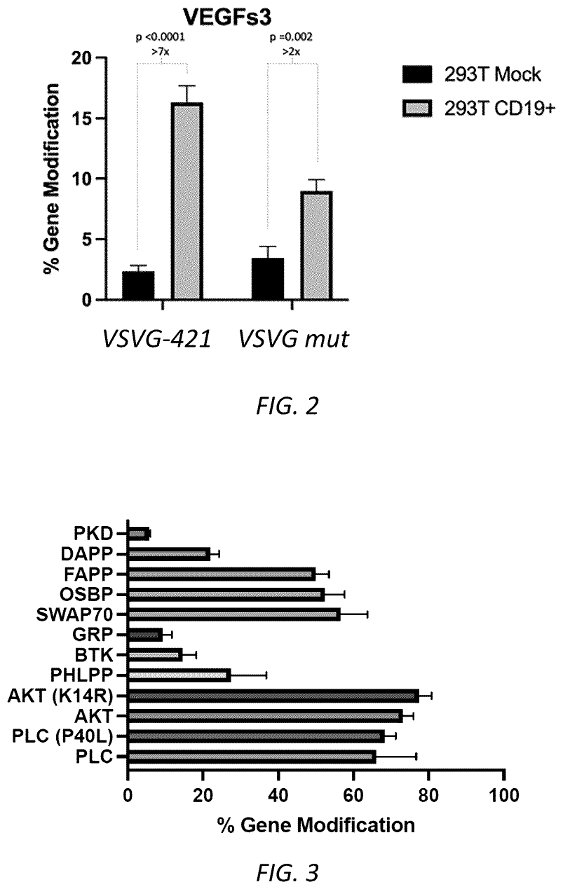

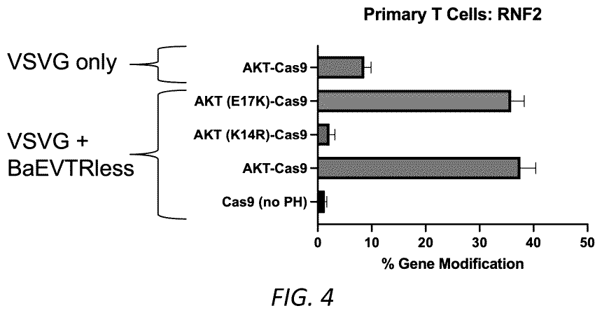

ptVLPs (illustrated in FIGS. 1A and 1C) were produced by transient plasmid transfection of HEK293T cells as described above. These ptVLPs (FIGS. 1B and 1D) were purified and concentrated 100-fold by filtration and PEG precipitation and applied to HEK293T cells that express or lack expression of CD19 for an incubation period of 48 hours. HEK293T cells were subsequently harvested and genomic DNA was extracted. Extracted genomic DNA was used to perform targeted amplicon sequencing of the genomic sites targeted by the cargos of the VLPs to quantify the frequencies of gene modification/gene edits (FIG. 2). The results showed that transduction efficiency (as measured by gene editing of the target site (VEGFs3)) was significantly enhanced in cells expressing the target antigen CD19 compared to cells lacking CD19 expression.

### Example 1.2

FIGS. 3 and 4 show that different phospholipid bilayer recruitment domains are capable of delivering cargo in previously described eVLPs (WO 2022/020800). For FIG. 3, eVLPs were produced by transient transfection of HEK293T cells, purified and concentrated 100-fold by filtration and PEG precipitation, and normalized based on total Cas9 within the particles determined by ELISA prior to transducing HEK293T cells so that the same pmol of Cas9 was applied in each well and comparisons could be made between different PH domains. Gene modification/gene editing frequencies induced at the endogenous VEGF target site were determined by targeted amplicon sequencing (FIG. 3). These eVLPs were pseudotyped with VSVG. The results showed that various PH domain and mutant PH domain fusions to cargos can mediate variable cargo delivery efficiencies and thereby variable frequencies of targeted gene modification in the target recipient cells.

For FIG. 4, different mutant PH-Cas9 fusions (and Cas9 lacking a fusion to a PH domain) were packaged in eVLPs (made as described in WO 2022/020800), purified and concentrated 100-fold by PEG precipitation, and normalized by total Cas9 within the particles determined by ELISA so that 5 pmol of Cas9 was added to 15,000 primary T cells per well. Gene modification/gene editing frequencies induced at the endogenous RNF2 target site were determined by targeted amplicon sequencing (FIG. 4). These eVLPs were pseudotyped with VSVG or a combination of VSVG and BaEVTRless. The results showed that various PH domain and mutant PH domain fusions to cargos resulted in variable cargo delivery efficiencies and variable frequencies of targeted gene modification in target recipient cells. In addition, different pseudotype combinations also affected delivery efficiency.

### REFERENCES

1. Parseval, N. et al. Survey of human genes of retroviral origin identification and transcriptome of the genes with coding capacity for complete envelope proteins. Journal of Virology 77, 10414-10422, (2003).
2. Okimoto, T. et al. VSV-G envelope glycoprotein forms complexes with plasmid DNA and MLV retrovirus-like particles in cell-free conditions and enhances DNA transfection. Molecular Therapy 4, 232-238, (2001).
3. Mangeot, P. et al. Protein transfer into human cells by VSV-G-induced nanovesicles. Molecular Therapy 19, 1656-1666, (2011).
4. Wagner, D. et al. High prevalence of Streptococcus pyogenes Cas9-reactive T cells within the adult human population. Nature Medicine 25, 242-248 (2019)
5. Kim, S. et al. CRISPR RNAs trigger innate immune responses in human cells. Genome Research 28, 1-7 (2018).
6. Charlesworth, C. et al. Identification of preexisting adaptive immunity to Cas9 proteins in humans. Nature Medicine 25, 249-254 (2019)
7. Ferdosi, S. et al. Multifunctional CRISPR-Cas9 with engineered immunosilenced human T cell epitopes. Nature Communications 10, Article number 1842 (2019).
8. Wang, D. et al. Adenovirus-mediated somatic genome editing of Pten by CRISPR/Cas9 in mouse liver in spite of Cas9-specific immune responses. Human Gene Therapy 26, 432-442 (2015).
9. Devanabanda, M. et al. Immunotoxic effects of gold and silver nanoparticles Inhibition of mitogen-induced proliferative responses and viability of human and murine lymphocytes in vitro. Journal of Immunotoxicology 13, 1547-6901 (2016).
10. Mout, R. et al. Direct cytosolic delivery of CRISPR/Cas9-ribonucleoprotein for efficient gene editing. ACS Nano 11, 2452-2458 (2017).
11. Yin, H. et al. structure-guided chemical modification of guide RNA enables potent non-viral in vivo genome editing. Nature Biotechnology 35, 1179-1187 (2017).
12. Qiao, J. et al. Cytosolic delivery of CRISPR/Cas9 ribonucleoproteins for genome editing using chitosan-coated red fluorescent protein. Chemical Communications 55, 4707-4710 (2019).
13. Li, L. et al. A rationally designed semiconducting polymer brush for NIR-II imaging guided light-triggered remote control of CRISPR/Cas9 genome editing. Advanced Materials 1901187, 1-9 (2019).
14. Gao, X. et al. Treatment of autosomal dominant hearing loss by in vivo delivery of genome editing agents. Nature 553, 217-221 (2018)
15. Lee, K. et al. Nanoparticle delivery of Cas9 ribonucleoprotein and donor DNA in vivo induces homology-directed DNA repair. Nature Biomedical Engineering 1, 889-901 (2017).
16. Staahl, B. et al. Efficient genome editing in the mouse brain by local delivery of engineered Cas9 ribonucleoprotein complexes. Nature Biotechnology 35, 431-433 (2017).
17. Zuris, J. et al. Cationic lipid-mediated delivery of proteins enables efficient protein-based genome editing in vitro and in vivo. Nature Biotechnology 33, 73-79 (2015).
18. Finn, J. et al. A single administration of CRISPR/Cas9 lipid nanoparticles achieves robust and persistent in vivo genome editing. Cell Reports 22, 2227-2235 (2018).
19. Wang, H. et al. Nonviral gene editing via CRISPR/Cas9 delivery by membrane-disruptive and endosomolytic helical polypeptide. PNAS 115, 4903-4908 (2018).
20. Del'Guidice, T. et al. Membrane permeabilizing amphiphilic peptide delivers recombinant transcription factor and CRISPR-Cas9/Cpf1 ribonucleoproteins in hard-to-modify cells. PLOS ONE 13, e0195558 (2018).
21. Colella, P. et al. Emerging Issues in AAV-Mediated In Vivo Gene Therapy. Molecular Therapy Methods & Clinical Development 8, 87-104 (2018).
22. Naso, F. et al. Adeno-Associated Virus (AAV) as a Vector for Gene Therapy. BioDrugs 31, 317-334 (2017).
23. Handel, E. et al. Versatile and efficient genome editing in human cells by combining zinc-finger nucleases with adeno-associated viral vectors. Human Gene Therapy 23, 321-329 (2012).
24. Chadwick, A. et al. Reduced Blood Lipid Levels With In Vivo CRISPR-Cas9 Base Editing of ANGPTL3. Circulation 137, 975-977 (2018).
25. Schenkwein, D. et al. Production of HIV-1 Integrase Fusion Protein-Carrying Lentiviral Vectors for Gene Therapy and Protein Transduction. Human Gene Therapy 21, 589-602 (2010).
26. Cai, Y. et al. Targeted genome editing by lentiviral protein transduction of zinc-finger and TAL-effector nucleases. eLife 3, e01911 (2014).
27. Choi, J. et al. Lentivirus pre-packed with Cas9 protein for safer gene editing. Gene Therapy 23, 627-633 (2016).
28. Meyer, C. et al. Pseudotyping exosomes for enhanced protein delivery in mammalian cells. International Journal of Nanomedicine 12, 3153-3170 (2017).
29. Mangeot, P. et al. Genome editing in primary cells and in vivo using viral-derived Nanoblades loaded with Cas9-sgRNA ribonucleoproteins. Nature Communications 10, Article number 45 (2019).
30. Lu, B. et al. Delivering SaCas9 mRNA by lentivirus-like bionanoparticles for transient expression and efficient genome editing. Nucleic Acids Research 47, e44 (2019).
31. Wang, Q. et al. ARMMs as a versatile platform for intracellular delivery of macromolecules. Nature Communications 9, 1-7 (2018).
32. Lainscek, D. et al. Delivery of an Artificial Transcription Regulator dCas9-VPR by Extracellular Vesicles for Therapeutic Gene Activation. ACS Synthetic Biology 7, 2715-2725 (2018).
33. Fuchs, J. et al. First-in-Human Evaluation of the Safety and Immunogenicity of a Recombinant Vesicular Stomatitis Virus Human Immunodeficiency Virus-1 gag Vaccine (HVTN 090). Open Forum Infectious Diseases 2, 1-9, (2015).
34. Cong, L. et al. Multiplex Genome Engineering Using CRISPR/Cas Systems. Science 339, 819-823, (2013).
35. Ran, F. et al. In vivo genome editing using Staphylococcus aureus Cas9. Nature 520, 186-191, (2015).
36. Zetsche, B. et al. Cpf1 Is a Single RNA-Guided Endonuclease of a Class 2 CRISPR-Cas System. Cell 163, 759-771, (2015).
37. Komor, A. et al. Programmable editing of a target base in genomic DNA without double-stranded DNA cleavage. Nature 533, 420-424, (2016).
38. Gaudelli, N. et al. Programmable base editing of A•T to G•C in genomic DNA without DNA cleavage. Nature 551, 464-471, (2017).
39. Voelkel, C. et al. Protein transduction from retroviral Gag precursors. Proc Natl Acad Sci USA 107, 7805-7810, (2010).
40. Kaczmarczyk, S. et al. Protein delivery using engineered virus-like particles. Proc Natl Acad Sci USA 108, 16998-17003, (2011).
41. Ebner, M. et al. PI(3,4,5)P3 Engagement Restricts Akt Activity to Cellular Membranes. Mol Cell 65, 416-431, (2017).
42. Urano, E. et al. Substitution of the myristoylation signal of human immunodeficiency virus type 1 Pr55Gag with the phospholipase C-dl pleckstrin homology domain results in infectious pseudovirion production. J. Gen Virology 89, 3144-3149, (2008).
43. Pastuzyn, E. et al. The Neuronal Gene Arc Encodes a Repurposed Retrotransposon Gag Protein that Mediates Intercellular RNA Transfer. Cell 172, 275-288, (2018).
44. Lukacs, G. et al. Size-dependent DNA Mobility in Cytoplasm and Nucleus. Journal of Biological Chemistry 275, 1625-1629, (1999).
45. Kreiss, P. et al. Plasmid DNA size does not affect the physicochemical properties of lipoplexes but modulates gene transfer efficiency. Nucleic Acids Research 27, 3792-3798 (1999).
46. Nafissi, N. et al. DNA Ministrings Highly Safe and Effective Gene Delivery Vectors. Molecular Therapy—Nucleic Acids 3, e165, (2014).
47. Munch, R. et al. Displaying High-affinity Ligands on Adeno-associated Viral Vectors Enables Tumor Cell-specific and Safe Gene Transfer. Molecular Therapy 21, 109-118 (2013).
48. Koide, S. et al. Chapter six—Target-Binding Proteins Based on the 10th Human Fibronectin Type III Domain (10Fn3). Methods in Enzymology 503, 5-156 (2012).
49. Leach, A. et al. Anti-DLL4 VNAR targeted nanoparticles for targeting of both tumour and tumour associated vasculature. Nanoscale 12, 14751-14763 (2020).
50. Hamann, M. et al. Improved targeting of human CD4+ T cells by nanobody-modified AAV2 gene therapy vectors. PLOS ONE 16(12) e0261269 (2021).
51. Dobson, C. et al. Antigen identification and high-throughput interaction mapping by reprogramming viral entry. Nature Methods 19, 449-460 (2022).
52. Sati, S. et al. RGD Peptide as a Targeting Moiety for Theranostic Purpose An Update Study. International Journal of Peptide Research and Therapeutics 25, 49-65 (2019).
53. Bannas, P. et al. Nanobodies and Nanobody-Based Human Heavy Chain Antibodies As Antitumor Therapeutics. Front. Immunol. 81603. doi 10.3389/fimmu.2017.01603 (2017).
54. Fujimoto, T. et al. Selective EGLN Inhibition Enables Ablative Radiotherapy and Improves Survival in Unresectable Pancreatic Cancer. Cancer Research 79, 2327-2338 (2019).
55. Tai, S. et al. Differential Expression of Metallothionein 1 and 2 Isoforms in Breast Cancer Lines with Different Invasive Potential Identification of a Novel Nonsilent Metallothionein-1H Mutant Variant. American Journal of Pathology 163, 2009-2019 (2003).
56. Caussinus, E. et al. Fluorescent fusion protein knockout mediated by anti-GFP nanobody. Nature Structural & Molecular Biology 19, 117-121, (2012).
57. Zhao, W. et al. Quantitatively Predictable Control of Cellular Protein Levels through Proteasomal Degradation. ACS Synthetic Biology 7, 540-552, (2018).
58. Clift, D. et al. A Method for the Acute and Rapid Degradation of Endogenous Proteins. Cell 171, 1692-1706, (2017).
59. Balla, T. & Várnai, T. Visualizing Cellular Phosphoinositide Pools with GFP-Fused Protein-Modules. SCIENCE'S STKE 2002, p. p13, DOI 10.1126/stke.2002.125.pl3 (2002).
60. Carpten, J. et al. A transforming mutation in the pleckstrin homology domain of AKT1 in cancer. Nature 448, 439-445, (2007).
61. Várnai, P. et al. Selective cellular effects of overexpressed pleckstrin-homology domains that recognize PtdIns(3,4,5)P3 suggest their interaction with protein binding partners. Journal of Cell Science 118, 4879-4888, (2005).
62. Parikh, C. et al. Disruption of PH-kinase domain interactions leads to oncogenic activation of AKT in human cancers. PNAS 109, 19368-19373, (2012).
63. Jo, H. et al. Small molecule-induced cytosolic activation of protein kinase Akt rescues ischemia-elicited neuronal death. PNAS 109 (26) 10581-10586, (2012).
64. Han, F. et al. The critical role of AMPK in driving Akt activation under stress, tumorigenesis and drug resistance. Nat Commun 9, 4728 (2018). doi.org/10.1038/s41467-018-07188-9
65. Li, X. et al. Autophosphorylation of Akt at Threonine 72 and Serine 246 A POTENTIAL MECHANISM OF REGULATION OF Akt KINASE ACTIVITY*. Journal of Biological Chemistry 281, 13837-13843, (2006).
66. Liao, Y. et al. Peptidyl-prolyl cis/trans isomerase Pin1 is critical for the regulation of PKB/Akt stability and activation phosphorylation. Oncogene 28(26)2436-45, DOI10.1038/onc.2009.98 (2009).
67. Chu, N. et al. Akt Kinase Activation Mechanisms Revealed Using Protein Semisynthesis. Cell 174(4) 897-907.e14. (2018).
68. Lučić, I. et al. Conformational sampling of membranes by Akt controls its activation and inactivation. Proc Natl Acad Sci USA 115(17) E3940-E3949, (2018).
69. Baranov, M. et al. SWAP70 Organizes the Actin Cytoskeleton and Is Essential for Phagocytosis. Cell Reports 17, p1518-1531 (2016).
70. Morsut L, Roybal K T, Xiong X, Gordley R M, Coyle S M, Thomson M, Lim W A. Engineering Customized Cell Sensing and Response Behaviors Using Synthetic Notch Receptors. Cell. 2016 Feb. 11; 164(4)780-91. doi 10.1016/j.cell.2016.01.012. Epub 2016 Jan. 28. PMID 26830878; PMCID PMC4752866.
71. Trahtenherts A, Benhar I. An internalizing antibody specific for the human asialoglycoprotein receptor. Hybridoma (Larchmt). 2009 Aug.; 28(4)225-33. doi 10.1089/hyb.2009.0019. PMID 19663694.
72. Tillotson B J, Goulatis L I, Parenti I, Duxbury E, Shusta E V. Engineering an Anti-Transferrin Receptor ScFv for pH-Sensitive Binding Leads to Increased Intracellular Accumulation. PLoS One. 2015 Dec. 29; 10(12)e0145820. doi 10.1371/journal.pone.0145820. PMID 26713870; PMCID PMC4694649.
73. Wang J H, Forterre A V, Zhao J, Frimannsson D O, Delcayre A, Antes T J, Efron B, Jeffrey S S, Pegram M D, Matin A C. Anti-HER2 scFv-Directed Extracellular Vesicle-Mediated mRNA-Based Gene Delivery Inhibits Growth of HER2-Positive Human Breast Tumor Xenografts by Prodrug Activation. Mol Cancer Ther. 2018 May; 17(5)1133-1142. doi 10.1158/1535-7163.MCT-17-0827. Epub 2018 Feb. 26. PMID 29483213; PMCID PMC5932266.
74. Benedict C A, MacKrell A J, Anderson W F. Determination of the binding affinity of an anti-CD34 single-chain antibody using a novel, flow cytometry based assay. J Immunol Methods. 1997 Feb. 28; 201(2)223-31. doi 10.1016/s0022-1759(96)00227-x. PMID 9050944.
75. Babaei A, Zarkesh-Esfahani S H, Gharagozloo M. Production of a recombinant anti-human CD4 single-chain variable-fragment antibody using phage display technology and its expression in Escherichia coli. J Microbiol Biotechnol. 2011 May; 21(5)529-35. doi 10.4014/jmb.1010.10022. PMID 21617352.
76. Dehbashi M, Hojati Z, Motovali-Bashi M, Ganjalikhany M R, Cho W C, Shimosaka A, Navabi P, Ganjalikhani-Hakemi M. A Novel CAR Expressing NK Cell Targeting CD25 With the Prospect of Overcoming Immune Escape Mechanism in Cancers. Front Oncol. 2021 May 14; 11649710. doi 10.3389/fonc.2021.649710. PMID 34055618; PMCID PMC8160382.
77. Hosseinzadeh F, Mohammadi S, Nejatollahi F. Production and Evaluation of Specific Single-Chain Antibodies against CTLA-4 for Cancer-Targeted Therapy. Rep Biochem Mol Biol. 2017 Oct.; 6(1)8-14. PMID 29090224; PMCID PMC5643449.
78. Lu P, Qiu S, Pan Y, Shi S, Yu Q, Yu F, Miao L, Wang H, Chen K. Discovery of an Heparin-Binding Epidermal Growth Factor Domain Antibody from a Phage Library and Analysis of Its Inhibitory Effects in SKOV3 Cells. Cancer Biother Radiopharm. 2021 Sep. 16. doi 10.1089/cbr.2021.0123. Epub ahead of print. PMID 34529926.
79. Chen Y, Zhang Y N, Yan R, Wang G, Zhang Y, Zhang Z R, Li Y, Ou J, Chu W, Liang Z, Wang Y, Chen Y L, Chen G, Wang Q, Zhou Q, Zhang B, Wang C. ACE2-targeting monoclonal antibody as potent and broad-spectrum coronavirus blocker. Signal Transduct Target Ther. 2021 Aug. 25; 6(1)315. doi 10.1038/s41392-021-00740-y. PMID 34433803; PMCID PMC8385704.
80. Oesch-Bartlomowicz B, Huelster A, Wiss O, Antoniou-Lipfert P, Dietrich C, Arand M, Weiss C, Bockamp E, Oesch F. Aryl hydrocarbon receptor activation by cAMP vs. dioxin divergent signaling pathways. Proc Natl Acad Sci USA. 2005 Jun. 28; 102(26)9218-23. doi 10.1073/pnas.0503488102. Epub 2005 Jun. 21.
81. Moll R, Dhouailly D, Sun T T. Expression of keratin 5 as a distinctive feature of epithelial and biphasic mesotheliomas. An immunohistochemical study using monoclonal antibody AE14. Virchows Arch B Cell Pathol Incl Mol Pathol. 1989; 58(2)129-45. doi 10.1007/BF02890064. PMID 2482572.
82. Fan J, Shen Z, Wang G, Yang H, Liu Y. Secretory expression of human ScFv against keratin in Pichia pastoris and its effects on cultured keratinocytes. Arch Dermatol Res. 2009 June; 301(5)367-72. doi 10.1007/s00403-008-0908-4. Epub 2008 Oct. 21. Erratum in Arch Dermatol Res. 2009 June; 301(5)395. PMID 18936942.
83. Stausbol-Gron B, Jensen K B, Jensen K H, Jensen M O, Clark B F. De novo identification of cell-type specific antibody-antigen pairs by phage display subtraction. Isolation of a human single chain antibody fragment against human keratin 14. Eur J Biochem. 2001 May; 268(10)3099-107. doi 10.1046/j.1432-1327.2001.02210.x. PMID 11358530.
84. Malecha M J, Miettinen M. Expression of keratin 13 in human epithelial neoplasms. Virchows Arch A Pathol Anat Histopathol. 1991; 418(3)249-54. doi 10.1007/BF01606063. PMID 1706547.
85. Wang Y, Loers G, Pan H C, Gouveia R, Zhao W J, Shen Y Q, Kleene R, Costa J, Schachner M. Antibody fragments directed against different portions of the human neural cell adhesion molecule L1 act as inhibitors or activators of L1 function. PLoS One. 2012; 7(12)e52404. doi 10.1371/journal.pone.0052404. Epub 2012 Dec. 18. PMID 23272240; PMCID PMC3525558.
86. Martin-Otal C, Lasarte-Cia A, Serrano D, Casares N, Conde E, Navarro F, Sánchez-Moreno I, Gorraiz M, Sarrión P, Calvo A, De Andrea C E, Echeveste J, Vilas A, Rodriguez-Madoz J R, San Miguel J, Prosper F, Hervas-Stubbs S, Lasarte J J, Lozano T. Targeting the extra domain A of fibronectin for cancer therapy with CAR-T cells. J Immunother Cancer. 2022 Aug.; 10(8)e004479. doi 10.1136/jitc-2021-004479. PMID 35918123; PMCID PMC9351345.
87. Kimizuka F, Taguchi Y, Ohdate Y, Kawase Y, Shimojo T, Hashino K, Kato I, Sekiguchi K, Titani K. Production and characterization of functional domains of human fibronectin expressed in Escherichia coli. J Biochem. 1991 August; 110(2)284-91. doi 10.1093/oxfordjournals.jbchem.al23572. PMID 1761524.
88. Tiwari A, Kumar R, Ram J, Sharma M, Luthra-Guptasarma M. Control of fibrotic changes through the synergistic effects of anti-fibronectin antibody and an RGDS-tagged form of the same antibody. Sci Rep. 2016 Aug. 3; 630872. doi 10.1038/srep30872. PMID 27484779; PMCID PMC4971484.
89. Sebollela A, Cline E N, Popova I, Luo K, Sun X, Ahn J, Barcelos M A, Bezerra V N, Lyra E Silva N M, Patel J, Pinheiro N R, Qin L A, Kamel J M, Weng A, DiNunno N, Bebenek A M, Velasco P T, Viola K L, Lacor P N, Ferreira S T, Klein W L. A human scFv antibody that targets and neutralizes high molecular weight pathogenic amyloid-β oligomers. J Neurochem. 2017 September; 142(6)934-947. doi 10.1111/jnc.14118. Epub 2017 Aug. 2. PMID 28670737; PMCID PMC5752625.
90. Rafiq S, Yeku O O, Jackson H J, Purdon T J, van Leeuwen D G, Drakes D J, Song M, Miele M M, Li Z, Wang P, Yan S, Xiang J, Ma X, Seshan V E, Hendrickson R C, Liu C, Brentjens R J. Targeted delivery of a PD-1-blocking scFv by CAR-T cells enhances anti-tumor efficacy in vivo. Nat Biotechnol. 2018 October; 36(9)847-856. doi 10.1038/nbt.4195. Epub 2018 Aug. 13. PMID 30102295; PMCID PMC6126939.
91. Harrasser M, Gohil S H, Lau H, Della Peruta M, Muczynski V, Patel D, Miranda E, Grigoriadis K, Grigoriadis A, Granger D, Evans R, Nathwani A C. Inducible localized delivery of an anti-PD-1 scFv enhances anti-tumor activity of ROR1 CAR-T cells in TNBC. Breast Cancer Res. 2022 Jun. 3; 24(1)39. doi 10.1186/s13058-022-01531-1. PMID 35659040; PMCID PMC9166313.
92. Zhang C, Helmsing S, Zagrebelsky M, Schirrmann T, Marschall A L, Schüngel M, Korte M, Hust M, Dübel S. Suppression of p75 neurotrophin receptor surface expression with intrabodies influences Bcl-xL mRNA expression and neurite outgrowth in PC12 cells. PLoS One. 2012; 7(1)e30684. doi 10.1371/journal.pone.0030684. Epub 2012 Jan. 24.
93. Moazen B, Zarrinhaghighi A, Nejatollahi F. Selection and Evaluation of Specific Single Chain Antibodies against CD90, a Marker for Mesenchymal and Cancer Stem Cells. Rep Biochem Mol Biol. 2018 October; 7(1)45-51. PMID 30324117; PMCID PMC6175597.
94. Hao H, Zhen Y, Wang Z, Chen F, Xie X. A novel therapeutic drug for colon cancer EpCAM scFv-truncated protamine (tp)-siRNA. Cell Biol Int. 2013 Aug; 37(8)860-4. doi 10.1002/cbin.10112. Epub 2013 Apr. 30.
95. Sato Y, Mustafina K R, Luo Y, Martini C, Thomas D Y, Wiseman P W, Hanrahan J W. Nonspecific binding of common anti-CFTR antibodies in ciliated cells of human airway epithelium. Sci Rep. 2021 Dec. 1; 11(1)23256. doi 10.1038/s41598-021-02420-x.
96. Federica Toffalini, Jean-Baptiste Demoulin; The Transmembrane Domain of PDGFR-β Plays An Important Role in ETV6-PDGFR-β Activation. Blood 2008; 112 (11) 5320. doi https//doi.org/10.1182/blood.V112.11.5320.5320
97. Verweij F J, Bebelman M P, Jimenez C R, Garcia-Vallejo J J, Janssen H, Neefjes J, Knol J C, de Goeij-de Haas R, Piersma S R, Baglio S R, Verhage M, Middeldorp J M, Zomer A, van Rheenen J, Coppolino M G, Hurbain I, Raposo G, Smit M J, Toonen R F G, van Niel G, Pegtel D M. Quantifying exosome secretion from single cells reveals a modulatory role for GPCR signaling. J Cell Biol. 2018 Mar. 5; 217(3)1129-1142. doi 10.1083/jcb.201703206. Epub 2018 Jan. 16. Erratum in J Cell Biol. 2018 Jan. 23.
98. Leddon S A, Fettis M M, Abramo K, Kelly R, Oleksyn D, Miller J. The CD28 Transmembrane Domain Contains an Essential Dimerization Motif Front Immunol. 2020 Jul. 16; 111519. doi 10.3389/fimmu.2020.01519.
99. Roselli E, Boucher J C, Li G, Kotani H, Spitler K, Reid K, Cervantes E V, Bulliard Y, Tu N, Lee S B, Yu B, Locke F L, Davila M L. 4-1BB and optimized CD28 co-stimulation enhances function of human mono-specific and bi-specific third-generation CAR T cells. J Immunother Cancer. 2021 October; 9(10)e003354. doi 10.1136/jitc-2021-003354.
100. Parrish H L, Glassman C R, Keenen M M, Deshpande N R, Bronnimann M P, Kuhns M S. A Transmembrane Domain GGxxG Motif in CD4 Contributes to Its Lck-Independent Function but Does Not Mediate CD4 Dimerization. PLoS One. 2015 Jul. 6; 10(7)e0132333. doi 10.1371/journal.pone.0132333. Erratum in PLoS One. 2016; 11(3)e0150876.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A fusion protein comprising
(i) a programmable tropism glycoprotein or envelope protein (ptENV) comprising a virus-derived glycoprotein or envelope protein fused to a targeting domain,
optionally wherein the targeting domain is at the C terminus of the glycoprotein or envelope protein, at the N terminus, or is inserted immediately after a signal sequence, or
(ii) a membrane-anchored targeting domain comprising a targeting domain fused to a transmembrane domain;
optionally wherein the Targeting Domain comprises a peptide, single chain variable fragment (scFv), nanobody, fibronectin type 3 domain (FN3), arginylglycylaspartic acid motif (RGD), single variable domain on a heavy chain/nanobody (VHH), variable domain of new antigen receptor (VNAR), darpin or other targeting ligand.

2. The fusion protein of claim 1, wherein the Targeting Domain binds to human CD19, CD4, CD34, ASGR1, TfR1, HER2, CD25, CTLA-4, HB-EGF, ACE2, Aryl hydrocarbon receptor (AhR), keratin 5 (KRT5), KRT13, Fibronectin (FN1), Amyloid precursor protein (APP), neurotrophin receptor (p75NTR), Thy-1/CD90, EpCAM, and/or CFTR.

3. The fusion protein of claim 1, wherein the signal sequence comprises MKCLLYLAFLFIGVNCK (SEQ ID NO:1) or a secretion signal sequence that is derived from VSVG (optionally MKCLLYLAFLFIGVNC, SEQ ID NO:2).

4. The fusion protein of claim 1, comprising a sequence that is at least 95% identical to a sequence set forth herein, optionally a ptENV comprising a glycoprotein or envelope protein in Table 1, plus a targeting domain.

5. A nucleic acid sequence encoding the fusion protein of claim 1.

6. A vector comprising the nucleic acid sequence of claim 5, optionally operably linked to a promoter for expression of the fusion protein.

7. A host cell comprising the nucleic acid sequence of claim 5, and optionally expressing the fusion protein.

8. A virus-like particle (VLP) comprising the fusion protein of claim 1, and optionally, a cargo disposed in the core of the VLP, wherein the cargo is optionally fused to a phospholipid bilayer recruitment domain.

9. The VLP of claim 8, which is a programmable tropism virus-like particle (ptVLP), comprising
(a) a membrane comprising a phospholipid bilayer and
(b) the fusion protein comprising a ptENV, or
a glycoprotein or envelope protein (optionally as listed in Table 1) and the fusion protein comprising a membrane-anchored targeting domain; and
(c) optionally, a cargo disposed in the core of the ptVLP, wherein the cargo is optionally fused to a phospholipid bilayer recruitment domain; and,
optionally, wherein the ptVLP does not comprise an exogenous gag, pro and/or pol protein.

10. The VLP of claim 8, wherein the cargo is a therapeutic or diagnostic protein and/or nucleic acid encoding a therapeutic or diagnostic protein, and/or a chemical, optionally a small molecule therapeutic or diagnostic.

11. The VLP of claim 8, wherein the cargo is a gene editing or epigenetic modulating reagent.

12. The VLP of claim 8, wherein the gene editing or epigenetic modulating reagent comprises a zinc finger (ZF), transcription activator-like effector (TALE), and/or CRISPR-Cas protein, variant, or fusion thereof; a nucleic acid encoding a zinc finger (ZF), transcription activator-like effector (TALE), and/or CRISPR-Cas protein, variant, or fusion thereof; a guide RNA and/or crRNA; or a ribonucleoprotein complex (RNP) comprising a CRISPR-Cas protein, variant, or fusion thereof and/or optionally a guide RNA and/or crRNA.

13. The VLP or ptVLP of claim 12, wherein the cargo is selected from the proteins listed in Tables 2, 3, 4 & 5, or that is at least 95% identical to a sequence set forth herein, optionally in Tables 2, 3, 4, and 5.

14. The VLP or ptVLP of claim 12, wherein the cargo comprises a CRISPR-Cas protein, and the ptVLP further comprises one or more guide RNAs and/or crRNAs that bind to and direct the CRISPR-Cas protein to a target nucleic acid sequence.

15. The VLP or of claim 9, wherein the cargo comprises a fusion to a phospholipid bilayer recruitment domain, preferably as shown in Table 6, or that is at least 95% identical to a sequence set forth herein in Table 6.

16. A method of delivering a cargo to a target cell, optionally a cell in vivo or in vitro, the method comprising contacting the cell with the VLP or ptVLP of claim 8 comprising the cargo.

17. A method of producing a VLP or a ptVLP comprising a cargo, the method comprising
providing a cell expressing (i) the fusion protein comprising ptENV of claim 1 part (i) or (ii) a glycoprotein or envelope protein (optionally as listed in Table 1) and a membrane-anchored targeting domain of claim 1, part (ii); and
optionally also expressing a cargo, optionally wherein the cell does not express an exogenous gag, pro, or pol protein; and
maintaining the cell under conditions such that the cells produce the VLPs or ptVLPs.

18. The method of claim 17, further comprising harvesting and optionally purifying and/or concentrating the produced VLPs or ptVLPs.

19. The method of claim 17, wherein the cargo is a therapeutic or diagnostic protein and/or nucleic acid encoding a therapeutic or diagnostic protein, and/or a small molecule, optionally a therapeutic or diagnostic small molecule.

20. The method of claim 17, wherein the cargo is a gene editing or epigenetic modulating reagent.

21. The method of claim 17, wherein the gene editing or epigenetic modulating reagent comprises a zinc finger (ZF), transcription activator-like effector (TALE), and/or CRISPR-Cas protein, variant, or fusion thereof; a nucleic acid encoding a zinc finger (ZF), transcription activator-like effector (TALE), and/or CRISPR-Cas protein, variant, or fusion thereof; a guide RNA and/or crRNA; or a ribonucleoprotein complex (RNP) comprising a CRISPR-Cas protein, variant, or fusion thereof and optionally a guide RNA and/or crRNA.

22. The method of claim 21, wherein the cargo reagent is selected from the proteins listed in Tables 2, 3, 4 & 5, or that is at least 95% identical to a sequence set forth herein, optionally in Tables 2, 3, 4, and 5.

23. The method of claim 21, wherein the cargo reagent comprises a CRISPR-Cas protein, variant, or fusion thereof and the ptVLP further comprises one or more guide RNAs and/or crRNAs that bind to and direct the CRISPR-based genome editing or modulating protein to a target sequence.

24. The method of claim 17, wherein the cargo comprises a fusion to a phospholipid bilayer recruitment domain, preferably as shown in Table 6, or that is at least 95% identical to a sequence set forth herein in Table 6.

25. A cell expressing (i) a fusion protein comprising the ptENV of claim 1, part (i) or (ii) a glycoprotein or envelope protein (optionally as listed in Table 1) and a fusion protein comprising the membrane-anchored targeting domain of claim 1, part (ii);
and optionally a cargo, wherein the cargo is optionally fused to a phospholipid bilayer recruitment domain; and,
optionally wherein the cell does not express an exogenous gag, pro and/or pol protein.

26. The cell of claim 25, wherein the cargo is a therapeutic or diagnostic protein and/or nucleic acid encoding a therapeutic or diagnostic protein, and/or a small molecule, optionally a therapeutic or diagnostic small molecule.

27. The cell of claim 25, wherein the cargo is a gene editing or epigenetic modulating reagent.

28. The cell of claim 25, wherein the gene editing or epigenetic modulating reagent comprises a zinc finger (ZF), transcription activator-like effector (TALE), and/or CRISPR-Cas protein, variant, or fusion thereof; a nucleic acid encoding a zinc finger (ZF), transcription activator-like effector (TALE), and/or CRISPR-Cas protein, variant, or fusion thereof; a guide RNA and/or crRNA; or a ribonucleoprotein complex (RNP) comprising a CRISPR-Cas protein, variant, or fusion thereof and optionally a guide RNA.

29. The cell of claim 28, wherein the cargo reagent is selected from the proteins listed in Tables 2, 3, 4, & 5, or that is at least 95% identical to a sequence set forth herein, optionally in Tables 2, 3, 4, and 5.

30. The cell of claim 28, wherein the gene editing or epigenetic modulating reagent comprises a CRISPR-Cas protein, and the ptVLP further comprises one or more guide RNAs and/or crRNAs that bind to and direct the CRISPR-Cas protein to a target sequence.

31. The cells of claim 25, wherein the cargo comprises a fusion to a phospholipid bilayer recruitment domain, preferably as shown in Table 6, or that is at least 95% identical to a sequence set forth herein in Table 6.

32. The cells of claim 25, wherein the cells are primary or stable human cell lines.

33. The cells of claim 32, which are Human Embryonic Kidney (HEK) 293 cells or HEK293 T cells.


SEQUENCE LISTING (245 sequences)

SEQ ID NO: 1 - protein, 17 aa, unidentified
MKCLLYLAFLFIGVNCK

SEQ ID NO: 2 - protein, 16 aa, unidentified
MKCLLYLAFLFIGVNC

SEQ ID NO: 3 - protein, 25 aa, Homo sapiens
MGVLLTQRTLLSLVLALLFPSMASM

SEQ ID NO: 4 - protein, 19 aa, Homo sapiens
MGWSCIILFLVATATGVHS

SEQ ID NO: 5 - protein, 21 aa, Homo sapiens
MWWRLWWLLLLLLLLWPMVWA

SEQ ID NO: 6 - protein, 22 aa, Homo sapiens
MDMRVPAQLLGLLLLWLRGARC

SEQ ID NO: 7 - protein, 16 aa, Homo sapiens
MPLLLLLPLLWAGALA

SEQ ID NO: 8 - protein, 23 aa, Homo sapiens
MDAMKRGLCCVLLLCGAVFVSPS

SEQ ID NO: 9 - protein, 18 aa, Homo sapiens
MAFLWLLSCWALLGTTFG

SEQ ID NO: 10 - protein, 15 aa, Homo sapiens
MNLLLILTFVAAAVA

SEQ ID NO: 11 - protein, 20 aa, Homo sapiens
MYRMQLLSCIALSLALVTNS

SEQ ID NO: 12 - protein, 16 aa, Homo sapiens
MKWVTFISLLFSSAYS

SEQ ID NO: 13 - protein, 24 aa, Homo sapiens
MALWMRLLPLLALLALWGPDPAAA

SEQ ID NO: 14 - protein, 24 aa, Homo sapiens
MPSSVSWGILLLAGLCCLVPVSLA

SEQ ID NO: 15 - protein, 1936 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLSGGSKRTADGSEPKKKRKVGS

SEQ ID NO: 16 - protein, 1710 aa, synthetic construct
MSSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLKSGSETPGTSESATPESDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSTNLSDIIEKETGKQLVIQESILMLPEEVEEVIGNKPESDILVHTAYDESTDENVMLLTSDAPEYKPWALVIQDSNGENKIKMLSGGSPKKKRKV

SEQ ID NO: 17 - protein, 198 aa, Homo sapiens [sequence omitted]

SEQ ID NO: 18 - protein, 191 aa, Homo sapiens
LMDPHIFTSNFNNGIGRHKTYLCYEVERLDSATSFSLDFGYLRNKNGCHVELLFLRYISDWDLDPGRCYRVTWFTSWSPCYDCARHVADFLRGNPNLSLRIFTARLYFCEDRKAEPEGLRRLHRAGVQIAIMTFKDYFYCWNTFVENHERTFKAWEGLHENSVRLSRQLRRILLPLYEVDDLRDAFRTLGL

SEQ ID NO: 19 - protein, 175 aa, Homo sapiens
MDPHIFTSNFNNGIGRHKTYLCYEVERLDSATSFSLDFGYLRNKNGCHVELLFLRYISDWDLDPGRCYRVTWFTSWSPCYDCARHVADFLRGNPNLSLRIFTARLYFCEDRKAEPEGLRRLHRAGVQIAIMTFKDYFYCWNTFVENHERTFKAWEGLHENSVRLSRQLRRILLPL

SEQ ID NO: 20 - protein, 229 aa, Rattus norvegicus [sequence omitted]

SEQ ID NO: 21 - protein, 397 aa, Mus musculus
MGPFCLGCSHRKCYSPIRNLISQETFKFHFKNLGYAKGRKDTFLCYEVTRKDCDSPVSLHHGVFKNKDNIHAEICFLYWFHDKVLKVLSPREEFKITWYMSWSPCFECAEQIVRFLATHHNLSLDIFSSRLYNVQDPETQQNLCRLVQEGAQVAAMDLYEFKKCWKKFVDNGGRRFRPWKRLLTNFRYQDSKLQEILRRMDPLSEEEFYSQFYNQRVKHLCYYHRMKPYLCYQLEQFNGQAPLKGCLLSEKGKQHAEILFLDKIRSMELSQVTITCYLTWSPCPNCAWQLAAFKRDRPDLILHIYTSRLYFHWKRPFQKGLCSLWQSGILVDVMDLPQFTDCWTNFVNPKRPFRPWKGLEIISRRTQRRLRRIKESWGLQDLVNDFGNLQLGPPMSN

SEQ ID NO: 22 - protein, 199 aa, Mus musculus
MGPFCLGCSHRKCYSPIRNLISQETFKFHFKNLGYAKGRKDTFLCYEVTRKDCDSPVSLHHGVFKNKDNIHAEICFLYWFHDKVLKVLSPREEFKITWYMSWSPCFECAEQIVRFLATHHNLSLDIFSSRLYNVQDPETQQNLCRLVQEGAQVAAMDLYEFKKCWKKFVDNGGRRFRPWKRLLTNFRYQDSKLQEILRR

SEQ ID NO: 23 - protein, 199 aa, Homo sapiens
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGN

SEQ ID NO: 24 - protein, 384 aa, Homo sapiens
MKPHFRNTVERMYRDTFSYNFYNRPILSRRNTVWLCYEVKTKGPSRPPLDAKIFRGQVYSELKYHPEMRFFHWFSKWRKLHRDQEYEVTWYISWSPCTKCTRDMATFLAEDPKVTLTIFVARLYYFWDPDYQEALRSLCQKRDGPRATMKIMNYDEFQHCWSKFVYSQRELFEPWNNLPKYYILLHIMLGEILRHSMDPPTFTFNFNNEPWVRGRHETYLCYEVERMHNDTWVLLNQRRGFLCNQAPHKHGFLEGRHAELCFLDVIPFWKLDLDQDYRVTCFTSWSPCFSCAQEMAKFISKNKHVSLCIFTARIYDDQGRCQEGLRTLAEAGAKISIMTYSEFKHCWDTFVDHQGCPFQPWDGLDEHSQDLSGRLRAILQNQEN

SEQ ID NO: 25 - protein, 186 aa, Homo sapiens
PPTFTFNFNNEPWVRGRHETYLCYEVERMHNDTWVLLNQRRGFLCNQAPHKHGFLEGRHAELCFLDVIPFWKLDLDQDYRVTCFTSWSPCFSCAQEMAKFISKNKHVSLCIFTARIYDDQGRCQEGLRTLAEAGAKISIMTYSEFKHCWDTFVDHQGCPFQPWDGLDEHSQDLSGRLRAILQNQEN

SEQ ID NO: 26 - protein, 200 aa, Homo sapiens
MALLTAETFRLQFNNKRRLRRPYYPRKALLCYQLTPQNGSTPTRGYFENKKKCHAEICFINEIKSMGLDETQCYQVTCYLTWSPCSSCAWELVDFIKAHDHLNLGIFASRLYYHWCKPQQKGLRLLCGSQVPVEVMGFPKFADCWENFVDHEKPLSFNPYKMLEELDKNSRAIKRRLERIKIPGVRAQGRYMDILCDAEV

SEQ ID NO: 27 - protein, 373 aa, Homo sapiens
MKPHFRNTVERMYRDTFSYNFYNRPILSRRNTVWLCYEVKTKGPSRPRLDAKIFRGQVYSQPEHHAEMCFLSWFCGNQLPAYKCFQITWFVSWTPCPDCVAKLAEFLAEHPNVTLTISAARLYYYWERDYRRALCRLSQAGARVKIMDDEEFAYCWENFVYSEGQPFMPWYKFDDNYAFLHRTLKEILRNPMEAMYPHIFYFHFKNLRKAYGRNESWLCFTMEVVKHHSPVSWKRGVFRNQVDPETHCHAERCFLSWFCDDILSPNTNYEVTWYTSWSPCPECAGEVAEFLARHSNVNLTIFTARLYYFWDTDYQEGLRSLSQEGASVEIMGYKDFKYCWENFVYNDDEPFKPWKGLKYNFLFLDSKLQEILE

SEQ ID NO: 28 - protein, 189 aa, Homo sapiens
KEILRNPMEAMYPHIFYFHFKNLRKAYGRNESWLCFTMEVVKHHSPVSWKRGVFRNQVDPETHCHAERCFLSWFCDDILSPNTNYEVTWYTSWSPCPECAGEVAEFLARHSNVNLTIFTARLYYFWDTDYQEGLRSLSQEGASVEIMGYKDFKYCWENFVYNDDEPFKPWKGLKYNFLFLDSKLQEILE

SEQ ID NO: 29 - skipped

SEQ ID NO: 30 - protein, 383 aa, Escherichia coli
MKRTADGSEFESPKKKRKVSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTDSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTD

SEQ ID NO: 31 - protein, 1226 aa, Homo sapiens
MNPRQGYSLSGYYTHPFQGYEHRQLRYQQPGPGSSPSSFLLKQIEFLKGQLPEAPVIGKQTPSLPPSLPGLRPRFPVLLASSTRGRQVDIRGVPRGVHLGSQGLQRGFQHPSPRGRSLPQRGVDCLSSHFQELSIYQDQEQRILKFLEELGEGKATTAHDLSGKLGTPKKEINRVLYSLAKKGKLQKEAGTPPLWKIAVSTQAWNQHSGVVRPDGHSQGAPNSDPSLEPEDRNSTSVSEDLLEPFIAVSAQAWNQHSGVVRPDSHSQGSPNSDPGLEPEDSNSTSALEDPLEFLDMAEIKEKICDYLFNVSDSSALNLAKNIGLTKARDINAVLIDMERQGDVYRQGTTPPIWHLTDKKRERMQIKRNTNSVPETAPAAIPETKRNAEFLTCNIPTSNASNNMVTTEKVENGQEPVIKLENRQEARPEPARLKPPVHYNGPSKAGYVDFENGQWATDDIPDDLNSIRAAPGEFRAIMEMPSFYSHGLPRCSPYKKLTECQLKNPISGLLEYAQFASQTCEFNMIEQSGPPHEPRFKFQVVINGREFPPAEAGSKKVAKQDAAMKAMTILLEEAKAKDSGKSEESSHYSTEKESEKTAESQTPTPSATSFFSGKSPVTTLLECMHKLGNSCEFRLLSKEGPAHEPKFQYCVAVGAQTFPSVSAPSKKVAKQMAAEEAMKALHGEATNSMASDNQPEGMISESLDNLESMMPNKVRKIGELVRYLNTNPVGGLLEYARSHGFAAEFKLVDQSGPPHEPKFVYQAKVGGRWFPAVCAHSKKQGKQEAADAALRVLIGENEKAERMGFTEVTPVTGASLRRTMLLLSRSPEAQPKTLPLTGSTFHDQIAMLSHRCFNTLTNSFQPSLLGRKILAAIIMKKDSEDMGVVVSLGTGNRCVKGDSLSLKGETVNDCHAEIISRRGFIRFLYSELMKYNSQTAKDSIFEPAKGGEKLQIKKTVSFHLYISTAPCGDGALFDKSCSDRAMESTESRHYPVFENPKQGKLRTKVENGEGTIPVESSDIVPTWDGIRLGERLRTMSCSDKILRWNVLGLQGALLTHFLQPIYLKSVTLGYLFSQGHLTRAICCRVTRDGSAFEDGLRHPFIVNHPKVGRVSIYDSKRQSGKTKETSVNWCLADGYDLEILDGTRGTVDGPRNELSRVSKKNIFLLFKKLCSFRYRRDLLRLSYGEAKKAARDYETAKNYFKKGLKDMGYGNWISKPQEEKNFYLCPV

SEQ ID NO: 32 - protein, 701 aa, Homo sapiens
MDIEDEENMSSSSTDVKENRNLDNVSPKDGSTPGPGEGSQLSNGGGGGPGRKRPLEEGSNGHSKYRLKKRRKTPGPVLPKNALMQLNEIKPGLQYTLLSQTGPVHAPLFVMSVEVNGQVFEGSGPTKKKAKLHAAEKALRSFVQFPNASEAHLAMGRTLSVNTDFTSDQADFPDTLFNGFETPDKAEPPFYVGSNGDDSFSSSGDLSLSASPVPASLAQPPLPVLPPFPPPSGKNPVMILNELRPGLKYDFLSESGESHAKSFVMSVVVDGQFFEGSGRNKKLAKARAAQSALAAIFNLHLDQTPSRQPIPSEGLQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPADRHPNRKARGQLRTKIESGQGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTP

SEQ ID NO: 33 - protein, 1419 aa, Streptococcus pyogenes
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGSGGGGSGKRTADGSEFEPKKKRKVSSGGDYKDHDGDYKDHDIDYKDDDDK

SEQ ID NO: 34 - protein, 1053 aa, Staphylococcus aureus
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 35 - protein, 984 aa, Campylobacter jejuni
MARILAFDIGISSIGWAFSENDELKDCGVRIFTKVENPKTGESLALPRRLARSARKRLARRKARLNHLKHLIANEFKLNYEDYQSFDESLAKAYKGSLISPYELRFRALNELLSKQDFARVILHIAKRRGYDDIKNSDDKEKGAILKAIKQNEEKLANYQSVGEYLYKEYFQKFKENSKEFTNVRNKKESYERCIAQSFLKDELKLIFKKQREFGFSFSKKFEEEVLSVAFYKRALKDFSHLVGNCSFFTDEKRAPKNSPLAFMFVALTRIINLLNNLKNTEGILYTKDDLNALLNEVLKNGTLTYKQTKKLLGLSDDYEFKGEKGTYFIEFKKYKEFIKALGEHNLSQDDLNEIAKDITLIKDEIKLKKALAKYDLNQNQIDSLSKLEFKDHLNISFKALKLVTPLMLEGKKYDEACNELNLKVAINEDKKDFLPAFNETYYKDEVTNPVVLRAIKEYRKVLNALLKKYGKVHKINIELAREVGKNHSQRAKIEKEQNENYKAKKDAELECEKLGLKINSKNILKLRLFKEQKEFCAYSGEKIKISDLQDEKMLEIDHIYPYSRSFDDSYMNKVLVFTKQNQEKLNQTPFEAFGNDSAKWQKIEVLAKNLPTKKQKRILDKNYKDKEQKNFKDRNLNDTRYIARLVLNYTKDYLDFLPLSDDENTKLNDTQKGSKVHVEAKSGMLTSALRHTWGFSAKDRNNHLHHAIDAVIIAYANNSIVKAFSDFKKEQESNSAELYAKKISELDYKNKRKFFEPFSGFRQKVLDKIDEIFVSKPERKKPSGALHEETFRKEEEFYQSYGGKEGVLKALELGKIRKVNGKIVKNGDMFRVDIFKHKKTNKFYAVPIYTMDFALKVLPNKAVARSKKGEIKDWILMDENYEFCFSLYKDSLILIQTKDMQEPEFVYYNAFTSSTVSLIVSKHDNKFETLSKNQKILFKNANEKEVIAKSIGIQNLKVFEKYIVSALGEVTKAEFRQREDFKK

SEQ ID NO: 36 - protein, 1082 aa, Neisseria meningitidis
MAAFKPNSINYILGLDIGIASVGWAMVEIDEEENPIRLIDLGVRVFERAEVPKTGDSLAMARRLARSVRRLTRRRAHRLLRTRRLLKREGVLQAANFDENGLIKSLPNTPWQLRAAALDRKLTPLEWSAVLLHLIKHRGYLSQRKNEGETADKELGALLKGVAGNAHALQTGDFRTPAELALNKFEKESGHIRNQRSDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSPELQDEIGTAFSLFKTDEDITGRLKDRIQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLGRLNEKGYVEIDHALPFSRTWDDSFNNKVLVLGSENQNKGNQTPYEYFNGKDNSREWQEFKARVETSRFPRSKKQRILLQKFDEDGFKERNLNDTRYVNRFLCQFVADRMRLTGKGKKRVFASNGQITNLLRGFWGLRKVRAENDRHHALDAVVVACSTVAMQQKITRFVRYKEMNAFDGKTIDKETGEVLHQKTHFPQPWEFFAQEVMIRVFGKPDGKPEFEEADTLEKLRTLLAEKLSSRPEAVHEYVTPLFVSRAPNRKMSGQGHMETVKSAKRLDEGVSVLRVPLTQLKLKDLEKMVNREREPKLYEALKARLEAHKDDPAKAFAEPFYKYDKAGNRTQQVKAVRVEQVQKTGVWVRNHNGIADNATMVRVDVFEKGDKYYLVPIYSWQVAKGILPDRAVVQGKDEEDWQLIDDSFNFKFSLHPNDLVEVITKKARMFGYFASCHRGTGNINIRIHDLDHKIGKNGILEGIGVKTALSFQKYQIDELGKEIRPCRLKKRPPVR

SEQ ID NO: 37 - protein, 1307 aa, Acidaminococcus sp.
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 38 - protein, 1228 aa, unidentified
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 39 - protein, 1389 aa, Leptotrichia shahii
MGNLFGHKRWYEVRDKKDFKIKRKVKVKRNYDGNKYILNINENNNKEKIDNNKFIRKYINYKKNDNILKEFTRKFHAGNILFKLKGKEGIIRIENNDDFLETEEVVLYIEAYGKSEKLKALGITKKKIIDEAIRQGITKDDKKIEIKRQENEEEIEIDIRDEYTNKTLNDCSIILRIIENDELETKKSIYEIFKNINMSLYKIIEKIIENETEKVFENRYYEEHLREKLLKDDKIDVILTNFMEIREKIKSNLEILGFVKFYLNVGGDKKKSKNKKMLVEKILNINVDLTVEDIADFVIKELEFWNITKRIEKVKKVNNEFLEKRRNRTYIKSYVLLDKHEKFKIERENKKDKIVKFFVENIKNNSIKEKIEKILAEFKIDELIKKLEKELKKGNCDTEIFGIFKKHYKVNFDSKKFSKKSDEEKELYKIIYRYLKGRIEKILVNEQKVRLKKMEKIEIEKILNESILSEKILKRVKQYTLEHIMYLGKLRHNDIDMTTVNTDDFSRLHAKEELDLELITFFASTNMELNKIFSRENINNDENIDFFGGDREKNYVLDKKILNSKIKIIRDLDFIDNKNNITNNFIRKFTKIGTNERNRILHAISKERDLQGTQDDYNKVINIIQNLKISDEEVSKALNLDVVFKDKKNIITKINDIKISEENNNDIKYLPSFSKVLPEILNLYRNNPKNEPFDTIETEKIVLNALIYVNKELYKKLILEDDLEENESKNIFLQELKKTLGNIDEIDENIIENYYKNAQISASKGNNKAIKKYQKKVIECYIGYLRKNYEELFDFSDFKMNIQEIKKQIKDINDNKTYERITVKTSDKTIVINDDFEYIISIFALLNSNAVINKIRNRFFATSVWLNTSEYQNIIDILDEIMQLNTLRNECITENWNLNLEEFIQKMKEIEKDFDDFKIQTKKEIFNNYYEDIKNNILTEFKDDINGCDVLEKKLEKIVIFDDETKFEIDKKSNILQDEQRKLSNINKKDLKKKVDQYIKDKDQEIKSKILCRIIFNSDFLKKYKKEIDNLIEDMESENENKFQEIYYPKERKNELYIYKKNLFLNIGNPNFDKIYGLISNDIKMADAKFLFNIDGKNIRKNKISEIDAILKNLNDKLNGYSKEYKEKYIKKLKENDDFFAKNIQNKNYKSFEKDYNRVSEYKKIRDLVEFNYLNKIESYLIDINWKLAIQMARFERDMHYIVNGLRELGIIKLSGYNTGISRAYPKRNGSDGFYTTTAYYKFFDEESYKKFEKICYGFGIDLSENSEINKPENESIRNYISHFYIVRNPFADYSIAEQIDRVSNLLSYSTRYNNSTYASVFEVFKKDVNLDYDELKKKFKLIGNNDILERLMKPKKVSVLELESYNSDYIKNLIIELLTKIENTNDTL

SEQ ID NO: 40 - protein, 1152 aa, Leptotrichia wadei
MKVTKVDGISHKKYIEEGKLVKSTSEENRTSERLSELLSIRLDIYIKNPDNASEEENRIRRENLKKFFSNKVLHLKDSVLYLKNRKEKNAVQDKNYSEEDISEYDLKNKNSFSVLKKILLNEDVNSEELEIFRKDVEAKLNKINSLKYSFEENKANYQKINENNVEKVGGKSKRNIIYDYYRESAKRNDYINNVQEAFDKLYKKEDIEKLFFLIENSKKHEKYKIREYYHKIIGRKNDKENFAKIIYEEIQNVNNIKELIEKIPDMSELKKSQVFYKYYLDKEELNDKNIKYAFCHFVEIEMSQLLKNYVYKRLSNISNDKIKRIFEYQNLKKLIENKLLNKLDTYVRNCGKYNYYLQVGEIATSDFIARNRQNEAFLRNIIGVSSVAYFSLRNILETENENGITGRMRGKTVKNNKGEEKYVSGEVDKIYNENKQNEVKENLKMFYSYDFNMDNKNEIEDFFANIDEAISSIRHGIVHFNLELEGKDIFAFKNIAPSEISKKMFQNEINEKKLKLKIFKQLNSANVFNYYEKDVIIKYLKNTKFNFVNKNIPFVPSFTKLYNKIEDLRNTLKFFWSVPKDKEEKDAQIYLLKNIYYGEFLNKFVKNSKVFFKITNEVIKINKQRNQKTGHYKYQKFENIEKTVPVEYLAIIQSREMINNQDKEEKNTYIDFIQQIFLKGFIDYLNKNNLKYIESNNNNDNNDIFSKIKIKKDNKEKYDKILKNYEKHNRNKEIPHEINEFVREIKLGKILKYTENLNMFYLILKLLNHKELTNLKGSLEKYQSANKEETFSDELELINLLNLDNNRVTEDFELEANEIGKFLDFNENKIKDRKELKKFDTNKIYFDGENIIKHRAFYNIKKYGMLNLLEKIADKAKYKISLKELKEYSNKKNEIEKNYTMQQNLHRKYARPKKDEKFNDEDYKEYEKAIGNIQKYTHLKNKVEFNELNLLQGLLLKILHRLVGYTSIWERDLRFRLKGEFPENHYIEEIFNFDNSKNVKYKSGQIVEKYINFYKELYKDNVEKRSIYSDKKVKKLKQEKKDLYIRNYIAHFNYIPHAEISLLEVLENLRKLLSYDRKLKNAIMKSIVDILKEYGFVATFKIGADKKIEIQTLESEKIVHLKNLKKKKLMTDRNSEELCELVKVMFEYKALE

SEQ ID NO: 41 - protein, 118 aa, Homo sapiens
NPDREGWLLKLGGGRVKTWKRRWFILTDNCLYYFEYTTDKEPRGIIPLENLSIREVDDPRKPNCFELYIPNNKGQLIKACKTEADGRVVEGNHMVYRISAPTQEEKDEWIKSIQAAVS

SEQ ID NO: 42 - protein, 118 aa, Homo sapiens
NPDREGWLLKLGGGRVKTWKCRWFILTDNCLYYFEYTTDKEPRGIIPLENLSIREVDDPRKPNCFELYIPNNKGQLIKACKTEADGRVVEGNHMVYRISAPTQEEKDEWIKSIQAAVS

SEQ ID NO: 43 - protein, 59 aa, Homo sapiens
DNEVQNCMACGKGFSVTVRRHHCRQCGNIFCAECSAKNALTPSSKKPVRVCDACFNDLQ

SEQ ID NO: 44 - protein, 59 aa, Homo sapiens
DNEVQNCMACGKGFSVTVRRHHCLQCGNIFCAECSAKNALTPSSKKPVRVCDACFNDLQ

SEQ ID NO: 45 - protein, 122 aa, unidentified
DVAISANIADIEEKRGFTSHFVFVIEVKTKGGSKYLIYRRYRQFHALQSKLEERFGPDSKSSALACTLPTLPAKVYVGVKQEIAEMRIPALNAYMKSLLSLPVWVLMDEDVRIFFYQSPYDS

SEQ ID NO: 46 - protein, 122 aa, unidentified
DVAISANIADIEEKRGFTSHFVFVIEVKTKGGSKYLIYLRYRQFHALQSKLEERFGPDSKSSALACTLPTLPAKVYVGVKQEIAEMRIPALNAYMKSLLSLPVWVLMDEDVRIFFYQSPYDS

SEQ ID NO: 47 - protein, 126 aa, Homo sapiens
MQTGRTEDDLVPTAPSLGTKEGYLTKQGGLVKTWKTRWFTLHRNELKYFKDQMSPEPIRILDLTECSAVQFDYSQERVNCFCLVFPFRTFYLCAKTGVEADEWIKILRWKLSQIRKQLNQGEGTIR

SEQ ID NO: 48 - protein, 153 aa, Homo sapiens
PFKIPEDDGNDLTHTFFNPDREGWLLKLGGRVKTWKRRWFILTDNCLYYFEYTTDKEPRGIIPLENLSIREVEDPRKPNCFELYNPSHKGQVIKACKTEADGRVVEGNHVVYRISAPSPEEKEEWMKSIKASISRDPFYDMLATRKRRIANKK

SEQ ID NO: 49 - protein, 154 aa, Homo sapiens
MPFKIPEDDGNDLTHTFFNPDREGWLLKLGGRVKTWKCRWFILTDNCLYYFEYTTDKEPRGIIPLENLSIREVEDPRKPNCFELYNPSHKGQVIKACKTEADGRVVEGNHVVYRISAPSPEEKEEWMKSIKASISRDPFYDMLATRKRRIANKK

SEQ ID NO: 50 - protein, 101 aa, Homo sapiens
MGSGSAREGWLFKWTNYIKGYQRRWFVLSNGLLSYYRSKAEMRHTCRGTINLATANITVEDSCNFIISNGGAQTYHLKASSEVERQRWVTALELAKAKAVK

SEQ ID NO: 51 - protein, 101 aa, Homo sapiens
MGSGSAREGWLFKWTNYIKGYQERWFVLSNGLLSYYRSKAEMRHTCRGTINLATANITVEDSCNFIISNGGAQTYHLKASSEVERQRWVTALELAKAKAVK

SEQ ID NO: 52 - protein, 177 aa, Homo sapiens
MAAVILESIFLKRSQQKKKTSPLNFKKRLFLLTVHKLSYYEYDFERGRRGSKKGSIDVEKITCVETVVPEKNPPPERQIPRRGEESSEMEQISIIERFPYPFQVVYDEGPLYVFSPTEELRKRWIHQLKNVIRYNSDLVQKYHPCFWIDGQYLCCSQTAKNAMGCQILENRNGSLKP

SEQ ID NO: 53 - protein, 177 aa, Homo sapiens
MAAVILESIFLKRSQQKKKTSPLNFKKCLFLLTVHKLSYYEYDFERGRRGSKKGSIDVEKITCVETVVPEKNPPPERQIPRRGEESSEMEQISIIERFPYPFQVVYDEGPLYVFSPTEELRKRWIHQLKNVIRYNSDLVQKYHPCFWIDGQYLCCSQTAKNAMGCQILENRNGSLKP

SEQ ID NO: 54 - protein, 98 aa, Homo sapiens
MEGVLYKWTNYLTGWQPRWFVLDNGILSYYDSQDDVCKGSKGSIKMAVCEIKVHSADNTRMELIIPGEQHFYMKAVNAAERQRWLVALGSSKACLTDT

SEQ ID NO: 55 - protein, 95 aa, Homo sapiens
PVERCGVLSKWTNYIHGWQDRWVVLKNNALSYYKSEDETEYGCRGSICLSKAVITPHDFDECRFDISVNDSVWYLRAQDPDHRQQWIDAIEQHKT

SEQ ID NO: 56 - protein, 102 aa, Homo sapiens
MRIQLSGMYNVRKGKMQLPVNRWTRRQVILCGTCLIVSSVKDSLTGKMHVLPLIGGKVEEVKKHQHCLAFSSSGPQSQTYYICFDTFTEYLRWLRQVSKVAS

SEQ ID NO: 57 - protein, 98 aa, Homo sapiens
MDVLKQGYMMKKGHRRKNWTERWFVLKPNIISYYVSEDLKDKKGDILLDENCCVESLPDKDGKKCLFLVKCFDKTFEISASDKKKKQEWIQAIHSTIH

SEQ ID NO: 58 - protein, 98 aa, Homo sapiens
MDVLKQGYMMKKGHEEKNWTERWFVLKPNIISYYVSEDLKDKKGDILLDENCCVESLPDKDGKKCLFLVKCFDKTFEISASDKKKKQEWIQAIHSTIH

SEQ ID NO: 59 - protein, 114 aa, Homo sapiens
MDMLSSHHYKSFKVSMIHRLRFTTDVQLGISGDKVEIDPVTNQKASTKFWIKQKPISIDSDLLCACDLAEEKSPSHAIFKLTYLSNHDYKHLYFESDAATVNEIVLKVNYILES

SEQ ID NO: 60 - protein, 122 aa, Homo sapiens
MGTVMKEGWMVHYTSKDTLRKRHYWRLDSKCITLFQNDTGSRYYKEIPLSEILSLEPVKTSALIPNGANPHCFEITTANVVYYVGENVVNPSSPSPNNSVLTSGVGADVARMWEIAIQHALM

SEQ ID NO: 61 - protein, 105 aa, Homo sapiens
FIMEGPLTRIGAKHERHIFLFDGLMISCKPNHGQTRLPGYSSAEYRLKEKFVMRKIQICDKEDTCEHKHAFELVSKDENSIIFAAKSAEEKNNWMAALISLHYRS

SEQ ID NO: 62 - protein, 107 aa, Homo sapiens
QGTNLPPSRQIVIRKGWLTISNIGIMKGGSKGYWFVLTAESLSWYKDDEEKEKKYMLPLDNLKVRDVEKSFMSSKHIFALFNTEQRNVYKDYRFLELACDSQEDVDS

SEQ ID NO: 63 - protein, 159 aa, Homo sapiens
QLLKDSFMVELVEGARKLRHVFLFTDLLLCTKLKKQSGGKTQQYDCKWYIPLTDLSFQMVDELEAVPNIPLVPDEELDALKIKISQIKNDIQREKRANKGSKATERLKKKLSEQESLLLLMSPSMAFRVHSRNGKSYTFLISSDYERAEWRENIREQQK

SEQ ID NO: 64 - protein, 117 aa, Homo sapiens
KLLMQGSFSVWTDHKRGHTKVKELARFKPMQRHLFLHEKAVLFCKKREENGEGYEKAPSYSYKQSLNMAAVGITENVKGDAKKFEIWYNAREEVYIVQAPTPEIKAAWVNEIRKVLT

SEQ ID NO: 65 - protein, 175 aa, Homo sapiens
MDSGRDFLTLHGLQDDEDLQALLKGSQLLKVKSSSWRRERFYKLQEDCKTIWQESRKVMRTPESQLFSIEDIQEVRMGHRTEGLEKFARDVPEDRCFSIVFKDQRNTLDLIAPSPADAQHWVLGLHKIIHHSGSMDQRQKLQHWIHSCLRKADKNKDNKMSFKELQNFLKELNIQ

SEQ ID NO: 66 - protein, 170 aa, Homo sapiens
MDSGRDFLTLHGLQDDEDLQALLKGSQLLKVKSTSWRRELFYKLQEDCKTIWQESRKVMRTPESQLFSIEDIQEVRMGHRTEGLEKFARDVPEDRCFSIVFKDQRNTLDLIAPSPADAQHWVLGLHKIIHHSGSMDQRQKLQHWIHSCLRKADKNKDNKMSFKELQNFLK

SEQ ID NO: 67 - protein, 168 aa, Homo sapiens
MSDVAIVKEGWLHKRGEYIKTWRPRYFLLKNDGTFIGYKERPQDVDQREAPLNNFSVAQCQLMKTERPRPNTFIIRCLQWTTVIERTFHVETPEEREEWTTAIQTVADGLKKQEEEEMDFRSGSPSDNSGAEEMEVSLAKPKHRVTMNEFEYLKLLGKGTFGKVDPPV

SEQ ID NO: 68 - protein, 168 aa, Homo sapiens
MSDVAIVKEGWLHKRGKYIKTWRPRYFLLKNDGTFIGYKERPQDVDQREAPLNNFSVAQCQLMKTERPRPNTFIIRCLQWTTVIERTFHVETPEEREEWTTAIQTVADGLKKQEEEEMDFRSGSPSDNSGAEEMEVSLAKPKHRVTMNEFEYLKLLGKGTFGKVDPPV

SEQ ID NO: 69 - protein, 168 aa, Homo sapiens
MSDVAIVKEGWLHRRGEYIKTWRPRYFLLKNDGTFIGYKERPQDVDQREAPLNNFSVAQCQLMKTERPRPNTFIIRCLQWTTVIERTFHVETPEEREEWTTAIQTVADGLKKQEEEEMDFRSGSPSDNSGAEEMEVSLAKPKHRVTMNEFEYLKLLGKGTFGKVDPPV

SEQ ID NO: 70 - protein, 168 aa, Homo sapiens
MSDVAIVREGWLHKRGEYIKTWRPRYFLLKNDGTFIGYKERPQDVDQREAPLNNFSVAQCQLMKTERPRPNTFIIRCLQWTTVIERTFHVETPEEREEWTTAIQTVADGLKKQEEEEMDFRSGSPSDNSGAEEMEVSLAKPKHRVTMNEFEYLKLLGKGTFGKVDPPV

SEQ ID NO: 71 - protein, 168 aa, Homo sapiens
MSDVAIVKEGWLHKRGEYIKTWRPRYFLLKNDGTFIGYKERPQDVDQREAPLNNFSVAQCQLMKTERPRPNAFIIRCLQWTTVIERTFHVETPEEREEWTTAIQTVADGLKKQEEEEMDFRSGSPSDNSGAEEMEVSLAKPKHRVTMNEFEYLKLLGKGTFGKVDPPV

SEQ ID NO: 72 - protein, 168 aa, Homo sapiens
MSDVAIVKEGWLHKRGEYIKTWRPRYFLLKNDGTFIGYKERPQDVDQREAPLNNFSVAQCQLMKTERPRPNTFIIRCLQWTTVIERTFHVEAPEEREEWTTAIQTVADGLKKQEEEEMDFRSGSPSDNSGAEEMEVSLAKPKHRVTMNEFEYLKLLGKGTFGKVDPPV

SEQ ID NO: 73 - protein, 168 aa, Homo sapiens
MSDVAIVKEGWLHKRGEYIKTWRPCYFLLKNDGTFIGYKERPQDVDQREAPLNNFSVAQCQLMKTERPRPNTFIIRCLQWTTVIERTFHVETPEEREEWTTAIQTVADGLKKQEEEEMDFRSGSPSDNSGAEEMEVSLAKPKHRVTMNEFEYLKLLGKGTFGKVDPPV

SEQ ID NO: 74 - protein, 168 aa, Homo sapiens
MSDVAIVKEGWLHKRGEYIKTWRPRYFLLKNDGDFIGYKERPQDVDQREAPLNNFSVAQCQLMKTERPRPNTFIIRCLQWTTVIERTFHVETPEEREEWTTAIQTVADGLKKQEEEEMDFRSGSPSDNSGAEEMEVSLAKPKHRVTMNEFEYLKLLGKGTFGKVDPPV

SEQ ID NO: 75 - protein, 168 aa, Homo sapiens
MSDVAIVKEGWLHKRGEYIKTWRPRYFLLKNDGFFIGYKERPQDVDQREAPLNNFSVAQCQLMKTERPRPNTFIIRCLQWTTVIERTFHVETPEEREEWTTAIQTVADGLKKQEEEEMDFRSGSPSDNSGAEEMEVSLAKPKHRVTMNEFEYLKLLGKGTFGKVDPPV

SEQ ID NO: 76 - protein, 168 aa, Homo sapiens
MSDVAIVKEGWLHKRGEYIKTWRPRYFLLKNDGLFIGYKERPQDVDQREAPLNNFSVAQCQLMKTERPRPNTFIIRCLQWTTVIERTFHVETPEEREEWTTAIQTVADGLKKQEEEEMDFRSGSPSDNSGAEEMEVSLAKPKHRVTMNEFEYLKLLGKGTFGKVDPPV

SEQ ID NO: 77 - protein, 168 aa, Homo sapiens
MSDVAIVKEGWLHKRGEYIKTWRPRYFLLKNDGTFIGYKERPQDVDQREAPLNNFSVAQCQLMKTERPRPNTFIIRCLQWYTVIERTFHVETPEEREEWTTAIQTVADGLKKQEEEEMDFRSGSPSDNSGAEEMEVSLAKPKHRVTMNEFEYLKLLGKGTFGKVDPPV

SEQ ID NO: 78 - protein, 168 aa, Homo sapiens
MSDVAIVKEGWLHKRGEYIKTWRPRYFLLKNDGTFIGYKERPQDVDQREAPLNNFSVAQCQLMKTERPRPNTFIIRCLQWTTVIERTFHVETPEEREEWTTAIQTVADGLKKQEEEEMDFRSGSPSDNSGAEEMEVSLAKPAAAVTMNEFEYLKLLGKGTFGKVDPPV

SEQ ID NO: 79 - protein, 168 aa, Homo sapiens
MSDVAIVKEGWLHKRGEYIKTWRPRYFLLKNDGTFIGYKERPQDVDQREAPLNNFSVAQCQLMKTERPRPNTFIIRCLQWTTVIERTFHVETPEEREEWTCAIQTVADGLKKQEEEEMDFRSGSPSDNSGAEEMEVSLAKPKHRVTMNEFEYLKLLGKGTFGKVDPPV

SEQ ID NO: 80 - protein, 92 aa, Homo sapiens
KMGPVDKRKGLFARRRQLLLTEGPHLYYVDPVNKVLKGEIPWSQELRPEAKNFKTFFVHTPNRTYYLMDPSGNAHKWCRKIQEVWRQRYQSH

SEQ ID NO: 81 - protein, 130 aa, synthetic construct
MASNFTQFVLVDNGGTGDVTVAPSNFANGIAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIY

SEQ ID NO: 82 - protein, 62 aa, synthetic construct
MKSIRCKNCNKLLFKADSFDHIEIRCPRCKRHIIMLNACEHPTEKHCGKREKITHSDETVRY

SEQ ID NO: 83 - protein, 131 aa, synthetic construct
MAKTIVLAVGEATRTLTEIQSTADRQIFEEKVGPLVGRLRLTASLRQNGAKTAYRVNLKLDQADVVDASTSVAGELPKVRYTQVWSHDVTIVANSTEASRKSLYDLTKSLVATSQVEDLVVNLVPLGRSLE

SEQ ID NO: 84 - protein, 102 aa, synthetic construct
MAVPETRPNHTIYINNLNSKIKKDELKKSLYAIFSQFGQILDILVPRQRTPRGQAFVIFKEVSSATNALRSMQGFPFYDKPMRIQYAKTDKRIPAKMKGTFV

SEQ ID NO: 85 - protein, 74 aa, Homo sapiens
MADFETDESVLMRRQKQINYGKNTIAYDRYIKEVPRHLRQPGIHPKTPNKFKKYSRRSWDQQIKLWKVALHFWD

SEQ ID NO: 86 - protein, 38 aa, Human alphaherpesvirus 1
PTDALDDFDLDMLPADALDDFDLDMLPADALDDFDLDM

SEQ ID NO: 87 - protein, 53 aa, synthetic construct
GRADALDDFDLDMLGSDALDDFDLDMLGSDALDDFDLDMLGSDALDDFDLDML

SEQ ID NO: 88 - protein, 261 aa, Homo sapiens
SQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALL

SEQ ID NO: 89 - protein, 190 aa, Human gammaherpesvirus 8
RDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLF

SEQ ID NO: 90 - protein, 72 aa, Homo sapiens
MDAKSLTAWSRTLVTFKDVFVDFTREEWKLLDTAQQIVYRNVMLENYKNLVSLGYQLTKPDVILRLEKGEEP

SEQ ID NO: 91 - protein, 289 aa, Homo sapiens
EASVQVKRVLEKSPGKLLVKMPFQASPGGKGEGGGATTSAQVMVIKRPGRKRKAEADPQAIPKKRGRKPGSVVAAAAAEAKKKAVKESSIRSVQETVLPIKKRKTRETVSIEVKEVVKPLLVSTLGEKSGKGLKTCKSPGRKSKESSPKGRSSSASSPPKKEHHHHHHHAESPKAPMPLLPPPPPPEPQSSEDPISPPEPQDLSSSICKEEKMPRAGSLESDGCPKEPAKTQPMVAAAATTTTTTTTTVAEKYKHRGEGERKDIVSSSMPRPNREEPVDSRTPVTERVS

SEQ ID NO: 92 - protein, 718 aa, Homo sapiens
LPTCSCLDRVIQKDKGPYYTHLGAGPSVAAVREIMENRYGQKGNAIRIEIVVYTGKEGKSSHGCPIAKWVLRRSSDEEKVLCLVRQRTGHHCPTAVMVVLIMVWDGIPLPMADRLYTELTENLKSYNGHPTDRRCTLNENRTCTCQGIDPETCGASFSFGCSWSMYFNGCKFGRSPSPRRFRIDPSSPLHEKNLEDNLQSLATRLAPIYKQYAPVAYQNQVEYENVARECRLGSKEGRPFSGVTACLDFCAHPHRDIHNMNNGSTVVCTLTREDNRSLGVIPQDEQLHVLPLYKLSDTDEFGSKEGMEAKIKSGAIEVLAPRRKKRTCFTQPVPRSGKKRAAMMTEVLAHKIRAVEKKPIPRIKRKNNSTTTNNSKPSSLPTLGSNTETVQPEVKSETEPHFILKSSDNTKTYSLMPSAPHPVKEASPGFSWSPKTASATPAPLKNDATASCGFSERSSTPHCTMPSGRLSGANAAAADGPGISQLGEVAPLPTLSAPVMEPLINSEPSTGVTEPLTPHQPNHQPSFLTSPQDLASSPMEEDEQHSEADEPPSDEPLSDDPLSPAEEKLPHIDEYWSDSEHIFLDANIGGVAIAPAHGSVLIECARRELHATTPVEHPNRNHPTRLSLVFYQHKNLNKPQHGFELNKIKFEAKEAKNKKMKASEQKDQAANEGPEQSSEVNELNQIPSHKALTLTHDNVVTVSPYALTHVAGPYNHWV

SEQ ID NO: 93 - protein, 912 aa, Homo sapiens
MPAMPSSGPGDTSSSAAEREEDRKDGEEQEEPRGKEERQEPSTTARKVGRPGRKRKHPPVESGDTPKDPAVISKSPSMAQDSGASELLPNGDLEKRSEPQPEEGSPAGGQKGGAPAEGEGAAETLPEASRAVENGCCTPKEGRGAPAEAGKEQKETNIESMKMEGSRGRLRGGLGWESSLRQRPMPRLTFQAGDPYYISKRKRDEWLARWKREAEKKAKVIAGMNAVEENQGPGESQKVEEASPPAVQQPTDPASPTVATTPEPVGSDAGDKNATKAGDDEPEYEDGRGFGIGELVWGKLRGFSWWPGRIVSWWMTGRSRAAEGTRWVMWFGDGKFSVVCVEKLMPLSSFCSAFHQATYNKQPMYRKAIYEVLQVASSRAGKLFPVCHDSDESDTAKAVEVQNKPMIEWALGGFQPSGPKGLEPPEEEKNPYKEVYTDMWVEPEAAAYAPPPPAKKPRKSTAEKPKVKEIIDERTRERLVYEVRQKCRNIEDICISCGSLNVTLEHPLFVGGMCQNCKNCFLECAYQYDDDGYQSYCTICCGGREVLMCGNNNCCRCFCVECVDLLVGPGAAQAAIKEDPWNCYMCGHKGTYGLLRRREDWPSRLQMFFANNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACV

SEQ ID NO: 94 - protein, 511 aa, unidentified
MKCLLYLAFLFIGVNCKFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 95 - protein, 511 aa, unidentified
MKCLLYLAFLFIGVNCKFTIVFPHNQKGNWKNVPSNYHYCPSSSDLNWHNDLIGTALQVKMPASHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 96 - protein, 511 aa, unidentified
MKCLLYLAFLFIGVNCKFTIVFPHNQKGNWKNVPSNYHYCPSSSDLNWHNDLIGTALQVKMPESHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 97 - protein, 511 aa, unidentified
MKCLLYLAFLFIGVNCKFTIVFPHNQKGNWKNVPSNYHYCPSSSDLNWHNDLIGTALQVKMPGSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 98 - protein, 511 aa, unidentified
MKCLLYLAFLFIGVNCKFTIVFPHNQKGNWKNVPSNYHYCPSSSDLNWHNDLIGTALQVKMPQSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 99 - protein, 511 aa, unidentified
MKCLLYLAFLFIGVNCKFTIVFPHNQKGNWKNVPSNYHYCPSSSDLNWHNDLIGTALQVKMPWSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 100 - protein, 511 aa, unidentified
MKCLLYLAFLFIGVNCKFTIVFPHNQKGNWKNVPSNYHYCPSSSDLNWHNDLIGTALQVKMPASHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEAELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 101 - protein, 511 aa, unidentified
MKCLLYLAFLFIGVNCKFTIVFPHNQKGNWKNVPSNYHYCPSSSDLNWHNDLIGTALQVKMPESHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEAELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 102 - protein, 511 aa, unidentified
MKCLLYLAFLFIGVNCKFTIVFPHNQKGNWKNVPSNYHYCPSSSDLNWHNDLIGTALQVKMPGSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEAELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 103 - protein, 511 aa, unidentified
MKCLLYLAFLFIGVNCKFTIVFPHNQKGNWKNVPSNYHYCPSSSDLNWHNDLIGTALQVKMPQSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEAELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 104 - protein, 511 aa, unidentified
MKCLLYLAFLFIGVNCKFTIVFPHNQKGNWKNVPSNYHYCPSSSDLNWHNDLIGTALQVKMPWSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEAELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 105 - protein, 511 aa, synthetic construct; identical to SEQ ID NO: 94

SEQ ID NO: 106 - protein, 108 aa, unidentified
MKCLLYLAFLFIGVNCKFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 107 - protein, 89 aa, unidentified
MKCLLYLAFLFIGVNCKFFGDTGLSKNPIELVEGWFSSWKSSIASFFFIIGLIIGLFLVLRVGIHLCIKLKHTKKRQIYTDIEMNRLGK

SEQ ID NO: 108 - protein, 81 aa, unidentified
MKCLLYLAFLFIGVNCKKNPIELVEGWFSSWKSSIASFFFIIGLIIGLFLVLRVGIHLCIKLKHTKKRQIYTDIEMNRLGK

SEQ ID NO: 109 - protein, 108 aa, synthetic construct; identical to SEQ ID NO: 106

SEQ ID NO: 110 - protein, 89 aa, synthetic construct; identical to SEQ ID NO: 107

SEQ ID NO: 111 - protein, 81 aa, synthetic construct; identical to SEQ ID NO: 108

SEQ ID NO: 112 - protein, 654 aa, Murine leukemia virus
MARSTLSKPPQDKINPWKPLIVMGVLLGVGMAESPHQVFNVTWRVTNLMTGRTANATSLLGTVQDAFPKLYFDLCDLVGEEWDPSDQEPYVGYGCKYPAGRQRTRTFDFYVCPGHTVKSGCGGPGEGYCGKWGCETTGQAYWKPTSSWDLISLKRGNTPWDTGCSKVACGPCYDLSKVSNSFQGATRGGRCNPLVLEFTDAGKKANWDGPKSWGLRLYRTGTDPITMFSLTRQVLNVGPRVPIGPNPVLPDQRLPSSPIEIVPAPQPPSPLNTSYPPSTTSTPSTSPTSPSVPQPPPGTGDRLLALVKGAYQALNLTNPDKTQECWLCLVSGPPYYEGVAVVGTYTNHSTAPANCTATSQHKLTLSEVTGQGLCMGAVPKTHQALCNTTQSAGSGSYYLAAPAGTMWACSTGLTPCLSTTVLNLTTDYCVLVELWPRVIYHSPDYMYGQLEQRTKYKREPVSLTLALLLGGLTMGGIAAGIGTGTTALIKTQQFEQLHAAIQTDLNEVEKSITNLEKSLTSLSEVVLQNRRGLDLLFLKEGGLCAALKEECCFYADHTGLVRDSMAKLRERLNQRQKLFETGQGWFEGLFNRSPWFTTLISTIMGPLIVLLLILLFGPCILNRLVQFVKDRISVVQALVLTQQYHQLKPIEYEP

SEQ ID NO: 113 - protein, 654 aa, Murine leukemia virus
MARSTLSKPPQDKINPWKPLIVMGVLLGVGMAESPHQVFNVTWRVTNLMTGRTANATSLLGTVQDAFPKLYFDLCDLVGEEWDPSKQEPYVGYGCKYPAGRQRTRTFDFYVCPGHTVKSGCGGPGEGYCGKWGCETTGQAYWKPTSSWDLISLKRGNTPWDTGCSKVACGPCYDLSKVSNSFQGATRGGRCNPLVLEFTDAGKKANWDGPKSWGLRLYRTGTDPITMFSLTRQVLNVGPRVPIGPNPVLPDQRLPSSPIEIVPAPQPPSPLNTSYPPSTTSTPSTSPTSPSVPQPPPGTGDRLLALVKGAYQALNLTNPDKTQECWLCLVSGPPYYEGVAVVGTYTNHSTAPANCTATSQHKLTLSEVTGQGLCMGAVPKTHQALCNTTQSAGSGSYYLAAPAGTMWACSTGLTPCLSTTVLNLTTDYCVLVELWPRVIYHSPDYMYGQLEQRTKYKREPVSLTLALLLGGLTMGGIAAGIGTGTTALIKTQQFEQLHAAIQTDLNEVEKSITNLEKSLTSLSEVVLQNRRGLDLLFLKEGGLCAALKEECCFYADHTGLVRDSMAKLRERLNQRQKLFETGQGWFEGLFNRSPWFTTLISTIMGPLIVLLLILLFGPCILNRLVQFVKDRISVVQALVLTQQYHQLKPIEYEP

SEQ ID NO: 114 - protein, 637 aa, Murine leukemia virus
MARSTLSKPPQDKINPWKPLIVMGVLLGVGMAESPHQVFNVTWRVTNLMTGRTANATSLLGTVQDAFPKLYFDLCDLVGEEWDPSDQEPYVGYGCKYPAGRQRTRTFDFYVCPGHTVKSGCGGPGEGYCGKWGCETTGQAYWKPTSSWDLISLKRGNTPWDTGCSKVACGPCYDLSKVSNSFQGATRGGRCNPLVLEFTDAGKKANWDGPKSWGLRLYRTGTDPITMFSLTRQVLNVGPRVPIGPNPVLPDQRLPSSPIEIVPAPQPPSPLNTSYPPSTTSTPSTSPTSPSVPQPPPGTGDRLLALVKGAYQALNLTNPDKTQECWLCLVSGPPYYEGVAVVGTYTNHSTAPANCTATSQHKLTLSEVTGQGLCMGAVPKTHQALCNTTQSAGSGSYYLAAPAGTMWACSTGLTPCLSTTVLNLTTDYCVLVELWPRVIYHSPDYMYGQLEQRTKYKREPVSLTLALLLGGLTMGGIAAGIGTGTTALIKTQQFEQLHAAIQTDLNEVEKSITNLEKSLTSLSEVVLQNRRGLDLLFLKEGGLCAALKEECCFYADHTGLVRDSMAKLRERLNQRQKLFETGQGWFEGLFNRSPWFTTLISTIMGPLIVLLLILLFGPCILNRLVQFVKDRISVVQA

SEQ ID NO: 115 - protein, 654 aa, Murine leukemia virus
MARSTLSKPPQDKINPWKPLIVMGVLLGVGMAESPHQVFNVTWRVTNLMTGRTANATSLLGTVQDAFPKLYFDLCDLVGEEWDPSDQEPYVGYGCKYPAGRQRTRTFDFYVCPGHTVKSGCGGPGEGYCGKWGCETTGQAYWKPTSSWDLISLKRGNTPWDTGCSKVACGPCYDLSKVSNSFQGATRGGRCNPLVLEFTDAGKKANWDGPKSWGLRLYRTGTDPITMFSLTRQVLNVGPRVPIGPNPVLPDQRLPSSPIEIVPAPQPPSPLNTSYPPSTTSTPSTSPTSPSVPQPPPGTGDRLLALVKGAYQALNLTNPDKTQECWLCLVSGPPYYEGVAVVGTYTNHSTAPANCTATSQHKLTLSEVTGQGLCMGAVPKTHQALCNTTQSAGSGSYYLAAPAGTMWACSTGLTPCLSTTVLNLTTDYCVLVELWPRVIYHSPDYMYGQLEQRTIEGREPVSLTLALLLGGLTMGGIAAGIGTGTTALIKTQQFEQLHAAIQTDLNEVEKSITNLEKSLTSLSEVVLQNRRGLDLLFLKEGGLCAALKEECCFYADHTGLVRDSMAKLRERLNQRQKLFETGQGWFEGLFNRSPWFTTLISTIMGPLIVLLLILLFGPCILNRLVQFVKDRISVVQALVLTQQYHQLKPIEYEP

SEQ ID NO: 116 - protein, 654 aa, Murine leukemia virus
MARSTLSKPPQDKINPWKPLIVMGVLLGVGMAESPHQVFNVTWRVTNLMTGRTANATSLLGTVQDAFPKLYFDLCDLVGEEWDPSDQEPYVGYGCKYPAGRQRTRTFDFYVCPGHTVKSGCGGPGEGYCGKWGCETTGQAYWKPTSSWDLISLKRGNTPWDTGCSKVACGPCYDLSKVSNSFQGATRGGRCNPLVLEFTDAGKKANWDGPKSWGLRLYRTGTDPITMFSLTRQVLNVGPRVPIGPNPVLPDQRLPSSPIEIVPAPQPPSPLNTSYPPSTTSTPSTSPTSPSVPQPPPGTGDRLLALVKGAYQALNLTNPDKTQECWLCLVSGPPYYEGVAVVGTYTNHSTAPANCTATSQHKLTLSEVTGQGLCMGAVPKTHQALCNTTQSAGSGSYYLAAPAGTMWACSTGLTPCLSTTVLNLTTDYCVLVELWPRVIYHSPDYMYGQLEQRTKYKREPVSLTLALLLGGLTMGGIAAGIGTGTTALIKTQQFEQLHAAIQTDLNEVEKSITNLEKSLTSLSEVVLQNRRGLDLLFLKEGGLCAALKEECCFYADHTGLVRDSMAKLRERLNQRQKLFETGQGWFEGLFNRSPWFTTLISTIMGPLIVLLLILLFGPCILNRLVQFVKDRISVVQALVATQQYHQLKPIEYEP

SEQ ID NO: 117 - protein, 654 aa, Murine leukemia virus
MARSTLSKPPQDKINPWKPLIVMGVLLGVGMAESPHQVFNVTWRVTNLMTGRTANATSLLGTVQDAFPKLYFDLCDLVGEEWDPSDQEPYVGYGCKYPAGRQRTRTFDFYVCPGHTVKSGCGGPGEGYCGKWGCETTGQAYWKPTSSWDLISLKRGNTPWDTGCSKVACGPCYDLSKVSNSFQGATRGGRCNPLVLEFTDAGKKANWDGPKSWGLRLYRTGTDPITMFSLTRQVLNVGPRVPIGPNPVLPDQRLPSSPIEIVPAPQPPSPLNTSYPPSTTSTPSTSPTSPSVPQPPPGTGDRLLALVKGAYQALNLTNPDKTQECWLCLVSGPPYYEGVAVVGTYTNHSTAPANCTATSQHKLTLSEVTGQGLCMGAVPKTHQALCNTTQSAGSGSYYLAAPAGTMWACSTGLTPCLSTTVLNLTTDYCVLVELWPRVIYHSPDYMYGQLEQRTKYKREPVSLTLALLLGGLTMGGIAAGIGTGTTALIKTQQFEQLHAAIQTDLNEVEKSITNLEKSLTSLSEVVLQNRRGLDLLFLKEGGLCAALKEECCFYADHTGLVRDSMAKLRERLNQRQKLFETGQGWFEGLFNRSPWFTTLISTIMGPLIVLLLILLFGPCILNRLVQFVKDRISVVQALVLTQQAHQLKPIEYEP

SEQ ID NO: 118 - protein, 654 aa, synthetic construct; identical to SEQ ID NO: 112

SEQ ID NO: 119 - protein, 645 aa, Murine leukemia virus
MARSTLSKPLKDKINPWKSLMVMGVLLRVGMAESPHQVFNVTWRVTNLMTGRTANATSLLGTVQDAFPRLYFDLCDLVGEEWDPSDQEPYVGYGCKYPGGRKRTRTFDFYVCPGHTVKSGCGGPREGYCGEWGCETTGQAYWKPTSSWDLISLKRGNTPWDTGCSKMACGPCYDLSKVSNSFQGATRGGRCNPLVLEFTDAGKKANWDGPKSWGLRLYRTGTDPITMFSLTRQVLNIGPRIPIGPNPVITGQLPPSRPVQIRLPRPPQPPPTGAASIVPETAPPSQQPGTGDRLLNLVEGAYRALNLTNPDKTQECWLCLVSGPPYYEGVAVVGTYTNHSTAPASCTATSQHKLTLSEVTGQGLCMGAVPKTHQALCNTTQSAGSGSYYLAAPAGTMWACSTGLTPCLSTTMLNLTTDYCVLVELWPRIIYHSPDYMYGQLEQRTKYKREPVSLTLALLLGGLTMGGIAAGIGTGTTALIKTQQFEQLHAAIQTDLNEVEKSITNLEKSLTSLSEVVLQNRRGLDLLFLKEGGLCAALKEECCFYADHTGLVRDSMAKLRERLNQRQKLFESGQGWFEGLFNRSPWFTTLISTIMGPLIVLLLILLFGPCILNRLVQFVKDRISVVQALVLTQQYHQLKPIEYEP

SEQ ID NO: 120 - protein, 645 aa, Murine leukemia virus
MARSTLSKPLKDKINPWKSLMVMGVLLRVGMAESPHQVFNVTWRVTNLMTGRTANATSLLGTVQDAFPRLYFDLCDLVGEEWDPSKQEPYVGYGCKYPGGRKRTRTFDFYVCPGHTVKSGCGGPREGYCGEWGCETTGQAYWKPTSSWDLISLKRGNTPWDTGCSKMACGPCYDLSKVSNSFQGATRGGRCNPLVLEFTDAGKKANWDGPKSWGLRLYRTGTDPITMFSLTRQVLNIGPRIPIGPNPVITGQLPPSRPVQIRLPRPPQPPPTGAASIVPETAPPSQQPGTGDRLLNLVEGAYRALNLTNPDKTQECWLCLVSGPPYYEGVAVVGTYTNHSTAPASCTATSQHKLTLSEVTGQGLCMGAVPKTHQALCNTTQSAGSGSYYLAAPAGTMWACSTGLTPCLSTTMLNLTTDYCVLVELWPRIIYHSPDYMYGQLEQRTKYKREPVSLTLALLLGGLTMGGIAAGIGTGTTALIKTQQFEQLHAAIQTDLNEVEKSITNLEKSLTSLSEVVLQNRRGLDLLFLKEGGLCAALKEECCFYADHTGLVRDSMAKLRERLNQRQKLFESGQGWFEGLFNRSPWFTTLISTIMGPLIVLLLILLFGPCILNRLVQFVKDRISVVQALVLTQQYHQLKPIEYEP

SEQ ID NO: 121 - protein, 628 aa, Murine leukemia virus
MARSTLSKPLKDKINPWKSLMVMGVLLRVGMAESPHQVFNVTWRVTNLMTGRTANATSLLGTVQDAFPRLYFDLCDLVGEEWDPSDQEPYVGYGCKYPGGRKRTRTFDFYVCPGHTVKSGCGGPREGYCGEWGCETTGQAYWKPTSSWDLISLKRGNTPWDTGCSKMACGPCYDLSKVSNSFQGATRGGRCNPLVLEFTDAGKKANWDGPKSWGLRLYRTGTDPITMFSLTRQVLNIGPRIPIGPNPVITGQLPPSRPVQIRLPRPPQPPPTGAASIVPETAPPSQQPGTGDRLLNLVEGAYRALNLTNPDKTQECWLCLVSGPPYYEGVAVVGTYTNHSTAPASCTATSQHKLTLSEVTGQGLCMGAVPKTHQALCNTTQSAGSGSYYLAAPAGTMWACSTGLTPCLSTTMLNLTTDYCVLVELWPRIIYHSPDYMYGQLEQRTKYKREPVSLTLALLLGGLTMGGIAAGIGTGTTALIKTQQFEQLHAAIQTDLNEVEKSITNLEKSLTSLSEVVLQNRRGLDLLFLKEGGLCAALKEECCFYADHTGLVRDSMAKLRERLNQRQKLFESGQGWFEGLFNRSPWFTTLISTIMGPLIVLLLILLFGPCILNRLVQFVKDRISVVQA

SEQ ID NO: 122 - protein, 645 aa, Murine leukemia virus
MARSTLSKPLKDKINPWKSLMVMGVLLRVGMAESPHQVFNVTWRVTNLMTGRTANATSLLGTVQDAFPRLYFDLCDLVGEEWDPSDQEPYVGYGCKYPGGRKRTRTFDFYVCPGHTVKSGCGGPREGYCGEWGCETTGQAYWKPTSSWDLISLKRGNTPWDTGCSKMACGPCYDLSKVSNSFQGATRGGRCNPLVLEFTDAGKKANWDGPKSWGLRLYRTGTDPITMFSLTRQVLNIGPRIPIGPNPVITGQLPPSRPVQIRLPRPPQPPPTGAASIVPETAPPSQQPGTGDRLLNLVEGAYRALNLTNPDKTQECWLCLVSGPPYYEGVAVVGTYTNHSTAPASCTATSQHKLTLSEVTGQGLCMGAVPKTHQALCNTTQSAGSGSYYLAAPAGTMWACSTGLTPCLSTTMLNLTTDYCVLVELWPRIIYHSPDYMYGQLEQRTIEGREPVSLTLALLLGGLTMGGIAAGIGTGTTALIKTQQFEQLHAAIQTDLNEVEKSITNLEKSLTSLSEVVLQNRRGLDLLFLKEGGLCAALKEECCFYADHTGLVRDSMAKLRERLNQRQKLFESGQGWFEGLFNRSPWFTTLISTIMGPLIVLLLILLFGPCILNRLVQFVKDRISVVQALVLTQQYHQLKPIEYEP

SEQ ID NO: 123 - protein, 645 aa, Murine leukemia virus
MARSTLSKPLKDKINPWKSLMVMGVLLRVGMAESPHQVFNVTWRVTNLMTGRTANATSLLGTVQDAFPRLYFDLCDLVGEEWDPSDQEPYVGYGCKYPGGRKRTRTFDFYVCPGHTVKSGCGGPREGYCGEWGCETTGQAYWKPTSSWDLISLKRGNTPWDTGCSKMACGPCYDLSKVSNSFQGATRGGRCNPLVLEFTDAGKKANWDGPKSWGLRLYRTGTDPITMFSLTRQVLNIGPRIPIGPNPVITGQLPPSRPVQIRLPRPPQPPPTGAASIVPETAPPSQQPGTGDRLLNLVEGAYRALNLTNPDKTQECWLCLVSGPPYYEGVAVVGTYTNHSTAPASCTATSQHKLTLSEVTGQGLCMGAVPKTHQALCNTTQSAGSGSYYLAAPAGTMWACSTGLTPCLSTTMLNLTTDYCVLVELWPRIIYHSPDYMYGQLEQRTKYKREPVSLTLALLLGGLTMGGIAAGIGTGTTALIKTQQFEQLHAAIQTDLNEVEKSITNLEKSLTSLSEVVLQNRRGLDLLFLKEGGLCAALKEECCFYADHTGLVRDSMAKLRERLNQRQKLFESGQGWFEGLFNRSPWFTTLISTIMGPLIVLLLILLFGPCILNRLVQFVKDRISVVQALVATQQYHQLKPIEYEP

SEQ ID NO: 124 - protein, 645 aa, Murine leukemia virus
MARSTLSKPLKDKINPWKSLMVMGVLLRVGMAESPHQVFNVTWRVTNLMTGRTANATSLLGTVQDAFPRLYFDLCDLVGEEWDPSDQEPYVGYGCKYPGGRKRTRTFDFYVCPGHTVKSGCGGPREGYCGEWGCETTGQAYWKPTSSWDLISLKRGNTPWDTGCSKMACGPCYDLSKVSNSFQGATRGGRCNPLVLEFTDAGKKANWDGPKSWGLRLYRTGTDPITMFSLTRQVLNIGPRIPIGPNPVITGQLPPSRPVQIRLPRPPQPPPTGAASIVPETAPPSQQPGTGDRLLNLVEGAYRALNLTNPDKTQECWLCLVSGPPYYEGVAVVGTYTNHSTAPASCTATSQHKLTLSEVTGQGLCMGAVPKTHQALCNTTQSAGSGSYYLAAPAGTMWACSTGLTPCLSTTMLNLTTDYCVLVELWPRIIYHSPDYMYGQLEQRTKYKREPVSLTLALLLGGLTMGGIAAGIGTGTTALIKTQQFEQLHAAIQTDLNEVEKSITNLEKSLTSLSEVVLQNRRGLDLLFLKEGGLCAALKEECCFYADHTGLVRDSMAKLRERLNQRQKLFESGQGWFEGLFNRSPWFTTLISTIMGPLIVLLLILLFGPCILNRLVQFVKDRISVVQALVLTQQAHQLKPIEYEP

SEQ ID NO: 125 - protein, 645 aa, synthetic construct; identical to SEQ ID NO: 119

SEQ ID NO: 126 - protein, 563 aa, Influenza A virus
MNTQILVFALVAVIPTNADKICLGHHAVSNGTKVNTLTERGVEVVNATETVERTNIPKICSKGKRTTDLGQCGLLGTITGPPQCDQFLEFSADLIIERREGNDVCYPGKFVNEEALRQILRGSGGIDKETMGFTYSGIRTNGTTSACRRSGSSFYAEMEWLLSNTDNASFPQMTKSYKNTRRESALIVWGIHHSGSTTEQTKLYGSGNKLITVGSSKYHQSFVPSPGTRPQINGQSGRIDFHWLILDPNDTVTFSFNGAFIAPNRASFLRGKSMGIQSDVQVDANCEGECYHSGGTITSRLPFQNINSRAVGKCPRYVKQESLLLATGMKNVPEPSKKRKKRGLFGAIAGFIENGWEGLVDGWYGFRHQNAQGEGTAADYKSTQSAIDQITGKLNRLIEKTNQQFELIDNEFTEVEKQIGNLINWTKDSITEVWSYNAELIVAMENQHTIDLADSEMNRLYERVRKQLRENAEEDGTGCFEIFHKCDDDCMASIRNNTYDHSKYREEAMQNRIQIDPVKLSSGYKDVILWFSFGASCFLLLAIAMGLVFICVKNGNMRCTICI

SEQ ID NO: 127 - protein, 563 aa, Influenza A virus
MNTQILVFALVAVIPTNADKICLGHHAVSNGTKVNTLTERGVEVVNATETVERTNIPKICSKGKRTTDLGQCGLLGTITGPPQCDQFLEFSADLIIERREGNDVCFPGKFVNEEALRQILRGSGGIDKETMGFTYSGIRTNGTTSACRRSGSSFYAEMEWLLSNTDNASFPQMTKSYKNTRRESALIVWGIHHSGSTTQQTKLYGSGNKLITVGSSKYHQSFVPSPGTRPQINGQSKRIDFHWLILDPNDTVTFSFNGAFIAPNRASFLRGKSMGIQSDVQVDANCEGECYHSGGTITSRLPFQNINSRAVGKCPRYVKQESLLLATGMKNVPEPSKKRKKRGLFGAIAGFIENGWEGLVDGWYGFRHQNAQGEGTAADYKSTQSAIDQITGKLNRLIEKTNQQFELIDNEFTEVEKQIGNLINWTKDSITEVWSYNAELIVAMENQHTIDLADSEMNRLYERVRKQLRENAEEDGTGCFEIFHKCDDDCMASIRNNTYDHSKYREEAMQNRIQIDPVKLSSGYKDVILWFSFGASCFLLLAIAMGLVFICVKNGNMRCTICI

SEQ ID NO: 128 - protein, 564 aa, Influenza A virus
MNTQILVFALVAVIPTNADKICLGHHAVSNGTKVNTLTERGVEVVNATETVERTNIPKICSKGKRTTDLGQCGLLGTITGPPQCDQFLEFSADLIIERREGNDVCYPGKFVNEEALRQILRGSGGIDKETMGFTYSGIRTNGTTSACRRSGSSFYAEMEWLLSNTDNASFPQMTKSYKNTRRESALIVWGIHHSGSTTEQTKLYGSGNKLITVGSSKYHQSFVPSPGTRPQINGQSGRIDFHWLILDPNDTVTFSFNGAFIAPNRASFLRGKSMGIQSDVQVDANCEGECYHSGGTITSRLPFQNINSRAVGKCPRYVKQESLLLATGMKNVPEPSKKRKGKRGLFGAIAGFIENGWEGLVDGWYGFRHQNAQGEGTAADYKSTQSAIDQITGKLNRLIEKTNQQFELIDNEFTEVEKQIGNLINWTKDSITEVWSYNAELIVAMENQHTIDLADSEMNRLYERVRKQLRENAEEDGTGCFEIFHKCDDDCMASIRNNTYDHSKYREEAMQNRIQIDPVKLSSGYKDVILWFSFGASCFLLLAIAMGLVFICVKNGNMRCTICI

SEQ ID NO: 129 - protein, 454 aa, Influenza A virus
MNPNQKIITIGSICLVVGLISLILQIGNIISIWISHSIQTGSQNHTGICNQNIITYKNSTWVKDTTSVILTGNSSLCPIRGWAIYSKDNSIRIGSKGDVFVIREPFISCSHLECRTFFLTQGALLNDKHSSGTVKDRSPYRALMSCPVGEAPSPYNSRFESVAWSASACHDGMGWLTIGISGPDNGAVAVLKYNGIITETIKSWRKKILRTQESECACVNGSCFTIMTDGPSDGLASYKIFKIEKGKVTKSIELNAPNSHYEECSCYPDTGKVMCVCRDNWHGSNRPWVSFDQNLDYQIGYICSGVFGDNPRPEDGTGSCGPVYVDGANGVKGFSYRYGNGVWIGRTKSHSSRHGFEMIWDPNGWTETDSKFSVRQDVVAMTDWSGYSGSFVQHPELTGLDCMRPCFWVELIRGRPKEKTIWTSASSISFCGVNSDTVDWSWPDGAELPFSIDK

SEQ ID NO: 130 - protein, 454 aa, Influenza A virus
MNPNQKIITIGSICLVVGLISLILQIGNIISIWISHSIQTGSQNHTGICNQNIIAYKNSTWVKDTTSVILTGNSSLCPIRGWAIYSKDNSIRIGSKGDVFVIREPFISCSHLECRTFFLTQGALLNDKHSSGTVKDRSPYRALMSCPVGEAPSPYNSRFESVAWSASACHDGMGWLTIGISGPDNGAVAVLKYNGIITETIKSWRKKILRTQESECACVNGSCFTIMTDGPSDGLASYKIFKIEKGKVTKSIELNAPNSHYEECSCYPDTGKVMCVCRDNWHGSNRPWVSFDQNLDYQIGYICSGVFGDNPRPEDGTGSCGPVYVDGANGVKGFSYRYGNGVWIGRTKSHSSRHGFEMIWDPNGWTETDSKFSVRQDVVAMTDWSGYSGSFVQHPELTGLDCMRPCFWVELIRGRPKEKTIWTSASSISFCGVNSDTVDWSWPDGAELPFSIDK

SEQ ID NO: 131 - protein, 981 aa, Sindbis Virus
SAAPLVTAMCLLGNVSFPCNRPPTCYTREPSRALDILEENVNHEAYDTLLNAILRCGSSGRSKRSVTDDFTLTSPYLGTCSYCHHTEPCFSPIKIEQVWDEADDNTIRIQTSAQFGYDQSGAASSNKYRYMSLEQDHTVKEGTMDDIKISTSGPCRRLSYKGYFLLAKCPPGDSVTVSIASSNSATSCTMARKIKPKFVGREKYDLPPVHGKKIPCTVYDRLKETTAGYITMHRPGPHAYTSYLEESSGKVYAKPPSGKNITYECKCGDYKTGTVTTRTEITGCTAIKQCVAYKSDQTKWVFNSPDLIRHADHTAQGKLHLPFKLIPSTCMVPVAHAPNVVHGFKHISLQLDTDHLTLLTTRRLGANPEPTTEWIIGKTVRNFTVDRDGLEYIWGNHEPVRVYAQESAPGDPHGWPHEIVQHYYHRHPVYTILAVASAAVAMMIGVTVAALCACKARRECLTPYALAPNAVIPTSLALLCCVRSANAETFTETMSYLWSNSQPFFWVQLCIPLAAVIVLMRCCSCCLPFLVVAGAYLAKVDAYEHATTVPNVPQIPYKALVERAGYAPLNLEITVMSSEVLPSTNQEYITCKFTTVVPSPKVKCCGSLECQPAAHADYTCKVFGGVYPFMWGGAQCFCDSENSQMSEAYVELSADCATDHAQAIKVHTAAMKVGLRIVYGNTTSFLDVYVNGVTPGTSKDLKVIAGPISASFTPFDHKVVIHRGLVYNYDFPEYGAMKPGAFGDIQATSLTSKDLIASTDIRLLKPSAKNVHVPYTQAASGFEMWKNNSGRPLQETAPFGCKIAVNPLRAVDCSYGNIPISIDIPNAAFIRTSDAPLVSTVKCDVSECTYSADFGGMATLQYVSDREGQCPVHSHSSTATLQESTVHVLEKGAVTVHFSTASPQANFIVSLCGKKTTCNAECKPPADHIVSTPHKNDQEFQAAISKTSWSWLFALFGGASSLLIIGLMIFACSMMLTSTRR

SEQ ID NO: 132 - protein, 977 aa, Sindbis Virus
SAAPLVTAMCLLGNVSFPCNRPPTCYTREPSRALDILEENVNHEAYDTLLNAILRCGSSGSVTDDFTLTSPYLGTCSYCHHTEPCFSPIKIEQVWDEADDNTIRIQTSAQFGYDQSGAASSNKYRYMAAAADHTVKEGTMDDIKISTSGPCRRLSYKGYFLLAKCPPGDSVTVSIASSNSATSCTMARKIKPKFVGREKYDLPPVHGKKIPCTVYDRLAATTAGYITMHRPGPHAYTSYLEESSGKVYAKPPSGKNITYECKCGDYKTGTVTTRTEITGCTAIKQCVAYKSDQTKWVFNSPDLIRHADHTAQGKLHLPFKLIPSTCMVPVAHAPNVVHGFKHISLQLDTDHLTLLTTRRLGANPEPTTEWIIGKTVRNFTVDRDGLEYIWGNHEPVRVYAQESAPGDPHGWPHEIVQHYYHRHPVYTILAVASAAVAMMIGVTVAALCACKARRECLTPYALAPNAVIPTSLALLCCVRSANAETFTETMSYLWSNSQPFFWVQLCIPLAAVIVLMRCCSCCLPFLVVAGAYLAKVDAYEHATTVPNVPQIPYKALVERAGYAPLNLEITVMSSEVLPSTNQEYITCKFTTVVPSPKVKCCGSLECQPAAHADYTCKVFGGVYPFMWGGAQCFCDSENSQMSEAYVELSADCATDHAQAIKVHTAAMKVGLRIVYGNTTSFLDVYVNGVTPGTSKDLKVIAGPISASFTPFDHKVVIHRGLVYNYDFPEYGAMKPGAFGDIQATSLTSKDLIASTDIRLLKPSAKNVHVPYTQAASGFEMWKNNSGRPLQETAPFGCKIAVNPLRAVDCSYGNIPISIDIPNAAFIRTSDAPLVSTVKCDVSECTYSADFGGMATLQYVSDREGQCPVHSHSSTATLQESTVHVLEKGAVTVHFSTASPQANFIVSLCGKKTTCNAECKPPADHIVSTPHKNDQEFQAAISKTSWSWLFALFGGASSLLIIGLMIFACSMMLTSTRR

SEQ ID NO: 133 - protein, 985 aa, Sindbis Virus
SAAPLVTAMCLLGNVSFPCNRPPTCYTREPSRALDILEENVNHEAYDTLLNAILRCGSSGSVTDDFTLTSPYLGTCSYCHHTEPCFSPIKIEQVWDEADDNTIRIQTSAQFGYDQSGAASSNKYRYMAAAAMYPYDVPDYATVKEGTMDDIKISTSGPCRRLSYKGYFLLAKCPPGDSVTVSIASSNSATSCTMARKIKPKFVGREKYDLPPVHGKKIPCTVYDRLAATTAGYITMHRPGPHAYTSYLEESSGKVYAKPPSGKNITYECKCGDYKTGTVTTRTEITGCTAIKQCVAYKSDQTKWVFNSPDLIRHADHTAQGKLHLPFKLIPSTCMVPVAHAPNVVHGFKHISLQLDTDHLTLLTTRRLGANPEPTTEWIIGKTVRNFTVDRDGLEYIWGNHEPVRVYAQESAPGDPHGWPHEIVQHYYHRHPVYTILAVASAAVAMMIGVTVAALCACKARRECLTPYALAPNAVIPTSLALLCCVRSANAETFTETMSYLWSNSQPFFWVQLCIPLAAVIVLMRCCSCCLPFLVVAGAYLAKVDAYEHATTVPNVPQIPYKALVERAGYAPLNLEITVMSSEVLPSTNQEYITCKFTTVVPSPKVKCCGSLECQPAAHADYTCKVFGGVYPFMWGGAQCFCDSENSQMSEAYVELSADCATDHAQAIKVHTAAMKVGLRIVYGNTTSFLDVYVNGVTPGTSKDLKVIAGPISASFTPFDHKVVIHRGLVYNYDFPEYGAMKPGAFGDIQATSLTSKDLIASTDIRLLKPSAKNVHVPYTQAASGFEMWKNNSGRPLQETAPFGCKIAVNPLRAVDCSYGNIPISIDIPNAAFIRTSDAPLVSTVKCDVSECTYSADFGGMATLQYVSDREGQCPVHSHSSTATLQESTVHVLEKGAVTVHFSTASPQANFIVSLCGKKTTCNAECKPPADHIVSTPHKNDQEFQAAISKTSWSWLFALFGGASSLLIIGLMIFACSMMLTSTRR

SEQ ID NO: 134 - protein, 975 aa, Sindbis Virus
SAAPLVTAMCLLGNVSFPCNRPPTCYTREPSRALDILEENVNHEAYDTLLNAILRCGSSGSVTDDFTLTSPYLGTCSYCHHTEPCFSPIKIEQVWDEADDNTIRIQTSAQFGYDQSGAASSNKYRYMAAAATVKEGTMDDIKISTSGPCRRLSYKGYFLLAKCPPGDSVTVSIASSNSATSCTMARKIKPKFVGREKYDLPPVHGKKIPCTVYDRLAATTAGYITMHRPGPHAYTSYLEESSGKVYAKPPSGKNITYECKCGDYKTGTVTTRTEITGCTAIKQCVAYKSDQTKWVFNSPDLIRHADHTAQGKLHLPFKLIPSTCMVPVAHAPNVVHGFKHISLQLDTDHLTLLTTRRLGANPEPTTEWIIGKTVRNFTVDRDGLEYIWGNHEPVRVYAQESAPGDPHGWPHEIVQHYYHRHPVYTILAVASAAVAMMIGVTVAALCACKARRECLTPYALAPNAVIPTSLALLCCVRSANAETFTETMSYLWSNSQPFFWVQLCIPLAAVIVLMRCCSCCLPFLVVAGAYLAKVDAYEHATTVPNVPQIPYKALVERAGYAPLNLEITVMSSEVLPSTNQEYITCKFTTVVPSPKVKCCGSLECQPAAHADYTCKVFGGVYPFMWGGAQCFCDSENSQMSEAYVELSADCATDHAQAIKVHTAAMKVGLRIVYGNTTSFLDVYVNGVTPGTSKDLKVIAGPISASFTPFDHKVVIHRGLVYNYDFPEYGAMKPGAFGDIQATSLTSKDLIASTDIRLLKPSAKNVHVPYTQAASGFEMWKNNSGRPLQETAPFGCKIAVNPLRAVDCSYGNIPISIDIPNAAFIRTSDAPLVSTVKCDVSECTYSADFGGMATLQYVSDREGQCPVHSHSSTATLQESTVHVLEKGAVTVHFSTASPQANFIVSLCGKKTTCNAECKPPADHIVSTPHKNDQEFQAAISKTSWSWLFALFGGASSLLIIGLMIFACSMMLTSTRR

SEQ ID NO: 135 - protein, 977 aa, Sindbis Virus
SAAPLVTAMCLLGNVSFPCDRPPTCYTREPSRALDILEENVNHEAYDTLLNAILRCGSSGSVIDDFTLTSPYLGTCSYCHHTEPCFSPVKIEQVWDEADDNTIRIQTSAQFGYDQSGAASANKYRYMAAAADHTVKEGTMDDIKISTSGPCRRLSYKGYFLLAKCPPGDSVTVSIVSSNSATSCTLARKIKPKFVGREKYDLPPVHGKKIPCTVYDRLAATTAGYITMHRPGPHAYTSYLEESSGKVYAKPPSGKNITYECKCGDYKTGTVSTRTEITGCTAIKQCVAYKSDQTKWVFNSPDLIRHDDHTVQGKLHLPFKLIPSTCMVPVAHAPNVIHGFKHISLQLDTDHLTLLTTRRLGANPEPTTEWIVGKTVRNFTVDRDGLEYIWGNHEPVRVYAQESAPGDPHGWPHEIVQHYYHRHPVYTILAVASATVAMMIGVTVAVLCACKARRECLTPYALAPNAVIPTSLALLCCVRSANAETFTETMSYLWSNSQPFFWVQLCIPLAAFIVLMRCCSCCLPFLVVAGAYLAKVDAYEHATTVPNVPQIPYKALVERAGYAPLNLEITVMSSEVLPSTNQEYITCKFTTVVPSPKIKCCGSLECQPAAHAGYTCKVFGGVYPFMWGGAQCFCDSENSQMSEAYVELSADCASDHAQAIKVHTAAMKVGLRIVYGNTTSFLDVYVNGVTPGTSKDLKVIAGPISASFTPFDHKVVIHRGLVYNYDFPEYGAMKPGAFGDIQATSLTSKDLIASTDIRLLKPSAKNVHVPYTQASSGFEMWKNNSGRPLQETAPFGCKIAVNPLRAVDCSYGNIPISIDIPNAAFIRTSDAPLVSTVKCEVSECTYSADFGGMATLQYVSDREGQCPVHSHSSTATLQESTVHVLEKGAVTVHFSTASPQANFIVSLCGKKTTCNAECKPPADHIVSTPHKNDQEFQAAISKTSWSWLFALFGGASSLLIIGLMIFACSMMLTSTRR

SEQ ID NO: 136 - protein, 985 aa, Sindbis Virus
SAAPLVTAMCLLGNVSFPCDRPPTCYTREPSRALDILEENVNHEAYDTLLNAILRCGSSGSVIDDFTLTSPYLGTCSYCHHTEPCFSPVKIEQVWDEADDNTIRIQTSAQFGYDQSGAASANKYRYMAAAAMYPYDVPDYATVKEGTMDDIKISTSGPCRRLSYKGYFLLAKCPPGDSVTVSIVSSNSATSCTLARKIKPKFVGREKYDLPPVHGKKIPCTVYDRLAATTAGYITMHRPGPHAYTSYLEESSGKVYAKPPSGKNITYECKCGDYKTGTVSTRTEITGCTAIKQCVAYKSDQTKWVFNSPDLIRHDDHTVQGKLHLPFKLIPSTCMVPVAHAPNVIHGFKHISLQLDTDHLTLLTTRRLGANPEPTTEWIVGKTVRNFTVDRDGLEYIWGNHEPVRVYAQESAPGDPHGWPHEIVQHYYHRHPVYTILAVASATVAMMIGVTVAVLCACKARRECLTPYALAPNAVIPTSLALLCCVRSANAETFTETMSYLWSNSQPFFWVQLCIPLAAFIVLMRCCSCCLPFLVVAGAYLAKVDAYEHATTVPNVPQIPYKALVERAGYAPLNLEITVMSSEVLPSTNQEYITCKFTTVVPSPKIKCCGSLECQPAAHAGYTCKVFGGVYPFMWGGAQCFCDSENSQMSEAYVELSADCASDHAQAIKVHTAAMKVGLRIVYGNTTSFLDVYVNGVTPGTSKDLKVIAGPISASFTPFDHKVVIHRGLVYNYDFPEYGAMKPGAFGDIQATSLTSKDLIASTDIRLLKPSAKNVHVPYTQASSGFEMWKNNSGRPLQETAPFGCKIAVNPLRAVDCSYGNIPISIDIPNAAFIRTSDAPLVSTVKCEVSECTYSADFGGMATLQYVSDREGQCPVHSHSSTATLQESTVHVLEKGAVTVHFSTASPQANFIVSLCGKKTTCNAECKPPADHIVSTPHKNDQEFQAAISKTSWSWLFALFGGASSLLIIGLMIFACSMMLTSTRR

SEQ ID NO: 137 - protein, 975 aa, synthetic construct
SAAPLVTAMCLLGNVSFPCDRPPTCYTREPSRALDILEENVNHEAYDTLLNAILRCGSSGSVIDDFTLTSPYLGTCSYCHHTEPCFSPVKIEQVWDEADDNTIRIQTSAQFGYDQSGAASANKYRYMAAAATVKEGTMDDIKISTSGPCRRLSYKGYFLLAKCPPGDSVTVSIVSSNSATSCTLARKIKPKFVGREKYDLPPVHGKKIPCTVYDRLAATTAGYITMHRPGPHAYTSYLEESSGKVYAKPPSGKNITYECKCGDYKTGTVSTRTEITGCTAIKQCVAYKSDQTKWVFNSPDLIRHDDHTVQGKLHLPFKLIPSTCMVPVAHAPNVIHGFKHISLQLDTDHLTLLTTRRLGANPEPTTEWIVGKTVRNFTVDRDGLEYIWGNHEPVRVYAQESAPGDPHGWPHEIVQHYYHRHPVYTILAVASATVAMMIGVTVAVLCACKARRECLTPYALAPNAVIPTSLALLCCVRSANAETFTETMSYLWSNSQPFFWVQLCIPLAAFIVLMRCCSCCLPFLVVAGAYLAKVDAYEHATTVPNVPQIPYKALVERAGYAPLNLEITVMSSEVLPSTNQEYITCKFTTVVPSPKIKCCGSLECQPAAHAGYTCKVFGGVYPFMWGGAQCFCDSENSQMSEAYVELSADCASDHAQAIKVHTAAMKVGLRIVYGNTTSFLDVYVNGVTPGTSKDLKVIAGPISASFTPFDHKVVIHRGLVYNYDFPEYGAMKPGAFGDIQATSLTSKDLIASTDIRLLKPSAKNVHVPYTQASSGFEMWKNNSGRPLQETAPFGCKIAVNPLRAVDCSYGNIPISIDIPNAAFIRTSDAPLVSTVKCEVSECTYSADFGGMATLQYVSDREGQCPVHSHSSTATLQESTVHVLEKGAVTVHFSTASPQANFIVSLCGKKTTCNAECKPPADHIVSTPHKNDQEFQAAISKTSWSWLFALFGGASSLLIIGLMIFACSMMLTSTRR

SEQ ID NO: 138 - protein, 977 aa, Sindbis Virus
SAAPLVTAMCLLGNVSFPCNRPPTCYTREPSRALDILEENVNHEAYDTLLNAILRCGSSGSVTDDFTLTSPYLGTCSYCHHTEPCFSPIKIEQVWDEADDNTIRIQTSAQFGYDQSGAASSNKYRYMAAAADHTVKEGTMDDIKISTSGPCRRLSYKGYFLLAKCPPGDSVTVSIASSNSATSCTMARKIKPKFVGREKYDLPPVHGKKIPCTVYDRLAATTAGYITMHRPGPHAYTSYLEESSGKVYAKPPSGKNITYECKCGDYKTGTVTTRTEITGCTAIKQCVAYKSDQTKWVFNSPDLIRHADHTAQGKLHLPFKLIPSTCMVPVAHAPNVVHGFKHISLQLDTDHLTLLTTRRLGANPEPTTEWIIGKTVRNFTVDRDGLEYIWGNHEPVRVYAQESAPGDPHGWPHEIVQHYYHRHPVYTILAVASAAVAMMIGVTVAALCACKARRECLTPYALAPNAVIPTSLALLCCVRSANAETFTETMSYLWSNSQPFFWVQLCIPLAAVIVLMRCCSCCLPFLVVAGAYLAKVDAYEHATTVPNVPQIPYKALVERAGYAPLNLEITVMSSEVLPSTNQEYITCKFTTVVPSPKVKCCGSLECQPAAHADYTCKVFGGVYPFMWGGAQCFCDSENSQMSEAYVELSADCATDHAQAIKVHTAAMKVGLRIVYGNTTSFLDVYVNGVTPGTSKDLKVIAGPISASFTPFDHKVVIHRGLVYNYDFPEYGAMKPGAFGDIQATSLTSKDLIASTDIRLLKPSSGNVHVPYTQAASGFEMWKNNSGRPLQETAPFGCKIAVNPLRAVDCSYGNIPISIDIPNAAFIRTSDAPLVSTVKCDVSECTYSADFGGMATLQYVSDREGQCPVHSHSSTATLQESTVHVLEKGAVTVHFSTASPQANFIVSLCGKKTTCNAECKPPADHIVSTPHKNDQEFQAAISKTSWSWLFALFGGASSLLIIGLMIFACSMMLTSTRR

SEQ ID NO: 139 - protein, 985 aa, Sindbis Virus
SAAPLVTAMCLLGNVSFPCNRPPTCYTREPSRALDILEENVNHEAYDTLLNAILRCGSSGSVTDDFTLTSPYLGTCSYCHHTEPCFSPIKIEQVWDEADDNTIRIQTSAQFGYDQSGAASSNKYRYMAAAAMYPYDVPDYATVKEGTMDDIKISTSGPCRRLSYKGYFLLAKCPPGDSVTVSIASSNSATSCTMARKIKPKFVGREKYDLPPVHGKKIPCTVYDRLAATTAGYITMHRPGPHAYTSYLEESSGKVYAKPPSGKNITYECKCGDYKTGTVTTRTEITGCTAIKQCVAYKSDQTKWVFNSPDLIRHADHTAQGKLHLPFKLIPSTCMVPVAHAPNVVHGFKHISLQLDTDHLTLLTTRRLGANPEPTTEWIIGKTVRNFTVDRDGLEYIWGNHEPVRVYAQESAPGDPHGWPHEIVQHYYHRHPVYTILAVASAAVAMMIGVTVAALCACKARRECLTPYALAPNAVIPTSLALLCCVRSANAETFTETMSYLWSNSQPFFWVQLCIPLAAVIVLMRCCSCCLPFLVVAGAYLAKVDAYEHATTVPNVPQIPYKALVERAGYAPLNLEITVMSSEVLPSTNQEYITCKFTTVVPSPKVKCCGSLECQPAAHADYTCKVFGGVYPFMWGGAQCFCDSENSQMSEAYVELSADCATDHAQAIKVHTAAMKVGLRIVYGNTTSFLDVYVNGVTPGTSKDLKVIAGPISASFTPFDHKVVIHRGLVYNYDFPEYGAMKPGAFGDIQATSLTSKDLIASTDIRLLKPSSGNVHVPYTQAASGFEMWKNNSGRPLQETAPFGCKIAVNPLRAVDCSYGNIPISIDIPNAAFIRTSDAPLVSTVKCDVSECTYSADFGGMATLQYVSDREGQCPVHSHSSTATLQESTVHVLEKGAVTVHFSTASPQANFIVSLCGKKTTCNAECKPPADHIVSTPHKNDQEFQAAISKTSWSWLFALFGGASSLLIIGLMIFACSMMLTSTRR

SEQ ID NO: 140 - protein, 975 aa, synthetic construct
SAAPLVTAMCLLGNVSFPCNRPPTCYTREPSRALDILEENVNHEAYDTLLNAILRCGSSGSVTDDFTLTSPYLGTCSYCHHTEPCFSPIKIEQVWDEADDNTIRIQTSAQFGYDQSGAASSNKYRYMAAAATVKEGTMDDIKISTSGPCRRLSYKGYFLLAKCPPGDSVTVSIASSNSATSCTMARKIKPKFVGREKYDLPPVHGKKIPCTVYDRLAATTAGYITMHRPGPHAYTSYLEESSGKVYAKPPSGKNITYECKCGDYKTGTVTTRTEITGCTAIKQCVAYKSDQTKWVFNSPDLIRHADHTAQGKLHLPFKLIPSTCMVPVAHAPNVVHGFKHISLQLDTDHLTLLTTRRLGANPEPTTEWIIGKTVRNFTVDRDGLEYIWGNHEPVRVYAQESAPGDPHGWPHEIVQHYYHRHPVYTILAVASAAVAMMIGVTVAALCACKARRECLTPYALAPNAVIPTSLALLCCVRSANAETFTETMSYLWSNSQPFFWVQLCIPLAAVIVLMRCCSCCLPFLVVAGAYLAKVDAYEHATTVPNVPQIPYKALVERAGYAPLNLEITVMSSEVLPSTNQEYITCKFTTVVPSPKVKCCGSLECQPAAHADYTCKVFGGVYPFMWGGAQCFCDSENSQMSEAYVELSADCATDHAQAIKVHTAAMKVGLRIVYGNTTSFLDVYVNGVTPGTSKDLKVIAGPISASFTPFDHKVVIHRGLVYNYDFPEYGAMKPGAFGDIQATSLTSKDLIASTDIRLLKPSSGNVHVPYTQAASGFEMWKNNSGRPLQETAPFGCKIAVNPLRAVDCSYGNIPISIDIPNAAFIRTSDAPLVSTVKCDVSECTYSADFGGMATLQYVSDREGQCPVHSHSSTATLQESTVHVLEKGAVTVHFSTASPQANFIVSLCGKKTTCNAECKPPADHIVSTPHKNDQEFQAAISKTSWSWLFALFGGASSLLIIGLMIFACSMMLTSTRR

SEQ ID NO: 141 - protein, 977 aa, Sindbis Virus
SAAPLVTAMCLLGNVSFPCDRPPTCYTREPSRALDILEENVNHEAYDTLLNAILRCGSSGSVIDDFTLTSPYLGTCSYCHHTEPCFSPVKIEQVWDEADDNTIRIQTSAQFGYDQSGAASANKYRYMAAAADHTVKEGTMDDIKISTSGPCRRLSYKGYFLLAKCPPGDSVTVSIVSSNSATSCTLARKIKPKFVGREKYDLPPVHGKKIPCTVYDRLAATTAGYITMHRPGPHAYTSYLEESSGKVYAKPPSGKNITYECKCGDYKTGTVSTRTEITGCTAIKQCVAYKSDQTKWVFNSPDLIRHDDHTVQGKLHLPFKLIPSTCMVPVAHAPNVIHGFKHISLQLDTDHLTLLTTRRLGANPEPTTEWIVGKTVRNFTVDRDGLEYIWGNHEPVRVYAQESAPGDPHGWPHEIVQHYYHRHPVYTILAVASATVAMMIGVTVAVLCACKARRECLTPYALAPNAVIPTSLALLCCVRSANAETFTETMSYLWSNSQPFFWVQLCIPLAAFIVLMRCCSCCLPFLVVAGAYLAKVDAYEHATTVPNVPQIPYKALVERAGYAPLNLEITVMSSEVLPSTNQEYITCKFTTVVPSPKIKCCGSLECQPAAHAGYTCKVFGGVYPFMWGGAQCFCDSENSQMSEAYVELSADCASDHAQAIKVHTAAMKVGLRIVYGNTTSFLDVYVNGVTPGTSKDLKVIAGPISASFTPFDHKVVIHRGLVYNYDFPEYGAMKPGAFGDIQATSLTSKDLIASTDIRLLKPSSGNVHVPYTQASSGFEMWKNNSGRPLQETAPFGCKIAVNPLRAVDCSYGNIPISIDIPNAAFIRTSDAPLVSTVKCEVSECTYSADFGGMATLQYVSDREGQCPVHSHSSTATLQESTVHVLEKGAVTVHFSTASPQANFIVSLCGKKTTCNAECKPPADHIVSTPHKNDQEFQAAISKTSWSWLFALFGGASSLLIIGLMIFACSMMLTSTRR

SEQ ID NO: 142 - protein, 985 aa, Sindbis Virus
SAAPLVTAMCLLGNVSFPCDRPPTCYTREPSRALDILEENVNHEAYDTLLNAILRCGSSGSVIDDFTLTSPYLGTCSYCHHTEPCFSPVKIEQVWDEADDNTIRIQTSAQFGYDQSGAASANKYRYMAAAAMYPYDVPDYATVKEGTMDDIKISTSGPCRRLSYKGYFLLAKCPPGDSVTVSIVSSNSATSCTLARKIKPKFVGREKYDLPPVHGKKIPCTVYDRLAATTAGYITMHRPGPHAYTSYLEESSGKVYAKPPSGKNITYECKCGDYKTGTVSTRTEITGCTAIKQCVAYKSDQTKWVFNSPDLIRHDDHTVQGKLHLPFKLIPSTCMVPVAHAPNVIHGFKHISLQLDTDHLTLLTTRRLGANPEPTTEWIVGKTVRNFTVDRDGLEYIWGNHEPVRVYAQESAPGDPHGWPHEIVQHYYHRHPVYTILAVASATVAMMIGVTVAVLCACKARRECLTPYALAPNAVIPTSLALLCCVRSANAETFTETMSYLWSNSQPFFWVQLCIPLAAFIVLMRCCSCCLPFLVVAGAYLAKVDAYEHATTVPNVPQIPYKALVERAGYAPLNLEITVMSSEVLPSTNQEYITCKFTTVVPSPKIKCCGSLECQPAAHAGYTCKVFGGVYPFMWGGAQCFCDSENSQMSEAYVELSADCASDHAQAIKVHTAAMKVGLRIVYGNTTSFLDVYVNGVTPGTSKDLKVIAGPISASFTPFDHKVVIHRGLVYNYDFPEYGAMKPGAFGDIQATSLTSKDLIASTDIRLLKPSSGNVHVPYTQASSGFEMWKNNSGRPLQETAPFGCKIAVNPLRAVDCSYGNIPISIDIPNAAFIRTSDAPLVSTVKCEVSECTYSADFGGMATLQYVSDREGQCPVHSHSSTATLQESTVHVLEKGAVTVHFSTASPQANFIVSLCGKKTTCNAECKPPADHIVSTPHKNDQEFQAAISKTSWSWLFALFGGASSLLIIGLMIFACSMMLTSTRR

SEQ ID NO: 143 - protein, 975 aa, synthetic construct
SAAPLVTAMCLLGNVSFPCDRPPTCYTREPSRALDILEENVNHEAYDTLLNAILRCGSSGSVIDDFTLTSPYLGTCSYCHHTEPCFSPVKIEQVWDEADDNTIRIQTSAQFGYDQSGAASANKYRYMAAAATVKEGTMDDIKISTSGPCRRLSYKGYFLLAKCPPGDSVTVSIVSSNSATSCTLARKIKPKFVGREKYDLPPVHGKKIPCTVYDRLAATTAGYITMHRPGPHAYTSYLEESSGKVYAKPPSGKNITYECKCGDYKTGTVSTRTEITGCTAIKQCVAYKSDQTKWVFNSPDLIRHDDHTVQGKLHLPFKLIPSTCMVPVAHAPNVIHGFKHISLQLDTDHLTLLTTRRLGANPEPTTEWIVGKTVRNFTVDRDGLEYIWGNHEPVRVYAQESAPGDPHGWPHEIVQHYYHRHPVYTILAVASATVAMMIGVTVAVLCACKARRECLTPYALAPNAVIPTSLALLCCVRSANAETFTETMSYLWSNSQPFFWVQLCIPLAAFIVLMRCCSCCLPFLVVAGAYLAKVDAYEHATTVPNVPQIPYKALVERAGYAPLNLEITVMSSEVLPSTNQEYITCKFTTVVPSPKIKCCGSLECQPAAHAGYTCKVFGGVYPFMWGGAQCFCDSENSQMSEAYVELSADCASDHAQAIKVHTAAMKVGLRIVYGNTTSFLDVYVNGVTPGTSKDLKVIAGPISASFTPFDHKVVIHRGLVYNYDFPEYGAMKPGAFGDIQATSLTSKDLIASTDIRLLKPSSGNVHVPYTQASSGFEMWKNNSGRPLQETAPFGCKIAVNPLRAVDCSYGNIPISIDIPNAAFIRTSDAPLVSTVKCEVSECTYSADFGGMATLQYVSDREGQCPVHSHSSTATLQESTVHVLEKGAVTVHFSTASPQANFIVSLCGKKTTCNAECKPPADHIVSTPHKNDQEFQAAISKTSWSWLFALFGGASSLLIIGLMIFACSMMLTSTRR

SEQ ID NO: 144 - protein, 617 aa, Measles morbillivirus
MSPQRDRINAFYKDNPHPKGSRIVINREHLMIDRPYVLLAVLFVMFLSLIGLLAIAGIRLHRAAIYTAEIHKSLSTNLDVTNSIEHQVKDVLTPLFKIIGDEVGLRTPQRFTDLVKFISDKIKFLNPDREYDFRDLTWCINPPERIKLDYDQYCADVAAEELMNALVNSTLLETRTTNQFLAVSKGNCSGPTTIRGQFSNMSLSLLDLYLGRGYNVSSIVTMTSQGMYGGTYLVEKPNLSSKRSELSQLSMYRVFEVGVIRNPGLGAPVFHMTNYLEQPVSNDLSNCMVALGELKLAALCHGEDSITIPYQGSGKGVSFQLVKLGVWKSPTDMQSWVPLSTDDPVIDRLYLSSHRGVIADNQAKWAVPTTRTDDKLRMETCFQQACKGKIQALCENPEWAPLKDNRIPSYGVLSVDLSLTVELKIKIASGFGPLITHGSGMDLYKSNHNNVYWLTIPPMKNLALGVINTLEWIPRFKVSPYLFNVPIKEAGEDCHAPTYLPAEVDGDVKLSSNLVILPGQDLQYVLATYDTSRVEHAVVYYVYSPSRSFSYFYPFRLPIKGVPIELQVECFTWDQKLWCRHFCVLADSESGGHITHSGMEGMGVSCTVTREDGTNRR

SEQ ID NO: 145 - protein, 599 aa, Measles morbillivirus
MGSRIVINREHLMIDRPYVLLAVLFVMFLSLIGLLAIAGIRLHRAAIYTAEIHKSLSTNLDVTNSIEHQVKDVLTPLFKIIGDEVGLRTPQRFTDLVKFISDKIKFLNPDREYDFRDLTWCINPPERIKLDYDQYCADVAAEELMNALVNSTLLETRTTNQFLAVSKGNCSGPTTIRGQFSNMSLSLLDLYLGRGYNVSSIVTMTSQGMYGGTYLVEKPNLSSKRSELSQLSMYRVFEVGVIRNPGLGAPVFHMTNYLEQPVSNDLSNCMVALGELKLAALCHGEDSITIPYQGSGKGVSFQLVKLGVWKSPTDMQSWVPLSTDDPVIDRLYLSSHRGVIADNQAKWAVPTTRTDDKLRMETCFQQACKGKIQALCENPEWAPLKDNRIPSYGVLSVDLSLTVELKIKIASGFGPLITHGSGMDLYKSNHNNVYWLTIPPMKNLALGVINTLEWIPRFKVSPYLFTVPIKEAGGDCHAPTYLPAEVDGDVKLSSNLVILPGQDLQYVLATYDTSRVEHAVVYYVYSPSRSFSYFYPFRLPIKGVPIELQVECFTWDQKLWCRHFCVLADSESGGHITHSGMVGMGVSCTVTREDGTNRR

SEQ ID NO: 146 - protein, 599 aa, Measles morbillivirus
MGSRIVINREHLMIDRPYVLLAVLFVMFLSLIGLLAIAGIRLHRAAIYTAEIHKSLSTNLDVTNSIEHQVKDVLTPLFKIIGDEVGLRTPQRFTDLVKFISDKIKFLNPDREYDFRDLTWCINPPERIKLDYDQYCADVAAEELMNALVNSTLLETRTTNQFLAVSKGNCSGPTTIRGQFSNMSLSLLDLYLGRGYNVSSIVTMTSQGMYGGTYLVEKPNLSSKRSELSQLSMYRVFEVGVIRNPGLGAPVFHMTNYLEQPVSNDLSNCMVALGELKLAALCHGEDSITIPYQGSGKGVSFQLVKLGVWKSPTDMQSWVPLSTDDPVIDRLYLSSHRGVIADNQAKWAVPTTRTDDKLRMETCFQQACKGKIQALCENPEWAPLKDNRIPSYGVLSVDLSLTVELKIKIASGFGPLITHGSGMDLYKSNHNNVYWLTIPPMKNLALGVINTLEWIPRFKVSPALFNVPIKEAGGDCHAPTYLPAEVDGDVKLSSNLVILPGQDLQYVLATYDTSAVEHAVVYYVYSPSRSFSYFYPFRLPIKGVPIELQVECFTWDQKLWCRHFCVLADSESGGHITHSGMVGMGVSCTVTREAAARGS

SEQ ID NO: 147 - protein, 599 aa, synthetic construct; identical to SEQ ID NO: 146

SEQ ID NO: 148 - protein, 599 aa, Measles morbillivirus
MGSRIVINREHLMIDRPYVLLAVLFVMFLSLIGLLAIAGIRLHRAAIYTAEIHKSLSTNLDVTNSIEHQVKDVLTPLFKIIGDEVGLRTPQRFTDLVKFISDKIKFLNPDREYDFRDLTWCINPPERIKLDYDQYCADVAAEELMNALVNSTLLETRTTNQFLAVSKGNCSGPTTIRGQFSNMSLSLLDLYLGRGYNVSSIVTMTSQGMYGGTYLVEKPNLSSKRSELSQLSMYRVFEVGVIRNPGLGAPVFHMTNYLEQPVSNDLSNCMVALGELKLAALCHGEDSITIPYQGSGKGVSFQLVKLGVWKSPTDMQSWVPLSTDDPVIDRLYLSSHRGVIADNQAKWAVPTTRTDDKLRMETCFQQACKGKIQALCENPEWAPLKDNRIPSYGVLSVDLSLTVELKIKIASGFGPLITHGSGMDLYKSNHNNVYWLTIPPMKNLALGVINTLEWIPRFKVSPALFNVPIKEAGGDCHAPTYLPAEVDGDVKLSSNLVILPGQDLQYVLATYDTSAVEHAVVYYVYSPSRLSSYFYPFRLPIKGVPIELQVECFTWDQKLWCRHFCVLADSESGGHITHSGMVGMGVSCTVTREAAARGS

SEQ ID NO: 149 - protein, 599 aa, synthetic construct; identical to SEQ ID NO: 148

SEQ ID NO: 150 - protein, 598 aa, Measles morbillivirus
MSRIVINREHLMIDRPYVLLAVLFVMFLSLIGLLAIAGIRLHRAAIYTAEIHKSLSTNLDVTNSIEHQVKDVLTPLFKIIGDEVGLRTPQRFTDLVKFISDKIKFLNPDREYDFRDLTWCINPPERIKLDYDQYCADVAAEELMNALVNSTLLETRTTNQFLAVSKGNCSGPTTIRGQFSNMSLSLLDLYLGRGYNVSSIVTMTSQGMYGGTYLVEKPNLSSKRSELSQLSMYRVFEVGVIRNPGLGAPVFHMTNYLEQPVSNDLSNCMVALGELKLAALCHGEDSITIPYQGSGKGVSFQLVKLGVWKSPTDMQSWVPLSTDDPVIDRLYLSSHRGVIADNQAKWAVPTTRTDDKLRMETCFQQACKGKIQALCENPEWAPLKDNRIPSYGVLSVDLSLTVELKIKIASGFGPLITHGSGMDLYKSNHNNVYWLTIPPMKNLALGVINTLEWIPRFKVSPYLFTVPIKEAGGDCHAPTYLPAEVDGDVKLSSNLVILPGQDLQYVLATYDTSRVEHAVVYYVYSPSRSFSYFYPFRLPIKGVPIELQVECFTWDQKLWCRHFCVLADSESGGHITHSGMVGMGVSCTVTREDGTNRR

SEQ ID NO: 151 - protein, 598 aa, Measles morbillivirus
MSRIVINREHLMIDRPYVLLAVLFVMFLSLIGLLAIAGIRLHRAAIYTAEIHKSLSTNLDVTNSIEHQVKDVLTPLFKIIGDEVGLRTPQRFTDLVKFISDKIKFLNPDREYDFRDLTWCINPPERIKLDYDQYCADVAAEELMNALVNSTLLETRTTNQFLAVSKGNCSGPTTIRGQFSNMSLSLLDLYLGRGYNVSSIVTMTSQGMYGGTYLVEKPNLSSKRSELSQLSMYRVFEVGVIRNPGLGAPVFHMTNYLEQPVSNDLSNCMVALGELKLAALCHGEDSITIPYQGSGKGVSFQLVKLGVWKSPTDMQSWVPLSTDDPVIDRLYLSSHRGVIADNQAKWAVPTTRTDDKLRMETCFQQACKGKIQALCENPEWAPLKDNRIPSYGVLSVDLSLTVELKIKIASGFGPLITHGSGMDLYKSNHNNVYWLTIPPMKNLALGVINTLEWIPRFKVSPALFNVPIKEAGGDCHAPTYLPAEVDGDVKLSSNLVILPGQDLQYVLATYDTSAVEHAVVYYVYSPSRSFSYFYPFRLPIKGVPIELQVECFTWDQKLWCRHFCVLADSESGGHITHSGMVGMGVSCTVTREAAARGS

SEQ ID NO: 152 - protein, 598 aa, synthetic construct; identical to SEQ ID NO: 151

SEQ ID NO: 153 - protein, 598 aa, Measles morbillivirus
MSRIVINREHLMIDRPYVLLAVLFVMFLSLIGLLAIAGIRLHRAAIYTAEIHKSLSTNLDVTNSIEHQVKDVLTPLFKIIGDEVGLRTPQRFTDLVKFISDKIKFLNPDREYDFRDLTWCINPPERIKLDYDQYCADVAAEELMNALVNSTLLETRTTNQFLAVSKGNCSGPTTIRGQFSNMSLSLLDLYLGRGYNVSSIVTMTSQGMYGGTYLVEKPNLSSKRSELSQLSMYRVFEVGVIRNPGLGAPVFHMTNYLEQPVSNDLSNCMVALGELKLAALCHGEDSITIPYQGSGKGVSFQLVKLGVWKSPTDMQSWVPLSTDDPVIDRLYLSSHRGVIADNQAKWAVPTTRTDDKLRMETCFQQACKGKIQALCENPEWAPLKDNRIPSYGVLSVDLSLTVELKIKIASGFGPLITHGSGMDLYKSNHNNVYWLTIPPMKNLALGVINTLEWIPRFKVSPALFNVPIKEAGGDCHAPTYLPAEVDGDVKLSSNLVILPGQDLQYVLATYDTSAVEHAVVYYVYSPSRLSSYFYPFRLPIKGVPIELQVECFTWDQKLWCRHFCVLADSESGGHITHSGMVGMGVSCTVTREAAARGS

SEQ ID NO: 154 - protein, 598 aa, synthetic construct; identical to SEQ ID NO: 153

SEQ ID NO: 155 - protein, 597 aa, Measles morbillivirus
MAAAANREHLMIDRPYVLLAVLFVMFLSLIGLLAIAGIRLHRAAIYTAEIHKSLSTNLDVTNSIEHQVKDVLTPLFKIIGDEVGLRTPQRFTDLVKFISDKIKFLNPDREYDFRDLTWCINPPERIKLDYDQYCADVAAEELMNALVNSTLLETRTTNQFLAVSKGNCSGPTTIRGQFSNMSLSLLDLYLGRGYNVSSIVTMTSQGMYGGTYLVEKPNLSSKRSELSQLSMYRVFEVGVIRNPGLGAPVFHMTNYLEQPVSNDLSNCMVALGELKLAALCHGEDSITIPYQGSGKGVSFQLVKLGVWKSPTDMQSWVPLSTDDPVIDRLYLSSHRGVIADNQAKWAVPTTRTDDKLRMETCFQQACKGKIQALCENPEWAPLKDNRIPSYGVLSVDLSLTVELKIKIASGFGPLITHGSGMDLYKSNHNNVYWLTIPPMKNLALGVINTLEWIPRFKVSPYLFTVPIKEAGGDCHAPTYLPAEVDGDVKLSSNLVILPGQDLQYVLATYDTSRVEHAVVYYVYSPSRSFSYFYPFRLPIKGVPIELQVECFTWDQKLWCRHFCVLADSESGGHITHSGMVGMGVSCTVTREDGTNRR

SEQ ID NO: 156 - protein, 597 aa, Measles morbillivirus
MAAAANREHLMIDRPYVLLAVLFVMFLSLIGLLAIAGIRLHRAAIYTAEIHKSLSTNLDVTNSIEHQVKDVLTPLFKIIGDEVGLRTPQRFTDLVKFISDKIKFLNPDREYDFRDLTWCINPPERIKLDYDQYCADVAAEELMNALVNSTLLETRTTNQFLAVSKGNCSGPTTIRGQFSNMSLSLLDLYLGRGYNVSSIVTMTSQGMYGGTYLVEKPNLSSKRSELSQLSMYRVFEVGVIRNPGLGAPVFHMTNYLEQPVSNDLSNCMVALGELKLAALCHGEDSITIPYQGSGKGVSFQLVKLGVWKSPTDMQSWVPLSTDDPVIDRLYLSSHRGVIADNQAKWAVPTTRTDDKLRMETCFQQACKGKIQALCENPEWAPLKDNRIPSYGVLSVDLSLTVELKIKIASGFGPLITHGSGMDLYKSNHNNVYWLTIPPMKNLALGVINTLEWIPRFKVSPALFNVPIKEAGGDCHAPTYLPAEVDGDVKLSSNLVILPGQDLQYVLATYDTSAVEHAVVYYVYSPSRSFSYFYPFRLPIKGVPIELQVECFTWDQKLWCRHFCVLADSESGGHITHSGMVGMGVSCTVTREAAARGS

SEQ ID NO: 157 - protein, 597 aa, synthetic construct; identical to SEQ ID NO: 156

SEQ ID NO: 158 - protein, 597 aa, Measles morbillivirus
MAAAANREHLMIDRPYVLLAVLFVMFLSLIGLLAIAGIRLHRAAIYTAEIHKSLSTNLDVTNSIEHQVKDVLTPLFKIIGDEVGLRTPQRFTDLVKFISDKIKFLNPDREYDFRDLTWCINPPERIKLDYDQYCADVAAEELMNALVNSTLLETRTTNQFLAVSKGNCSGPTTIRGQFSNMSLSLLDLYLGRGYNVSSIVTMTSQGMYGGTYLVEKPNLSSKRSELSQLSMYRVFEVGVIRNPGLGAPVFHMTNYLEQPVSNDLSNCMVALGELKLAALCHGEDSITIPYQGSGKGVSFQLVKLGVWKSPTDMQSWVPLSTDDPVIDRLYLSSHRGVIADNQAKWAVPTTRTDDKLRMETCFQQACKGKIQALCENPEWAPLKDNRIPSYGVLSVDLSLTVELKIKIASGFGPLITHGSGMDLYKSNHNNVYWLTIPPMKNLALGVINTLEWIPRFKVSPALFNVPIKEAGGDCHAPTYLPAEVDGDVKLSSNLVILPGQDLQYVLATYDTSAVEHAVVYYVYSPSRLSSYFYPFRLPIKGVPIELQVECFTWDQKLWCRHFCVLADSESGGHITHSGMVGMGVSCTVTREAAARGS

SEQ ID NO: 159 - protein, 597 aa, synthetic construct; identical to SEQ ID NO: 158

SEQ ID NO: 160 - protein, 526 aa, Measles morbillivirus
MGLKVNVSAIFMAVLLTLQTPTGQIHWGNLSKIGVVGIGSASYKVMTRSSHQSLVIKLMPNITLLNNCTRVEIAEYRRLLRTVLEPIRDALNAMTQNIRPVQSVASSRRHKRFAGVVLAGAALGVATAAQITAGIALHQSMLNSQAIDNLRASLETTNQAIEAIRQAGQEMILAVQGVQDYINNELIPSMNQLSCDLIGQKLGLKLLRYYTEILSLFGPSLRDPISAEISIQALSYALGGDINKVLEKLGYSGGDLLGILESRGIKARITHVDTESYFIVLSIAYPTLSEIKGVIVHRLEGVSYNIGSQEWYTTVPKYVATQGYLISNFDESSCTFMPEGTVCSQNALYPMSPLLQECLRGSTKSCARTLVSGSFGNRFILSQGNLIANCASILCKCYTTGTIINQDPDKILTYIAADHCPVVEVNGVTIQVGSRRYPDAVYLHRIDLGPPISLERLDVGTNLGNAIAKLEDAKELLESSDQILRSMKGLSSTSIVYILIAVCLGGLIGIPALICCCRGRCNKKGE

SEQ ID NO: 161 - protein, 526 aa, Measles morbillivirus
MGLKVNVSAIFMAVLLTLQTPTGQIHWGNLSKIGVVGIGSASYKVMTRSSHQSLVIKLMPNITLLNNCTRVEIAEYRRLLRTVLEPIRDALNAMTQNIRPVQSVASSRRHKRFAGVVLAGAALGVATAAQITAGIALHQSMLNSQAIDNLRASLETTNQAIEAIRQAGQEMILAVQGVQDYINNELIPSMNQLSCDLIGQKLGLKLLRYYTEILSLFGPSLRDPISAEISIQALSYALGGDINKVLEKLGYSGGDLLGILESRGIKARITHVDTESYFIVLSIAYPTLSEIKGVIVHRLEGVSYNIGSQEWYTTVPKYVATQGYLISNFDESSCTFMPEGTVCSQNALYPMSPLLQECLRGSTKSCARTLVSGSFGNRFILSQGNLIANCASILCKCYTTGTIINQDPDKILTYIAADHCPVVEVNGVTIQVGSRRYPDAVYLHRIDLGPPISLERLDVGINLGNAIAKLEDAKELLESSDQILRSMKGLSSTSIVYILIAVCLGGLIGIPALICCCRGRCNKKGE

SEQ ID NO: 162 - protein, 520 aa, Measles morbillivirus
MGLKVNVSAIFMAVLLTLQTPTGQIHWGNLSKIGVVGIGSASYKVMTRSSHQSLVIKLMPNITLLNNCTRVEIAEYRRLLRTVLEPIRDALNAMTQNIRPVQSVASSRRHKRFAGVVLAGAALGVATAAQITAGIALHQSMLNSQAIDNLRASLETTNQAIEAIRQAGQEMILAVQGVQDYINNELIPSMNQLSCDLIGQKLGLKLLRYYTEILSLFGPSLRDPISAEISIQALSYALGGDINKVLEKLGYSGGDLLGILESRGIKARITHVDTESYFIVLSIAYPTLSEIKGVIVHRLEGVSYNIGSQEWYTTVPKYVATQGYLISNFDESSCTFMPEGTVCSQNALYPMSPLLQECLRGSTKSCARTLVSGSFGNRFILSQGNLIANCASILCKCYTTGTIINQDPDKILTYIAADHCPVVEVNGVTIQVGSRRYPDAVYLHRIDLGPPISLERLDVGTNLGNAIAKLEDAKELLESSDQILRSMKGLSSTSIVYILIAVCLGGLIGIPALICCCRGR

SEQ ID NO: 163 - protein, 520 aa, Measles morbillivirus
MGLKVNVSAIFMAVLLTLQTPTGQIHWGNLSKIGVVGIGSASYKVMTRSSHQSLVIKLMPNITLLNNCTRVEIAEYRRLLRTVLEPIRDALNAMTQNIRPVQSVASSRRHKRFAGVVLAGAALGVATAAQITAGIALHQSMLNSQAIDNLRASLETTNQAIEAIRQAGQEMILAVQGVQDYINNELIPSMNQLSCDLIGQKLGLKLLRYYTEILSLFGPSLRDPISAEISIQALSYALGGDINKVLEKLGYSGGDLLGILESRGIKARITHVDTESYFIVLSIAYPTLSEIKGVIVHRLEGVSYNIGSQEWYTTVPKYVATQGYLISNFDESSCTFMPEGTVCSQNALYPMSPLLQECLRGSTKSCARTLVSGSFGNRFILSQGNLIANCASILCKCYTTGTIINQDPDKILTYIAADHCPVVEVNGVTIQVGSRRYPDAVYLHRIDLGPPISLERLDVGINLGNAIAKLEDAKELLESSDQILRSMKGLSSTSIVYILIAVCLGGLIGIPALICCCRGR

SEQ ID NO: 164 - protein, 665 aa, unidentified
MDYHSHTTQTGSNETLYQDPLQSQSGSRDTLDGPPSTLQHYSNPPPYSEEDQGIDGPQRSQPLSTPHQYDRYYGVNIQHTRVYNHLGTIYKGLKLAFQILGWVSVIITMIITVTTLKKMSDGNSQDSAMLKSLDENFDAIQEVANLLDNEVRPKLGVTMTQTTFQLPKELSEIKRYLLRLERNCPVCGTEATPQGSKGNASGDTAFCPPCLTRQCSEDSTHDQGPGVEGTSRNHKGKINFPHILQSDDCGRSDNLIVYSINLVPGLSFIQLPSGTKHCIIDVSYTFSDTLAGYLIVGGVDGCQLHNKAIIYLSLGYYKTKMIYPPDYIAIATYTYDLVPNLRDCSIAVNQTSLAAICTSKKTKENQDFSTSGVHPFYIFTLNTDGIFTVTVIEQSQLKLDYQYAALYPATGPGIFIGDHLVFLMWGGLMTKAEGDAYCQASGCNDAHRTSCNIAQMPSAYGHRQLVNGLLMLPIKELGSHLIQPSLETISPKINWAGGHGRLYYNWEINTTYIYIEGKTWRSRPNLGIISWSKPLSIRWIDHSVARRPGARPCDSANDCPEDCLVGGYYDMFPMSSDYKTAITIIPTHHQWPSSPALKLFNTNREVRVVMILRPPNNVKKTTISCIRIMQTNWCLGFIIFKEGNNAWGQIYSYIYQVESTCPNTK

SEQ ID NO: 165 - protein, 642 aa, unidentified
MGPPSTLQHYSNPPPYSEEDQGIDGPQRSQPLSTPHQYDRYYGVNIQHTRVYNHLGTIYSGLKLAFQILGWVSVIITMIITVTTLKKMSDGNSQDSAMLKSLDENFDAIQEVANLLDNEVRPKLGVTMTQTTFQLPKELSEIKRYLLRLERNCPVCGTEATPQGSKGNASGDTAFCPPCLTRQCSEDSTHDQGPGVEGTSRNHKGKINFPHILQSDDCGRSDNLIVYSINLVPGLSFIQLPSGTKHCIIDVSYTFSDTLAGYLIVGGVDGCQLHNKAIIYLSLGYYKTKMIYPPDYIAIATYTYDLVPNLRDCSIAVNQTSLAAICTSKKTKENQDFSTSGVHPFYIFTLNTDGIFTVTVIEQSQLKLDYQYAALYPATGPGIFIGDHLVFLMWGGLMTKAEGDAYCQASGCNDAHRTSCNIAQMPSAYGHRQLVNGLLMLPIKELGSHLIQPSLETISPKINWAGGHGRLYYNWEINTTYIYIEGKTWRSRPNLGIISWSKPLSIRWIDHSVARRPGARPCDSANDCPEDCLVGGYYDMFPMSSDYKTAITIIPTHHQWPSSPALKLFNTNREVRVVMILRPPNNVKKTTISCIRIMQTNWCLGFIIFKEGNNAWGQIYSYIYQVESTCPNTKTAARGTGS

SEQ ID NO: 166 - protein, 642 aa, synthetic construct; identical to SEQ ID NO: 165

SEQ ID NO: 167 - protein, 586 aa, unidentified
MRVYNHLGTIYKGLKLAFQILGWVSVIITMIITVTTLKKMSDGNSQDSAMLKSLDENFDAIQEVANLLDNEVRPKLGVTMTQTTFQLPKELSEIKRYLLRLERNCPVCGTEATPQGSKGNASGDTAFCPPCLTRQCSEDSTHDQGPGVEGTSRNHKGKINFPHILQSDDCGRSDNLIVYSINLVPGLSFIQLPSGTKHCIIDVSYTFSDTLAGYLIVGGVDGCQLHNKAIIYLSLGYYKTKMIYPPDYIAIATYTYDLVPNLRDCSIAVNQTSLAAICTSKKTKENQDFSTSGVHPFYIFTLNTDGIFTVTVIEQSQLKLDYQYAALYPATGPGIFIGDHLVFLMWGGLMTKAEGDAYCQASGCNDAHRTSCNIAQMPSAYGHRQLVNGLLMLPIKELGSHLIQPSLETISPKINWAGGHGRLYYNWEINTTYIYIEGKTWRSRPNLGIISWSKPLSIRWIDHSVARRPGARPCDSANDCPEDCLVGGYYDMFPMSSDYKTAITIIPTHHQWPSSPALKLFNTNREVRVVMILRPPNNVKKTTISCIRIMQTNWCLGFIIFKEGNNAWGQIYSYIYQVESTCPNTK

SEQ ID NO: 168 - protein, 594 aa, synthetic construct
MRVYNHLGTIYKGLKLAFQILGWVSVIITMIITVTTLKKMSDGNSQDSAMLKSLDENFDAIQEVANLLDNEVRPKLGVTMTQTTFQLPKELSEIKRYLLRLERNCPVCGTEATPQGSKGNASGDTAFCPPCLTRQCSEDSTHDQGPGVEGTSRNHKGKINFPHILQSDDCGRSDNLIVYSINLVPGLSFIQLPSGTKHCIIDVSYTFSDTLAGYLIVGGVDGCQLHNKAIIYLSLGYYKTKMIYPPDYIAIATYTYDLVPNLRDCSIAVNQTSLAAICTSKKTKENQDFSTSGVHPFYIFTLNTDGIFTVTVIEQSQLKLDYQYAALYPATGPGIFIGDHLVFLMWGGLMTKAEGDAYCQASGCNDAHRTSCNIAQMPSAYGHRQLVNGLLMLPIKELGSHLIQPSLETISPKINWAGGHGRLYYNWEINTTYIYIEGKTWRSRPNLGIISWSKPLSIRWIDHSVARRPGARPCDSANDCPEDCLVGGYYDMFPMSSDYKTAITIIPTHHQWPSSPALKLFNTNREVRVVMILRPPNNVKKTTISCIRIMQTNWCLGFIIFKEGNNAWGQIYSYIYQVESTCPNTKTAARGTGS

SEQ ID NO: 169 - protein, 553 aa, unidentified
MASLLKTICYIYLITYAKLEPTPKSQLDLDSLASIGVVDAGKYNYKLMTTGSEKLMVIKLVPNITYATNCNLTAHTAYTKMIERLLTPINQSLYEMRSVITERDGGTIFWGAIIAGAALGVATAAAITAGVALHRAEQNARNIAALKDALRNSNEAIQHLKDAQGHTVLAIQGLQEQINNNIIPKLKESHCLGVNNQLGLLLNQYYSEILTVFGPNLQNPVSASLTIQAIAKAFNGDFNSLMTNLNYDPTDLLDILESNSINGRIIDVNLNEKYIALSIEIPNFITLTDAKIQTFNRITYGYGSNEWLTLIPDNILEYGNLISNVDLTSCVKTKSSYICNQDTSYPISSELTRCLRGDTSSCPRTPVVNSRAPTFALSGGHIYANCAKAACRCEKPPMAIVQPATSTLTFLTEKECQEVVIDQINIQLAPNRLNKTIITDGIDLGPEVIINPIDVSAELGNIELEMDKTQKALDRSNKILDSMITEVTPDKLLIAMIVVFGILLLWLFGVSYYAFKIWSKLHFLDSYVYSLRNPSHHRSNGHQNHSFSTDISG

SEQ ID NO: 170 - protein, 521 aa, unidentified
MASLLKTICYIYLITYAKLEPTPKSQLDLDSLASIGVVDAGKYNYKLMTTGSEKLMVIKLVPNITYATNCNLTAHTAYTKMIERLLTPINQSLYEMRSVITERDGGTIFWGAIIAGAALGVATAAAITAGVALHRAEQNARNIAALKDALRNSNEAIQHLKDAQGHTVLAIQGLQEQINNNIIPKLKESHCLGVNNQLGLLLNQYYSEILTVFGPNLQNPVSASLTIQAIAKAFNGDFNSLMTNLNYDPTDLLDILESNSINGRIIDVNLNEKYIALSIEIPNFITLTDAKIQTFNRITYGYGSNEWLTLIPDNILEYGNLISNVDLTSCVKTKSSYICNQDTSYPISSELTRCLRGDTSSCPRTPVVNSRAPTFALSGGHIYANCAKAACRCEKPPMAIVQPATSTLTFLTEKECQEVVIDQINIQLAPNRLNKTIITDGIDLGPEVIINPIDVSAELGNIELEMDKTQKALDRSNKILDSMITEVTPDKLLIAMIVVFGILLLWLFGVSYYAFKIWSKL

SEQ ID NO: 171 - protein, 604 aa, Morbillivirus canis
MLPYQDKVGAFYKDNARANSTKLSLVTEGHGGRRPPYLLFVLLILLVGILALLAITGVRFHQVSTSNMEFSRLLKEDMEKSEAVHHQVIDVLTPLFKIIGDEIGLRLPQKLNEIKQFILQKTNFFNPNREFDFRDLHWCINPPSTVKVNFTNYCESIGIRKAIASAANPILLSALSGGRGDIFPPHRCSGATTSVGKVFPLSVSLSMSLISRTSEVINMLTAISDGVYGKTYLLVPDDIEREFDTREIRVFEIGFIKRWLNDMPLLQTTNYMVLPKNSKAKVCTIAVGELTLASLCVEESTVLLYHDSSGSQDGILVVTLGIFWATPMDHIEEVIPVAHPSMKKIHITNHRGFIKDSIATWMVPALASEKQEEQKGCLESACQRKTYPMCNQASWEPFGGRQLPSYGRLTLPLDASVDLQLNISFTYGPVILNGDGMDYYESPLLNSGWLTIPPKDGTISGLINKAGRGDQFTVLPHVLTFAPRESSGNCYLPIQTSQIRDRDVLIESNIVVLPTQSIRYVIATYDISRSDHAIVYYVYDPIRTISYTHPFRLTTKGRPDFLRIECFVWDDNLWCHQFYRFEADIANSTTSVENLVRIRFSCNR

SEQ ID NO: 172 - protein, 606 aa, synthetic construct
MLPYQDKVGAFYKDNARANSTKLSLVTEGHGGRRPPYLLFVLLILLVGILALLAITGVRFHQVSTSNMEFSRLLKEDMEKSEAVHHQVIDVLTPLFKIIGDEIGLRLPQKLNEIKQFILQKTNFFNPNREFDFRDLHWCINPPSTVKVNFTNYCESIGIRKAIASAANPILLSALSGGRGDIFPPHRCSGATTSVGKVFPLSVSLSMSLISRTSEVINMLTAISDGVYGKTYLLVPDDIEREFDTREIRVFEIGFIKRWLNDMPLLQTTNYMVLPKNSKAKVCTIAVGELTLASLCVEESTVLLYHDSSGSQDGILVVTLGIFWATPMDHIEEVIPVAHPSMKKIHITNHRGFIKDSIATWMVPALASEKQEEQKGCLESACQRKTYPMCNQASWEPFGGRQLPSYGRLTLPLDASVDLQLNISFTYGPVILNGDGMDYYESPLLNSGWLTIPPKDGTISGLINKAGRGDQFTVLPHVLTFAPRESSGNCYLPIQTSQIRDRDVLIESNIVVLPTQSIRYVIATYDISRSDHAIVYYVYDPIRTISYTHPFRLTTKGRPDFLRIECFVWDDNLWCHQFYRFEADIANSTTSVENLVRIRFSCNRGS

SEQ ID NO: 173 - protein, 586 aa, Morbillivirus canis
MSTKLSLVTEGHGGRRPPYLLFVLLILLVGILALLAITGVRFHQVSTSNMEFSRLLKEDMEKSEAVHHQVIDVLTPLFKIIGDEIGLRLPQKLNEIKQFILQKTNFFNPNREFDFRDLHWCINPPSTVKVNFTNYCESIGIRKAIASAANPILLSALSGGRGDIFPPHRCSGATTSVGKVFPLSVSLSMSLISRTSEVINMLTAISDGVYGKTYLLVPDDIEREFDTREIRVFEIGFIKRWLNDMPLLQTTNYMVLPKNSKAKVCTIAVGELTLASLCVEESTVLLYHDSSGSQDGILVVTLGIFWATPMDHIEEVIPVAHPSMKKIHITNHRGFIKDSIATWMVPALASEKQEEQKGCLESACQRKTYPMCNQASWEPFGGRQLPSYGRLTLPLDASVDLQLNISFTYGPVILNGDGMDYYESPLLNSGWLTIPPKDGTISGLINKAGRGDQFTVLPHVLTFAPRESSGNCYLPIQTSQIRDRDVLIESNIVVLPTQSIRYVIATYDISRSDHAIVYYVYDPIRTISYTHPFRLTTKGRPDFLRIECFVWDDNLWCHQFYRFEADIANSTTSVENLVRIRFSCNR

SEQ ID NO: 174 - protein, 588 aa, synthetic construct
MSTKLSLVTEGHGGRRPPYLLFVLLILLVGILALLAITGVRFHQVSTSNMEFSRLLKEDMEKSEAVHHQVIDVLTPLFKIIGDEIGLRLPQKLNEIKQFILQKTNFFNPNREFDFRDLHWCINPPSTVKVNFTNYCESIGIRKAIASAANPILLSALSGGRGDIFPPHRCSGATTSVGKVFPLSVSLSMSLISRTSEVINMLTAISDGVYGKTYLLVPDDIEREFDTREIRVFEIGFIKRWLNDMPLLQTTNYMVLPKNSKAKVCTIAVGELTLASLCVEESTVLLYHDSSGSQDGILVVTLGIFWATPMDHIEEVIPVAHPSMKKIHITNHRGFIKDSIATWMVPALASEKQEEQKGCLESACQRKTYPMCNQASWEPFGGRQLPSYGRLTLPLDASVDLQLNISFTYGPVILNGDGMDYYESPLLNSGWLTIPPKDGTISGLINKAGRGDQFTVLPHVLTFAPRESSGNCYLPIQTSQIRDRDVLIESNIVVLPTQSIRYVIATYDISRSDHAIVYYVYDPIRTISYTHPFRLTTKGRPDFLRIECFVWDDNLWCHQFYRFEADIANSTTSVENLVRIRFSCNRGS

SEQ ID NO: 175 - protein, 585 aa, Morbillivirus canis
MTKLSLVTEGHGGRRPPYLLFVLLILLVGILALLAITGVRFHQVSTSNMEFSRLLKEDMEKSEAVHHQVIDVLTPLFKIIGDEIGLRLPQKLNEIKQFILQKTNFFNPNREFDFRDLHWCINPPSTVKVNFTNYCESIGIRKAIASAANPILLSALSGGRGDIFPPHRCSGATTSVGKVFPLSVSLSMSLISRTSEVINMLTAISDGVYGKTYLLVPDDIEREFDTREIRVFEIGFIKRWLNDMPLLQTTNYMVLPKNSKAKVCTIAVGELTLASLCVEESTVLLYHDSSGSQDGILVVTLGIFWATPMDHIEEVIPVAHPSMKKIHITNHRGFIKDSIATWMVPALASEKQEEQKGCLESACQRKTYPMCNQASWEPFGGRQLPSYGRLTLPLDASVDLQLNISFTYGPVILNGDGMDYYESPLLNSGWLTIPPKDGTISGLINKAGRGDQFTVLPHVLTFAPRESSGNCYLPIQTSQIRDRDVLIESNIVVLPTQSIRYVIATYDISRSDHAIVYYVYDPIRTISYTHPFRLTTKGRPDFLRIECFVWDDNLWCHQFYRFEADIANSTTSVENLVRIRFSCNR

SEQ ID NO: 176 - protein, 587 aa, synthetic construct
MTKLSLVTEGHGGRRPPYLLFVLLILLVGILALLAITGVRFHQVSTSNMEFSRLLKEDMEKSEAVHHQVIDVLTPLFKIIGDEIGLRLPQKLNEIKQFILQKTNFFNPNREFDFRDLHWCINPPSTVKVNFTNYCESIGIRKAIASAANPILLSALSGGRGDIFPPHRCSGATTSVGKVFPLSVSLSMSLISRTSEVINMLTAISDGVYGKTYLLVPDDIEREFDTREIRVFEIGFIKRWLNDMPLLQTTNYMVLPKNSKAKVCTIAVGELTLASLCVEESTVLLYHDSSGSQDGILVVTLGIFWATPMDHIEEVIPVAHPSMKKIHITNHRGFIKDSIATWMVPALASEKQEEQKGCLESACQRKTYPMCNQASWEPFGGRQLPSYGRLTLPLDASVDLQLNISFTYGPVILNGDGMDYYESPLLNSGWLTIPPKDGTISGLINKAGRGDQFTVLPHVLTFAPRESSGNCYLPIQTSQIRDRDVLIESNIVVLPTQSIRYVIATYDISRSDHAIVYYVYDPIRTISYTHPFRLTTKGRPDFLRIECFVWDDNLWCHQFYRFEADIANSTTSVENLVRIRFSCNRGS

SEQ ID NO: 177 - protein, 662 aa, Morbillivirus canis
MHRGIPKSSKTQTHTQQDRPPQPSTELEETRTSRARHSTTSAQRSTHYDPRTSDRPVSYTMNRTRSRKQTSHRLKNIPVHGNHEATIQHIPESVSKGARSQIERRQPNAINSGSHCTWLVLWCLGMASLFLCSKAQIHWDNLSTIGIIGTDNVHYKIMTRPSHQYLVIKLIPNASLIENCTKAELGEYEKLLNSVLEPINQALTLMTKNVKPLQSLGSGRRQRRFAGVVLAGVALGVATAAQITAGIALHQSNLNAQAIQSLRTSLEQSNKAIEEIREATQETVIAVQGVQDYVNNELVPAMQHMSCELVGQRLGLRLLRYYTELLSIFGPSLRDPISAEISIQALIYALGGEIHKILEKLGYSGSDMIAILESRGIKTKITHVDLPGKFIILSISYPTLSEVKGVIVHRLEAVSYNIGSQEWYTTVPRYIATNGYLISNFDESSCVFVSESAICSQNSLYPMSPLLQQCIRGDTSSCARTLVSGTMGNKFILSKGNIVANCASILCKCYSTSTIINQSPDKLLTFIASDTCPLVEIDGATIQVGGRQYPDMVYEGKVALGPAISLDRLDVGTNLGNALKKLDDAKVLIDSSNQILETVRRSSFNFGSLLSVPILSCTALALLLLIYCCKRRYQQTLKQHTKVDPAFKPDLTGTSKSYVRSL

SEQ ID NO: 178 - protein, 662 aa, Morbillivirus canis
MHRGIPKSSKTQTHTQQDRPPQPSTELEETRTSRARHSTTSAQRSTHYDPRTSDRPVSYTMNRTRSRKQTSHRLKNIPVHGNHEATIQHIPESVSKGARSQIERRQPNAINSGSHCTWLVLWCLGMASLFLCSKAQIHWDNLSTIGIIGTDNVHYKIMTRPSHQYLVIKLIPNASLIENCTKAELGEYEKLLNSVLEPINQALTLMTKNVKPLQSLGSGRRQRRFAGVVLAGVALGVATAAQITAGIALHQSNLNAQAIQSLRTSLEQSNKAIEEIREATQETVIAVQGVQDYVNNELVPAMQHMSCELVGQRLGLRLLRYYTELLSIFGPSLRDPISAEISIQALIYALGGEIHKILEKLGYSGSDMIAILESRGIKTKITHVDLPGKFIILSISYPTLSEVKGVIVHRLEAVSYNIGSQEWYTTVPRYIATNGYLISNFDESSCVFVSESAICSQNSLYPMSPLLQQCIRGDTSSCARTLVSGTMGNKFILSKGNIVANCASILCKCYSTSTIINQSPDKLLTFIASDTCPLVEIDGATIQVGGRQYPDMVYEGKVALGPAISLDRLDVGINLGNALKKLDDAKVLIDSSNQILETVRRSSFNFGSLLSVPILSCTALALLLLIYCCKRRYQQTLKQHTKVDPAFKPDLTGTSKSYVRSL

SEQ ID NO: 179 - protein, 638 aa, Morbillivirus canis
MHRGIPKSSKTQTHTQQDRPPQPSTELEETRTSRARHSTTSAQRSTHYDPRTSDRPVSYTMNRTRSRKQTSHRLKNIPVHGNHEATIQHIPESVSKGARSQIERRQPNAINSGSHCTWLVLWCLGMASLFLCSKAQIHWDNLSTIGIIGTDNVHYKIMTRPSHQYLVIKLIPNASLIENCTKAELGEYEKLLNSVLEPINQALTLMTKNVKPLQSLGSGRRQRRFAGVVLAGVALGVATAAQITAGIALHQSNLNAQAIQSLRTSLEQSNKAIEEIREATQETVIAVQGVQDYVNNELVPAMQHMSCELVGQRLGLRLLRYYTELLSIFGPSLRDPISAEISIQALIYALGGEIHKILEKLGYSGSDMIAILESRGIKTKITHVDLPGKFIILSISYPTLSEVKGVIVHRLEAVSYNIGSQEWYTTVPRYIATNGYLISNFDESSCVFVSESAICSQNSLYPMSPLLQQCIRGDTSSCARTLVSGTMGNKFILSKGNIVANCASILCKCYSTSTIINQSPDKLLTFIASDTCPLVEIDGATIQVGGRQYPDMVYEGKVALGPAISLDRLDVGTNLGNALKKLDDAKVLIDSSNQILETVRRSSFNFGSLLSVPILSCTALALLLLIYCCKRRYQQTLK

SEQ ID NO: 180 - protein, 638 aa, Morbillivirus canis
MHRGIPKSSKTQTHTQQDRPPQPSTELEETRTSRARHSTTSAQRSTHYDPRTSDRPVSYTMNRTRSRKQTSHRLKNIPVHGNHEATIQHIPESVSKGARSQIERRQPNAINSGSHCTWLVLWCLGMASLFLCSKAQIHWDNLSTIGIIGTDNVHYKIMTRPSHQYLVIKLIPNASLIENCTKAELGEYEKLLNSVLEPINQALTLMTKNVKPLQSLGSGRRQRRFAGVVLAGVALGVATAAQITAGIALHQSNLNAQAIQSLRTSLEQSNKAIEEIREATQETVIAVQGVQDYVNNELVPAMQHMSCELVGQRLGLRLLRYYTELLSIFGPSLRDPISAEISIQALIYALGGEIHKILEKLGYSGSDMIAILESRGIKTKITHVDLPGKFIILSISYPTLSEVKGVIVHRLEAVSYNIGSQEWYTTVPRYIATNGYLISNFDESSCVFVSESAICSQNSLYPMSPLLQQCIRGDTSSCARTLVSGTMGNKFILSKGNIVANCASILCKCYSTSTIINQSPDKLLTFIASDTCPLVEIDGATIQVGGRQYPDMVYEGKVALGPAISLDRLDVGINLGNALKKLDDAKVLIDSSNQILETVRRSSFNFGSLLSVPILSCTALALLLLIYCCKRRYQQTLK

SEQ ID NO: 181 - protein, 632 aa, Morbillivirus canis
MHRGIPKSSKTQTHTQQDRPPQPSTELEETRTSRARHSTTSAQRSTHYDPRTSDRPVSYTMNRTRSRKQTSHRLKNIPVHGNHEATIQHIPESVSKGARSQIERRQPNAINSGSHCTWLVLWCLGMASLFLCSKAQIHWDNLSTIGIIGTDNVHYKIMTRPSHQYLVIKLIPNASLIENCTKAELGEYEKLLNSVLEPINQALTLMTKNVKPLQSLGSGRRQRRFAGVVLAGVALGVATAAQITAGIALHQSNLNAQAIQSLRTSLEQSNKAIEEIREATQETVIAVQGVQDYVNNELVPAMQHMSCELVGQRLGLRLLRYYTELLSIFGPSLRDPISAEISIQALIYALGGEIHKILEKLGYSGSDMIAILESRGIKTKITHVDLPGKFIILSISYPTLSEVKGVIVHRLEAVSYNIGSQEWYTTVPRYIATNGYLISNFDESSCVFVSESAICSQNSLYPMSPLLQQCIRGDTSSCARTLVSGTMGNKFILSKGNIVANCASILCKCYSTSTIINQSPDKLLTFIASDTCPLVEIDGATIQVGGRQYPDMVYEGKVALGPAISLDRLDVGTNLGNALKKLDDAKVLIDSSNQILETVRRSSFNFGSLLSVPILSCTALALLLLIYCCKRR

SEQ ID NO: 182 - protein, 632 aa, Morbillivirus canis
MHRGIPKSSKTQTHTQQDRPPQPSTELEETRTSRARHSTTSAQRSTHYDPRTSDRPVSYTMNRTRSRKQTSHRLKNIPVHGNHEATIQHIPESVSKGARSQIERRQPNAINSGSHCTWLVLWCLGMASLFLCSKAQIHWDNLSTIGIIGTDNVHYKIMTRPSHQYLVIKLIPNASLIENCTKAELGEYEKLLNSVLEPINQALTLMTKNVKPLQSLGSGRRQRRFAGVVLAGVALGVATAAQITAGIALHQSNLNAQAIQSLRTSLEQSNKAIEEIREATQETVIAVQGVQDYVNNELVPAMQHMSCELVGQRLGLRLLRYYTELLSIFGPSLRDPISAEISIQALIYALGGEIHKILEKLGYSGSDMIAILESRGIKTKITHVDLPGKFIILSISYPTLSEVKGVIVHRLEAVSYNIGSQEWYTTVPRYIATNGYLISNFDESSCVFVSESAICSQNSLYPMSPLLQQCIRGDTSSCARTLVSGTMGNKFILSKGNIVANCASILCKCYSTSTIINQSPDKLLTFIASDTCPLVEIDGATIQVGGRQYPDMVYEGKVALGPAISLDRLDVGINLGNALKKLDDAKVLIDSSNQILETVRRSSFNFGSLLSVPILSCTALALLLLIYCCKRR

SEQ ID NO: 183 - protein, 556 aa, Morbillivirus canis
MNAINSGSHCTWLVLWCLGMASLFLCSKAQIHWDNLSTIGIIGTDNVHYKIMTRPSHQYLVIKLIPNASLIENCTKAELGEYEKLLNSVLEPINQALTLMTKNVKPLQSLGSGRRQRRFAGVVLAGVALGVATAAQITAGIALHQSNLNAQAIQSLRTSLEQSNKAIEEIREATQETVIAVQGVQDYVNNELVPAMQHMSCELVGQRLGLRLLRYYTELLSIFGPSLRDPISAEISIQALIYALGGEIHKILEKLGYSGSDMIAILESRGIKTKITHVDLPGKFIILSISYPTLSEVKGVIVHRLEAVSYNIGSQEWYTTVPRYIATNGYLISNFDESSCVFVSESAICSQNSLYPMSPLLQQCIRGDTSSCARTLVSGTMGNKFILSKGNIVANCASILCKCYSTSTIINQSPDKLLTFIASDTCPLVEIDGATIQVGGRQYPDMVYEGKVALGPAISLDRLDVGTNLGNALKKLDDAKVLIDSSNQILETVRRSSFNFGSLLSVPILSCTALALLLLIYCCKRRYQQTLKQHTKVDPAFKPDLTGTSKSYVRSL

SEQ ID NO: 184 - protein, 556 aa, Morbillivirus canis
MNAINSGSHCTWLVLWCLGMASLFLCSKAQIHWDNLSTIGIIGTDNVHYKIMTRPSHQYLVIKLIPNASLIENCTKAELGEYEKLLNSVLEPINQALTLMTKNVKPLQSLGSGRRQRRFAGVVLAGVALGVATAAQITAGIALHQSNLNAQAIQSLRTSLEQSNKAIEEIREATQETVIAVQGVQDYVNNELVPAMQHMSCELVGQRLGLRLLRYYTELLSIFGPSLRDPISAEISIQALIYALGGEIHKILEKLGYSGSDMIAILESRGIKTKITHVDLPGKFIILSISYPTLSEVKGVIVHRLEAVSYNIGSQEWYTTVPRYIATNGYLISNFDESSCVFVSESAICSQNSLYPMSPLLQQCIRGDTSSCARTLVSGTMGNKFILSKGNIVANCASILCKCYSTSTIINQSPDKLLTFIASDTCPLVEIDGATIQVGGRQYPDMVYEGKVALGPAISLDRLDVGINLGNALKKLDDAKVLIDSSNQILETVRRSSFNFGSLLSVPILSCTALALLLLIYCCKRRYQQTLKQHTKVDPAFKPDLTGTSKSYVRSL

SEQ ID NO: 185 - protein, 532 aa, Morbillivirus canis
MNAINSGSHCTWLVLWCLGMASLFLCSKAQIHWDNLSTIGIIGTDNVHYKIMTRPSHQYLVIKLIPNASLIENCTKAELGEYEKLLNSVLEPINQALTLMTKNVKPLQSLGSGRRQRRFAGVVLAGVALGVATAAQITAGIALHQSNLNAQAIQSLRTSLEQSNKAIEEIREATQETVIAVQGVQDYVNNELVPAMQHMSCELVGQRLGLRLLRYYTELLSIFGPSLRDPISAEISIQALIYALGGEIHKILEKLGYSGSDMIAILESRGIKTKITHVDLPGKFIILSISYPTLSEVKGVIVHRLEAVSYNIGSQEWYTTVPRYIATNGYLISNFDESSCVFVSESAICSQNSLYPMSPLLQQCIRGDTSSCARTLVSGTMGNKFILSKGNIVANCASILCKCYSTSTIINQSPDKLLTFIASDTCPLVEIDGATIQVGGRQYPDMVYEGKVALGPAISLDRLDVGTNLGNALKKLDDAKVLIDSSNQILETVRRSSFNFGSLLSVPILSCTALALLLLIYCCKRRYQQTLK

SEQ ID NO: 186 - protein, 532 aa, Morbillivirus canis
MNAINSGSHCTWLVLWCLGMASLFLCSKAQIHWDNLSTIGIIGTDNVHYKIMTRPSHQYLVIKLIPNASLIENCTKAELGEYEKLLNSVLEPINQALTLMTKNVKPLQSLGSGRRQRRFAGVVLAGVALGVATAAQITAGIALHQSNLNAQAIQSLRTSLEQSNKAIEEIREATQETVIAVQGVQDYVNNELVPAMQHMSCELVGQRLGLRLLRYYTELLSIFGPSLRDPISAEISIQALIYALGGEIHKILEKLGYSGSDMIAILESRGIKTKITHVDLPGKFIILSISYPTLSEVKGVIVHRLEAVSYNIGSQEWYTTVPRYIATNGYLISNFDESSCVFVSESAICSQNSLYPMSPLLQQCIRGDTSSCARTLVSGTMGNKFILSKGNIVANCASILCKCYSTSTIINQSPDKLLTFIASDTCPLVEIDGATIQVGGRQYPDMVYEGKVALGPAISLDRLDVGINLGNALKKLDDAKVLIDSSNQILETVRRSSFNFGSLLSVPILSCTALALLLLIYCCKRRYQQTLK

SEQ ID NO: 187 - protein, 526 aa, Morbillivirus canis
MNAINSGSHCTWLVLWCLGMASLFLCSKAQIHWDNLSTIGIIGTDNVHYKIMTRPSHQYLVIKLIPNASLIENCTKAELGEYEKLLNSVLEPINQALTLMTKNVKPLQSLGSGRRQRRFAGVVLAGVALGVATAAQITAGIALHQSNLNAQAIQSLRTSLEQSNKAIEEIREATQETVIAVQGVQDYVNNELVPAMQHMSCELVGQRLGLRLLRYYTELLSIFGPSLRDPISAEISIQALIYALGGEIHKILEKLGYSGSDMIAILESRGIKTKITHVDLPGKFIILSISYPTLSEVKGVIVHRLEAVSYNIGSQEWYTTVPRYIATNGYLISNFDESSCVFVSESAICSQNSLYPMSPLLQQCIRGDTSSCARTLVSGTMGNKFILSKGNIVANCASILCKCYSTSTIINQSPDKLLTFIASDTCPLVEIDGATIQVGGRQYPDMVYEGKVALGPAISLDRLDVGTNLGNALKKLDDAKVLIDSSNQILETVRRSSFNFGSLLSVPILSCTALALLLLIYCCKRR

SEQ ID NO: 188 - protein, 526 aa, Morbillivirus canis
MNAINSGSHCTWLVLWCLGMASLFLCSKAQIHWDNLSTIGIIGTDNVHYKIMTRPSHQYLVIKLIPNASLIENCTKAELGEYEKLLNSVLEPINQALTLMTKNVKPLQSLGSGRRQRRFAGVVLAGVALGVATAAQITAGIALHQSNLNAQAIQSLRTSLEQSNKAIEEIREATQETVIAVQGVQDYVNNELVPAMQHMSCELVGQRLGLRLLRYYTELLSIFGPSLRDPISAEISIQALIYALGGEIHKILEKLGYSGSDMIAILESRGIKTKITHVDLPGKFIILSISYPTLSEVKGVIVHRLEAVSYNIGSQEWYTTVPRYIATNGYLISNFDESSCVFVSESAICSQNSLYPMSPLLQQCIRGDTSSCARTLVSGTMGNKFILSKGNIVANCASILCKCYSTSTIINQSPDKLLTFIASDTCPLVEIDGATIQVGGRQYPDMVYEGKVALGPAISLDRLDVGINLGNALKKLDDAKVLIDSSNQILETVRRSSFNFGSLLSVPILSCTALALLLLIYCCKRR

SEQ ID NO: 189 - protein, 602 aa, unidentified
MPAENKKVRFENTTSDKGKIPSKVIKSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCT

SEQ ID NO: 190-198 - skipped (9 entries)

SEQ ID NO: 199 - protein, 604 aa, synthetic construct
MPAENKKVRFENTTSDKGKIPSKVIKSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCTGS

SEQ ID NO: 200 - protein, 570 aa, unidentified
MIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCT

SEQ ID NO: 201 - protein, 572 aa, synthetic construct
MIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCTGS

SEQ ID NO: 202 - protein, 569 aa, unidentified
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCT

SEQ ID NO: 203 - protein, 571 aa, synthetic construct
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCTGS

SEQ ID NO: 204 - protein, 546 aa, unidentified
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNTYSRLEDRRVRPTSSGDLYYIGT

SEQ ID NO: 205 - protein, 524 aa, unidentified
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 206 - protein, 521 aa, unidentified
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKK

SEQ ID NO: 207 - protein, 602 aa, unidentified
MPAENKKVRFENTTSDKGKIPSKVIKSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCT

SEQ ID NO: 208 - protein, 602 aa, unidentified
MPAENKKVRFENTTSDKGKIPSKVIKSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICAEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCT

SEQ ID NO: 209 - protein, 602 aa, unidentified
MPAENKKVRFENTTSDKGKIPSKVIKSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNATAENPVFTVFKDNEILYRAQLASEDTNAQKTITNCFLLKNKIWCISLVEIYDTGDNVIRPKLFAVKIPEQCT

SEQ ID NO: 210 - protein, 602 aa, unidentified
MPAENKKVRFENTTSDKGKIPSKVIKSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAANPVFTVFKDNEILYRAQLASEDTNAQKTITNCFLLKNKIWCISLVEIYDTGDNVIRPKLFAVKIPEQCT

SEQ ID NO: 211 - protein, 512 aa, Vesiculovirus cocal
MNFLLLTFIVLPLCSHAKFSIVFPQSQKGNWKNVPSSYHYCPSSSDQNWHNDLLGITMKVKMPKTHKAIQADGWMCHAAKWITTCDFRWYGPKYITHSIHSIQPTSEQCKESIKQTKQGTWMSPGFPPQNCGYATVTDSVAVVVQATPHHVLVDEYTGEWIDSQFPNGKCETEECETVHNSTVWYSDYKVTGLCDATLVDTEITFFSEDGKKESIGKPNTGYRSNYFAYEKGDKVCKMNYCKHAGVRLPSGVWFEFVDQDVYAAAKLPECPVGATISAPTQTSVDVSLILDVERILDYSLCQETWSKIRSKQPVSPVDLSYLAPKNPGTGPAFTIINGTLKYFETRYIRIDIDNPIISKMVGKISGSQTERELWTEWFPYEGVEIGPNGILKTPTGYKFPLFMIGHGMLDSDLHKTSQAEVFEHPHLAEAPKQLPEEETLFFGDTGISKNPVELIEGWFSSWKSTVVTFFFAIGVFILLYVVARIVIAVRYRYQGSNNKRIYNDIEMSRFRK

SEQ ID NO: 212 - protein, 512 aa, Vesiculovirus cocal
MNFLLLTFIVLPLCSHAKFSIVFPQSQKGNWKNVPSSYHYCPSSSDQNWHNDLLGITMKVKMPQTHKAIQADGWMCHAAKWITTCDFRWYGPKYITHSIHSIQPTSEQCKESIKQTKQGTWMSPGFPPQNCGYATVTDSVAVVVQATPHHVLVDEYTGEWIDSQFPNGKCETEECETVHNSTVWYSDYKVTGLCDATLVDTEITFFSEDGKKESIGKPNTGYRSNYFAYEKGDKVCKMNYCKHAGVRLPSGVWFEFVDQDVYAAAKLPECPVGATISAPTQTSVDVSLILDVERILDYSLCQETWSKIRSKQPVSPVDLSYLAPKNPGTGPAFTIINGTLKYFETRYIRIDIDNPIISKMVGKISGSQTERELWTEWFPYEGVEIGPNGILKTPTGYKFPLFMIGHGMLDSDLHKTSQAEVFEHPHLAEAPKQLPEEETLFFGDTGISKNPVELIEGWFSSWKSTVVTFFFAIGVFILLYVVARIVIAVRYRYQGSNNKRIYNDIEMSRFRK

SEQ ID NO: 213 - protein, 512 aa, Vesiculovirus cocal
MNFLLLTFIVLPLCSHAKFSIVFPQSQKGNWKNVPSSYHYCPSSSDQNWHNDLLGITMKVKMPKTHKAIQADGWMCHAAKWITTCDFRWYGPKYITHSIHSIQPTSEQCKESIKQTKQGTWMSPGFPPQNCGYATVTDSVAVVVQATPHHVLVDEYTGEWIDSQFPNGKCETEECETVHNSTVWYSDYKVTGLCDATLVDTEITFFSEDGKKESIGKPNTGYRSNYFAYEKGDKVCKMNYCKHAGVRLPSGVWFEFVDQDVYAAAKLPECPVGATISAPTQTSVDVSLILDVERILDYSLCQETWSKIRSKQPVSPVDLSYLAPKNPGTGPAFTIINGTLKYFETRYIRIDIDNPIISKMVGKISGSQTEAELWTEWFPYEGVEIGPNGILKTPTGYKFPLFMIGHGMLDSDLHKTSQAEVFEHPHLAEAPKQLPEEETLFFGDTGISKNPVELIEGWFSSWKSTVVTFFFAIGVFILLYVVARIVIAVRYRYQGSNNKRIYNDIEMSRFRK

SEQ ID NO: 214 - protein, 512 aa, Vesiculovirus cocal
MNFLLLTFIVLPLCSHAKFSIVFPQSQKGNWKNVPSSYHYCPSSSDQNWHNDLLGITMKVKMPQTHKAIQADGWMCHAAKWITTCDFRWYGPKYITHSIHSIQPTSEQCKESIKQTKQGTWMSPGFPPQNCGYATVTDSVAVVVQATPHHVLVDEYTGEWIDSQFPNGKCETEECETVHNSTVWYSDYKVTGLCDATLVDTEITFFSEDGKKESIGKPNTGYRSNYFAYEKGDKVCKMNYCKHAGVRLPSGVWFEFVDQDVYAAAKLPECPVGATISAPTQTSVDVSLILDVERILDYSLCQETWSKIRSKQPVSPVDLSYLAPKNPGTGPAFTIINGTLKYFETRYIRIDIDNPIISKMVGKISGSQTEAELWTEWFPYEGVEIGPNGILKTPTGYKFPLFMIGHGMLDSDLHKTSQAEVFEHPHLAEAPKQLPEEETLFFGDTGISKNPVELIEGWFSSWKSTVVTFFFAIGVFILLYVVARIVIAVRYRYQGSNNKRIYNDIEMSRFRK

SEQ ID NO: 215 - protein, 91 aa, synthetic construct
MALPVTALLLPLALLLHAARPEQKLISEEDLGSSGSGSAVSNAVGQDTQEVIVVPHSLPFKVVVISAILALVVLTIISLIILIMLWQKKPR

SEQ ID NO: 216 - protein, 243 aa, synthetic construct
MLTRTLAVRSFAATMSPVKGGTKCIKYLLFGFNFIFWLAGIAVLAIGLWLRFDSQTKSIFEQETNNNNSSFYTGVYILIGAGALMMLVGFLGCCGAVQESQCMLGLFFGFLLVIFAIEIAAAIWGYSHKDEVIKEVQEFYKDTYNKLKTKDEPQRETLKAIHYALNCCGLAGGVEQFISDICPKKDVLETFTVKSCPDAIKEVFDNKFHIIGAVGIGIAVVMIFGMIFSMILCCAIRRNREMV

SEQ ID NO: 217 - protein, 79 aa, synthetic construct
MLTRTLAVRSFAATMALPVTALLLPLALLLHAARPEQKLISEEDLTGKLFWALVVVAGVLFCYGLLVTVALCVIWVRSG

SEQ ID NO: 218 - protein, 98 aa, synthetic construct
MLTRTLAVRSFAATMALPVTALLLPLALLLHAARPEQKLISEEDLIYIWAPLAGTCGVLLLSLVITLYCNHRNRRRVCKCPRPVVKSGDKPSLSARYV

SEQ ID NO: 219 - protein, 107 aa, synthetic construct
MLTRTLAVRSFAATMALPVTALLLPLALLLHAARPEQKLISEEDLMALIVLGGVAGLLLFIGLGIFFCVRCRHRRRQAERMSQIKRLLSEKKTCQCPHRFQKTCSPI

SEQ ID NO: 220 - protein, 252 aa, synthetic construct
MLTRTLAVRSFAATMAVEGGMKCVKFLLYVLLLAFCACAVGLIAVGVGAQLVLSQTIIQGATPGSLLPVVIIAVGVFLFLVAFVGCCGACKENYCLMITFAIFLSLIMLVEVAAAIAGYVFRDKVMSEFNNNFRQQMENYPKNNHTASILDRMQADFKCCGAANYTDWEKIPSMSKNRVPDSCCINVTVGCGINFNEKAIHKEGCVEKIGGWLRKNVLVVAAAALGIAFVEVLGIVFACCLVKSIRSGYEVM

SEQ ID NO: 221 - protein, 250 aa, synthetic construct
MLTRTLAVRSFAATMGVEGCTKCIKYLLFVFNFVFWLAGGVILGVALWLRHDPQTTNLLYLELGDKPAPNTFYVGIYILIAVGAVMMFVGFLGCYGAIQESQCLLGTFFTCLVILFACEVAAGIWGFVNKDQIAKDVKQFYDQALQQAVVDDDANNAKAVVKTFHETLDCCGSSTLTALTTSVLKNNLCPSGSNIISNLFKEDCHQKIDDLFSGKLYLIGIAAIVVAVIMIFEMILSMVLCCGIRNSSVY

SEQ ID NO: 222 - protein, 82 aa, synthetic construct
MLTRTLAVRSFAATMALPVTALLLPLALLLHAARPEQKLISEEDLPPDHIPWITAVLPTVIICVMVFCLILWKWKKKKRPRS

SEQ ID NO: 223 - protein, 378 aa, synthetic construct
MLTRTLAVRSFAATMALPVTALLLPLALLLHAARPEQKLISEEDLILDYSFTGGAGRDIPPPQIEEACELPECQVDAGNKVCNLQCNNHACGWDGGDCSLNFNDPWKNCTQSLQCWKYFSDGHCDSQCNSAGCLFDGFDCQLTEGQCNPLYDQYCKDHFSDGHCDQGCNSAECEWDGLDCAEHVPERLAAGTLVLVVLLPPDQLRNNSFHFLRELSHVLHTNVVFKRDAQGQQMIFPYYGHEEELRKHPIKRSTVGWATSSLLPGTSGGRQRRELDPMDIRGSIVYLEIDNRQCVQSSSQCFQSATDVAAFLGALASLGSLNIPYKIEAVKSEPVEPPLPSQLHLMYVAAAAFVLLFFVGCGVLLSRKRRRQLCIQKL

SEQ ID NO: 224 - protein, 19 aa, synthetic construct
CKNEKKNKIERNNKLKQPP

SEQ ID NO: 225 - protein, 19 aa, synthetic construct
DNEKLRKPKHKKLKQPADG

SEQ ID NO: 226 - protein, 9 aa, synthetic construct
RLTRKRGLK

SEQ ID NO: 227 - protein, 8 aa, synthetic construct
CGRGDSPC

SEQ ID NO: 228 - protein, 5 aa, synthetic construct
RGDYK

SEQ ID NO: 229 - protein, 5 aa, synthetic construct
RGDFK

SEQ ID NO: 230 - protein, 6 aa, synthetic construct
PHSCNK

SEQ ID NO: 231 - protein, 8 aa, synthetic construct
CSRNLIDC

SEQ ID NO: 232 - protein, 12 aa, synthetic construct
VHWDFRQWWQPS

SEQ ID NO: 233 - protein, 17 aa, synthetic construct
CHPREVDVELYSTVFGH

SEQ ID NO: 234 - protein, 40 aa, synthetic construct
CEPEAEADAEAGPAGIGAVLKVLTTGLPALISWIKRKRQQ

SEQ ID NO: 235 - protein, 7 aa, synthetic construct
RPARPAR

SEQ ID NO: 236 - protein, 9 aa, synthetic construct
CRGDKGPDC

SEQ ID NO: 237 - protein, 9 aa, synthetic construct
AKRGARSTA

SEQ ID NO: 238 - protein, 9 aa, synthetic construct
CKRGARSTC

SEQ ID NO: 239 - protein, 9 aa, synthetic construct
CGNKRTRGC

SEQ ID NO: 240 - protein, 31 aa, synthetic construct
HAEGTFTSDVSSYLEGQAAKEFIAWLVRGRG

SEQ ID NO: 241 - protein, 21 aa, synthetic construct
KNSRSLGENDDGNNEDNEKLR

SEQ ID NO: 242 - protein, 12 aa, synthetic construct
AQQAANVAATLK

SEQ ID NO: 243 - protein, 33 aa, synthetic construct
AQQAANVAATLKKNSRSLGENDDGNNEDNEKLR

SEQ ID NO: 244 - protein, 9 aa, synthetic construct
CDGRPDRAC

SEQ ID NO: 245 - protein, 14 aa, synthetic construct
GNSTMCDGRPDRAC